(19) United States
(12) Patent Application Publication
(10) Pub. No.: US 2023/0263874 A1
(43) Pub. Date: Aug. 24, 2023

(54) TUMOR ANTIGENS FOR LIVER CANCER IMMUNOTHERAPY

(71) Applicant: ISTITUTO NAZIONALE TUMORI IRCCS - FONDAZIONE G. PASCALE, Napoli (NA) (IT)

(72) Inventors: Maria TAGLIAMONTE, Napoli (NA) (IT); Luigi BUONAGURO, Napoli (NA) (IT); Beatrice CAVALLUZZO, Napoli (NA) (IT); Carmen MANOLIO, Napoli (NA) (IT); Angela MAURIELLO, Napoli (NA) (IT); Concetta RAGONE, Napoli (NA) (IT); Annacarmen PETRIZZO, Napoli (NA) (IT)

(73) Assignee: ISTITUTO NAZIONALE TUMORI IRCCS - FONDAZIONE G. PASCALE, Napoli (NA) (IT)

(21) Appl. No.: 17/916,067

(22) PCT Filed: Apr. 1, 2021
(86) PCT No.: PCT/EP2021/058675

§ 371 Date: Sep. 30, 2022

(30) Foreign Application Priority Data
Apr. 2, 2020 (IT) 102020000006973

(51) Int. Cl. A61K 39/00; A61P 35/00

(52) U.S. Cl. CPC A61K 39/001111; A61P 35/00

(57) ABSTRACT

The present invention relates to tumor antigens and their use in cancer immunotherapy. In particular, the present invention relates to HERV epitopes and tumor associated antigens (TAAs) expressed by liver cancer cells and which, preferably in combination with epigenetic drugs, can be used in tumor vaccination therapies in the treatment and prevention of liver cancer, preferably hepatocellularcarcinoma (HCC).

### TECHNICAL FIELD

The present invention relates to tumor antigens and their use in cancer immunotherapy. In particular, the present invention relates to HERV epitopes and tumor associated antigens (TAAs) expressed by liver cancer cells and which, preferably in combination with epigenetic drugs, can be used in tumor vaccination therapies in the treatment and prevention of liver cancer, preferably hepatocellularcarcinoma (HCC) .

### BACKGROUND

Cancer immunotherapy can be classified into passive and active strategies, the latter being specific or non-specific [1]. Passive or “adoptive” immunotherapy is based on the administration of anticancer antibodies or the transfer of tumor reactive lymphocytes. Active immunotherapy is aimed both at stimulating a de novo specific immune response in the host against selected tumor antigens, using therapeutic anti-cancer vaccines, and at amplifying the existing anti-tumor immune response, by administering non-specific pro-inflammatory molecules or adjuvants. In this context, considering the disappointing results obtained so far with these strategies, the search for specific and selective tumor antigens for the development of tumor vaccines is the main objective for several research groups and leading companies in the health sector.

In this perspective, the search for human tumor antigens as potential targets for cancer immunotherapy has led to the discovery of several molecules mainly or selectively expressed on cancer cells. In fact, only in this way can the tumor antigens used in vaccines be able to break immune tolerance, without inducing pathological autoimmune side reactions [2,3].

Tumor antigens are distinguished into Tumor associated antigens (TAAs) and Tumor specific antigens (TSAs). Antigens derived from the aberrantly overexpressed self-antigens in tumor cells compared to normal cells are defined as tumor-associated antigens (TAAs) and might represent universal antigens among patients with the same malignancy. Besides the overexpressed antigens, TAAs can include: cell lineage differentiation antigens, which are normally not expressed in adult tissue; and cancer/germline antigens (also known as cancer/testis), which are normally expressed only in immune privileged germline cells [Buonaguro & Tagliamonte, Selecting Target Antigens for Cancer Vaccine Development. Vaccines (Basel). 2020 Oct 17;8(4):615. doi: 10.3390/vaccines8040615, PMID: 33080888]. Overexpressed and tissue differentiation antigens are able to induce an antitumor immune response when high levels of expression of these proteins reach the threshold for T cell recognition, breaking immunological tolerance. One of the possible limitations of TAAs is that they can also be present on normal cells, albeit with low levels of expression and, consequently, they can be subject to both central and peripheral immune tolerance mechanisms [4]. Therefore, peptide analogs should be designed by substituting one or two amino acid residues in the epitope sequence to improve its antigenicity and immunogenicity [5].

On the other hand, TSAs are antigens present only on cancer cells because they derive from specific biological processes of the neoplastic process. A particular group of TSAs are neoantigens resulting from cancer-related non-synonymous mutations or other genetic alterations that result in mutated peptides presented by the human leukocyte antigen (HLA) on the cell surface to the immune system. Neoantigens are theoretically the most interesting therapeutic targets because they are different from the germ line and seen as “non self” by the immune system. Consequently, immune reactions directed towards neoantigens are not subject to central and peripheral tolerance [6]. However, such mutated neoantigens are strictly individual (private) and their identification requires a combination of genomic, proteomic and immunomic screening procedures that currently cannot be applied on a large scale [7-9]. Furthermore, the effectiveness of such a highly personalized approach is probably reduced by the high mutational rate of tumors which determines the constant generation of new target mutated neoantigens and a consequent immune evasion of the cancer.

A different category of TSAs includes antigens derived from the transcription of endogenous retrovirus (HERV) genes. In fact, HERVs, although they represent about 8% of the entire cellular genome, are normally transcriptionally inactive. Hence, their antigens are considered non-self by our immune system and are not subject to the phenomenon of immune tolerance [10]. Consequently, TSAs derived from HERVs, compared to those derived from mutations, would have the essential advantage of not being personalized because they are shared among all patients affected by the same oncological pathology or by different oncological pathologies in which the same HERV are expressed [11].

Retroviruses are RNA viruses that replicate through a DNA intermediate and permanently integrate into a host’s genome. After initial integration, the provirus is also capable of retrotransposition, which has the effect of amplifying the copy number (100-1000) of the provirus in the host genome. Such proviruses can become incompetent for replication due to the accumulation of deletions and nonsense mutations within the viral genome. In this way, the provirus fixes itself in the human genome and the “endogenized” retroviruses are no longer able to cause horizontal infection of another host and are reclassified as HERV. In addition to acquiring large deletions and mutations within the proviral genome, HERVs undergo transcriptional silencing through DNA methylation and the remodeling of chromatin [12].

There are approximately 40 independent and phylogenetically distinct groups of HERVs categorized in the human genome [13]. Studies are still underway to understand whether HERVs play an active role in the onset or progression of cancer or if they are the result of a secondary effect of epigenetic change within the tumor cell [14]. In general, it is likely that the differential regulation of HERV in cancer cells is a result of the global genomic instability of tumors that leads to DNA hypomethylation and chromatin remodeling. Such epigenetic changes can expose and make available the Long Terminal Repeats (LTRs) of HERVs, which are normally transcriptionally silent, resulting in increased transcription and translation of retroviral genes [15].

Although HERVs are no longer competent for replication, they can have significant effects on transcription levels of cellular genes and oncogenic pathways by inserting LTRs near proto-oncogenes [16]. Indeed, it has been shown that HERV proteins interact with other cytosolic proteins including zinc fingers motifs on tumor suppressor genes [17] and are involved in tumor immune evasion [18]. Furthermore, HERV proteins are also immunogenic, as demonstrated by a significant activation of the adaptive immune response against HERVs proteins in tumor tissue and in the serum of cancer patients.

It is reported in the literature that the treatment of tumor cells with epigenetic drugs, such as DNA methyltransferase inhibitors or histone deacetylase inhibitors, deregulates the cellular transcriptional pattern, with the activation of different genes, including those of HERV, which are normally inactive. This effect is not evident in normal cells because they are not actively replicating.

Consequently, the transcriptional activation induced by treatment with epigenetic drugs induces in tumor cells the expression of a broad spectrum of “neo-antigens” (antigens not normally expressed by cells) deriving both from the sequences of the HERV genes (gag, pol, env) and by the cellular gene sequences regulated by HERV LTRs. The second category of genes is not predictable due to the random insertion of LTRs into the genome, and therefore would be patient specific neo-antigens. On the other hand, the neoantigens deriving from the sequences of the HERV genes can represent an optimal target shared among all patients affected by the same oncological pathology or among all patients affected by different oncological pathologies in which the same HERV are expressed [11].

Different classes of HERV have been associated with different oncological diseases for which they can play a role both as potential tumor markers and as immunological targets. Breast cancer and melanoma are the cancers for which there is the strongest experimental evidence, but documented evidence is also available on the role of HERV activity in colorectal cancer, pancreatic cancer, hepatocellular cancer, prostate cancer and ovarian cancer [19]. As for liver cancer, the only work in the literature describes the expression of HERV-K (HML-2) which appears to be correlated with the prognosis of the disease [20].

Among the cancers listed above, hepatocellular carcinoma (HCC) is the third leading cause of cancer death globally. The age-standardized incidence rate (ASR) of HCC in men in Europe, adjusted for the standard European population, is approximately 8 in 100,000 (http://globocan.iarc.fr/). Chronic infection with hepatitis B and C virus (HBV and HCV) is the main risk factor for the development of HCC [21].

HCC therapies depend on the stage of the disease with an extremely variable 5-year survival rate [22]. Surgery is the standard treatment for HCC in the early stages with a 5-year survival rate of 70% among treated patients [23,24]. Non-surgical loco-regional therapies are implemented for HCC patients who show more advanced disease, with survival rates ranging from 3 to 5 years [25] and tumor recurrence in more than 50% of patients at 5 years post treatment [25]. 26]. Sorafenib and Lenvatinib represent the only approved systemic therapy in advanced unresectable HCC, which provides a very limited survival benefit [27]. Recently, the RESORCE study (Regorafenib after Sorafenib in patients with hepatocellular carcinoma) demonstrated that Regorafenib can be used as a second-line treatment in patients with advanced liver cancer who have not responded to sorafenib treatment. Although the study showed only a modest 2.8-month improvement in overall survival, with a 38% reduction risk of death, this was the first study to show the benefits of second-line treatment in HCC [28]. In such a scenario, immunotherapy strategies for HCC may represent a key therapeutic tool for improving clinical outcome in patients with hepatocellular carcinoma. In particular, therapeutic cancer vaccines could hold great promise. This is still an open field of research, given that only very few studies on the HCC cancer vaccine have been conducted so far with still modest results [29]. WO2014/004385 provides tumor-associated antigens and their use as vaccines for treating and limiting cancers in a patient. In specific aspects, HERV type K envelope proteins and peptides are provided. Such proteins and peptides can be used to elicit specific immune responses, both humoral and cellular, that target tumor cells expressing the HERV type K envelope protein. HERV-K derived antigens are also disclosed in WO2020/049169 as tumor antigens for cancer vaccine.

In the light of the foregoing, the need for new anticancer therapies and compounds capable of overcoming the disadvantages of known therapies and compounds appears evident.

### DESCRIPTION OF THE INVENTION

According to the present invention, new tumor associated antigens (TAAs) as well as new tumor-specific antigens (TSAs) have now been identified. The antigens of the present invention show the advantage of not being strictly specific for a single patient, but of being shared among all patients affected by the same oncological pathology, in the specific case hepatocellular carcinoma (HCC).

The antigens, and in particular the epitopes, according to the present invention, are able to bind the molecules of MHC (major histocompatibility complex), in particular HLA. Specifically, according to the present invention, TAAs and HERV TSAs have been identified capable of binding MHC, more specifically HLA and, more specifically, different types of class I HLA.

As described in the experimental part, the specific TAAs and HERV TSAs associated with different class I HLAs according to the present invention were identified by means of an antigenic prediction analysis for the most frequent class I HLAs in the world population (carried out with the algorithm NetMHCPan http://www.cbs.dtu.dk/services/NetMHCpan/).

According to the present invention, the HERV TSAs and the TAAs identified can be advantageously used in anti-tumor vaccine therapies, also in combination with epigenetic drugs. In short, the administration of a vaccine comprising one or more peptides according to the present invention would have the effect of stimulating the immune system against such peptides. Subsequently, the administration of an epigenetic drug would induce a reprogramming of the transcriptional and translational profile of the tumor cell, inducing the expression of these epitopes by the tumor cell, which would then be readily recognized by the immune system with consequent effective elimination by the vaccine-induced antitumor immunity.

Advantageously, according to the present invention, peptides as such, or peptides in optimized forms, such as for example fused to antibodies capable of recognizing antigen presenting cells (APC), such as dendritic cells, can be administered as a vaccine. Peptides can be administered in alternative forms, such as for example nucleic acids coding for said peptides, or vectors comprising said nucleic acids or directly to cells, for example APC, which express the peptides.

Furthermore, according to the present invention, said epitopes or peptides can advantageously be used to stimulate T cells ex vivo and then re-infuse said T cells in the patient either after the administration of the epigenetic drug or in the absence of any specific pre-treatment.

Finally, according to the present invention it is possible to administer a T cell engineered to express a receptor (CAR-T cell) capable of recognizing tumor-specific antigens expressed either following the administration of an epigentic drug or in the absence of any specific any pre-treatment.

### DESCRIPTION OF THE INVENTION

The present invention relates to a composition comprising at least one peptide or at least one expression vector that induces the expression of said at least one peptide, said peptide consisting of an amino acid (aa) sequence from 9 to 30 aa residues and comprising or consisting of at least one of the epitopes sequences selected from: MLAGNAFTA (SEQ ID NO. 119), ALMAFTSAV (SEQ ID NO. 120), DYFRNQFKI (SEQ ID NO. 121), MLAGNEFQV (SEQ ID NO. 108), TMLNILALV (SEQ ID NO. 109), YYYQRALEI (SEQ ID NO. 110), KITEYLERV (SEQ ID NO. 111), VYSACSFTF (SEQ ID NO. 112), TLLKVTLEV (SEQ ID NO. 113), LYSGTAADF (SEQ ID NO. 114), MVMSFVNLV (SEQ ID NO. 115), FYFRNHFCI (SEQ ID NO. 116), LLNQQICPV (SEQ ID NO. 117), VYVAFSEFF (SEQ ID NO. 118), and a pharmaceutically acceptable vehicle or excipient.

Preferably said at least one of the epitopes sequences is selected from: MLAGNAFTA (SEQ ID NO. 119), ALMAFTSAV (SEQ ID NO. 120), DYFRNQFKI (SEQ ID NO. 121).

The invention also provides the a composition as defined above further comprising or expressing at least one peptide, said peptide consisting of an aa sequence of 9 to 30 aa and comprising or consisting of at least one of the epitopes sequences selected from: ALLALTSAV (SEQ ID NO. 122), ALSNHLNAV (SEQ ID NO. 123), RQYSVGFEV (SEQ ID NO. 124), IYTVSSFSI (SEQ ID NO. 125), IFNIIPSTF (SEQ ID NO. 126), YYDVIYLSW (SEQ ID NO. 127), RIVCLFWGV (SEQ ID NO. 128), LYACGTGAF (SEQ ID NO. 129), FMLDLYNAL (SEQ ID NO. 130), FLISIYQVL (SEQ ID NO. 131), FQFSASLHV (SEQ ID NO. 132), VYTYIQSRF (SEQ ID NO. 133), VHMKDFFYF (SEQ ID NO. 134), ALFGALFLA (SEQ ID NO. 135), LLNSLLDTV (SEQ ID NO. 136), TYFGSIVLL (SEQ ID NO. 137), LYHAGTVLL (SEQ ID NO. 138), LLLTLLLLL (SEQ ID NO. 139), LLGPLLVLL(SEQ ID NO. 140).

Preferably the above composition further comprises or expresses at least one peptide, said peptide consisting of an aa sequence of 9 to 30 aa and comprising or consisting of at least one of the epitopes sequences selected from: FINSGAWQI (SEQ ID NO.17), KLTTWILPK (SEQ ID NO.43), KTKIFQFLK (SEQ ID NO. 50), PYMLVVGNI (SEQ ID NO. 65), SMDDQLNQL (SEQ ID NO. 83), WQIGLANFV (SEQ ID NO. 96).

Still preferably the above composition further comprises or expresses at least one peptide of 9 to 30 aminoacids comprising or consisting of at least one of the epitopes sequences selected from SEQ ID NO. 4-16, SEQ ID NO. 18-42, SEQ ID NO. 44-49, SEQ ID NO. 51-64, SEQ ID NO. 66-82, SEQ ID NO. 84-95 and SEQ ID NO. 97-107.

In a preferred embodiment the invention relates to a composition as defined above comprising or consisting of:
a. at least one of the epitopes sequences selected from: MLAGNAFTA (SEQ ID NO. 119), ALMAFTSAV (SEQ ID NO. 120), DYFRNQFKI (SEQ ID NO. 121); and
b. at least one of the epitopes sequences selected from: MLAGNEFQV (SEQ ID NO. 108), TMLNILALV (SEQ ID NO. 109), YYYQRALEI (SEQ ID NO. 110), KITEYLERV (SEQ ID NO. 111), VYSACSFTF (SEQ ID NO. 112), TLLKVTLEV (SEQ ID NO. 113), LYSGTAADF (SEQ ID NO. 114), MVMSFVNLV (SEQ ID NO. 115), FYFRNHFCI (SEQ ID NO. 116), LLNQQICPV (SEQ ID NO. 117), VYVAFSEFF (SEQ ID NO. 118); and
c. at least one of the epitopes sequences selected from: FINSGAWQI (SEQ ID NO.17), KLTTWILPK (SEQ ID NO.43), KTKIFQFLK (SEQ ID NO. 50), PYMLVVGNI (SEQ ID NO. 65), SMDDQLNQL (SEQ ID NO. 83), WQIGLANFV (SEQ ID NO. 96).

Preferably the at least one peptide of the invention consists of an amino acid (aa) sequence from 9 to 30 aa residues, preferably 9 to 20, more preferably 9 aa residues.

The invention further relates to an engineered cell comprising a recombinant protein, or a polynucleotide encoding a recombinant protein, preferably said recombinant protein being a recombinant receptor, more preferably a receptor expressed on the surface of the immune cell receptor preferably, wherein the recombinant receptor specifically binds to at least one peptide of the composition as described above, preferably wherein the recombinant receptor is a recombinant T cell receptor (TCR) or a chimeric antigen receptor (CAR).

The composition according to the present invention is for use in the treatment of cancer, preferably for use in the treatment or prevention of liver cancer, even more preferably for use in the treatment or prevention of hepatocellular carcinoma. Preferably said composition also is administered in combination with an anticancer therapeutic agent. Preferably said anticancer therapeutic agent is an epigenetic drug and/or a check point inhibitor; more preferably an epigenetic drug.

The invention further provides a vaccine or immunogenic composition comprising the composition as defined above and a pharmaceutically acceptable vehicle or excipient and preferably an adjuvant. Preferably said vaccine or immunogenic composition is for use in the treatment or prevention of cancer, preferably for use in the treatment or prevention of liver cancer, even more preferably for use in the treatment or prevention of hepatocellular carcinoma.

It is a further object of the invention an isolated peptide selected from the group consisting of the peptides having aa sequences selected from SEQ ID NO. 4-140 or from the group of the peptides having an aa sequence of 9 to 30 aa and comprising at least one of SEQ ID NO 108-140, characterized in that said isolated peptide is able to bind MHC class I and/or MHC class II.

According to the invention, an “expression vector” that induces the expression of a peptide comprises a nucleic acid, DNA or RNA, coding for the peptide. The vector may be a RNA vector, a DNA vector, a viral vector or a bacterial vector.

According to the invention the term “peptide” includes peptide variants retaining the ability to bind to MHC class I and/or MHC class II molecule, for example peptides having one or two amino acids substituted by amino acids belonging to the same group.

As further example peptides of the invention have sequence homology of at least 80%, preferably at least 90%, with respect to the sequence of one or more peptides, comprising or consisting of an amino acid sequence chosen from the sequences from SEQ ID NO: 4 to SEQ ID NO: 107 or from SEQ ID NO: 108 to SEQ ID NO: 140 and retain the ability to binding MHC class I and / or MHC class II.

In particular, when the peptide comprising or consisting of an amino acid sequence chosen from the sequences from SEQ ID NO: 4 to SEQ ID NO: 107 or from SEQ ID NO: 108 to SEQ ID NO: 140 is linked to class I MHC, the peptide is able to be recognized by T cells, in particular by CD8 T cells. Or, when the above binds MHC class II is bound to MHC class II, the peptide is able to be recognized by TCD4 + cells (T helper cells).

A further example of “variant” relates to a peptide wherein a given amino acid is altered by modifying it with the side chain of another naturally occurring amino acid residue or some other side chain, such that the peptide is still able to bind to an MHC class I or class II molecule in substantially the same way as a peptide consisting of the given amino acid sequence in consisting of SEQ ID NO: 4 to SEQ ID NO: 140. For example, a peptide may be modified so that it at least maintains, if not improves, the ability to interact with and bind to the binding groove of a suitable MHC molecule.

Furthermore, the composition of the invention comprises or expresses a single peptide as defined above, or a combination of some or all of the peptides, variants, or a mixture of at least two of the peptides and variants mentioned above, or at least three of the peptides mentioned above. Preferably said mixture comprises or consists of at least three peptides, wherein one peptide is an HERV epitope (Table 3) comprising or consisting of anyone of SEQ ID NO.17, SEQ ID NO.43, SEQ ID NO. 50, SEQ ID NO. 65, SEQ ID NO. 83, SEQ ID NO. 96; one peptide is a tumor associated antigens (TAAs) derived from the 9 overexpressed proteins associated with poor prognosis in HCC (Table 7) and comprising or consisting of anyone of SEQ ID NO. 108-118; and one peptide is one of the peptides selected from a broad range of human viruses as sharing high homology with those derived from the HCC proteins (Table 8) and comprising or consisting of anyone of SEQ ID. NO. 119 - 121.

As previously indicated, the composition of the invention comprises peptides with a length from 9 amino acids to 30 amino acids, preferably from 9 amino acids to 20 amino acids, more preferably of 9 amino acids.

In detail, the peptides of 9 amino acids are optimal for binding the MHC of class I, while to bind the MHC of class II the peptides must be of at least 15 amino acids, preferably 20-30 amino acids. Advantageously, when the peptide according to the present invention or the peptides according to the present invention are able to bind MHC of class II, the latter have a length of at least 15 amino acids, preferably from 20 amino acids to 30 amino acids.

As used herein MHC class II indicates HLA class II, preferably HLA-DR, more preferably HLA-DR1, HLA-DR3, HLA-DR4 and / or HLA-DR11.

As used herein, MHC of class I indicates HLA of class I, preferably HLA-A and /or HLA-B, more preferably HLA-A.

In detail HLA-A is chosen from the group consisting of HLA-A * 02: 01, HLA-A * 01: 01, HLA-A * 26: 01, HLA-A * 03: 01 and / or HLA-A * 24:02, preferably HLA-A * 02: 01 and / or HLA-A * 24: 02.

Furthermore, HLA-B is chosen in the group consisting of HLA-B * 40: 01, HLA-B * 15: 01, HLA-B * 27: 05, HLA-B * 07: 02, HLA-B * 58: 01, HLA-B * 08: 01 and / or HLA-B * 39: 01, preferably HLA-B * 40: 01, HLA-B * 15: 01 and / or HLA-B * 08: 01.

As used herein one or more peptides can be also comprised in sequences including non-peptide bonds and / or in which one or more peptides are part of a fusion protein, for example, comprising N-terminal amino acids of the invariable chain associated with the HLA-DR antigen.

The peptide according to the present invention, or the peptide variant as defined above, may comprise additional portions of amino acids at the N-terminus or C-terminus, which are not necessarily part of the peptide portion that serves as an epitope for the MHC molecules.

However, these additional parts can be important to provide an efficient introduction of the peptide according to the present invention into the cells. In one embodiment of the present invention, the peptide is part of a fusion protein comprising, for example, the 80 N-terminal amino acids of the invariable chain associated with the HLA-DR antigen (p33) as derived from the NCBI database, GenBank_Accession number X00497.

In other embodiments of the peptides, the peptides of the present invention are bound to an antibody as described below, or a functional part of the antibody itself, in particular peptides can be inserted into a sequence of an antibody, so as to be specifically carried by the antibody or, for example, can be fused to an antibody, or inserted into an antibody, which is specific for dendritic cells.

Furthermore, the peptide or variant according to the present invention can be further modified or modified to improve the stability and / or the binding with the MHC molecules, in order to obtain a stronger immune response. Methods for optimizing a peptide sequence are well known in the art and include, for example, the introduction of reverse peptide bonds or non-peptide bonds.

In a reverse peptide bond the amino acid residues are not joined by peptide bonds (—CO—NH—), but the peptide bond is reversed. Such retro-inverse peptidomimetic peptides can be made using methods known in the art, such as those described in Meziere et al (1997) J. Immunol. 159, 3230-3237, incorporated herein by reference. This approach involves the creation of pseudopeptides containing changes that involve the skeleton and not the orientation of the side chains. Meziere et al (1997) show that these pseudopeptides are useful for MHC binding and helper T cell responses.

Retro-inverse peptides, which contain NH—CO bonds instead of CO—NH peptide bonds, are much more resistant to proteolysis.

A non-peptide bond can be, for example, one of the following: —CH2—NH, —CH2S—, —CH2CH2—, —CH═CH—, —COCH2—, —CH (OH) CH2— and —CH2SO—. U.S. Pat. 4,897,445 provides a method for the solid phase synthesis of non-peptide bonds (—CH2—NH) in polypeptide chains involving the use of polypeptides synthesized by standard procedures and the non-peptide bond synthesized by the reaction of an amino-aldehyde and an amino acid in the presence of NaCNBH3.

The peptides comprising the sequences described above can be synthesized with further chemical groups present at their amino and/or carboxyl end groups, to improve the stability, bioavailability and / or affinity of the peptides. For example, hydrophobic groups such as the carbobenzyloxy, dansyl or t-butyloxycarbonyl groups can be added to the amino terminals of the peptides. Similarly, an acetyl group or a 9-fluorenylmethoxycarbonyl group can be placed at the amino terminals of the peptides. Furthermore, the hydrophobic group, the t-butyloxycarbonyl or an amide group can be added to the carboxy-terminus of the peptides.

Furthermore, the peptides according to the present invention can be synthesized in such a way as to alter their steric configuration. For example, the D isomer of one or more of the amino acid residues of the peptide can be used, rather than the usual L isomer. Further, at least one of the amino acid residues of the peptides of the invention can be replaced by one of the well known amino acid residues, not present in nature. Alterations such as these can serve to increase the stability, bioavailability and / or binding action of the peptides of the invention.

Similarly, a peptide or a variant of the peptide according to the present invention can be chemically modified or modified by reacting specific amino acids before or after the synthesis of the peptide. Examples for such modifications are well known in the art and are summarized for example in R. Lundblad, Chemical Reagents for Protein Modification, 3rd ed. CRC Press, 2005, which is incorporated herein by reference.

Chemical modification of amino acids includes, but is not limited to: modification by acylation, amidation, pyridoxylation of lysine, reductive alkylation, trinitrobenzylation of amino groups with 2,4,6-trinitrobenzenesulfonic acid (TNBS), amide modification of carboxylic groups and sulfhydryl modification by oxidation with performic acid of cysteine to cysteic acid, formation of mercurial derivatives, formation of mixed disulfides with other thiol compounds, reaction with maleimide, carboxymethylation with iodoacetic acid or iodoacetamide and carbamylation with cyanate at alkaline pH. In this regard, the methodologies for the chemical modification of proteins are well known to one skilled in the art.

In short, the modification of arginyl residues in proteins is often based on the reaction of vicinal dicarbonyl compounds such as phenylglyoxal, 2,3-butanedione and 1,2-cyclohexanedione to form an adduct. Another example is the reaction of methylglyoxal with arginine residues. Cysteine can be modified without the concomitant modification of other nucleophilic sites such as lysine and histidine.

Consequently, a large number of cysteine modification reagents are available. The websites of companies such as Sigma-Aldrich (http://www.sigma-aldrich.com) provide information on specific reagents.

The selective reduction of disulfide bonds in proteins is also well known. Disulfide bonds can be formed and oxidized during the heat treatment of biopharmaceutical products. Woodward’s K reagent can be used to modify specific glutamic acid residues. N- (3- (dimethylamino) propyl) -N′-ethylcarbodiimide can be used to form intra-molecular connections between a lysine residue and a glutamic acid residue. For example, diethyl pyrocarbonate is a reagent for the modification of histidyl residues in proteins. Histidine can also be modified using 4-hydroxy-2-nonenal. The reaction of lysine residues and other α-amino groups is, for example, useful in the binding of peptides to surfaces or in the cross-linking of proteins / peptides. Lysine is the attachment site of poly (ethylene) glycol and the major modification site in protein glycosylation. The methionine residues in proteins can be modified for example with iodoacetamide, bromoethylamine and chloramine T.

Tetranitromethane and N-acetylimidazole can be used for the modification of tyrosyl residues. Cross-linking through the formation of di-tyrosine can be accomplished with hydrogen peroxide / copper ions.

N-bromosuccinimide, 2-hydroxy-5-nitrobenzyl bromide or 3-bromo-3-methyl-2- (2-nitrophenylmercapto) -3H-indole (BPNS-skatole) have been used in some tryptophan modification studies.

Successful modification of therapeutic proteins and peptides with PEG is often associated with a prolongation of the circulatory half-life, while cross-linking of proteins with glutaraldehyde, polyethylene glycol diacrylate and formaldehyde is used for the preparation of hydrogels. Chemical modification of allergens for immunotherapy is often achieved by carbamylation with potassium cyanate.

In general, peptides and variants (at least those containing peptide bonds between amino acid residues) can be synthesized by the Fmoc-polyamide mode of solid phase peptide synthesis, as disclosed by Lukas et al. (Synthesis of the solid phase peptide under continuous flow conditions Proc. Natl Acad Sci USA, May 1981; 78 (5): 2791-2795). Temporary protection of the N-amino group is provided by the 9-fluorenylmethyloxycarbonyl (Fmoc) group.

In a further embodiment, the present invention relates to an antibody conjugated with one or more peptides, and /or variants and/or salts and in which the antibody is capable of recognizing antigen presenting cells (APCs), preferably dendritic cells.

The antibody according to the present invention is able to recognize type C lectin receptors such as DC-SIGN (CD209) on dendritic cells. In this way the share of peptide captured by the dendritic cells is significantly increased and the peptide is presented more effectively to T cells.

In another embodiment, the present invention relates to a T lymphocyte receptor capable of binding one or more peptides as previously defined in which the receptor optionally comprises one or more co-stimulatory domains. The receptor is, for example, a chimeric receptor for the antigen (CAR).

In another embodiment, the present invention relates to a nucleotide sequence (DNA or RNA) which codes for a peptide as described above or for an antibody as described above.

In a further embodiment, the present invention relates to a nucleotide sequence that codes for T lymphocyte receptor capable of binding one or more peptides as previously defined as previously defined.

In a further embodiment, the present invention relates to an expression vector comprising a nucleotide sequence as previously defined.

It is specified that in the context of this discussion, the expression pharmaceutical composition also refers indiscriminately to an immunogenic composition or to a vaccine composition.

Preferably, such immunogenic composition or vaccine composition comprises one or more peptides selected from the group comprising or consisting of: MLAGNEFQV (SEQ ID NO. 108), TMLNILALV (SEQ ID NO. 109), YYYQRALEI (SEQ ID NO. 110), KITEYLERV (SEQ ID NO. 111), VYSACSFTF (SEQ ID NO. 112), TLLKVTLEV (SEQ ID NO. 113), LYSGTAADF (SEQ ID NO. 114), MVMSFVNLV (SEQ ID NO. 115), FYFRNHFCI (SEQ ID NO. 116), LLNQQICPV (SEQ ID NO. 117), VYVAFSEFF (SEQ ID NO. 118), MLAGNAFTA (SEQ ID NO. 119), ALMAFTSAV (SEQ ID NO. 120), DYFRNQFKI (SEQ ID NO. 121). Preferably the immunogenic composition or vaccine composition comprises one or more peptides selected from the group comprising or consisting of MLAGNAFTA (SEQ ID NO. 119), ALMAFTSAV (SEQ ID NO. 120), DYFRNQFKI (SEQ ID NO. 121). Still in a further preferred embodiment, the immunogenic composition or vaccine composition further comprises one or more peptides selected from the group comprising or consisting of FINSGAWQI (SEQ ID NO.17), KLTTWILPK (SEQ ID NO.43), KTKIFQFLK (SEQ ID NO. 50), PYMLVVGNI (SEQ ID NO. 65), SMDDQLNQL (SEQ ID NO. 83), WQIGLANFV (SEQ ID NO. 96).

This pharmaceutical composition can be advantageously used before administering an epigenetic drug. The pharmaceutical composition has the function of activating the immune response, while the epigenetic drug has the function of making the tumor cells express the HERV proteins, and therefore the epitopes. Such epitopes will then be recognized by the immune system previously activated by the pharmaceutical composition according to the present invention.

Further, in another embodiment, the present invention relates to a pharmaceutical composition comprising or consisting of one or more of the following active ingredients:
a receptor according to the present invention;
a nucleotide sequence in accordance with the present invention;
a vector in accordance with the present invention or a cell comprising a vector comprising the nucleotide sequence in accordance with the present invention, and optionally one or more immune checkpoint inhibitor drugs, together with one or more pharmaceutically acceptable excipients and / or adjuvants .

This composition can be advantageously administered after administering an epigenetic drug. As mentioned above, the epigenetic drug has the function of making the tumor cells express the HERV proteins, and therefore the epitopes. These epitopes will therefore be recognized by the active ingredients of the pharmaceutical composition according to the present invention just mentioned.

Similarly, ie after the administration of an epigenetic drug, a pharmaceutical composition according to the present invention can be administered.

In accordance with a further embodiment, the present invention relates to a pharmaceutical composition comprising or consisting of T cells in accordance with the present invention, and optionally one or more immune checkpoint inhibitor drugs, together with one or more excipients and / or pharmaceutically acceptable adjuvants.

Again, the present invention relates to one or more peptides according to the present invention, an antibody according to the present invention, a receptor according to the present invention, a nucleotide sequence according to the present invention, a vector according to the present invention, a cell according to the present invention, a T cell according to the present invention, a pharmaceutical composition according to the present invention, for use as a medicament.

In a further embodiment, the present invention relates to:
one or more peptides according to the present invention;
an antibody according to the present invention;
a receptor according to the present invention;
a nucleotide sequence in accordance with the present invention;
a vector according to the present invention;
a cell according to the present invention;
a T cell according to the present invention;
a pharmaceutical composition in accordance with the present invention;
for use in the treatment of cancers, such as hepatocellular cancer, breast cancer, melanoma, colon cancer, pancreatic cancer, kidney cancer, prostate cancer, lung cancer in which:
one or more peptides according to the present invention;
the antibody in accordance with the present invention;
the nucleotide sequence according to the present invention;
the vector according to the present invention which comprises the nucleotide sequence according to the present invention;
the cell comprising the peptide or vector comprising the nucleotide sequence according to the present invention or the pharmaceutical composition according to the present invention;
they are administered before an epigenetic drug capable of inducing the expression of HERV (Human endogenous retroviruses) sequences in tumor cells; while the receptor in accordance with the present invention, the T cell in accordance with the present invention, the nucleotide sequence in accordance with the present invention, the vector comprising the nucleotide sequence in accordance with the present invention, the cell comprising the receptor either carrier according to the present invention or the pharmaceutical composition according to the present invention; it is administered after an epigenetic drug capable of inducing the expression of HERV sequences in tumor cells, in which said epigenetic drug is preferably a DNA methyltransferase inhibitor.

Finally, the present invention relates to a combination comprising or consisting of one or more peptides according to the present invention, an antibody according to the present invention, a receptor according to the present invention, a nucleotide sequence according to the present invention, a vector according to the present invention, a cell according to the present invention, a T cell according to the present invention and / or a pharmaceutical composition according to the present invention, with an epigenetic drug capable of inducing the “ expression of HERV sequences in cancer cells, for sequential use in the treatment of tumors, such as hepatocellular carcinoma, breast cancer, melanoma, colon cancer, pancreatic cancer, kidney cancer, prostate cancer, lung cancer, in which the epigenetic drug is preferably a DNA methyltransferase inhibitor. Preferably, for sequential use in the treatment of hepatocellular carcinoma, breast cancer, melanoma, colon cancer, pancreatic cancer, kidney cancer, prostate cancer, lung cancer.

It is specified that in the context of this discussion the expression “sequential use” means the subsequent administration of the elements of the combination, that is, the peptides, the antibody, the receptor, the nucleotide sequence, the vector, the cell, the cell T, of the epigenetic drug, in distinct and possibly different pharmaceutical forms.

The present invention is described by way of illustration, but not of limitation, according to its preferred embodiments, but it is to be understood that variations and / or modifications may be made by those skilled in the art without thereby departing from the relative scope of protection, as defined from the attached claims.

### DETAILED AND EXEMPLIFICATIVE DESCRIPTION OF THE INVENTION

The present invention will be described, for illustrative but not limitative purposes, according to its preferred embodiments, with particular reference to the figures of the attached drawings, in which:

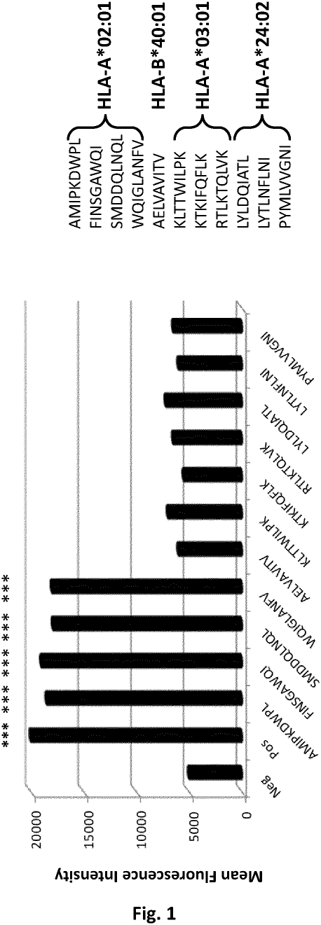

FIG. 1. Binding Assay to HLA-A * 02: 01. HERV peptides predicted to bind to the indicated HLAs were reacted with T2 cells, positive for HLA * 02: 01, in which the pathway for intracellular antigen presentation is not functional. The HLA is, therefore, able to bind only epitopes provided from outside. The results show that only the predicted HERV epitopes specific for HLA * 02: 01 bind to the HLA expressed by T2 cells, which is detectable by specific antibodies. Neg: negative control, indicates solvent without any added peptide; Pos: positive control, the known HLA*0201 peptide binder KLQAGTVFV (SEQ ID NO. 141) was used. Asterisks are by general convention an indication of statistical significance (the smaller the Pvalue, the greater the significance and the greater the number of asterisks).

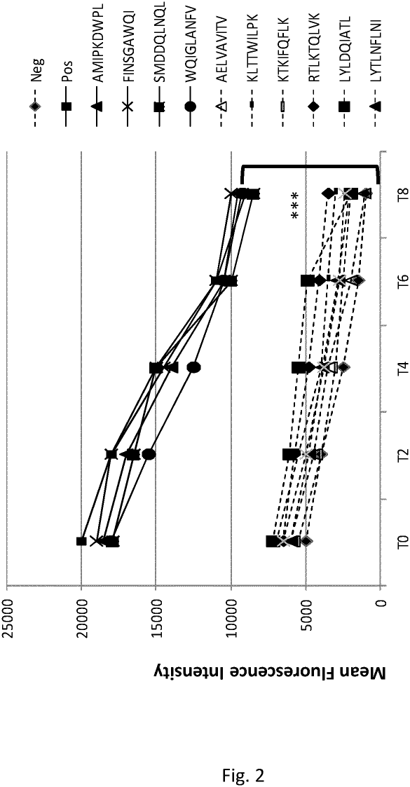

FIG. 2. Stability Assay at HLA-A * 02: 01. The stability of the binding of the predicted HERV peptides to the HLA * 02: 01 of T2 cells was verified by evaluating the expression levels of HLA on the cell surface over time. For all the HERV peptides predicted to bind HLA * 02: 01 there is a half-life of about 6 hours. Positive and negative controls are the same as in FIG. 1.

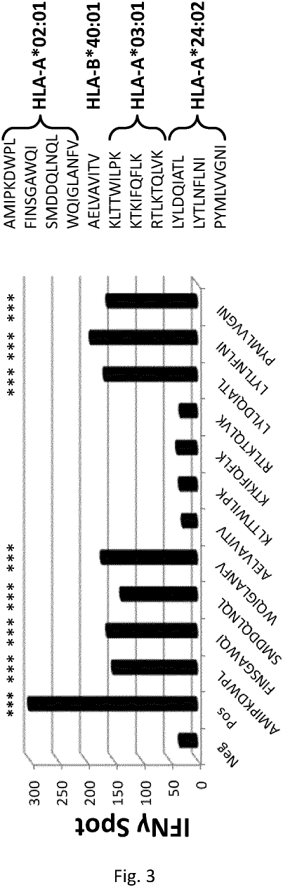

FIG. 3. IFNγ ELISpot Assay with cells from HCC HLA-A * 02: 01 patients. PBMCs from HCC patients positive for HLA-A * 02: 01 and for HLA-A * 24: 02 were purified and reacted with peptides predicted to be specific for the two alleles. The result of the IFNγ ELISpot assay shows that patients with HCC have a proportion of circulating T lymphocytes capable of recognizing only the HERVs peptides predicted for HLA * 02: 01 and for HLA-A * 24: 02.

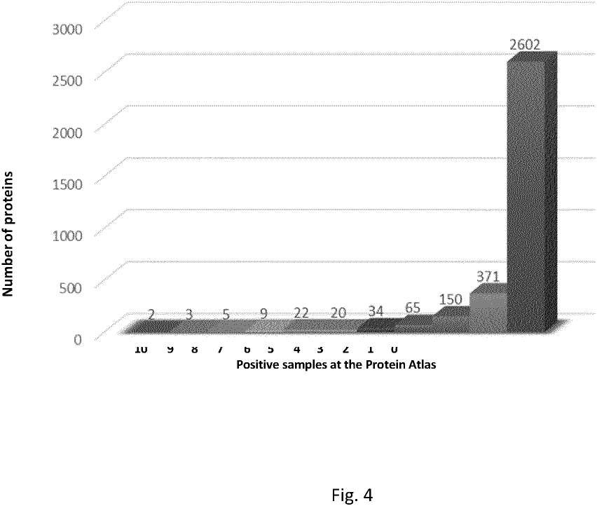

FIG. 4. Identification of HCC-related proteins. Number of HCC samples in the protein Atlas positive at Immunohistochemistry (IHC) for specific proteins.

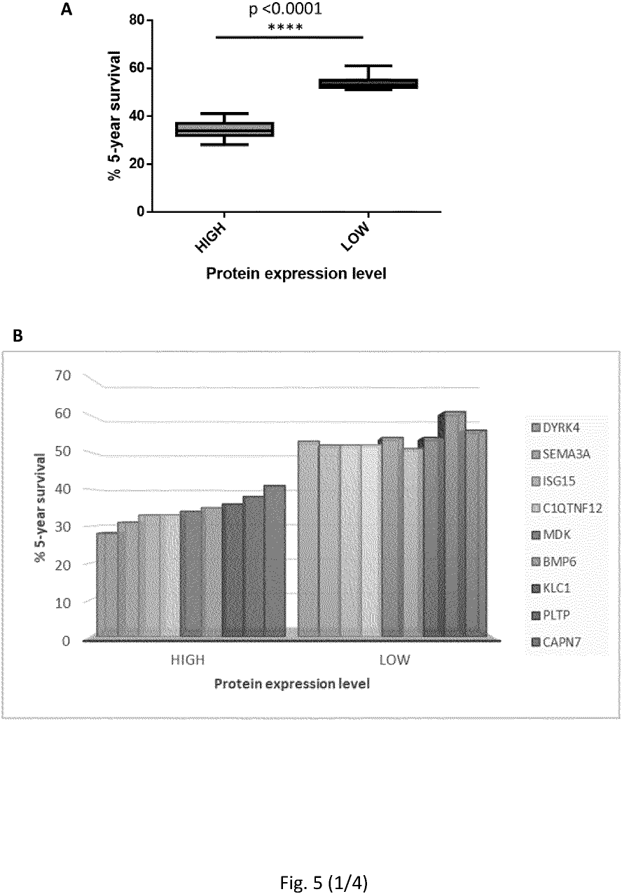

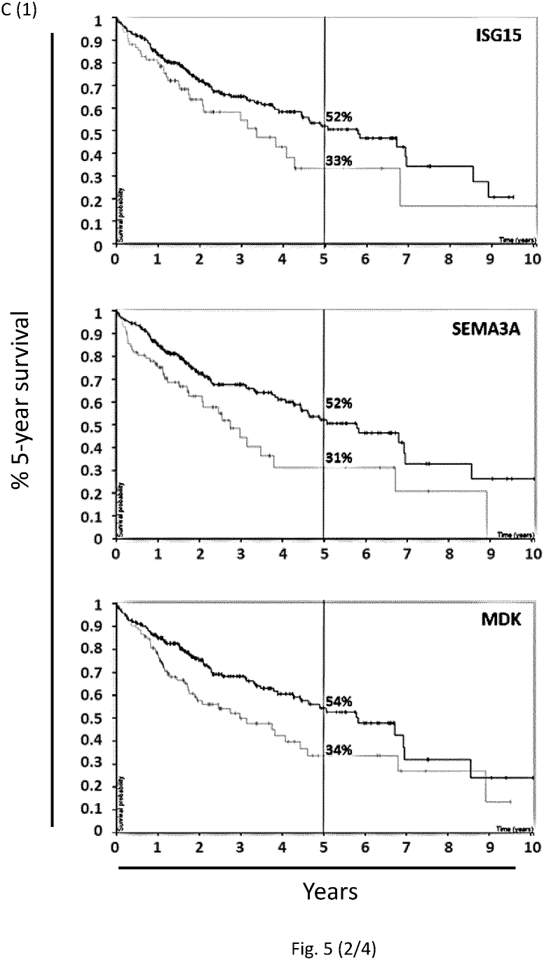

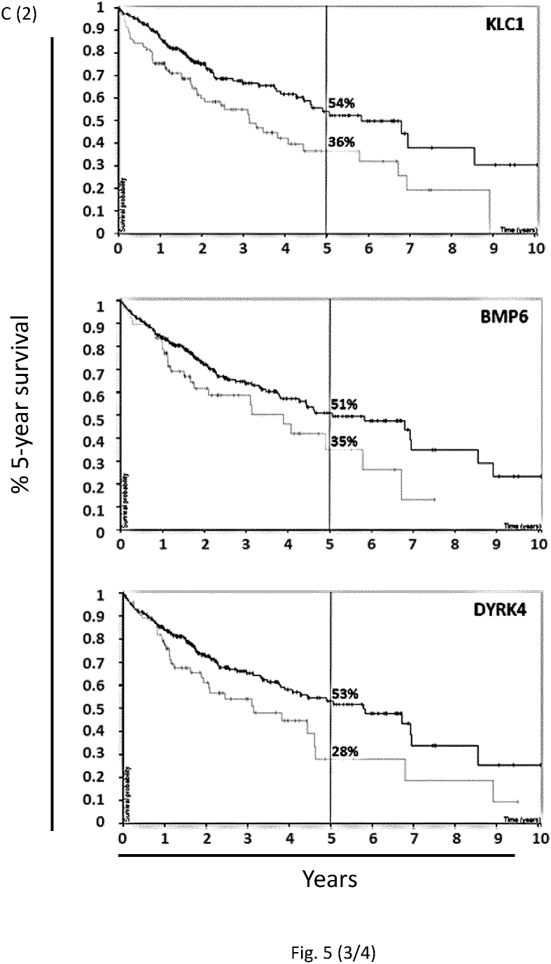

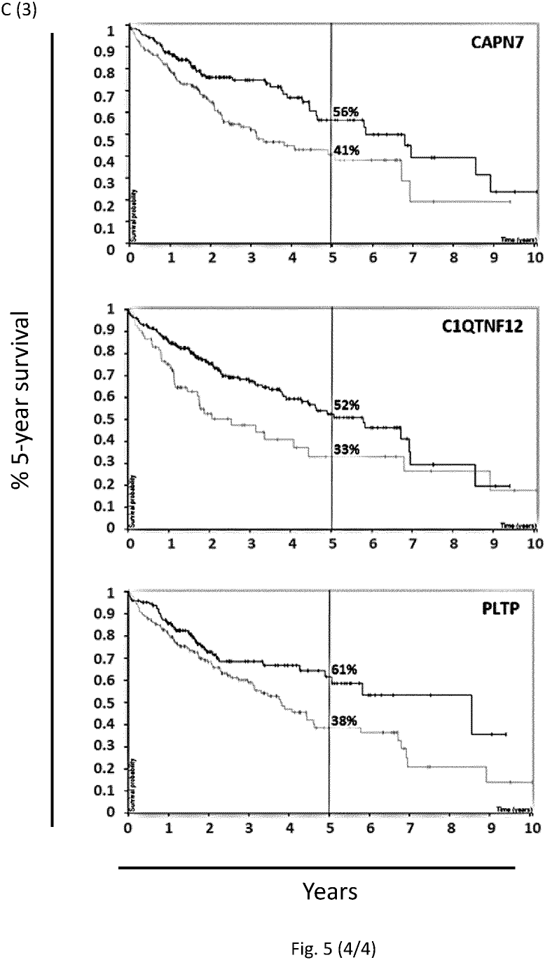

FIG. 5. Survival analysis. Correlation between gene expression and 5-year survival in HCC patients. (A) average of 5-year survival in patients with high or low protein expression of all nine proteins; (B) 5-year survival in patients with or low expression of each individual protein; (C) Kaplain-Meier survival curve in patients with or low expression of each individual protein.

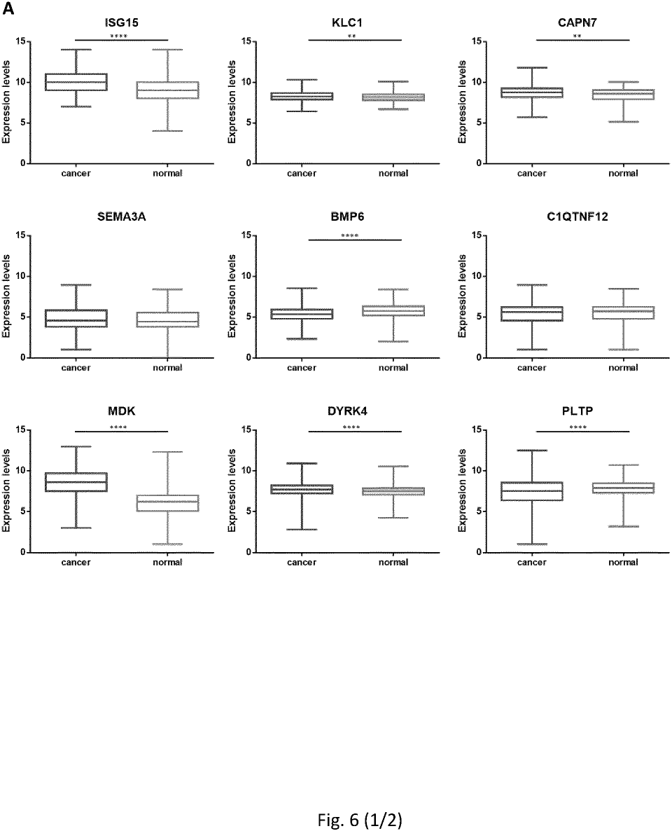

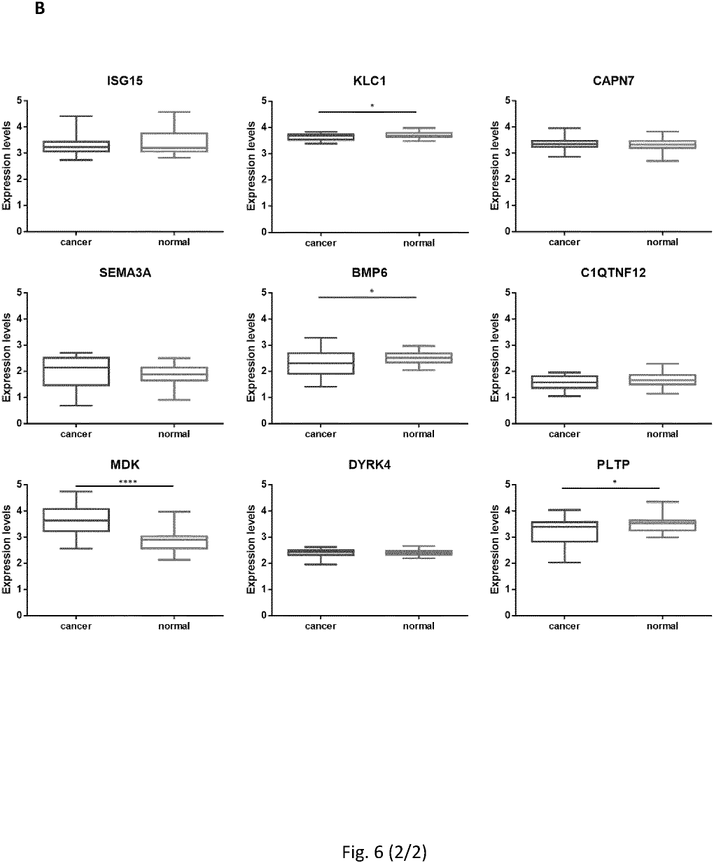

FIG. 6. Gene expression analysis. Expression levels of each gene in cancer (HCC) and paired non-tumoral liver tissues.

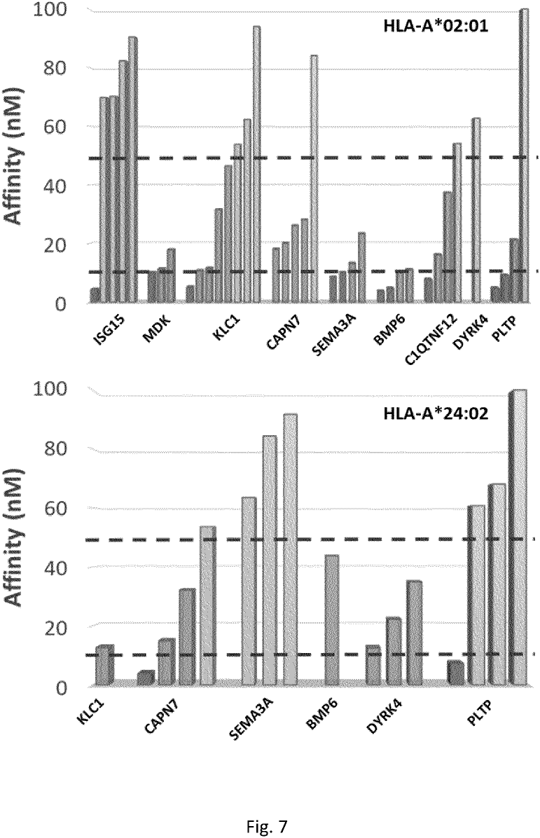

FIG. 7. Peptide prediction. Number of predicted epitopes per each protein with affinity values <10 nM, 10< affinity <50 nM, >50 nM.

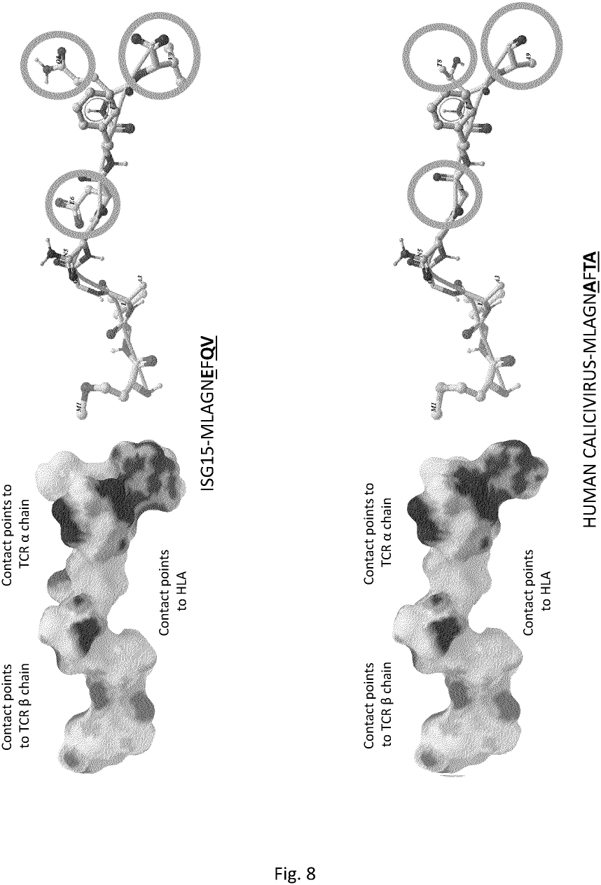

FIG. 8. Epitope modeling and molecular docking. The conformation of the paired viral and TAA peptides bound to the HLA-A*02:01 molecule is shown. Dark-grey areas = contact points with HLA molecule, TCR α chain and TCR β chain.

### MATERIALS AND METHODS

### Collection of Tissue Samples and Patients

14 chronic HCC patients infected with HCV undergoing liver resection were enrolled at the National Cancer Institute “Pascale” following reading and signing of the informed consent. A liver biopsy of the tumor and adjacent non-tumor liver tissue was obtained from each patient with histologically confirmed HCC at the time of surgery and stored at -80° C. until use in RNA stabilizing agent (RNAlater, Qiagen) for RNA sequencing. Fresh human peripheral blood mononuclear cells (PBMCs) were isolated by centrifugation of the Ficoll-Hypaque density gradient and cultured in appropriate medium consisting of RPMI 1640 (Capricorn Scientific GmbH) containing 2 mM L-Glutamine, supplemented with 10% serum heat inactivated human (Capricorn Scientific GmbH), 25 mM HEPES buffer solution (Capricorn Scientific GmbH), 50 IU / ml penicillin and 50 µg / ml streptomycin (Gibco Life Technologies), 20 µg / ml gentamicin (Capricorn Scientific GmbH). The PBMCs were kept in liquid nitrogen in 10% DMSO until the moment of use.

### RNA Extraction, Library Preparation and Sequencing

Liver samples were homogenized in the TRIzol reagent using the Tissue Lyser Disruption System (Qiagen), immediately frozen on dry ice, and the total RNA was purified according to the manufacturer’s protocol (Invitrogen). RNA samples were quantified by NanoDrop1000 spectrophotometer (Thermo Fisher Scientific) and showed a 260/280 absorbance ratio of approximately 1.8-2. RNA quality was assessed by digital electrophoresis on the Experion System using RNA StdSens Kit and RNA chip (Bio-Rad). The cDNA library sequencing preparation was performed on 4 µg of total RNA per sample using the TruSeq RNA stranded sample preparation kit (Illumina). The cDNA libraries were quantified by Qubit Fluorometer (Q32866; LifeTechnologies) and Qubit dsDNA High Sensay Kit. The overall quality of the libraries was assessed on the Experion System by DNA 1 K Analysis Kit and DNA chips (Bio-Rad). The paired libraries (100 × 2 bp) were sequenced at high coverage on Illumina HiSeq2000 NGS.

### RNA-Seq Data Analysis

Quality control was performed on the total number of raw readings using the FastQC tool (http://www.bioinformatics.babraham.ac.uk/projects/fastqc/). The high quality reads were mapped to the human reference transcriptome and human reference genome using TopHat2 v2.0.10 [30]. Only uniquely mapped reads were used to quantify gene expression in each sample and to calculate the differential expression between non-tumor and tumor samples [31]. The identification of the HERV sequences in the samples was carried out on the basis of the sequences deposited in the database https://herv.img.cas.cz/. The sequences that were selected for subsequent antigenic epitope prediction analyzes were those that showed a ≥2 ratio of tumor to adjacent non-tumor tissue.

### Selection of the Protein Sequences of the HERVs Genes

The sequences identified and selected in the database https://herv.img.cas.cz/ were translated using the tool ExPASy https://web.expasy.org/translate/ and aligned with Blast https://blast.ncbi.nlm.nih.gov/Blast.cgi to verify the specific identity of the HERV proteins.

### Analysis of the Prediction of HERVs Epitopes

Epitope prediction was performed on HERV protein sequences using prediction tools available at http://www.cbs.dtu.dk/services/. NetMHCpan version 4.0 http://www.cbs.dtu.dk/services/NetMHCpan/ was used to predict epitopes restricted to MHC class I alleles HLA-A01: 01, HLA-A02: 01, HLA-A03: 01, HLA-A24: 02, HLA-A26: 01, HLA-B07: 02, HLA-B08: 01, HLA-B27: 05, HLA-B39: 01, HLA-B40: 01, HLA-B58: 01, HLA-B15: 01. The entire HERV-K pol sequence was used to predict antigens with a length of 9 amino acids by scanning the overlapping peptides of the entire protein sequence. Antigens were selected for each class I HLA if with predicted affinity <100 nM.

### Homology Analysis of HERVs Epitopes

The Immune Epitope Database (IEDB; http://www.iedb.org/) was used for sequence homology analysis of predicted HERV epitopes with known human-derived antigens and experimentally validated pathogens. The homology analysis focused on the amino acid residues in the binding positions of the peptides to the Tcell receptor (positions 1, 4, 5 and 8).

### In Silico Analysis for HCC From the “Human Protein Atlas”

Starting from Downloadable data section (https://www.proteinatlas.org/about/download) of Human Protein Atlas database, Normal Tissue and Pathology dataset were downloaded. Normal tissue and Pathology dataset contain expression profiles of proteins of different human tissues samples, non tumoral and tumoral respectively, evaluated by immunohistochemistry on tissue micro array. Normal Tissue dataset is an extremely wide list containing information about the expression of several proteins into different cell types of a specific human non tumoral tissue. To identify tumor specific proteins, only those proteins who were defined as “NOT DETECTED” in hepatocytes and bile duct cells were selected. As consequential step, Pathology dataset was analyzed to verify if proteins that were defined as “NOT DETECTED” into Normal Tissue dataset could have been detected into tumoral tissue samples, in particular were selected all proteins that resulted to be absent in normal cell types but were detected into “LIVER CANCER” tissue samples at “HIGH”, “MEDIUM” or “LOW” expression level. All proteins defined as “NOT DETECTED” into liver tumor samples were excluded from the analysis; all other proteins were then divided into different groups, corresponding to number of samples with a high level of staining.

### Overall Survival Analysis in HCC

Survival analysis were obtained from The Human protein Atlas, section Pathology Atlas (https://www.proteinatlas.org/humanproteome/pathology) selecting liver cancer into “RNA EXPRESSION OVERVIEW” panel. Only proteins with p<0.05, whose high expression was correlated with poor survival rate, were selected for subsequent analysis.

### HLA Class I Epitope Prediction for Overexpressed Proteins in HCC and Comparison to Viral Antigens

In order to assess if overexpressed proteins (ISG15, KLC1, SEMA3A, BMP6, DYRK4 or PLTP) could lead to potential epitopes, FASTA format of protein sequences were downloaded from UniProt database (https://www.uniprot.org/) to perform an analysis of binding capability with NetMHCpan4.1a (http://www.cbs.dtu.dk/services/NetMHCpan/). This server predicts binding of peptides derived from an antigenic sequence to an MHC molecule; for this analysis, we focused our attention on HLA-A*02:01, the most common allele, and 9-mer peptides, selecting weak and strong binding peptides (WB; SB). To evaluate any potential homology of these predicted epitopes with molecules derived from pathogens, 9-mers were analyzed using Nucleotide BLAST (https://blast.ncbi.nlm.nih.govBlast.cgi?PROGRAM=blastn&PAGE_TYPE=BlastSearch&LIN K_LOC=blasthome) and the Immune Epitope Database (IEDB) (https://www.iedb.org/). Sequences with high similarity to the predicted epitopes were then aligned and processed with NetMHCpan4.1a to evaluate their binding affinity. (http://www.cbs.dtu.dk/services/NetMHCpan/).

### The 3D Structure of Epitopes for HLA Class I

The 3D structure of interaction between of HLA-HLA-A*02:01 with selected peptides was predicted using Pymol (https://pvmol.org/2/) and Molsoft ICM (http://www.molsoft.com/) software. The PDB format of complex between HLA-A*02:01 (1AO7), a viral peptide (TAX) and human T-cell receptor was downloaded from RCS Protein Data Bank (PDB) website (https://www.rcsb.org/). Viral peptide sequence was replaced with predicted epitopes using Pymol visualization system and Molsoft ICM software was used to visualize the molecular docking of HLA-A*02:01 with selected 9-mers.

### Peptide Synthesis

The peptides were synthesized with a purity> 95%. The lyophilized powder was dissolved in dimethyl sulfoxide (DMSO; Sigma-Aldrich), diluted in phosphate buffered saline (PBS 1X, Gibco Life Technologies) and stored at -80° C. until use.

### T2 Cell Line Deficient in TAP

The human TAP-deficient T2 cell line (174xCEM.T2; ATCC CRL 1992™) was acquired from the American Type Culture Collection (ATCC; https://www.atcc.org/). Cell line T2 was maintained in Iscove’s modified Dulbecco medium (IMDM; Gibco Life Technologies) containing 25 mM HEPES and 2 mM L-Glutamine, supplemented with 20% fetal bovine serum (FBS, Capricorn Scientific GmbH), 100 IU / ml of penicillin and 100 µg / ml of streptomycin (Gibco Life Technologies). The cells were kept at 37° C. in a humidified incubator with 5% CO2.

### Affinity Assays for Peptide Bond and BFA Decay

The affinity tests for the binding of the peptides to HLA-A * 02: 01 and the Brefeldin A decay test (BFA) were performed for each synthesized peptide. Briefly, T2 cells were seeded at a concentration of 3.5 × 10⁵ cells per well in 24-well plates and incubated overnight in a humidified incubator at 37° C. with 5% CO2 with the peptides (concentrations : 10 µM, 20 µM, 50 µM, 100 µM) in serum-free IMDM medium containing 3 µg / ml of β2 (Sigma -Aldrich) microglobulin. After incubation, the cells were harvested and centrifuged at 200 × g for 5 minutes. Subsequently, the cells were washed twice with phosphate buffered saline (PBS 1X, Gibco Life Technologies) and stained with R-PE conjugated anti-HLA A2 monoclonal antibodies (Cat. 343306; BioLegend), for 30 minutes at 4° C. and analyzed with the Attune™ NxT flow cytometer (Thermo Fisher Scientific). The OVA peptide was used as a negative control and T2 cells with no added peptide were used as a background control. The fluorescence index (FI) was calculated using the following formula: FI = [average fluorescence intensity (MFI) sample - MFI background] / MFI background, where the MFI background represents the value without peptide. An FI> 0.5 was set as a threshold to indicate peptides with affinity for the HLA molecule A * 02: 01. For the Brefeldin A decay test, T2 cells were seeded at a concentration of 5 × 10⁵ cells to well into 24-well plates and cultured overnight as described above with candidate peptides or control peptide (CAP 1 was used as control) at a final concentration of 50 µM. After incubation, the cells were washed and incubated with 1X BFA (brefeldin A solution, 420601, BioLegend) in serum-free IMDM medium, for 1 hour at 37° C. Cells were collected every two hours (T0, T2, T4, T6, T8), washed with phosphate buffered saline (PBS 1X, Gibco Life Technologies), stained with anti-HLA A * 02: 01 fluorescent monoclonal antibodies (Cat. 343306 ; BioLegend) and analyzed by flow cytometry. The stability of each HLA A2-bound peptide was measured as the DC₅₀ value, which was defined as an estimate of the time required for a 50% reduction in the MFI value recorded at time 0. The DC₅₀ value was calculated according to the formula : MFI at indicated time points / MFI at time 0 × 100. All experiments were performed in triplicate.

### IFN γ ELISpot

The IFNy ELISpot assay (BDTM human IFNγ ELISPOT Set) was performed with PBMCs from HCC patients, stimulating 4 × 10⁶ PBMC / ml / well with peptides at the final concentration of 10 µg / ml. On the third day, 10U / ml of IL 2 was added to each well. On the fifth day, half the volume of medium was replaced with fresh medium containing IL 2 at a final concentration of 10U / ml. On the seventh day, the PBMCs were again stimulated with each peptide. On day 10, cells were harvested for the ELISpot IFNγ assay. Each peptide was added to a final concentration of 10 µg / mL at 2 × 10⁵ PBMC per well in 100 µl of RPMI 1640 (Capricorn Scientific GmbH). The PBMCs were grown at 37° C. in a humidified incubator with 5% CO2 for 20 hours. Stimulation with 10 µg / ml PHA (PHA-K; Capricorn Scientific GmbH) was used as a positive control, PBMCs without peptide addition were used as negative control, RPMI 1640 medium (Capricorn Scientific GmbH) was used as basic control. The wells were read with an ELISpot Reader AID system (AID GmbH, Strassberg, Germany) and the mean was obtained from readings of the wells in triplicate. Data were analyzed by subtracting the mean number of spots in the wells with untreated cells. Spot formation units (SFUs) were calculated as the frequency for 1×10⁶ PBMC.

### Statistic Analysis

Comparison of the individual data points was performed with the unpaired two-sided Student’s t-test and ANOVA, as appropriate. All P-values were two-sided and considered significant if less than 0.05.

### RESULTS

### Example 1. Bioinformatic Analysis of the Expression of HERV Genes in Samples Of Patients With Hepatocellular Carcinoma (HCC) and Identification of Epitopes Associated With Different Classes of HLA Class I

### Identification of Over-Expressed ERV Sequences in HCC

The expression analysis of the HERV sequences in tumor samples (T) versus the non-tumor counterpart (NT) showed the over-expression in each analyzed sample of HERV sequences identified in the Human Endogenous Retrovirus Database (HERVd) https://herv.img.cas.cz / with a T / NT ratio ≥ 2. Such sequences include both LTR (L) and internal (I) regions. The HERV sequences identified in the HERVd database with a T / NT ratio greater than or equal to 2 (at least 2-fold expression in tumor tissue compared to non-tumor tissue) are shown below in Table 1. In particular, in the ID column there are references alphanumeric which, if entered in the HERVd database (in the “Entities” section, in the “Name equals” field) allow to obtain information and details of the sequences, as well as the sequences themselves.

TABLE 1

| SAMPLE | CHROM | START | STOP | ID | NAME | Sub type | NT | T | ratio T vs. NT |
|---|---|---|---|---|---|---|---|---|---|
| HLA072 | 1 | 349009 | 349814 | ERV_0000262 | MER31-int | I | 0.18 | 2.37 | 13.18 |
| HLA063 | 1 | 1382090 | 1386135 | ERV_0001122 | MER66-int,MER66C | LI | 2.37 | 4.83 | 2.03 |
| HLA078 | 1 | 1382090 | 1386135 | ERV_0001122 | MER66-int,MER66C | LI | 0.88 | 3.15 | 3.57 |
| HLA072 | 1 | 2637771 | 2638501 | ERV_0002190 | ERVL-E-int | I | 0.34 | 2.75 | 8.09 |
| HLA078 | 1 | 4257712 | 4259619 | ERV_0003162 | THE1C,THE1C-int | LI | 0.00 | 1.61 | 300.00 |
| HLA078 | 1 | 7960625 | 7960739 | ERV_0006438 | MER84-int | I | 0.00 | 3.17 | 300.00 |
| HLA076 | 1 | 13313592 | 13313729 | ERV_0012312 | MER34-int | I | 0.46 | 1.66 | 3.62 |
| HLA061 | 1 | 16605694 | 16605874 | ERV_0015940 | MLT1F-int | I | 5.14 | 12.38 | 2.41 |
| HLA069 | 1 | 16605694 | 16605874 | ERV_0015940 | MLT1F-int | I | 2.22 | 14.20 | 6.41 |
| HLA073 | 1 | 16605694 | 16605874 | ERV_0015940 | MLT1F-int | I | 0.43 | 11.62 | 27.17 |
| HLA076 | 1 | 16605694 | 16605874 | ERV_0015940 | MLT1F-int | I | 3.65 | 9.74 | 2.67 |
| HLA078 | 1 | 16605694 | 16605874 | ERV_0015940 | MLT1F-int | I | 9.07 | 41.71 | 4.60 |
| HLA053 | 1 | 16903668 | 16904443 | ERV_0016202 | LTR23-int,LTR56 | LI | 0.08 | 2.02 | 24.47 |
| HLA063 | 1 | 20154110 | 20160513 | ERV_0020143 | HERVE-int,LTR2C | LIL | 3.68 | 8.06 | 2.19 |
| HLA073 | 1 | 20154110 | 20160513 | ERV_0020143 | HERVE-int,LTR2C | LIL | 0.12 | 6.32 | 53.08 |
| HLA076 | 1 | 20154110 | 20160513 | ERV_0020143 | HERVE-int,LTR2C | LIL | 1.11 | 2.87 | 2.58 |
| HLA078 | 1 | 20154110 | 20160513 | ERV_0020143 | HERVE-int,LTR2C | LIL | 0.53 | 15.20 | 28.46 |
| HLA082 | 1 | 20154110 | 20160513 | ERV_0020143 | HERVE-int,LTR2C | LIL | 1.03 | 2.45 | 2.39 |
| HLA063 | 1 | 20160106 | 20160228 | ERV_0020144 | Harlequin-int | I | 3.68 | 8.06 | 2.19 |
| HLA073 | 1 | 20160106 | 20160228 | ERV_0020144 | Harlequin-int | I | 0.12 | 6.32 | 53.08 |
| HLA076 | 1 | 20160106 | 20160228 | ERV_0020144 | Harlequin-int | I | 1.11 | 2.87 | 2.58 |
| HLA078 | 1 | 20160106 | 20160228 | ERV_0020144 | Harlequin-int | I | 0.53 | 15.20 | 28.46 |
| HLA082 | 1 | 20160106 | 20160228 | ERV_0020144 | Harlequin-int | I | 1.03 | 2.45 | 2.39 |
| HLA078 | 1 | 21453079 | 21453988 | ERV_0021799 | ERVL-E-int,LTR33 | LI | 0.34 | 2.33 | 6.90 |
| HLA078 | 1 | 31458573 | 31466684 | ERV_0034498 | HERVIP10F-int | I | 0.07 | 33.86 | 470.34 |
| HLA053 | 1 | 31517845 | 31518912 | ERV_0034558 | HERVL-int,MLT2A1 | LI | 0.15 | 2.08 | 13.87 |
| HLA058 | 1 | 31517845 | 31518912 | ERV_0034558 | HERVL-int,MLT2A1 | LI | 0.00 | 1.54 | 300.00 |
| HLA078 | 1 | 31517845 | 31518912 | ERV_0034558 | HERVL-int,MLT2A1 | LI | 0.26 | 1.96 | 7.57 |
| HLA058 | 1 | 33325217 | 33327957 | ERV_0036879 | MLT1F,MLT1F-int | LIL | 2.63 | 5.45 | 2.07 |
| HLA078 | 1 | 33325217 | 33327957 | ERV_0036879 | MLT1F,MLT1F-int | LIL | 4.74 | 11.58 | 2.44 |
| HLA058 | 1 | 33325576 | 33325665 | ERV_0036880 | MLT1F-int | I | 2.63 | 5.45 | 2.07 |
| HLA078 | 1 | 33325576 | 33325665 | ERV_0036880 | MLT1F-int | I | 4.74 | 11.58 | 2.44 |
| HLA067 | 1 | 35982765 | 35982904 | ERV_0039746 | MLT1J-int | I | 4.27 | 8.98 | 2.10 |
| HLA078 | 1 | 35982765 | 35982904 | ERV_0039746 | MLT1J-int | I | 0.68 | 10.47 | 15.32 |
| HLA065 | 1 | 38959345 | 38962221 | ERV_0043236 | MLT1D,MLT1D-int | LI | 0.00 | 10.68 | 300.00 |
| HLA078 | 1 | 38959345 | 38962221 | ERV_0043236 | MLT1D,MLT1D-int | LI | 0.00 | 2.72 | 300.00 |
| HLA053 | 1 | 39543019 | 39543161 | ERV_0043877 | ERV3-16A3_I-int | I | 0.00 | 1.73 | 300.00 |
| HLA078 | 1 | 41436846 | 41441007 | ERV_0045987 | ERVL-E-int | I | 0.10 | 1.54 | 15.05 |
| HLA058 | 1 | 44206493 | 44206571 | ERV_0048686 | ERVL-E-int | I | 0.00 | 2.00 | 300.00 |
| HLA058 | 1 | 45189915 | 45191639 | ERV_0049750 | ERV3-16A3_I-int,LTR16 | LI | 1.09 | 10.99 | 10.09 |
| HLA061 | 1 | 45189915 | 45191639 | ERV_0049750 | ERV3-16A3_I-int,LTR16 | LI | 0.36 | 7.30 | 20.03 |
| HLA072 | 1 | 45189915 | 45191639 | ERV_0049750 | ERV3-16A3_I-int,LTR16 | LI | 0.10 | 4.26 | 43.93 |
| HLA078 | 1 | 45189915 | 45191639 | ERV_0049750 | ERV3-16A3_I-int,LTR16 | LI | 0.00 | 2.99 | 300.00 |
| HLA082 | 1 | 46056993 | 46059302 | ERV_0050693 | THE1A,THE1A-int | LIL | 0.13 | 1.55 | 11.86 |
| HLA067 | 1 | 47541526 | 47543804 | ERV_0052168 | THE1B,THE1B-int | LIL | 0.59 | 3.15 | 5.36 |
| HLA082 | 1 | 47541526 | 47543804 | ERV_0052168 | THE1B,THE1B-int | LIL | 0.13 | 28.31 | 220.09 |
| HLA058 | 1 | 53204634 | 53207286 | ERV_0058440 | THE1D,THE1D-int | LIL | 0.89 | 2.96 | 3.34 |
| HLA065 | 1 | 53204634 | 53207286 | ERV_0058440 | THE1D,THE1D-int | LIL | 0.46 | 2.03 | 4.45 |
| HLA067 | 1 | 53204634 | 53207286 | ERV_0058440 | THE1D,THE1D-int | LIL | 1.19 | 5.82 | 4.91 |
| HLA072 | 1 | 53204634 | 53207286 | ERV_0058440 | THE1D,THE1D-int | LIL | 0.46 | 1.53 | 3.29 |
| HLA053 | 1 | 53892592 | 53892626 | ERV_0059244 | ERVL-E-int | I | 0.00 | 2.76 | 300.00 |
| HLA066 | 1 | 53892592 | 53892626 | ERV_0059244 | ERVL-E-int | I | 0.00 | 2.00 | 300.00 |
| HLA058 | 1 | 54101969 | 54105569 | ERV_0059490 | ERV3-16A3_I-int,LTR16A | LI | 1.89 | 4.29 | 2.27 |
| HLA061 | 1 | 54101969 | 54105569 | ERV_0059490 | ERV3-16A3_I-int,LTR16A | LI | 2.55 | 6.09 | 2.39 |
| HLA063 | 1 | 54101969 | 54105569 | ERV_0059490 | ERV3-16A3_I-int,LTR16A | LI | 2.00 | 5.82 | 2.91 |
| HLA065 | 1 | 54101969 | 54105569 | ERV_0059490 | ERV3-16A3_I-int,LTR16A | LI | 1.20 | 4.46 | 3.70 |
| HLA069 | 1 | 54101969 | 54105569 | ERV_0059490 | ERV3-16A3_I-int,LTR16A | LI | 1.55 | 4.39 | 2.82 |
| HLA073 | 1 | 54101969 | 54105569 | ERV_0059490 | ERV3-16A3_I-int,LTR16A | LI | 0.75 | 3.78 | 5.04 |
| HLA078 | 1 | 54101969 | 54105569 | ERV_0059490 | ERV3-16A3_I-int,LTR16A | LI | 1.66 | 8.01 | 4.81 |
| HLA067 | 1 | 55030953 | 55033231 | ERV_0060553 | THE1A,THE1A-int | LIL | 0.98 | 3.05 | 3.12 |
| HLA071 | 1 | 55030953 | 55033231 | ERV_0060553 | THE1A,THE1A-int | LIL | 0.17 | 1.67 | 9.82 |
| HLA082 | 1 | 55030953 | 55033231 | ERV_0060553 | THE1A,THE1A-int | LIL | 0.77 | 3.19 | 4.15 |
| HLA053 | 1 | 56492595 | 56494136 | ERV_0061992 | MLT1D,MLT1D-int | LI | 17.65 | 51.56 | 2.92 |
| HLA078 | 1 | 58629676 | 58633792 | ERV_0064431 | MSTD,MSTD-int | LIL | 0.70 | 1.96 | 2.81 |
| HLA082 | 1 | 58629676 | 58633792 | ERV_0064431 | MSTD,MSTD-int | LIL | 0.76 | 1.56 | 2.05 |
| HLA063 | 1 | 59514549 | 59515871 | ERV_0065445 | THE1A,THE1A-int | LIL | 1.32 | 3.63 | 2.74 |
| HLA066 | 1 | 59514549 | 59515871 | ERV_0065445 | THE1A,THE1A-int | LIL | 10.45 | 27.88 | 2.67 |
| HLA067 | 1 | 59514549 | 59515871 | ERV_0065445 | THE1A,THE1A-int | LIL | 19.43 | 87.73 | 4.51 |
| HLA069 | 1 | 59514549 | 59515871 | ERV_0065445 | THE1A,THE1A-int | LIL | 3.52 | 34.16 | 9.71 |
| HLA073 | 1 | 59514549 | 59515871 | ERV_0065445 | THE1A,THE1A-int | LIL | 3.05 | 6.56 | 2.15 |
| HLA076 | 1 | 59514549 | 59515871 | ERV_0065445 | THE1A,THE1A-int | LIL | 1.57 | 6.27 | 4.00 |
| HLA082 | 1 | 59514549 | 59515871 | ERV_0065445 | THE1A,THE1A-int | LIL | 5.99 | 21.78 | 3.63 |
| HLA063 | 1 | 59515081 | 59515522 | ERV_0065446 | THE1A-int | I | 1.32 | 3.63 | 2.74 |
| HLA066 | 1 | 59515081 | 59515522 | ERV_0065446 | THE1A-int | I | 10.45 | 27.88 | 2.67 |
| HLA067 | 1 | 59515081 | 59515522 | ERV_0065446 | THE1A-int | I | 19.43 | 87.73 | 4.51 |
| HLA069 | 1 | 59515081 | 59515522 | ERV_0065446 | THE1A-int | I | 3.52 | 34.16 | 9.71 |
| HLA073 | 1 | 59515081 | 59515522 | ERV_0065446 | THE1A-int | I | 3.05 | 6.56 | 2.15 |
| HLA076 | 1 | 59515081 | 59515522 | ERV_0065446 | THE1A-int | I | 1.57 | 6.27 | 4.00 |
| HLA082 | 1 | 59515081 | 59515522 | ERV_0065446 | THE1A-int | I | 5.99 | 21.78 | 3.63 |
| HLA078 | 1 | 64186607 | 64192762 | ERV_0070158 | HERVL18-int,LTR18A | LIL | 0.00 | 2.80 | 300.00 |
| HLA072 | 1 | 67207422 | 67209695 | ERV_0072969 | THE1A,THE1A-int | LIL | 0.74 | 4.95 | 6.66 |
| HLA078 | 1 | 67207422 | 67209695 | ERV_0072969 | THE1A,THE1A-int | LIL | 0.00 | 3.03 | 300.00 |
| HLA053 | 1 | 67515309 | 67515734 | ERV_0073337 | LTR49-int | I | 0.24 | 2.59 | 10.77 |
| HLA073 | 1 | 67515309 | 67515734 | ERV_0073337 | LTR49-int | I | 0.19 | 1.99 | 10.59 |
| HLA078 | 1 | 67515309 | 67515734 | ERV_0073337 | LTR49-int | I | 0.58 | 1.82 | 3.16 |
| HLA078 | 1 | 68086244 | 68086394 | ERV_0073918 | MLT1J1-int | I | 0.00 | 1.73 | 300.00 |
| HLA065 | 1 | 70281241 | 70282782 | ERV_0075697 | MLT1J,MLT1J-int | LI | 2.45 | 6.23 | 2.54 |
| HLA072 | 1 | 70281241 | 70282782 | ERV_0075697 | MLT1J,MLT1J-int | LI | 1.33 | 7.45 | 5.58 |
| HLA073 | 1 | 70367629 | 70374024 | ERV_0075815 | HERVH-int,LTR7 | LIL | 1.38 | 3.09 | 2.25 |
| HLA078 | 1 | 70367629 | 70374024 | ERV_0075815 | HERVH-int,LTR7 | LIL | 0.83 | 3.95 | 4.77 |
| HLA073 | 1 | 70372371 | 70373591 | ERV_0075816 | HERVH-int | I | 1.38 | 3.09 | 2.25 |
| HLA078 | 1 | 70372371 | 70373591 | ERV_0075816 | HERVH-int | I | 0.83 | 3.95 | 4.77 |
| HLA053 | 1 | 77706472 | 77708145 | ERV_0081829 | ERVL-E-int | I | 2.07 | 8.45 | 4.08 |
| HLA058 | 1 | 77706472 | 77708145 | ERV_0081829 | ERVL-E-int | I | 0.16 | 2.34 | 14.57 |
| HLA067 | 1 | 77706472 | 77708145 | ERV_0081829 | ERVL-E-int | I | 1.43 | 3.82 | 2.68 |
| HLA053 | 1 | 78293370 | 78295657 | ERV_0082398 | THE1A,THE1A-int | LIL | 1.20 | 3.67 | 3.05 |
| HLA058 | 1 | 78293370 | 78295657 | ERV_0082398 | THE1A,THE1A-int | LIL | 2.81 | 6.42 | 2.28 |
| HLA058 | 1 | 78646295 | 78646353 | ERV_0082651 | MamGyp-int | I | 0.00 | 1.79 | 300.00 |
| HLA066 | 1 | 78646295 | 78646353 | ERV_0082651 | MamGyp-int | I | 0.00 | 1.78 | 300.00 |
| HLA067 | 1 | 78646295 | 78646353 | ERV_0082651 | MamGyp-int | I | 0.00 | 2.52 | 300.00 |
| HLA072 | 1 | 78646295 | 78646353 | ERV_0082651 | MamGyp-int | I | 0.00 | 5.34 | 300.00 |
| HLA082 | 1 | 78646295 | 78646353 | ERV_0082651 | MamGyp-int | I | 0.86 | 2.95 | 3.42 |
| HLA065 | 1 | 83669802 | 83670488 | ERV_0086738 | THE1B,THE1B-int | LI | 0.00 | 3.78 | 300.00 |
| HLA067 | 1 | 83669802 | 83670488 | ERV_0086738 | THE1B,THE1B-int | LI | 0.21 | 2.96 | 14.01 |
| HLA058 | 1 | 84102016 | 84103764 | ERV_0087089 | MLT1G,MLT1G-int | LI | 1.37 | 4.10 | 2.98 |
| HLA061 | 1 | 84102016 | 84103764 | ERV_0087089 | MLT1G,MLT1G-int | LI | 0.87 | 3.26 | 3.73 |
| HLA072 | 1 | 84102016 | 84103764 | ERV_0087089 | MLT1G,MLT1G-int | LI | 0.34 | 2.01 | 5.91 |
| HLA053 | 1 | 84536109 | 84539258 | ERV_0087444 | ERVL-E-int | I | 0.85 | 3.42 | 4.03 |
| HLA053 | 1 | 85398062 | 85399467 | ERV_0088338 | MER65-int | I | 0.39 | 2.06 | 5.30 |
| HLA072 | 1 | 85398062 | 85399467 | ERV_0088338 | MER65-int | I | 0.54 | 2.62 | 4.85 |
| HLA058 | 1 | 87808368 | 87808808 | ERV_0090418 | LTR52,LTR52-int | LI | 0.00 | 9.94 | 300.00 |
| HLA067 | 1 | 87808368 | 87808808 | ERV_0090418 | LTR52,LTR52-int | LI | 0.00 | 1.93 | 300.00 |
| HLA069 | 1 | 87808368 | 87808808 | ERV_0090418 | LTR52,LTR52-int | LI | 0.33 | 2.55 | 7.68 |
| HLA071 | 1 | 87808368 | 87808808 | ERV_0090418 | LTR52,LTR52-int | LI | 0.71 | 3.66 | 5.18 |
| HLA073 | 1 | 87808368 | 87808808 | ERV_0090418 | LTR52,LTR52-int | LI | 0.00 | 1.78 | 300.00 |
| HLA078 | 1 | 87808368 | 87808808 | ERV_0090418 | LTR52,LTR52-int | LI | 0.26 | 1.57 | 6.00 |
| HLA053 | 1 | 88924529 | 88931881 | ERV_0091394 | HERV17-int,LTR17 | LIL | 0.64 | 2.16 | 3.39 |
| HLA053 | 1 | 89099124 | 89101806 | ERV_0091544 | MER89-int | I | 0.49 | 1.58 | 3.24 |
| HLA067 | 1 | 89099124 | 89101806 | ERV_0091544 | MER89-int | I | 0.66 | 1.68 | 2.53 |
| HLA066 | 1 | 89179228 | 89179558 | ERV_0091594 | HERV4_I-int | I | 0.00 | 1.75 | 300.00 |
| HLA063 | 1 | 89203974 | 89206780 | ERV_0091617 | MSTA,MSTA-int | LIL | 1.16 | 4.00 | 3.45 |
| HLA065 | 1 | 89203974 | 89206780 | ERV_0091617 | MSTA,MSTA-int | LIL | 0.70 | 1.87 | 2.68 |
| HLA067 | 1 | 89203974 | 89206780 | ERV_0091617 | MSTA,MSTA-int | LIL | 0.74 | 2.03 | 2.74 |
| HLA071 | 1 | 89203974 | 89206780 | ERV_0091617 | MSTA,MSTA-int | LIL | 0.84 | 1.70 | 2.03 |
| HLA073 | 1 | 89203974 | 89206780 | ERV_0091617 | MSTA,MSTA-int | LIL | 0.69 | 1.56 | 2.27 |
| HLA058 | 1 | 89386836 | 89387381 | ERV_0091736 | HERV16-int | I | 0.54 | 1.81 | 3.33 |
| HLA063 | 1 | 89386836 | 89387381 | ERV_0091736 | HERV16-int | I | 0.00 | 4.20 | 300.00 |
| HLA065 | 1 | 89386836 | 89387381 | ERV_0091736 | HERV16-int | I | 0.00 | 2.35 | 300.00 |
| HLA053 | 1 | 94252279 | 94252466 | ERV_0096209 | HERVL-int | I | 0.00 | 1.98 | 300.00 |
| HLA067 | 1 | 94428539 | 94430735 | ERV_0096364 | THE1B,THE1B-int | LIL | 0.15 | 2.28 | 14.96 |
| HLA058 | 1 | 94444820 | 94450789 | ERV_0096376 | HERVK9-int,MER9a3 | LIL | 0.61 | 2.92 | 4.81 |
| HLA065 | 1 | 94444820 | 94450789 | ERV_0096376 | HERVK9-int,MER9a3 | LIL | 0.40 | 1.67 | 4.23 |
| HLA067 | 1 | 94444820 | 94450789 | ERV_0096376 | HERVK9-int,MER9a3 | LIL | 2.21 | 8.96 | 4.05 |
| HLA076 | 1 | 96140122 | 96153437 | ERV_0097749 | HERV9N-int,LTR12,LTR12_ | LIL | 0.00 | 1.62 | 300.00 |
| HLA058 | 1 | 97407534 | 97409154 | ERV_0098789 | HERVL-int,MLT2A2 | LI | 0.00 | 2.45 | 300.00 |
| HLA058 | 1 | 97765650 | 97767760 | ERV_0099129 | MLT1H,MLT1H-int | LIL | 0.84 | 2.28 | 2.70 |
| HLA072 | 1 | 97765650 | 97767760 | ERV_0099129 | MLT1H,MLT1H-int | LIL | 0.09 | 3.48 | 40.77 |
| HLA078 | 1 | 1E+08 | 1E+08 | ERV_0101272 | THE1B,THE1B-int | LI | 0.09 | 3.16 | 35.53 |
| HLA058 | 1 | 1.02E+08 | 1.02E+08 | ERV_0103119 | THE1D,THE1D-int | LIL | 0.00 | 2.08 | 300.00 |
| HLA053 | 1 | 1.08E+08 | 1.08E+08 | ERV_0107695 | MamGyp-int | I | 0.56 | 3.46 | 6.20 |
| HLA082 | 1 | 1.08E+08 | 1.08E+08 | ERV_0107695 | MamGyp-int | I | 0.49 | 1.86 | 3.78 |
| HLA072 | 1 | 1.09E+08 | 1.09E+08 | ERV_0108082 | MER50-int | I | 0.00 | 1.90 | 300.00 |
| HLA053 | 1 | 1.1E+08 | 1.1E+08 | ERV_0109261 | HERVK11-int,MER11A | LIL | 0.43 | 2.34 | 5.42 |
| HLA071 | 1 | 1.1E+08 | 1.1E+08 | ERV_0109261 | HERVK11-int,MER11A | LIL | 1.67 | 4.77 | 2.86 |
| HLA078 | 1 | 1.1E+08 | 1.1E+08 | ERV_0109261 | HERVK11-int,MER11A | LIL | 0.29 | 1.53 | 5.19 |
| HLA053 | 1 | 1.11E+08 | 1.11E+08 | ERV_0110695 | ERVL-E-int,LTR33 | LI | 0.54 | 2.56 | 4.71 |
| HLA076 | 1 | 1.11E+08 | 1.11E+08 | ERV_0110695 | ERVL-E-int,LTR33 | LI | 0.62 | 1.67 | 2.69 |
| HLA065 | 1 | 1.13E+08 | 1.13E+08 | ERV_0112431 | MER31-int,MER31A | LI | 0.74 | 2.04 | 2.75 |
| HLA058 | 1 | 1.18E+08 | 1.18E+08 | ERV_0116684 | MSTA,MSTA-int | LIL | 0.21 | 1.92 | 9.09 |
| HLA078 | 1 | 1.18E+08 | 1.18E+08 | ERV_0116684 | MSTA,MSTA-int | LIL | 0.28 | 1.59 | 5.64 |
| HLA058 | 1 | 1.19E+08 | 1.19E+08 | ERV_0117849 | HERV9NC-int,LTR12C | LIL | 0.71 | 2.16 | 3.06 |
| HLA058 | 1 | 1.2E+08 | 1.2E+08 | ERV_0118349 | ERVL-B4-int,MLT2D | LI | 0.00 | 1.64 | 300.00 |
| HLA058 | 1 | 1.2E+08 | 1.2E+08 | ERV_0118835 | PRIMA4-int | I | 0.37 | 1.68 | 4.59 |
| HLA065 | 1 | 1.2E+08 | 1.2E+08 | ERV_0118835 | PRIMA4-int | I | 0.79 | 1.78 | 2.24 |
| HLA078 | 1 | 1.2E+08 | 1.2E+08 | ERV_0118933 | ERVL-E-int,LTR33 | LI | 2.23 | 8.82 | 3.95 |
| HLA066 | 1 | 1.21E+08 | 1.21E+08 | ERV_0119810 | ERVL-B4-int | I | 0.00 | 4.48 | 300.00 |
| HLA072 | 1 | 1.21E+08 | 1.21E+08 | ERV_0119892 | MER89-int | I | 0.00 | 3.93 | 300.00 |
| HLA058 | 1 | 1.21E+08 | 1.21E+08 | ERV_0119893 | ERV24_Prim-int | I | 0.30 | 1.58 | 5.20 |
| HLA067 | 1 | 1.21E+08 | 1.21E+08 | ERV_0119893 | ERV24_Prim-int | I | 1.20 | 2.92 | 2.43 |
| HLA072 | 1 | 1.21E+08 | 1.21E+08 | ERV_0119893 | ERV24_Prim-int | I | 0.06 | 1.98 | 33.45 |
| HLA067 | 1 | 1.22E+08 | 1.22E+08 | ERV_0119903 | HERV9N-int,LTR12_ | LIL | 1.09 | 2.44 | 2.23 |
| HLA063 | 1 | 1.44E+08 | 1.44E+08 | ERV_0120563 | LTR49,LTR49-int | LI | 1.25 | 6.24 | 4.98 |
| HLA071 | 1 | 1.44E+08 | 1.44E+08 | ERV_0120563 | LTR49,LTR49-int | LI | 1.20 | 7.74 | 6.43 |
| HLA073 | 1 | 1.44E+08 | 1.44E+08 | ERV_0120563 | LTR49,LTR49-int | LI | 1.38 | 5.78 | 4.18 |
| HLA076 | 1 | 1.44E+08 | 1.44E+08 | ERV_0120563 | LTR49,LTR49-int | LI | 0.81 | 2.26 | 2.80 |
| HLA065 | 1 | 1.44E+08 | 1.44E+08 | ERV_0121217 | MLT1E3,MLT1E3-int | LI | 1.32 | 6.67 | 5.04 |
| HLA067 | 1 | 1.44E+08 | 1.44E+08 | ERV_0121217 | MLT1E3,MLT1E3-int | LI | 3.72 | 13.70 | 3.68 |
| HLA069 | 1 | 1.44E+08 | 1.44E+08 | ERV_0121217 | MLT1E3,MLT1E3-int | LI | 2.40 | 5.12 | 2.13 |
| HLA078 | 1 | 1.44E+08 | 1.44E+08 | ERV_0121217 | MLT1E3,MLT1E3-int | LI | 1.54 | 16.07 | 10.41 |
| HLA053 | 1 | 1.45E+08 | 1.45E+08 | ERV_0121918 | MER66-int,MER66C | LI | 1.44 | 6.90 | 4.80 |
| HLA058 | 1 | 1.45E+08 | 1.45E+08 | ERV_0121918 | MER66-int,MER66C | LI | 0.78 | 2.30 | 2.96 |
| HLA066 | 1 | 1.45E+08 | 1.45E+08 | ERV_0121918 | MER66-int,MER66C | LI | 0.26 | 2.51 | 9.48 |
| HLA072 | 1 | 1.45E+08 | 1.45E+08 | ERV_0121918 | MER66-int,MER66C | LI | 0.19 | 2.45 | 12.90 |
| HLA053 | 1 | 1.45E+08 | 1.45E+08 | ERV_0121920 | MER66-int | I | 0.42 | 2.13 | 5.06 |
| HLA053 | 1 | 1.45E+08 | 1.45E+08 | ERV_0121923 | HERVP71A-int,LTR71B | LI | 0.42 | 4.09 | 9.74 |
| HLA072 | 1 | 1.45E+08 | 1.45E+08 | ERV_0121923 | HERVP71A-int,LTR71B | LI | 0.00 | 3.05 | 300.00 |
| HLA065 | 1 | 1.45E+08 | 1.45E+08 | ERV_0122124 | MLT1E3,MLT1E3-int | LI | 2.93 | 12.32 | 4.21 |
| HLA067 | 1 | 1.45E+08 | 1.45E+08 | ERV_0122124 | MLT1E3,MLT1E3-int | LI | 9.24 | 26.76 | 2.90 |
| HLA069 | 1 | 1.45E+08 | 1.45E+08 | ERV_0122124 | MLT1E3,MLT1E3-int | LI | 3.33 | 6.77 | 2.03 |
| HLA078 | 1 | 1.45E+08 | 1.45E+08 | ERV_0122124 | MLT1E3,MLT1E3-int | LI | 4.04 | 24.45 | 6.05 |
| HLA058 | 1 | 1.46E+08 | 1.46E+08 | ERV_0122759 | MER4B,MER4B-int | LI | 0.16 | 4.95 | 30.94 |
| HLA061 | 1 | 1.46E+08 | 1.46E+08 | ERV_0122759 | MER4B,MER4B-int | LI | 0.59 | 10.96 | 18.69 |
| HLA072 | 1 | 1.46E+08 | 1.46E+08 | ERV_0122759 | MER4B,MER4B-int | LI | 0.17 | 2.52 | 14.44 |
| HLA073 | 1 | 1.46E+08 | 1.46E+08 | ERV_0122759 | MER4B,MER4B-int | LI | 0.13 | 1.82 | 13.48 |
| HLA078 | 1 | 1.46E+08 | 1.46E+08 | ERV_0122759 | MER4B,MER4B-int | LI | 0.13 | 6.71 | 50.84 |
| HLA058 | 1 | 1.46E+08 | 1.46E+08 | ERV_0122761 | ERVL-E-int,MLT2F | LI | 0.16 | 4.95 | 30.94 |
| HLA061 | 1 | 1.46E+08 | 1.46E+08 | ERV_0122761 | ERVL-E-int,MLT2F | LI | 0.59 | 10.96 | 18.69 |
| HLA072 | 1 | 1.46E+08 | 1.46E+08 | ERV_0122761 | ERVL-E-int,MLT2F | LI | 0.17 | 2.52 | 14.44 |
| HLA073 | 1 | 1.46E+08 | 1.46E+08 | ERV_0122761 | ERVL-E-int,MLT2F | LI | 0.13 | 1.82 | 13.48 |
| HLA078 | 1 | 1.46E+08 | 1.46E+08 | ERV_0122761 | ERVL-E-int,MLT2F | LI | 0.13 | 6.71 | 50.84 |
| HLA065 | 1 | 1.47E+08 | 1.47E+08 | ERV_0123613 | ERVL-E-int,LTR33 | LI | 1.55 | 3.92 | 2.52 |
| HLA067 | 1 | 1.47E+08 | 1.47E+08 | ERV_0123613 | ERVL-E-int,LTR33 | LI | 4.18 | 14.66 | 3.50 |
| HLA069 | 1 | 1.47E+08 | 1.47E+08 | ERV_0123613 | ERVL-E-int,LTR33 | LI | 2.87 | 7.25 | 2.52 |
| HLA078 | 1 | 1.47E+08 | 1.47E+08 | ERV_0123613 | ERVL-E-int,LTR33 | LI | 0.90 | 3.61 | 4.03 |
| HLA071 | 1 | 1.48E+08 | 1.48E+08 | ERV_0124314 | MER21-int | I | 0.00 | 2.45 | 300.00 |
| HLA076 | 1 | 1.48E+08 | 1.48E+08 | ERV_0124314 | MER21-int | I | 0.00 | 5.89 | 300.00 |
| HLA078 | 1 | 1.48E+08 | 1.48E+08 | ERV_0124314 | MER21-int | I | 0.00 | 3.78 | 300.00 |
| HLA067 | 1 | 1.48E+08 | 1.48E+08 | ERV_0124584 | MLT1E2,MLT1E2-int | LI | 2.19 | 7.95 | 3.63 |
| HLA073 | 1 | 1.48E+08 | 1.48E+08 | ERV_0124584 | MLT1E2,MLT1E2-int | LI | 1.75 | 3.82 | 2.18 |
| HLA076 | 1 | 1.48E+08 | 1.48E+08 | ERV_0124584 | MLT1E2,MLT1E2-int | LI | 2.52 | 10.59 | 4.21 |
| HLA078 | 1 | 1.48E+08 | 1.48E+08 | ERV_0124584 | MLT1E2,MLT1E2-int | LI | 0.29 | 5.08 | 17.67 |
| HLA071 | 1 | 1.48E+08 | 1.48E+08 | ERV_0124695 | ERV24_Prim-int,MER87 | LI | 0.07 | 1.83 | 25.09 |
| HLA078 | 1 | 1.48E+08 | 1.48E+08 | ERV_0124695 | ERV24_Prim-int,MER87 | LI | 0.00 | 2.12 | 300.00 |
| HLA078 | 1 | 1.48E+08 | 1.48E+08 | ERV_0124778 | LOR1-int,LOR1b | LI | 0.00 | 5.42 | 300.00 |
| HLA065 | 1 | 1.48E+08 | 1.48E+08 | ERV_0124799 | MLT1E2,MLT1E2-int | LI | 0.00 | 1.51 | 300.00 |
| HLA067 | 1 | 1.49E+08 | 1.49E+08 | ERV_0125432 | MLT1E3,MLT1E3-int | LI | 12.93 | 26.48 | 2.05 |
| HLA069 | 1 | 1.49E+08 | 1.49E+08 | ERV_0125432 | MLT1E3,MLT1E3-int | LI | 4.50 | 9.71 | 2.16 |
| HLA078 | 1 | 1.49E+08 | 1.49E+08 | ERV_0125432 | MLT1E3,MLT1E3-int | LI | 4.29 | 22.23 | 5.18 |
| HLA072 | 1 | 1.5E+08 | 1.5E+08 | ERV_0125913 | ERVL-E-int | I | 0.22 | 1.57 | 7.02 |
| HLA072 | 1 | 1.5E+08 | 1.5E+08 | ERV_0125980 | LTR49,LTR49-int | LI | 0.42 | 1.52 | 3.59 |
| HLA058 | 1 | 1.51E+08 | 1.51E+08 | ERV_0127104 | HERVK-int,LTR5_Hs | LIL | 0.74 | 5.88 | 7.93 |
| HLA061 | 1 | 1.51E+08 | 1.51E+08 | ERV_0127104 | HERVK-int,LTR5_Hs | LIL | 3.54 | 8.32 | 2.35 |
| HLA063 | 1 | 1.51E+08 | 1.51E+08 | ERV_0127104 | HERVK-int,LTR5_Hs | LIL | 1.78 | 4.32 | 2.42 |
| HLA066 | 1 | 1.51E+08 | 1.51E+08 | ERV_0127104 | HERVK-int,LTR5_Hs | LIL | 0.79 | 4.08 | 5.18 |
| HLA067 | 1 | 1.51E+08 | 1.51E+08 | ERV_0127104 | HERVK-int,LTR5_Hs | LIL | 2.19 | 5.76 | 2.63 |
| HLA069 | 1 | 1.51E+08 | 1.51E+08 | ERV_0127104 | HERVK-int,LTR5_Hs | LIL | 1.11 | 2.71 | 2.43 |
| HLA073 | 1 | 1.51E+08 | 1.51E+08 | ERV_0127104 | HERVK-int,LTR5_Hs | LIL | 1.45 | 3.46 | 2.38 |
| HLA078 | 1 | 1.51E+08 | 1.51E+08 | ERV_0127104 | HERVK-int,LTR5_Hs | LIL | 0.66 | 13.60 | 20.63 |
| HLA063 | 1 | 1.51E+08 | 1.51E+08 | ERV_0127141 | HERVK9-int,MER9a3 | LI | 0.36 | 1.52 | 4.19 |
| HLA065 | 1 | 1.51E+08 | 1.51E+08 | ERV_0127674 | MER41-int | I | 0.21 | 4.20 | 20.13 |
| HLA072 | 1 | 1.51E+08 | 1.51E+08 | ERV_0127674 | MER41-int | I | 0.76 | 1.78 | 2.35 |
| HLA078 | 1 | 1.51E+08 | 1.51E+08 | ERV_0127674 | MER41-int | I | 0.34 | 2.01 | 5.95 |
| HLA058 | 1 | 1.52E+08 | 1.52E+08 | ERV_0128555 | MER61-int,MER61C | LI | 0.00 | 6.04 | 300.00 |
| HLA078 | 1 | 1.52E+08 | 1.52E+08 | ERV_0128555 | MER61-int,MER61C | LI | 0.33 | 2.11 | 6.34 |
| HLA058 | 1 | 1.54E+08 | 1.54E+08 | ERV_0129907 | HUERS-P3b-int,LTR9B | LIL | 7.20 | 19.81 | 2.75 |
| HLA066 | 1 | 1.54E+08 | 1.54E+08 | ERV_0129907 | HUERS-P3b-int,LTR9B | LIL | 5.80 | 12.15 | 2.10 |
| HLA073 | 1 | 1.54E+08 | 1.54E+08 | ERV_0129907 | HUERS-P3b-int,LTR9B | LIL | 4.43 | 10.69 | 2.41 |
| HLA078 | 1 | 1.54E+08 | 1.54E+08 | ERV_0129907 | HUERS-P3b-int,LTR9B | LIL | 7.87 | 22.33 | 2.84 |
| HLA058 | 1 | 1.54E+08 | 1.54E+08 | ERV_0129908 | MER61-int | I | 7.20 | 19.81 | 2.75 |
| HLA066 | 1 | 1.54E+08 | 1.54E+08 | ERV_0129908 | MER61-int | I | 5.80 | 12.15 | 2.10 |
| HLA073 | 1 | 1.54E+08 | 1.54E+08 | ERV_0129908 | MER61-int | I | 4.43 | 10.69 | 2.41 |
| HLA078 | 1 | 1.54E+08 | 1.54E+08 | ERV_0129908 | MER61-int | I | 7.87 | 22.33 | 2.84 |
| HLA053 | 1 | 1.55E+08 | 1.55E+08 | ERV_0131153 | HERV9NC-int,LTR12C | LIL | 0.13 | 2.04 | 15.25 |
| HLA058 | 1 | 1.55E+08 | 1.55E+08 | ERV_0131153 | HERV9NC-int,LTR12C | LIL | 0.06 | 167.41 | 2788.25 |
| HLA069 | 1 | 1.55E+08 | 1.55E+08 | ERV_0131153 | HERV9NC-int,LTR12C | LIL | 0.00 | 89.94 | 300.00 |
| HLA071 | 1 | 1.55E+08 | 1.55E+08 | ERV_0131153 | HERV9NC-int,LTR12C | LIL | 0.71 | 12.60 | 17.84 |
| HLA078 | 1 | 1.55E+08 | 1.55E+08 | ERV_0131153 | HERV9NC-int,LTR12C | LIL | 0.01 | 8.24 | 1412.03 |
| HLA082 | 1 | 1.55E+08 | 1.55E+08 | ERV_0131153 | HERV9NC-int,LTR12C | LIL | 0.42 | 59.02 | 141.46 |
| HLA063 | 1 | 1.55E+08 | 1.55E+08 | ERV_0131872 | MER41-int, MER41A,MER41B | LIL | 0.00 | 2.06 | 300.00 |
| HLA067 | 1 | 1.55E+08 | 1.55E+08 | ERV_0131872 | MER41-int, MER41A,MER41B | LIL | 0.00 | 3.09 | 300.00 |
| HLA072 | 1 | 1.55E+08 | 1.55E+08 | ERV_0131872 | MER41-int, MER41A,MER41B | LIL | 0.72 | 4.22 | 5.84 |
| HLA078 | 1 | 1.55E+08 | 1.55E+08 | ERV_0131872 | MER41-int, MER41A,MER41B | LIL | 0.33 | 1.97 | 5.94 |
| HLA063 | 1 | 1.55E+08 | 1.55E+08 | ERV_0131873 | MER41-int | I | 0.00 | 2.06 | 300.00 |
| HLA067 | 1 | 1.55E+08 | 1.55E+08 | ERV_0131873 | MER41-int | I | 0.00 | 3.09 | 300.00 |
| HLA072 | 1 | 1.55E+08 | 1.55E+08 | ERV_0131873 | MER41-int | I | 0.72 | 4.22 | 5.84 |
| HLA078 | 1 | 1.55E+08 | 1.55E+08 | ERV_0131873 | MER41-int | I | 0.33 | 1.97 | 5.94 |
| HLA053 | 1 | 1.58E+08 | 1.58E+08 | ERV_0134296 | THE1D,THE1D-int | LIL | 0.13 | 2.67 | 20.00 |
| HLA069 | 1 | 1.58E+08 | 1.58E+08 | ERV_0134740 | MER57A-int,MER57C1 | LI | 0.28 | 1.99 | 7.05 |
| HLA069 | 1 | 1.58E+08 | 1.58E+08 | ERV_0134742 | MER34B-int | I | 0.28 | 1.99 | 7.05 |
| HLA058 | 1 | 1.59E+08 | 1.59E+08 | ERV_0135678 | ERV3-16A3_I-int, ERV3-16A3_LTR | LI | 0.00 | 1.59 | 300.00 |
| HLA071 | 1 | 1.59E+08 | 1.59E+08 | ERV_0135678 | ERV3-16A3_I-int, ERV3-16A3_LTR | LI | 0.56 | 4.86 | 8.64 |
| HLA076 | 1 | 1.59E+08 | 1.59E+08 | ERV_0135678 | ERV3-16A3_I-int, ERV3-16A3_LTR | LI | 0.00 | 2.01 | 300.00 |
| HLA067 | 1 | 1.61E+08 | 1.61E+08 | ERV_0136991 | ERVL-E-int | I | 0.00 | 1.70 | 300.00 |
| HLA058 | 1 | 1.62E+08 | 1.62E+08 | ERV_0137703 | MER34B-int,MER57F | LIL | 2.61 | 6.45 | 2.47 |
| HLA066 | 1 | 1.62E+08 | 1.62E+08 | ERV_0137703 | MER34B-int,MER57F | LIL | 2.16 | 12.89 | 5.97 |
| HLA078 | 1 | 1.62E+08 | 1.62E+08 | ERV_0137703 | MER34B-int,MER57F | LIL | 8.24 | 54.00 | 6.56 |
| HLA058 | 1 | 1.62E+08 | 1.62E+08 | ERV_0137704 | MER34-int | I | 2.61 | 6.45 | 2.47 |
| HLA066 | 1 | 1.62E+08 | 1.62E+08 | ERV_0137704 | MER34-int | I | 2.16 | 12.89 | 5.97 |
| HLA078 | 1 | 1.62E+08 | 1.62E+08 | ERV_0137704 | MER34-int | I | 8.24 | 54.00 | 6.56 |
| HLA058 | 1 | 1.62E+08 | 1.62E+08 | ERV_0137705 | MER57-int | I | 2.61 | 6.45 | 2.47 |
| HLA066 | 1 | 1.62E+08 | 1.62E+08 | ERV_0137705 | MER57-int | I | 2.16 | 12.89 | 5.97 |
| HLA078 | 1 | 1.62E+08 | 1.62E+08 | ERV_0137705 | MER57-int | I | 8.24 | 54.00 | 6.56 |
| HLA058 | 1 | 1.62E+08 | 1.62E+08 | ERV_0137783 | MER34B-int,MER57F | LIL | 1.14 | 7.75 | 6.81 |
| HLA066 | 1 | 1.62E+08 | 1.62E+08 | ERV_0137783 | MER34B-int,MER57F | LIL | 0.80 | 7.47 | 9.29 |
| HLA078 | 1 | 1.62E+08 | 1.62E+08 | ERV_0137783 | MER34B-int,MER57F | LIL | 1.44 | 12.54 | 8.74 |
| HLA058 | 1 | 1.62E+08 | 1.62E+08 | ERV_0137784 | LTR57-int | I | 1.14 | 7.75 | 6.81 |
| HLA066 | 1 | 1.62E+08 | 1.62E+08 | ERV_0137784 | LTR57-int | I | 0.80 | 7.47 | 9.29 |
| HLA078 | 1 | 1.62E+08 | 1.62E+08 | ERV_0137784 | LTR57-int | I | 1.44 | 12.54 | 8.74 |
| HLA058 | 1 | 1.62E+08 | 1.62E+08 | ERV_0137785 | MER34-int | I | 1.14 | 7.75 | 6.81 |
| HLA066 | 1 | 1.62E+08 | 1.62E+08 | ERV_0137785 | MER34-int | I | 0.80 | 7.47 | 9.29 |
| HLA078 | 1 | 1.62E+08 | 1.62E+08 | ERV_0137785 | MER34-int | I | 1.44 | 12.54 | 8.74 |
| HLA058 | 1 | 1.62E+08 | 1.62E+08 | ERV_0137786 | MER57-int | I | 1.14 | 7.75 | 6.81 |
| HLA066 | 1 | 1.62E+08 | 1.62E+08 | ERV_0137786 | MER57-int | I | 0.80 | 7.47 | 9.29 |
| HLA078 | 1 | 1.62E+08 | 1.62E+08 | ERV_0137786 | MER57-int | I | 1.44 | 12.54 | 8.74 |
| HLA058 | 1 | 1.68E+08 | 1.68E+08 | ERV_0143869 | ERV3-16A3_I-int | I | 0.00 | 2.70 | 300.00 |
| HLA058 | 1 | 1.68E+08 | 1.68E+08 | ERV_0143903 | MSTA,MSTA-int | LIL | 0.53 | 2.69 | 5.10 |
| HLA067 | 1 | 1.7E+08 | 1.7E+08 | ERV_0145224 | THE1B,THE1B-int | LIL | 0.79 | 6.27 | 7.99 |
| HLA069 | 1 | 1.7E+08 | 1.7E+08 | ERV_0145224 | THE1B,THE1B-int | LIL | 5.22 | 31.40 | 6.01 |
| HLA071 | 1 | 1.7E+08 | 1.7E+08 | ERV_0145224 | THE1B,THE1B-int | LIL | 3.16 | 7.43 | 2.35 |
| HLA073 | 1 | 1.7E+08 | 1.7E+08 | ERV_0145224 | THE1B,THE1B-int | LIL | 2.09 | 10.92 | 5.23 |
| HLA076 | 1 | 1.7E+08 | 1.7E+08 | ERV_0145224 | THE1B,THE1B-int | LIL | 2.92 | 18.50 | 6.34 |
| HLA078 | 1 | 1.7E+08 | 1.7E+08 | ERV_0145224 | THE1B,THE1B-int | LIL | 2.20 | 10.65 | 4.84 |
| HLA063 | 1 | 1.71E+08 | 1.71E+08 | ERV_0146196 | PRIMA4-int | I | 1.73 | 5.86 | 3.40 |
| HLA067 | 1 | 1.71E+08 | 1.71E+08 | ERV_0146196 | PRIMA4-int | I | 2.85 | 8.85 | 3.11 |
| HLA065 | 1 | 1.71E+08 | 1.71E+08 | ERV_0146200 | MLT1D,MLT1D-int | LI | 4.44 | 19.81 | 4.46 |
| HLA067 | 1 | 1.71E+08 | 1.71E+08 | ERV_0146200 | MLT1D,MLT1D-int | LI | 66.35 | 182.81 | 2.76 |
| HLA071 | 1 | 1.71E+08 | 1.71E+08 | ERV_0146200 | MLT1D,MLT1D-int | LI | 66.87 | 159.96 | 2.39 |
| HLA073 | 1 | 1.71E+08 | 1.71E+08 | ERV_0146200 | MLT1D,MLT1D-int | LI | 36.57 | 116.54 | 3.19 |
| HLA078 | 1 | 1.73E+08 | 1.73E+08 | ERV_0147624 | HERV9N-int,LTR12,LTR12_ | LIL | 0.01 | 1.75 | 158.70 |
| HLA078 | 1 | 1.73E+08 | 1.73E+08 | ERV_0147625 | HERV9-int | I | 0.01 | 1.75 | 158.70 |
| HLA065 | 1 | 1.73E+08 | 1.73E+08 | ERV_0148108 | ERVL-B4-int,MLT2B1 | LI | 0.07 | 3.33 | 47.45 |
| HLA072 | 1 | 1.73E+08 | 1.73E+08 | ERV_0148108 | ERVL-B4-int,MLT2B1 | LI | 0.50 | 2.13 | 4.29 |
| HLA053 | 1 | 1.73E+08 | 1.73E+08 | ERV_0148199 | PABL_A-int | I | 0.05 | 3.36 | 66.32 |
| HLA069 | 1 | 1.73E+08 | 1.73E+08 | ERV_0148199 | PABL_A-int | I | 0.58 | 2.02 | 3.48 |
| HLA076 | 1 | 1.73E+08 | 1.73E+08 | ERV_0148199 | PABL_A-int | I | 3.14 | 6.51 | 2.07 |
| HLA078 | 1 | 1.73E+08 | 1.73E+08 | ERV_0148199 | PABL_A-int | I | 0.54 | 1.71 | 3.15 |
| HLA066 | 1 | 1.8E+08 | 1.8E+08 | ERV_0153998 | MLT1-int | I | 0.54 | 2.20 | 4.06 |
| HLA061 | 1 | 1.82E+08 | 1.82E+08 | ERV_0156094 | ERVL-B4-int | I | 0.00 | 2.05 | 300.00 |
| HLA078 | 1 | 1.82E+08 | 1.82E+08 | ERV_0156094 | ERVL-B4-int | I | 0.00 | 34.38 | 300.00 |
| HLA071 | 1 | 1.85E+08 | 1.85E+08 | ERV_0157983 | MLT1D,MLT1D-int | LI | 1.08 | 2.64 | 2.46 |
| HLA053 | 1 | 1.87E+08 | 1.87E+08 | ERV_0159933 | HERVH-int,LTR7 | LI | 0.69 | 1.87 | 2.68 |
| HLA065 | 1 | 1.87E+08 | 1.87E+08 | ERV_0159933 | HERVH-int,LTR7 | LI | 0.69 | 1.89 | 2.73 |
| HLA078 | 1 | 1.88E+08 | 1.88E+08 | ERV_0160543 | MER61-int,MER61A | LI | 0.00 | 1.58 | 300.00 |
| HLA066 | 1 | 1.9E+08 | 1.9E+08 | ERV_0162375 | MLTII,MLTII-int | LIL | 0.00 | 3.85 | 300.00 |
| HLA082 | 1 | 1.9E+08 | 1.9E+08 | ERV_0162375 | MLTII,MLTII-int | LIL | 0.00 | 1.75 | 300.00 |
| HLA058 | 1 | 1.91E+08 | 1.91E+08 | ERV_0163486 | MLTIJ-int | I | 0.00 | 4.94 | 300.00 |
| HLA058 | 1 | 1.91E+08 | 1.91E+08 | ERV_0163528 | MLT1A,MLT1A-int | LI | 0.00 | 5.92 | 300.00 |
| HLA058 | 1 | 1.91E+08 | 1.91E+08 | ERV_0163540 | MLT1G3,MLT1G3-int | LI | 0.00 | 3.47 | 300.00 |
| HLA058 | 1 | 1.93E+08 | 1.93E+08 | ERV_0164496 | ERV3-16A3-I-int,LTR16 | LI | 0.00 | 1.66 | 300.00 |
| HLA065 | 1 | 1.94E+08 | 1.94E+08 | ERV_0165789 | ERVL-B4-int | I | 0.00 | 3.04 | 300.00 |
| HLA082 | 1 | 1.94E+08 | 1.94E+08 | ERV_0165789 | ERVL-B4-int | I | 0.00 | 1.53 | 300.00 |
| HLA067 | 1 | 1.96E+08 | 1.96E+08 | ERV_0167468 | LTR53-int | I | 0.00 | 1.61 | 300.00 |
| HLA072 | 1 | 1.96E+08 | 1.96E+08 | ERV_0167468 | LTR53-int | I | 0.25 | 1.80 | 7.20 |
| HLA063 | 1 | 1.97E+08 | 1.97E+08 | ERV_0167859 | HUERS-P1-int | I | 159.08 | 509.20 | 3.20 |
| HLA065 | 1 | 1.97E+08 | 1.97E+08 | ERV_0167861 | PABL_B-int | I | 5.20 | 45.54 | 8.76 |
| HLA067 | 1 | 1.97E+08 | 1.97E+08 | ERV_0167861 | PABL_B-int | I | 8.62 | 45.42 | 5.27 |
| HLA072 | 1 | 1.97E+08 | 1.97E+08 | ERV_0167861 | PABL_B-int | I | 10.26 | 28.52 | 2.78 |
| HLA065 | 1 | 1.97E+08 | 1.97E+08 | ERV_0167863 | MER84-int | I | 0.00 | 2.10 | 300.00 |
| HLA072 | 1 | 1.97E+08 | 1.97E+08 | ERV_0167863 | MER84-int | I | 1.37 | 3.34 | 2.44 |
| HLA082 | 1 | 1.97E+08 | 1.97E+08 | ERV_0167863 | MER84-int | I | 1.22 | 12.87 | 10.56 |
| HLA067 | 1 | 1.97E+08 | 1.97E+08 | ERV_0167920 | THE1B,THE1B-int | LIL | 32.07 | 124.92 | 3.90 |
| HLA067 | 1 | 1.97E+08 | 1.97E+08 | ERV_0167929 | MER4B-int | I | 0.07 | 4.60 | 62.89 |
| HLA067 | 1 | 1.97E+08 | 1.97E+08 | ERV_0167930 | ERVL-E-int | I | 0.00 | 2.92 | 300.00 |
| HLA065 | 1 | 1.97E+08 | 1.97E+08 | ERV_0168018 | THE1B,THE1B-int | LIL | 4.16 | 10.03 | 2.41 |
| HLA067 | 1 | 1.97E+08 | 1.97E+08 | ERV_0168018 | THE1B,THE1B-int | LIL | 22.20 | 78.52 | 3.54 |
| HLA067 | 1 | 1.97E+08 | 1.97E+08 | ERV_0168028 | ERVL-E-int | I | 0.00 | 2.00 | 300.00 |
| HLA061 | 1 | 1.97E+08 | 1.97E+08 | ERV_0168046 | MER21-int | I | 28.39 | 79.13 | 2.79 |
| HLA067 | 1 | 1.97E+08 | 1.97E+08 | ERV_0168046 | MER21-int | I | 17.88 | 72.46 | 4.05 |
| HLA076 | 1 | 1.97E+08 | 1.97E+08 | ERV_0168046 | MER21-int | I | 3.78 | 47.56 | 12.58 |
| HLA053 | 1 | 1.97E+08 | 1.97E+08 | ERV_0168088 | MER4B-int | I | 0.47 | 12.89 | 27.28 |
| HLA065 | 1 | 1.97E+08 | 1.97E+08 | ERV_0168088 | MER4B-int | I | 0.50 | 5.17 | 10.44 |
| HLA067 | 1 | 1.97E+08 | 1.97E+08 | ERV_0168088 | MER4B-int | I | 5.38 | 12.30 | 2.29 |
| HLA069 | 1 | 1.97E+08 | 1.97E+08 | ERV_0168088 | MER4B-int | I | 6.08 | 17.84 | 2.94 |
| HLA076 | 1 | 1.97E+08 | 1.97E+08 | ERV_0168088 | MER4B-int | I | 4.32 | 74.23 | 17.17 |
| HLA078 | 1 | 1.97E+08 | 1.97E+08 | ERV_0168088 | MER4B-int | I | 10.23 | 58.37 | 5.71 |
| HLA082 | 1 | 1.97E+08 | 1.97E+08 | ERV_0168088 | MER4B-int | I | 4.90 | 14.73 | 3.01 |
| HLA053 | 1 | 1.97E+08 | 1.97E+08 | ERV_0168090 | ERVL47-int,LTR47A | LI | 0.47 | 12.89 | 27.28 |
| HLA065 | 1 | 1.97E+08 | 1.97E+08 | ERV_0168090 | ERVL47-int,LTR47A | LI | 0.50 | 5.17 | 10.44 |
| HLA067 | 1 | 1.97E+08 | 1.97E+08 | ERV_0168090 | ERVL47-int,LTR47A | LI | 5.38 | 12.30 | 2.29 |
| HLA069 | 1 | 1.97E+08 | 1.97E+08 | ERV_0168090 | ERVL47-int,LTR47A | LI | 6.08 | 17.84 | 2.94 |
| HLA076 | 1 | 1.97E+08 | 1.97E+08 | ERV_0168090 | ERVL47-int,LTR47A | LI | 4.32 | 74.23 | 17.17 |
| HLA078 | 1 | 1.97E+08 | 1.97E+08 | ERV_0168090 | ERVL47-int,LTR47A | LI | 10.23 | 58.37 | 5.71 |
| HLA082 | 1 | 1.97E+08 | 1.97E+08 | ERV_0168090 | ERVL47-int,LTR47A | LI | 4.90 | 14.73 | 3.01 |
| HLA071 | 1 | 2.04E+08 | 2.04E+08 | ERV_0175026 | MER101-int | I | 0.13 | 1.91 | 15.03 |
| HLA073 | 1 | 2.04E+08 | 2.04E+08 | ERV_0175026 | MER101-int | I | 0.13 | 1.79 | 13.67 |
| HLA071 | 1 | 2.04E+08 | 2.04E+08 | ERV_0175028 | LOR1-int | I | 0.13 | 1.91 | 15.03 |
| HLA073 | 1 | 2.04E+08 | 2.04E+08 | ERV_0175028 | LOR1-int | I | 0.13 | 1.79 | 13.67 |
| HLA071 | 1 | 2.04E+08 | 2.04E+08 | ERV_0175030 | LOR1-int | I | 0.00 | 2.16 | 300.00 |
| HLA073 | 1 | 2.04E+08 | 2.04E+08 | ERV_0175030 | LOR1-int | I | 0.00 | 1.63 | 300.00 |
| HLA065 | 1 | 2.04E+08 | 2.04E+08 | ERV_0175089 | MLT1C,MLT1C-int | LIL | 1.48 | 8.90 | 6.01 |
| HLA067 | 1 | 2.04E+08 | 2.04E+08 | ERV_0175089 | MLT1C,MLT1C-int | LIL | 2.25 | 5.85 | 2.60 |
| HLA072 | 1 | 2.04E+08 | 2.04E+08 | ERV_0175089 | MLT1C,MLT1C-int | LIL | 2.70 | 6.81 | 2.53 |
| HLA073 | 1 | 2.04E+08 | 2.04E+08 | ERV_0175089 | MLT1C,MLT1C-int | LIL | 0.72 | 1.97 | 2.75 |
| HLA078 | 1 | 2.04E+08 | 2.04E+08 | ERV_0175089 | MLT1C,MLT1C-int | LIL | 0.90 | 2.54 | 2.83 |
| HLA065 | 1 | 2.04E+08 | 2.04E+08 | ERV_0175090 | MLT1C-int | I | 1.48 | 8.90 | 6.01 |
| HLA067 | 1 | 2.04E+08 | 2.04E+08 | ERV_0175090 | MLT1C-int | I | 2.25 | 5.85 | 2.60 |
| HLA072 | 1 | 2.04E+08 | 2.04E+08 | ERV_0175090 | MLT1C-int | I | 2.70 | 6.81 | 2.53 |
| HLA073 | 1 | 2.04E+08 | 2.04E+08 | ERV_0175090 | MLT1C-int | I | 0.72 | 1.97 | 2.75 |
| HLA078 | 1 | 2.04E+08 | 2.04E+08 | ERV_0175090 | MLT1C-int | I | 0.90 | 2.54 | 2.83 |
| HLA053 | 1 | 2.06E+08 | 2.06E+08 | ERV_0176824 | HERVK9-int,MER9a2 | LIL | 0.07 | 6.66 | 91.55 |
| HLA058 | 1 | 2.06E+08 | 2.06E+08 | ERV_0176824 | HERVK9-int,MER9a2 | LIL | 1.66 | 3.66 | 2.20 |
| HLA065 | 1 | 2.06E+08 | 2.06E+08 | ERV_0176824 | HERVK9-int,MER9a2 | LIL | 1.38 | 2.90 | 2.10 |
| HLA067 | 1 | 2.06E+08 | 2.06E+08 | ERV_0176824 | HERVK9-int,MER9a2 | LIL | 1.00 | 9.17 | 9.20 |
| HLA078 | 1 | 2.06E+08 | 2.06E+08 | ERV_0176824 | HERVK9-int,MER9a2 | LIL | 0.74 | 6.31 | 8.56 |
| HLA058 | 1 | 2.06E+08 | 2.06E+08 | ERV_0176962 | Harlequin-int,LTR2B | LIL | 9.32 | 22.55 | 2.42 |
| HLA065 | 1 | 2.06E+08 | 2.06E+08 | ERV_0176962 | Harlequin-int,LTR2B | LIL | 7.69 | 23.62 | 3.07 |
| HLA078 | 1 | 2.07E+08 | 2.07E+08 | ERV_0178018 | HERV3-int,LTR61 | LI | 0.00 | 2.19 | 300.00 |
| HLA063 | 1 | 2.07E+08 | 2.07E+08 | ERV_0178111 | MLT1J-int | I | 0.52 | 2.34 | 4.47 |
| HLA067 | 1 | 2.11E+08 | 2.11E+08 | ERV_0181638 | HERVK11-int,MER11C | LI | 2.73 | 6.97 | 2.55 |
| HLA069 | 1 | 2.11E+08 | 2.11E+08 | ERV_0181638 | HERVK11-int,MER11C | LI | 0.59 | 2.26 | 3.84 |
| HLA076 | 1 | 2.11E+08 | 2.11E+08 | ERV_0181638 | HERVK11-int,MER11C | LI | 0.88 | 1.96 | 2.22 |
| HLA078 | 1 | 2.11E+08 | 2.11E+08 | ERV_0181638 | HERVK11-int,MER11C | LI | 1.18 | 4.41 | 3.73 |
| HLA078 | 1 | 2.14E+08 | 2.14E+08 | ERV_0183758 | THE1B,THE1B-int | LIL | 0.00 | 6.10 | 300.00 |
| HLA065 | 1 | 2.14E+08 | 2.14E+08 | ERV_0184071 | MLT1H1,MLT1H1-int | LI | 1.11 | 11.27 | 10.14 |
| HLA066 | 1 | 2.14E+08 | 2.14E+08 | ERV_0184071 | MLT1H1,MLT1H1-int | LI | 0.90 | 2.70 | 3.01 |
| HLA067 | 1 | 2.14E+08 | 2.14E+08 | ERV_0184071 | MLT1H1,MLT1H1-int | LI | 0.59 | 4.04 | 6.84 |
| HLA072 | 1 | 2.14E+08 | 2.14E+08 | ERV_0184071 | MLT1H1,MLT1H1-int | LI | 1.03 | 7.03 | 6.83 |
| HLA078 | 1 | 2.18E+08 | 2.18E+08 | ERV_0187236 | MLT1J,MLTIJ-int | LI | 0.00 | 1.70 | 300.00 |
| HLA061 | 1 | 2.19E+08 | 2.19E+08 | ERV_0188579 | THE1A,THE1A-int | LI | 0.77 | 1.61 | 2.10 |
| HLA063 | 1 | 2.19E+08 | 2.19E+08 | ERV_0188579 | THE1A,THE1A-int | LI | 0.36 | 1.94 | 5.37 |
| HLA067 | 1 | 2.19E+08 | 2.19E+08 | ERV_0188579 | THE1A,THE1A-int | LI | 0.67 | 3.31 | 4.92 |
| HLA078 | 1 | 2.19E+08 | 2.19E+08 | ERV_0188579 | THE1A,THE1A-int | LI | 1.14 | 8.53 | 7.50 |
| HLA078 | 1 | 2.2E+08 | 2.2E+08 | ERV_0189629 | MLT1J2,MLT1J2-int | LIL | 0.00 | 6.87 | 300.00 |
| HLA058 | 1 | 2.23E+08 | 2.23E+08 | ERV_0192066 | HERVP71A-int,LTR71B | LI | 0.14 | 1.74 | 12.69 |
| HLA076 | 1 | 2.25E+08 | 2.25E+08 | ERV_0193956 | ERVL-B4-int | I | 0.00 | 1.74 | 300.00 |
| HLA065 | 1 | 2.28E+08 | 2.28E+08 | ERV_0196301 | ERVL-int | I | 0.00 | 1.61 | 300.00 |
| HLA058 | 1 | 2.28E+08 | 2.28E+08 | ERV_0196345 | Harlequin-int,LTR2 | LI | 2.89 | 10.13 | 3.50 |
| HLA069 | 1 | 2.28E+08 | 2.28E+08 | ERV_0196345 | Harlequin-int,LTR2 | LI | 4.11 | 8.24 | 2.00 |
| HLA071 | 1 | 2.28E+08 | 2.28E+08 | ERV_0196345 | Harlequin-int,LTR2 | LI | 3.71 | 8.35 | 2.25 |
| HLA072 | 1 | 2.28E+08 | 2.28E+08 | ERV_0196345 | Harlequin-int,LTR2 | LI | 4.24 | 10.43 | 2.46 |
| HLA078 | 1 | 2.28E+08 | 2.28E+08 | ERV_0196345 | Harlequin-int,LTR2 | LI | 1.61 | 10.69 | 6.63 |
| HLA061 | 1 | 2.34E+08 | 2.34E+08 | ERV_0201504 | MLT1A0,MLT1A0-int | LI | 0.43 | 1.75 | 4.11 |
| HLA078 | 1 | 2.35E+08 | 2.35E+08 | ERV_0202266 | HERV17-int | I | 0.00 | 1.69 | 300.00 |
| HLA076 | 1 | 2.36E+08 | 2.36E+08 | ERV_0204120 | THE1A,THE1A-int | LIL | 0.08 | 3.07 | 36.69 |
| HLA078 | 1 | 2.36E+08 | 2.36E+08 | ERV_0204120 | THE1A,THE1A-int | LIL | 0.24 | 1.57 | 6.53 |
| HLA065 | 1 | 2.36E+08 | 2.36E+08 | ERV_0204212 | MLT1K,MLT1K-int | LI | 1.08 | 2.55 | 2.36 |
| HLA069 | 1 | 2.36E+08 | 2.36E+08 | ERV_0204212 | MLT1K,MLT1K-int | LI | 0.00 | 2.08 | 300.00 |
| HLA082 | 1 | 2.37E+08 | 2.37E+08 | ERV_0204667 | MLT1A,MLT1A-int | LI | 0.00 | 1.71 | 300.00 |
| HLA078 | 1 | 2.4E+08 | 2.4E+08 | ERV_0207418 | THE1B,THE1B-int | LI | 0.00 | 1.83 | 300.00 |
| HLA067 | 1 | 2.4E+08 | 2.4E+08 | ERV_0208012 | E RV3-16A3-1 -i nt, LTR 16 | LI | 1.29 | 3.12 | 2.41 |
| HLA076 | 1 | 2.4E+08 | 2.4E+08 | ERV_0208012 | E RV3-16A3-1 -i nt, LTR 16 | LI | 0.09 | 4.64 | 52.87 |
| HLA053 | 1 | 2.44E+08 | 2.44E+08 | ERV_0211765 | MER50,MER50-int | LI | 0.16 | 1.54 | 9.65 |
| HLA065 | 1 | 2.45E+08 | 2.45E+08 | ERV_0212111 | MER68,MER68-int | LI | 1.18 | 3.85 | 3.25 |
| HLA067 | 1 | 2.45E+08 | 2.45E+08 | ERV_0212111 | MER68,MER68-int | LI | 0.67 | 1.83 | 2.74 |
| HLA078 | 1 | 2.45E+08 | 2.45E+08 | ERV_0212111 | MER68,MER68-int | LI | 0.51 | 1.67 | 3.29 |
| HLA065 | 1 | 2.45E+08 | 2.45E+08 | ERV_0212115 | MER68,MER68-int | LI | 1.18 | 3.85 | 3.25 |
| HLA067 | 1 | 2.45E+08 | 2.45E+08 | ERV_0212115 | MER68,MER68-int | LI | 0.67 | 1.83 | 2.74 |
| HLA078 | 1 | 2.45E+08 | 2.45E+08 | ERV_0212115 | MER68,MER68-int | LI | 0.51 | 1.67 | 3.29 |
| HLA065 | 1 | 2.45E+08 | 2.45E+08 | ERV_0212118 | MLT1-int | I | 1.18 | 3.85 | 3.25 |
| HLA067 | 1 | 2.45E+08 | 2.45E+08 | ERV_0212118 | MLT1-int | I | 0.67 | 1.83 | 2.74 |
| HLA078 | 1 | 2.45E+08 | 2.45E+08 | ERV_0212118 | MLT1-int | I | 0.51 | 1.67 | 3.29 |
| HLA072 | 1 | 2.46E+08 | 2.46E+08 | ERV_0213461 | HUERS-P3-int,LTR9 | LIL | 0.14 | 4.59 | 32.62 |
| HLA053 | 1 | 2.47E+08 | 2.47E+08 | ERV_0214472 | HERVK14C-int,LTR14C | LI | 0.69 | 1.73 | 2.50 |
| HLA058 | 1 | 2.47E+08 | 2.47E+08 | ERV_0214472 | HERVK14C-int,LTR14C | LI | 0.47 | 1.98 | 4.22 |
| HLA061 | 1 | 2.47E+08 | 2.47E+08 | ERV_0214472 | HERVK14C-int,LTR14C | LI | 0.32 | 4.01 | 12.45 |
| HLA065 | 1 | 2.47E+08 | 2.47E+08 | ERV_0214472 | HERVK14C-int,LTR14C | LI | 0.55 | 2.34 | 4.29 |
| HLA069 | 1 | 2.47E+08 | 2.47E+08 | ERV_0214472 | HERVK14C-int,LTR14C | LI | 0.18 | 1.56 | 8.66 |
| HLA071 | 1 | 2.47E+08 | 2.47E+08 | ERV_0214472 | HERVK14C-int,LTR14C | LI | 1.08 | 3.31 | 3.06 |
| HLA078 | 1 | 2.47E+08 | 2.47E+08 | ERV_0214472 | HERVK14C-int,LTR14C | LI | 0.57 | 3.06 | 5.40 |
| HLA053 | 1 | 2.47E+08 | 2.47E+08 | ERV_0214473 | HERVK14C-int | I | 0.69 | 1.73 | 2.50 |
| HLA058 | 1 | 2.47E+08 | 2.47E+08 | ERV_0214473 | HERVK14C-int | I | 0.47 | 1.98 | 4.22 |
| HLA061 | 1 | 2.47E+08 | 2.47E+08 | ERV_0214473 | HERVK14C-int | I | 0.32 | 4.01 | 12.45 |
| HLA065 | 1 | 2.47E+08 | 2.47E+08 | ERV_0214473 | HERVK14C-int | I | 0.55 | 2.34 | 4.29 |
| HLA069 | 1 | 2.47E+08 | 2.47E+08 | ERV_0214473 | HERVK14C-int | I | 0.18 | 1.56 | 8.66 |
| HLA071 | 1 | 2.47E+08 | 2.47E+08 | ERV_0214473 | HERVK14C-int | I | 1.08 | 3.31 | 3.06 |
| HLA078 | 1 | 2.47E+08 | 2.47E+08 | ERV_0214473 | HERVK14C-int | I | 0.57 | 3.06 | 5.40 |
| HLA058 | 1 | 2.47E+08 | 2.47E+08 | ERV_0214495 | HERVL40-int | I | 1.11 | 2.72 | 2.45 |
| HLA061 | 1 | 2.47E+08 | 2.47E+08 | ERV_0214495 | HERVL40-int | I | 0.58 | 1.78 | 3.08 |
| HLA065 | 1 | 2.47E+08 | 2.47E+08 | ERV_0214495 | HERVL40-int | I | 0.72 | 1.94 | 2.70 |
| HLA072 | 1 | 2.47E+08 | 2.47E+08 | ERV_0214495 | HERVL40-int | I | 0.50 | 5.82 | 11.68 |
| HLA078 | 1 | 2.47E+08 | 2.47E+08 | ERV_0214495 | HERVL40-int | I | 0.28 | 1.64 | 5.85 |
| HLA058 | 1 | 2.47E+08 | 2.47E+08 | ERV_0214642 | MLT1B,MLT1B-int | LIL | 0.53 | 1.52 | 2.87 |
| HLA065 | 1 | 2.47E+08 | 2.47E+08 | ERV_0214642 | MLT1B,MLT1B-int | LIL | 1.12 | 2.56 | 2.29 |
| HLA058 | 1 | 2.47E+08 | 2.47E+08 | ERV_0214643 | MLT1B-int | I | 0.53 | 1.52 | 2.87 |
| HLA065 | 1 | 2.47E+08 | 2.47E+08 | ERV_0214643 | MLT1B-int | I | 1.12 | 2.56 | 2.29 |
| HLA069 | 1 | 2.48E+08 | 2.48E+08 | ERV_0214828 | MER89-int | I | 0.00 | 2.11 | 300.00 |
| HLA053 | 1 | 2.49E+08 | 2.49E+08 | ERV_0215688 | MER4-int,MER4A1 | LIL | 0.15 | 3.44 | 22.40 |
| HLA067 | 1 | 2.49E+08 | 2.49E+08 | ERV_0215688 | MER4-int,MER4A1 | LIL | 0.00 | 1.92 | 300.00 |
| HLA069 | 1 | 2.49E+08 | 2.49E+08 | ERV_0215688 | MER4-int,MER4A1 | LIL | 0.00 | 8.89 | 300.00 |
| HLA071 | 1 | 2.49E+08 | 2.49E+08 | ERV_0215688 | MER4-int,MER4A1 | LIL | 0.64 | 6.35 | 9.99 |
| HLA073 | 1 | 2.49E+08 | 2.49E+08 | ERV_0215688 | MER4-int,MER4A1 | LIL | 0.00 | 1.75 | 300.00 |
| HLA078 | 1 | 2.49E+08 | 2.49E+08 | ERV_0215688 | MER4-int,MER4A1 | LIL | 0.00 | 2.58 | 300.00 |
| HLA069 | 1 | 906474 | 906556 | ERV_0216289 | HERV16-int | I | 0.12 | 2.99 | 24.50 |
| HLA065 | 1 | 1275713 | 1280788 | ERV_0216432 | MER4B,MER4B-int | LIL | 0.52 | 2.61 | 5.00 |
| HLA071 | 1 | 1275713 | 1280788 | ERV_0216432 | MER4B,MER4B-int | LIL | 2.93 | 10.86 | 3.71 |
| HLA073 | 1 | 1275713 | 1280788 | ERV_0216432 | MER4B,MER4B-int | LIL | 2.52 | 8.41 | 3.34 |
| HLA078 | 1 | 1275713 | 1280788 | ERV_0216432 | MER4B,MER4B-int | LIL | 1.30 | 5.16 | 3.97 |
| HLA063 | 1 | 1384033 | 1384156 | ERV_0216462 | MER66-int | I | 2.37 | 4.83 | 2.03 |
| HLA078 | 1 | 1384033 | 1384156 | ERV_0216462 | MER66-int | I | 0.88 | 3.15 | 3.57 |
| HLA058 | 1 | 1412252 | 1418852 | ERV_0216485 | Harlequin-int,LTR2B | LIL | 0.33 | 3.60 | 11.08 |
| HLA063 | 1 | 1412252 | 1418852 | ERV_0216485 | Harlequin-int,LTR2B | LIL | 0.16 | 8.58 | 53.89 |
| HLA072 | 1 | 1412252 | 1418852 | ERV_0216485 | Harlequin-int,LTR2B | LIL | 0.81 | 5.83 | 7.19 |
| HLA073 | 1 | 1412252 | 1418852 | ERV_0216485 | Harlequin-int,LTR2B | LIL | 0.07 | 3.30 | 48.16 |
| HLA076 | 1 | 1412252 | 1418852 | ERV_0216485 | Harlequin-int,LTR2B | LIL | 0.73 | 2.16 | 2.97 |
| HLA078 | 1 | 1412252 | 1418852 | ERV_0216485 | Harlequin-int,LTR2B | LIL | 0.05 | 3.73 | 78.21 |
| HLA078 | 1 | 1498784 | 1511404 | ERV_0216552 | HERVIP10F-int,LTR10A | LIL | 0.23 | 1.66 | 7.27 |
| HLA078 | 1 | 1502812 | 1502909 | ERV_0216563 | HERV9N-int | I | 0.23 | 1.66 | 7.27 |
| HLA067 | 1 | 1911129 | 1911705 | ERV_0216860 | ERV3-16A3_I-int,ERV3-16A3_LTR | LI | 1.35 | 3.37 | 2.49 |
| HLA069 | 1 | 1911129 | 1911705 | ERV_0216860 | ERV3-16A3_I-int,ERV3-16A3_LTR | LI | 0.00 | 2.37 | 300.00 |
| HLA073 | 1 | 1911129 | 1911705 | ERV_0216860 | ERV3-16A3_I-int,ERV3-16A3_LTR | LI | 0.79 | 3.15 | 4.01 |
| HLA066 | 1 | 2035564 | 2038368 | ERV_0216920 | ERVL-E-int | I | 1.06 | 2.56 | 2.41 |
| HLA053 | 1 | 2383456 | 2383681 | ERV_0217092 | ERVL-E-int | I | 1.19 | 5.15 | 4.32 |
| HLA058 | 1 | 2383456 | 2383681 | ERV_0217092 | ERVL-E-int | I | 0.00 | 3.48 | 300.00 |
| HLA066 | 1 | 2383456 | 2383681 | ERV_0217092 | ERVL-E-int | I | 0.00 | 4.32 | 300.00 |
| HLA067 | 1 | 2383456 | 2383681 | ERV_0217092 | ERVL-E-int | I | 0.00 | 5.17 | 300.00 |
| HLA073 | 1 | 2383456 | 2383681 | ERV_0217092 | ERVL-E-int | I | 0.10 | 2.08 | 21.27 |
| HLA078 | 1 | 2383456 | 2383681 | ERV_0217092 | ERVL-E-int | I | 0.04 | 2.44 | 61.11 |
| HLA073 | 1 | 3658269 | 3662728 | ERV_0217438 | MER31-int,MER34C2 | LIL | 0.00 | 5.72 | 300.00 |
| HLA078 | 1 | 3658269 | 3662728 | ERV_0217438 | MER31-int,MER34C2 | LIL | 0.00 | 1.90 | 300.00 |
| HLA072 | 1 | 7790172 | 7792441 | ERV_0220051 | THE1C,THE1C-int | LIL | 0.37 | 1.83 | 5.01 |
| HLA058 | 1 | 10137357 | 10137879 | ERV_0222170 | ERVL-int | I | 0.33 | 2.24 | 6.87 |
| HLA058 | 1 | 16602708 | 16603618 | ERV_0227869 | ERVL-E-int,LTR33 | LI | 0.00 | 1.72 | 300.00 |
| HLA065 | 1 | 16602708 | 16603618 | ERV_0227869 | ERVL-E-int,LTR33 | LI | 4.11 | 11.98 | 2.91 |
| HLA066 | 1 | 16602708 | 16603618 | ERV_0227869 | ERVL-E-int,LTR33 | LI | 0.79 | 1.64 | 2.07 |
| HLA067 | 1 | 16602708 | 16603618 | ERV_0227869 | ERVL-E-int,LTR33 | LI | 0.27 | 3.33 | 12.47 |
| HLA069 | 1 | 16602708 | 16603618 | ERV_0227869 | ERVL-E-int,LTR33 | LI | 3.92 | 10.36 | 2.64 |
| HLA073 | 1 | 16602708 | 16603618 | ERV_0227869 | ERVL-E-int,LTR33 | LI | 1.65 | 7.53 | 4.55 |
| HLA076 | 1 | 16602708 | 16603618 | ERV_0227869 | ERVL-E-int,LTR33 | LI | 1.79 | 5.76 | 3.23 |
| HLA078 | 1 | 16602708 | 16603618 | ERV_0227869 | ERVL-E-int,LTR33 | LI | 5.94 | 16.77 | 2.82 |
| HLA063 | 1 | 19294272 | 19295902 | ERV_0230263 | HUERS-P3-int,LTR9D | LI | 6.67 | 21.82 | 3.27 |
| HLA067 | 1 | 19294272 | 19295902 | ERV_0230263 | HUERS-P3-int,LTR9D | LI | 5.55 | 11.12 | 2.00 |
| HLA069 | 1 | 19294272 | 19295902 | ERV_0230263 | HUERS-P3-int,LTR9D | LI | 6.89 | 14.45 | 2.10 |
| HLA078 | 1 | 19294272 | 19295902 | ERV_0230263 | HUERS-P3-int,LTR9D | LI | 3.19 | 9.88 | 3.10 |
| HLA053 | 1 | 20598507 | 20603714 | ERV_0231501 | MLT1A,MLT1A-int | LIL | 1.37 | 10.19 | 7.42 |
| HLA065 | 1 | 20598507 | 20603714 | ERV_0231501 | MLT1A,MLT1A-int | LIL | 1.93 | 8.10 | 4.19 |
| HLA067 | 1 | 20598507 | 20603714 | ERV_0231501 | MLT1A,MLT1A-int | LIL | 4.88 | 11.97 | 2.45 |
| HLA078 | 1 | 21450021 | 21455400 | ERV_0232188 | MLT1E3,MLT1E3-int | LIL | 0.34 | 2.33 | 6.90 |
| HLA063 | 1 | 24162011 | 24164755 | ERV_0234944 | MLT1E2,MLT1E2-int | LI | 8.29 | 21.43 | 2.59 |
| HLA065 | 1 | 24162011 | 24164755 | ERV_0234944 | MLT1E2,MLT1E2-int | LI | 4.63 | 22.97 | 4.96 |
| HLA066 | 1 | 24162011 | 24164755 | ERV_0234944 | MLT1E2,MLT1E2-int | LI | 1.68 | 5.14 | 3.06 |
| HLA067 | 1 | 24162011 | 24164755 | ERV_0234944 | MLT1E2,MLT1E2-int | LI | 8.04 | 30.41 | 3.78 |
| HLA072 | 1 | 24162011 | 24164755 | ERV_0234944 | MLT1E2,MLT1E2-int | LI | 4.63 | 18.33 | 3.96 |
| HLA063 | 1 | 24165062 | 24165488 | ERV_0234949 | MLTIE2-int | I | 8.45 | 20.44 | 2.42 |
| HLA065 | 1 | 24165062 | 24165488 | ERV_0234949 | MLTIE2-int | I | 0.00 | 3.49 | 300.00 |
| HLA067 | 1 | 24165062 | 24165488 | ERV_0234949 | MLT1E2-int | I | 8.83 | 28.34 | 3.21 |
| HLA069 | 1 | 24165062 | 24165488 | ERV_0234949 | MLT1E2-int | I | 1.37 | 7.10 | 5.18 |
| HLA072 | 1 | 24165062 | 24165488 | ERV_0234949 | MLTIE2-int | I | 7.02 | 14.99 | 2.14 |
| HLA066 | 1 | 25263315 | 25265225 | ERV_0236056 | THE1B,THE1B-int | LI | 2.16 | 5.05 | 2.34 |
| HLA067 | 1 | 25263315 | 25265225 | ERV_0236056 | THE1B,THE1B-int | LI | 1.98 | 7.43 | 3.76 |
| HLA072 | 1 | 25263315 | 25265225 | ERV_0236056 | THE1B,THE1B-int | LI | 7.42 | 19.42 | 2.62 |
| HLA078 | 1 | 25263315 | 25265225 | ERV_0236056 | THE1B,THE1B-int | LI | 3.69 | 9.58 | 2.60 |
| HLA065 | 1 | 25333641 | 25335551 | ERV_0236135 | THE1B,THE1B-int | LI | 3.27 | 9.59 | 2.93 |
| HLA066 | 1 | 25333641 | 25335551 | ERV_0236135 | THE1B,THE1B-int | LI | 2.33 | 5.39 | 2.32 |
| HLA067 | 1 | 25333641 | 25335551 | ERV_0236135 | THE1B,THE1B-int | LI | 1.09 | 2.59 | 2.38 |
| HLA078 | 1 | 25333641 | 25335551 | ERV_0236135 | THE1B,THE1B-int | LI | 0.48 | 3.22 | 6.69 |
| HLA072 | 1 | 25854336 | 25855305 | ERV_0236655 | MLT1F,MLT1F-int | LI | 0.00 | 1.66 | 300.00 |
| HLA082 | 1 | 26802679 | 26809760 | ERV_0237631 | MLT1J2,MLT1J2-int | LI | 0.23 | 4.96 | 21.36 |
| HLA053 | 1 | 26933800 | 26934662 | ERV_0237780 | THE1C,THE1C-int | LIL | 4.44 | 9.88 | 2.22 |
| HLA058 | 1 | 26933800 | 26934662 | ERV_0237780 | THE1C,THE1C-int | LIL | 4.31 | 9.55 | 2.22 |
| HLA076 | 1 | 26933800 | 26934662 | ERV_0237780 | THE1C,THE1C-int | LIL | 4.11 | 9.58 | 2.33 |
| HLA078 | 1 | 26933800 | 26934662 | ERV_0237780 | THE1C,THE1C-int | LIL | 4.98 | 10.68 | 2.15 |
| HLA078 | 1 | 27815788 | 27817699 | ERV_0238711 | MER21-int | I | 0.16 | 3.51 | 22.46 |
| HLA061 | 1 | 28586907 | 28589810 | ERV_0239574 | Harlequin-int,LTR2B | LI | 0.38 | 1.51 | 3.92 |
| HLA078 | 1 | 31453515 | 31457948 | ERV_0242310 | HERVIP10F-int,LTR10A | LI | 0.13 | 29.74 | 221.97 |
| HLA071 | 1 | 31467354 | 31471933 | ERV_0242312 | HERVIP10F-int,LTR10A | LI | 0.28 | 2.61 | 9.17 |
| HLA078 | 1 | 31467354 | 31471933 | ERV_0242312 | HERVIP10F-int,LTR10A | LI | 0.22 | 41.43 | 184.92 |
| HLA078 | 1 | 31472400 | 31474977 | ERV_0242316 | THE1A,THE1A-int | LIL | 0.00 | 4.70 | 300.00 |
| HLA058 | 1 | 32141732 | 32141852 | ERV_0242926 | LOR1-int | I | 0.00 | 2.66 | 300.00 |
| HLA072 | 1 | 32141732 | 32141852 | ERV_0242926 | LOR1-int | I | 0.06 | 1.88 | 32.29 |
| HLA078 | 1 | 32141732 | 32141852 | ERV_0242926 | LOR1-int | I | 0.00 | 1.62 | 300.00 |
| HLA067 | 1 | 32928071 | 32931352 | ERV_0243767 | ERVL-E-int | I | 1.05 | 2.78 | 2.64 |
| HLA078 | 1 | 32928071 | 32931352 | ERV_0243767 | ERVL-E-int | I | 0.94 | 3.03 | 3.21 |
| HLA072 | 1 | 33283860 | 33286875 | ERV_0244094 | LTR37-int | I | 0.56 | 1.76 | 3.15 |
| HLA067 | 1 | 35271325 | 35271364 | ERV_0245545 | LTR57-int | I | 0.00 | 1.56 | 300.00 |
| HLA058 | 1 | 38977684 | 38979895 | ERV_0248587 | HERV9N-int,LTR12 | LI | 1.00 | 5.06 | 5.08 |
| HLA065 | 1 | 43246702 | 43251577 | ERV_0252288 | LOR1-int,LOR1b | LI | 1.19 | 3.06 | 2.57 |
| HLA067 | 1 | 43246702 | 43251577 | ERV_0252288 | LOR1-int,LOR1b | LI | 9.41 | 19.03 | 2.02 |
| HLA065 | 1 | 43246769 | 43250804 | ERV_0252289 | LOR1-int | I | 1.19 | 3.06 | 2.57 |
| HLA067 | 1 | 43246769 | 43250804 | ERV_0252289 | LOR1-int | I | 9.41 | 19.03 | 2.02 |
| HLA065 | 1 | 43761082 | 43765435 | ERV_0252707 | MLT1J2,MLT1J2-int | LI | 0.48 | 1.91 | 3.96 |
| HLA072 | 1 | 43761082 | 43765435 | ERV_0252707 | MLT1J2,MLT1J2-int | LI | 0.28 | 2.64 | 9.39 |
| HLA058 | 1 | 45083697 | 45086348 | ERV_0253755 | MSTA,MSTA-int | LIL | 0.17 | 2.23 | 13.13 |
| HLA061 | 1 | 45083697 | 45086348 | ERV_0253755 | MSTA,MSTA-int | LIL | 0.47 | 4.24 | 9.06 |
| HLA053 | 1 | 45222079 | 45229003 | ERV_0253857 | ERVL-E-int,LTR41 | LI | 0.16 | 1.51 | 9.63 |
| HLA071 | 1 | 45222079 | 45229003 | ERV_0253857 | ERVL-E-int,LTR41 | LI | 0.50 | 2.23 | 4.48 |
| HLA078 | 1 | 45222079 | 45229003 | ERV_0253857 | ERVL-E-int,LTR41 | LI | 0.01 | 6.53 | 545.16 |
| HLA058 | 1 | 45575881 | 45580362 | ERV_0254173 | MER61-int | I | 0.57 | 3.29 | 5.72 |
| HLA069 | 1 | 45575881 | 45580362 | ERV_0254173 | MER61-int | I | 0.19 | 1.91 | 9.90 |
| HLA065 | 1 | 45896768 | 45899211 | ERV_0254500 | MER21-int | I | 0.23 | 2.44 | 10.74 |
| HLA058 | 1 | 46372033 | 46373850 | ERV_0254932 | MSTC,MSTC-int | LI | 0.27 | 1.62 | 6.04 |
| HLA061 | 1 | 47162601 | 47164328 | ERV_0255410 | MLT1D,MLT1D-int | LIL | 0.19 | 9.58 | 50.44 |
| HLA061 | 1 | 47164329 | 47164452 | ERV_0255411 | MLT1D-int | I | 0.00 | 1.67 | 300.00 |
| HLA072 | 1 | 47393395 | 47393603 | ERV_0255621 | HERV35I-int | I | 0.00 | 6.54 | 300.00 |
| HLA072 | 1 | 47393602 | 47393844 | ERV_0255622 | MER41-int,MER41B | LI | 0.00 | 6.54 | 300.00 |
| HLA065 | 1 | 51861646 | 51862212 | ERV_0258935 | MER57A-int | I | 0.00 | 3.29 | 300.00 |
| HLA078 | 1 | 51861646 | 51862212 | ERV_0258935 | MER57A-int | I | 0.00 | 2.07 | 300.00 |
| HLA067 | 1 | 54164134 | 54166960 | ERV_0261073 | THE1A,THE1A-int | LIL | 0.82 | 1.72 | 2.10 |
| HLA072 | 1 | 54164134 | 54166960 | ERV_0261073 | THE1A,THE1A-int | LIL | 0.68 | 1.89 | 2.79 |
| HLA073 | 1 | 54164134 | 54166960 | ERV_0261073 | THE1A,THE1A-int | LIL | 0.25 | 2.06 | 8.08 |
| HLA078 | 1 | 54164134 | 54166960 | ERV_0261073 | THE1A,THE1A-int | LIL | 0.61 | 1.99 | 3.28 |
| HLA072 | 1 | 56873115 | 56873184 | ERV_0263370 | ERVL-E-int | I | 1.39 | 14.75 | 10.60 |
| HLA073 | 1 | 56873115 | 56873184 | ERV_0263370 | ERVL-E-int | I | 1.00 | 3.86 | 3.86 |
| HLA078 | 1 | 56968918 | 56969527 | ERV_0263455 | MLT1H,MLT1H-int | LI | 0.00 | 1.99 | 300.00 |
| HLA066 | 1 | 58221070 | 58221140 | ERV_0264536 | MER4-int | I | 0.00 | 1.63 | 300.00 |
| HLA072 | 1 | 58221070 | 58221140 | ERV_0264536 | MER4-int | I | 0.00 | 1.64 | 300.00 |
| HLA067 | 1 | 59785413 | 59787672 | ERV_0265874 | THE1B,THE1B-int | LIL | 0.18 | 3.70 | 20.36 |
| HLA069 | 1 | 59785413 | 59787672 | ERV_0265874 | THE1B,THE1B-int | LIL | 0.63 | 2.42 | 3.84 |
| HLA071 | 1 | 59785413 | 59787672 | ERV_0265874 | THE1B,THE1B-int | LIL | 1.02 | 2.60 | 2.54 |
| HLA072 | 1 | 59785413 | 59787672 | ERV_0265874 | THE1B,THE1B-int | LIL | 0.35 | 2.18 | 6.30 |
| HLA073 | 1 | 59785413 | 59787672 | ERV_0265874 | THE1B,THE1B-int | LIL | 0.62 | 1.68 | 2.69 |
| HLA053 | 1 | 59888378 | 59890653 | ERV_0265938 | THE1B,THE1B-int | LIL | 1.31 | 3.99 | 3.04 |
| HLA063 | 1 | 61924481 | 61925948 | ERV_0267469 | LTR37-int,LTR37B | LI | 51.40 | 112.17 | 2.18 |
| HLA063 | 1 | 64974575 | 64976920 | ERV_0269958 | ERV3-16A3_I-int,LTR16B1 | LI | 0.34 | 2.59 | 7.70 |
| HLA067 | 1 | 64974575 | 64976920 | ERV_0269958 | ERV3-16A3_I-int,LTR16B1 | LI | 1.16 | 2.52 | 2.19 |
| HLA063 | 1 | 65581296 | 65582537 | ERV_0270527 | MLT1F2,MLT1F2-int | LI | 0.88 | 2.59 | 2.96 |
| HLA065 | 1 | 72636642 | 72636752 | ERV_0274840 | HERVIP10FH-int | I | 0.00 | 2.24 | 300.00 |
| HLA078 | 1 | 72636642 | 72636752 | ERV_0274840 | HERVIP10FH-int | I | 0.00 | 1.57 | 300.00 |
| HLA065 | 1 | 74725239 | 74727410 | ERV_0275834 | THE1B,THE1B-int | LIL | 1.29 | 7.58 | 5.87 |
| HLA076 | 1 | 74725239 | 74727410 | ERV_0275834 | THE1B,THE1B-int | LIL | 1.45 | 5.26 | 3.62 |
| HLA078 | 1 | 74725239 | 74727410 | ERV_0275834 | THE1B,THE1B-int | LIL | 2.69 | 7.06 | 2.62 |
| HLA053 | 1 | 74740033 | 74744894 | ERV_0275843 | HERV9N-int,LTR12,LTR12B | LIL | 0.84 | 2.22 | 2.64 |
| HLA053 | 1 | 74741115 | 74744614 | ERV_0275844 | HERV9-int | I | 0.84 | 2.22 | 2.64 |
| HLA058 | 1 | 75296224 | 75297084 | ERV_0276136 | MLT1G,MLT1G-int | LIL | 0.19 | 4.43 | 23.07 |
| HLA065 | 1 | 75296224 | 75297084 | ERV_0276136 | MLT1G,MLT1G-int | LIL | 0.00 | 7.59 | 300.00 |
| HLA065 | 1 | 77918963 | 77919582 | ERV_0277845 | THE1A,THE1A-int | LI | 0.00 | 2.10 | 300.00 |
| HLA072 | 1 | 78193438 | 78194358 | ERV_0278072 | MSTD,MSTD-int | LI | 0.00 | 1.67 | 300.00 |
| HLA058 | 1 | 78225095 | 78225460 | ERV_0278091 | ERVL-E-int | I | 1.80 | 14.16 | 7.88 |
| HLA067 | 1 | 78225095 | 78225460 | ERV_0278091 | ERVL-E-int | I | 0.92 | 6.71 | 7.34 |
| HLA058 | 1 | 78324273 | 78326208 | ERV_0278158 | ERVL-E-int | I | 0.30 | 1.77 | 5.88 |
| HLA065 | 1 | 78646414 | 78648605 | ERV_0278348 | THE1C,THE1C-int | LIL | 0.51 | 2.50 | 4.86 |
| HLA072 | 1 | 78646414 | 78648605 | ERV_0278348 | THE1C,THE1C-int | LIL | 1.39 | 2.90 | 2.09 |
| HLA053 | 1 | 86376510 | 86378854 | ERV_0282458 | THE1D,THE1D-int | LIL | 0.68 | 2.68 | 3.95 |
| HLA078 | 1 | 86692599 | 86694874 | ERV_0282631 | HUERS-P3b-int,LTR9B | LI | 0.00 | 6.19 | 300.00 |
| HLA058 | 1 | 87800644 | 87803070 | ERV_0283324 | ERVL-B4-int,MLT2C1 | LI | 0.36 | 5.56 | 15.33 |
| HLA071 | 1 | 87800644 | 87803070 | ERV_0283324 | ERVL-B4-int,MLT2C1 | LI | 0.38 | 1.56 | 4.10 |
| HLA072 | 1 | 87800644 | 87803070 | ERV_0283324 | ERVL-B4-int,MLT2C1 | LI | 0.00 | 1.66 | 300.00 |
| HLA065 | 1 | 93261861 | 93264299 | ERV_0286811 | THE1D,THE1D-int | LIL | 3.04 | 10.58 | 3.48 |
| HLA078 | 1 | 93261861 | 93264299 | ERV_0286811 | THE1D,THE1D-int | LIL | 5.01 | 10.11 | 2.02 |
| HLA065 | 1 | 93263886 | 93263973 | ERV_0286812 | THE1D-int | I | 3.04 | 10.58 | 3.48 |
| HLA078 | 1 | 93263886 | 93263973 | ERV_0286812 | THE1D-int | I | 5.01 | 10.11 | 2.02 |
| HLA061 | 1 | 93385118 | 93385630 | ERV_0286902 | MLT1J1,MLT1J1-int | LI | 0.00 | 2.49 | 300.00 |
| HLA063 | 1 | 93385118 | 93385630 | ERV_0286902 | MLT1J1,MLT1J1-int | LI | 0.00 | 2.52 | 300.00 |
| HLA078 | 1 | 93385118 | 93385630 | ERV_0286902 | MLT1J1,MLT1J1-int | LI | 0.00 | 1.97 | 300.00 |
| HLA058 | 1 | 94161627 | 94167221 | ERV_0287406 | HERVL-int,MLT2A1 | LIL | 0.67 | 4.44 | 6.63 |
| HLA069 | 1 | 94161627 | 94167221 | ERV_0287406 | HERVL-int,MLT2A1 | LIL | 1.00 | 2.21 | 2.21 |
| HLA078 | 1 | 94161627 | 94167221 | ERV_0287406 | HERVL-int,MLT2A1 | LIL | 0.00 | 1.50 | 300.00 |
| HLA082 | 1 | 94161627 | 94167221 | ERV_0287406 | HERVL-int,MLT2A1 | LIL | 0.70 | 2.21 | 3.15 |
| HLA058 | 1 | 94661344 | 94669497 | ERV_0287708 | MER4-int,MER4C | LIL | 0.17 | 2.02 | 11.93 |
| HLA058 | 1 | 94666404 | 94666979 | ERV_0287709 | MER4B-int | I | 0.17 | 2.02 | 11.93 |
| HLA058 | 1 | 94667118 | 94669028 | ERV_0287711 | MER4-int | I | 0.17 | 2.02 | 11.93 |
| HLA065 | 1 | 95250622 | 95254066 | ERV_0288131 | ERVL-E-int | I | 0.69 | 1.91 | 2.76 |
| HLA066 | 1 | 95250622 | 95254066 | ERV_0288131 | ERVL-E-int | I | 0.55 | 1.95 | 3.54 |
| HLA072 | 1 | 95250622 | 95254066 | ERV_0288131 | ERVL-E-int | I | 0.63 | 1.67 | 2.64 |
| HLA072 | 1 | 1E+08 | 1E+08 | ERV_0291157 | THE1B,THE1B-int | LI | 0.00 | 2.01 | 300.00 |
| HLA053 | 1 | 1.01E+08 | 1.01E+08 | ERV_0291425 | THE1D,THE1D-int | LI | 0.83 | 2.00 | 2.41 |
| HLA076 | 1 | 1.01E+08 | 1.01E+08 | ERV_0291425 | THE1D,THE1D-int | LI | 0.73 | 1.85 | 2.52 |
| HLA058 | 1 | 1.02E+08 | 1.02E+08 | ERV_0292179 | THE1B,THE1B-int | LIL | 0.00 | 4.85 | 300.00 |
| HLA067 | 1 | 1.04E+08 | 1.04E+08 | ERV_0292792 | THE1B,THE1B-int | LI | 0.65 | 3.86 | 5.89 |
| HLA058 | 1 | 1.04E+08 | 1.04E+08 | ERV_0293028 | THE1D,THE1D-int | LI | 1.58 | 12.96 | 8.21 |
| HLA078 | 1 | 1.04E+08 | 1.04E+08 | ERV_0293205 | HERVE_a-int | I | 0.04 | 4.82 | 131.07 |
| HLA078 | 1 | 1.05E+08 | 1.05E+08 | ERV_0293262 | ERVL-E-int | I | 0.00 | 2.19 | 300.00 |
| HLA058 | 1 | 1.06E+08 | 1.06E+08 | ERV_0293695 | LTR38B,MER83A-int | LI | 0.00 | 6.82 | 300.00 |
| HLA053 | 1 | 1.08E+08 | 1.08E+08 | ERV_0294968 | THE1A,THE1A-int | LIL | 0.14 | 1.76 | 12.80 |
| HLA061 | 1 | 1.13E+08 | 1.13E+08 | ERV_0297844 | MLT1B,MLT1B-int | LI | 0.73 | 1.92 | 2.64 |
| HLA053 | 1 | 1.13E+08 | 1.13E+08 | ERV_0298108 | LTR54B,MER57A-int | LI | 1.18 | 2.78 | 2.35 |
| HLA076 | 1 | 1.13E+08 | 1.13E+08 | ERV_0298108 | LTR54B,MER57A-int | LI | 0.09 | 1.58 | 18.08 |
| HLA053 | 1 | 1.13E+08 | 1.13E+08 | ERV_0298109 | HERV35I-int | I | 1.18 | 2.78 | 2.35 |
| HLA076 | 1 | 1.13E+08 | 1.13E+08 | ERV_0298109 | HERV35I-int | I | 0.09 | 1.58 | 18.08 |
| HLA053 | 1 | 1.13E+08 | 1.13E+08 | ERV_0298111 | LTR49-int | I | 1.18 | 2.78 | 2.35 |
| HLA076 | 1 | 1.13E+08 | 1.13E+08 | ERV_0298111 | LTR49-int | I | 0.09 | 1.58 | 18.08 |
| HLA067 | 1 | 1.13E+08 | 1.13E+08 | ERV_0298181 | MLT1A0,MLT1A0-int | LI | 0.17 | 3.14 | 18.49 |
| HLA058 | 1 | 1.14E+08 | 1.14E+08 | ERV_0298689 | THE1A,THE1A-int | LIL | 1.73 | 8.54 | 4.95 |
| HLA072 | 1 | 1.14E+08 | 1.14E+08 | ERV_0298689 | THE1A,THE1A-int | LIL | 1.74 | 4.59 | 2.63 |
| HLA053 | 1 | 1.14E+08 | 1.14E+08 | ERV_0298815 | MLT1H-int | I | 0.28 | 2.47 | 8.71 |
| HLA058 | 1 | 1.17E+08 | 1.17E+08 | ERV_0300843 | MamRep605,THE1B,THE 1B-int | LIL | 0.15 | 2.64 | 17.92 |
| HLA066 | 1 | 1.17E+08 | 1.17E+08 | ERV_0300843 | MamRep605,THE1B,THE 1B-int | LIL | 0.83 | 2.30 | 2.76 |
| HLA067 | 1 | 1.17E+08 | 1.17E+08 | ERV_0300843 | MamRep605,THE1B,THE 1B-int | LIL | 2.34 | 4.95 | 2.12 |
| HLA069 | 1 | 1.17E+08 | 1.17E+08 | ERV_0300843 | MamRep605,THE1B,THE 1B-int | LIL | 0.48 | 3.36 | 6.95 |
| HLA071 | 1 | 1.17E+08 | 1.17E+08 | ERV_0300843 | MamRep605,THE1B,THE 1B-int | LIL | 1.21 | 4.91 | 4.05 |
| HLA076 | 1 | 1.17E+08 | 1.17E+08 | ERV_0300843 | MamRep605,THE1B,THE 1B-int | LIL | 1.69 | 16.42 | 9.75 |
| HLA078 | 1 | 1.17E+08 | 1.17E+08 | ERV_0300843 | MamRep605,THE1B,THE 1B-int | LIL | 2.39 | 6.42 | 2.68 |
| HLA082 | 1 | 1.17E+08 | 1.17E+08 | ERV_0300843 | MamRep605,THE1B,THE 1B-int | LIL | 1.83 | 6.30 | 3.45 |
| HLA078 | 1 | 1.18E+08 | 1.18E+08 | ERV_0301402 | HERVL18-int,LTR18C | LIL | 0.00 | 1.74 | 300.00 |
| HLA058 | 1 | 1.19E+08 | 1.19E+08 | ERV_0302116 | HERV17-int,LTR17 | LIL | 0.71 | 2.16 | 3.06 |
| HLA053 | 1 | 1.19E+08 | 1.19E+08 | ERV_0302235 | MER4-int,MER4A1 | LIL | 0.41 | 5.40 | 13.10 |
| HLA069 | 1 | 1.19E+08 | 1.19E+08 | ERV_0302235 | MER4-int,MER4A1 | LIL | 3.66 | 10.18 | 2.78 |
| HLA053 | 1 | 1.19E+08 | 1.19E+08 | ERV_0302237 | MER4-int | I | 0.41 | 5.40 | 13.10 |
| HLA069 | 1 | 1.19E+08 | 1.19E+08 | ERV_0302237 | MER4-int | I | 3.66 | 10.18 | 2.78 |
| HLA053 | 1 | 1.19E+08 | 1.19E+08 | ERV_0302249 | THE1A,THE1A-int | LI | 0.38 | 4.30 | 11.25 |
| HLA067 | 1 | 1.19E+08 | 1.19E+08 | ERV_0302249 | THE1A,THE1A-int | LI | 1.60 | 3.26 | 2.04 |
| HLA069 | 1 | 1.19E+08 | 1.19E+08 | ERV_0302249 | THE1A,THE1A-int | LI | 1.93 | 6.29 | 3.26 |
| HLA069 | 1 | 1.19E+08 | 1.19E+08 | ERV_0302257 | THE1B,THE1B-int | LIL | 0.66 | 1.56 | 2.36 |
| HLA066 | 1 | 1.19E+08 | 1.19E+08 | ERV_0302300 | HUERS-P2-int,LTR1A2 | LI | 0.00 | 4.94 | 300.00 |
| HLA067 | 1 | 1.2E+08 | 1.2E+08 | ERV_0302873 | MLT1E3,MLT1E3-int | LI | 3.11 | 9.30 | 2.99 |
| HLA078 | 1 | 1.2E+08 | 1.2E+08 | ERV_0302873 | MLT1E3,MLT1E3-int | LI | 2.81 | 9.46 | 3.37 |
| HLA058 | 1 | 1.21E+08 | 1.21E+08 | ERV_0303126 | ERVL-E-int,LTR33 | LI | 1.07 | 3.33 | 3.10 |
| HLA072 | 1 | 1.21E+08 | 1.21E+08 | ERV_0303126 | ERVL-E-int,LTR33 | LI | 0.46 | 1.74 | 3.77 |
| HLA058 | 1 | 1.21E+08 | 1.21E+08 | ERV_0303136 | HERVP71A-int,LTR71B | LI | 1.07 | 3.33 | 3.10 |
| HLA072 | 1 | 1.21E+08 | 1.21E+08 | ERV_0303136 | HERVP71A-int,LTR71B | LI | 0.46 | 1.74 | 3.77 |
| HLA065 | 1 | 1.44E+08 | 1.44E+08 | ERV_0304455 | ERVL-E-int,LTR33 | LI | 2.24 | 17.56 | 7.86 |
| HLA067 | 1 | 1.44E+08 | 1.44E+08 | ERV_0304455 | ERVL-E-int,LTR33 | LI | 12.97 | 62.53 | 4.82 |
| HLA069 | 1 | 1.44E+08 | 1.44E+08 | ERV_0304455 | ERVL-E-int,LTR33 | LI | 2.75 | 8.60 | 3.13 |
| HLA078 | 1 | 1.44E+08 | 1.44E+08 | ERV_0304455 | ERVL-E-int,LTR33 | LI | 3.58 | 27.68 | 7.74 |
| HLA072 | 1 | 1.45E+08 | 1.45E+08 | ERV_0304537 | LTR23-int,LTR56 | LI | 0.08 | 2.41 | 31.64 |
| HLA078 | 1 | 1.45E+08 | 1.45E+08 | ERV_0304537 | LTR23-int,LTR56 | LI | 0.05 | 3.82 | 81.84 |
| HLA072 | 1 | 1.45E+08 | 1.45E+08 | ERV_0304539 | LTR39-int | I | 0.08 | 2.41 | 31.64 |
| HLA078 | 1 | 1.45E+08 | 1.45E+08 | ERV_0304539 | LTR39-int | I | 0.05 | 3.82 | 81.84 |
| HLA053 | 1 | 1.45E+08 | 1.45E+08 | ERV_0305070 | MER70-int | I | 0.00 | 1.82 | 300.00 |
| HLA053 | 1 | 1.47E+08 | 1.47E+08 | ERV_0306095 | MLT1E2,MLT1E2-int | LI | 2.05 | 6.38 | 3.11 |
| HLA065 | 1 | 1.47E+08 | 1.47E+08 | ERV_0306095 | MLT1E2,MLT1E2-int | LI | 1.12 | 5.57 | 4.96 |
| HLA076 | 1 | 1.47E+08 | 1.47E+08 | ERV_0306095 | MLT1E2,MLT1E2-int | LI | 2.81 | 8.40 | 2.99 |
| HLA078 | 1 | 1.47E+08 | 1.47E+08 | ERV_0306095 | MLT1E2,MLT1E2-int | LI | 0.85 | 5.31 | 6.26 |
| HLA063 | 1 | 1.48E+08 | 1.48E+08 | ERV_0306827 | ERVL-E-int,LTR33 | LI | 4.55 | 11.28 | 2.48 |
| HLA067 | 1 | 1.48E+08 | 1.48E+08 | ERV_0306827 | ERVL-E-int,LTR33 | LI | 7.54 | 16.68 | 2.21 |
| HLA069 | 1 | 1.48E+08 | 1.48E+08 | ERV_0306827 | ERVL-E-int,LTR33 | LI | 0.55 | 3.37 | 6.10 |
| HLA076 | 1 | 1.48E+08 | 1.48E+08 | ERV_0306827 | ERVL-E-int,LTR33 | LI | 6.99 | 15.52 | 2.22 |
| HLA078 | 1 | 1.48E+08 | 1.48E+08 | ERV_0306827 | ERVL-E-int,LTR33 | LI | 0.95 | 16.70 | 17.56 |
| HLA078 | 1 | 1.48E+08 | 1.48E+08 | ERV_0306937 | Harlequin-int,LTR2B | LI | 0.00 | 5.42 | 300.00 |
| HLA078 | 1 | 1.48E+08 | 1.48E+08 | ERV_0306938 | HERVE_a-int | I | 0.00 | 5.42 | 300.00 |
| HLA058 | 1 | 1.48E+08 | 1.48E+08 | ERV_0306999 | ERVL-E-int,LTR33 | LI | 0.67 | 2.45 | 3.67 |
| HLA072 | 1 | 1.48E+08 | 1.48E+08 | ERV_0306999 | ERVL-E-int,LTR33 | LI | 0.47 | 3.58 | 7.60 |
| HLA058 | 1 | 1.48E+08 | 1.48E+08 | ERV_0307009 | HERVP71A-int,LTR71B | LI | 0.67 | 2.45 | 3.67 |
| HLA072 | 1 | 1.48E+08 | 1.48E+08 | ERV_0307009 | HERVP71A-int,LTR71B | LI | 0.47 | 3.58 | 7.60 |
| HLA058 | 1 | 1.48E+08 | 1.48E+08 | ERV_0307027 | MER4-int,MER4C | LI | 13.14 | 28.66 | 2.18 |
| HLA063 | 1 | 1.48E+08 | 1.48E+08 | ERV_0307027 | MER4-int,MER4C | LI | 1.62 | 3.34 | 2.06 |
| HLA065 | 1 | 1.48E+08 | 1.48E+08 | ERV_0307027 | MER4-int,MER4C | LI | 4.62 | 15.67 | 3.39 |
| HLA072 | 1 | 1.48E+08 | 1.48E+08 | ERV_0307027 | MER4-int,MER4C | LI | 2.56 | 17.99 | 7.03 |
| HLA078 | 1 | 1.48E+08 | 1.48E+08 | ERV_0307027 | MER4-int,MER4C | LI | 0.29 | 2.16 | 7.42 |
| HLA058 | 1 | 1.48E+08 | 1.48E+08 | ERV_0307046 | MER66-int,MER66C | LI | 9.99 | 35.80 | 3.58 |
| HLA067 | 1 | 1.48E+08 | 1.48E+08 | ERV_0307046 | MER66-int,MER66C | LI | 8.19 | 17.08 | 2.09 |
| HLA069 | 1 | 1.48E+08 | 1.48E+08 | ERV_0307046 | MER66-int,MER66C | LI | 4.45 | 11.20 | 2.52 |
| HLA072 | 1 | 1.48E+08 | 1.48E+08 | ERV_0307046 | MER66-int,MER66C | LI | 8.47 | 20.58 | 2.43 |
| HLA073 | 1 | 1.48E+08 | 1.48E+08 | ERV_0307046 | MER66-int,MER66C | LI | 3.69 | 11.09 | 3.01 |
| HLA078 | 1 | 1.48E+08 | 1.48E+08 | ERV_0307046 | MER66-int,MER66C | LI | 6.45 | 25.53 | 3.96 |
| HLA065 | 1 | 1.49E+08 | 1.49E+08 | ERV_0307444 | ERVL-E-int,LTR33 | LI | 1.33 | 7.63 | 5.74 |
| HLA067 | 1 | 1.49E+08 | 1.49E+08 | ERV_0307444 | ERVL-E-int,LTR33 | LI | 4.37 | 14.85 | 3.40 |
| HLA078 | 1 | 1.49E+08 | 1.49E+08 | ERV_0307444 | ERVL-E-int,LTR33 | LI | 1.95 | 14.77 | 7.59 |
| HLA058 | 1 | 1.49E+08 | 1.49E+08 | ERV_0307509 | PRIMA4-int | I | 0.65 | 2.38 | 3.65 |
| HLA067 | 1 | 1.49E+08 | 1.49E+08 | ERV_0307509 | PRIMA4-int | I | 0.29 | 2.23 | 7.59 |
| HLA067 | 1 | 1.49E+08 | 1.49E+08 | ERV_0307511 | ERV24_Prim-int | I | 0.00 | 1.53 | 300.00 |
| HLA063 | 1 | 1.5E+08 | 1.5E+08 | ERV_0307746 | ERVL-E-int,LTR33 | LI | 0.76 | 1.97 | 2.58 |
| HLA071 | 1 | 1.5E+08 | 1.5E+08 | ERV_0307746 | ERVL-E-int,LTR33 | LI | 1.03 | 3.27 | 3.17 |
| HLA072 | 1 | 1.5E+08 | 1.5E+08 | ERV_0307746 | ERVL-E-int,LTR33 | LI | 0.33 | 1.60 | 4.85 |
| HLA073 | 1 | 1.5E+08 | 1.5E+08 | ERV_0307746 | ERVL-E-int,LTR33 | LI | 1.65 | 3.65 | 2.21 |
| HLA063 | 1 | 1.5E+08 | 1.5E+08 | ERV_0307756 | HERVP71A-int,LTR71B | LI | 0.76 | 1.97 | 2.58 |
| HLA071 | 1 | 1.5E+08 | 1.5E+08 | ERV_0307756 | HERVP71A-int,LTR71B | LI | 1.03 | 3.27 | 3.17 |
| HLA072 | 1 | 1.5E+08 | 1.5E+08 | ERV_0307756 | HERVP71A-int,LTR71B | LI | 0.33 | 1.60 | 4.85 |
| HLA073 | 1 | 1.5E+08 | 1.5E+08 | ERV_0307756 | HERVP71A-int,LTR71B | LI | 1.65 | 3.65 | 2.21 |
| HLA058 | 1 | 1.5E+08 | 1.5E+08 | ERV_0307804 | MER66-int,MER66C | LI | 0.08 | 2.35 | 27.84 |
| HLA065 | 1 | 1.5E+08 | 1.5E+08 | ERV_0307804 | MER66-int,MER66C | LI | 0.88 | 3.26 | 3.71 |
| HLA069 | 1 | 1.5E+08 | 1.5E+08 | ERV_0307804 | MER66-int,MER66C | LI | 0.00 | 1.73 | 300.00 |
| HLA071 | 1 | 1.5E+08 | 1.5E+08 | ERV_0307804 | MER66-int,MER66C | LI | 0.84 | 3.16 | 3.76 |
| HLA072 | 1 | 1.5E+08 | 1.5E+08 | ERV_0307804 | MER66-int,MER66C | LI | 0.68 | 3.21 | 4.70 |
| HLA058 | 1 | 1.51E+08 | 1.51E+08 | ERV_0308616 | HERVK-int | I | 0.74 | 5.88 | 7.93 |
| HLA061 | 1 | 1.51E+08 | 1.51E+08 | ERV_0308616 | HERVK-int | I | 3.54 | 8.32 | 2.35 |
| HLA063 | 1 | 1.51E+08 | 1.51E+08 | ERV_0308616 | HERVK-int | I | 1.78 | 4.32 | 2.42 |
| HLA066 | 1 | 1.51E+08 | 1.51E+08 | ERV_0308616 | HERVK-int | I | 0.79 | 4.08 | 5.18 |
| HLA067 | 1 | 1.51E+08 | 1.51E+08 | ERV_0308616 | HERVK-int | I | 2.19 | 5.76 | 2.63 |
| HLA069 | 1 | 1.51E+08 | 1.51E+08 | ERV_0308616 | HERVK-int | I | 1.11 | 2.71 | 2.43 |
| HLA073 | 1 | 1.51E+08 | 1.51E+08 | ERV_0308616 | HERVK-int | I | 1.45 | 3.46 | 2.38 |
| HLA078 | 1 | 1.51E+08 | 1.51E+08 | ERV_0308616 | HERVK-int | I | 0.66 | 13.60 | 20.63 |
| HLA065 | 1 | 1.51E+08 | 1.51E+08 | ERV_0308973 | MER41-int,MER41A | LI | 0.21 | 4.20 | 20.13 |
| HLA072 | 1 | 1.51E+08 | 1.51E+08 | ERV_0308973 | MER41-int,MER41A | LI | 0.76 | 1.78 | 2.35 |
| HLA078 | 1 | 1.51E+08 | 1.51E+08 | ERV_0308973 | MER41-int,MER41A | LI | 0.34 | 2.01 | 5.95 |
| HLA058 | 1 | 1.51E+08 | 1.51E+08 | ERV_0309149 | MLT1F2,MLT1F2-int | LIL | 1.01 | 3.30 | 3.25 |
| HLA063 | 1 | 1.51E+08 | 1.51E+08 | ERV_0309149 | MLT1F2,MLT1F2-int | LIL | 1.83 | 3.94 | 2.16 |
| HLA065 | 1 | 1.51E+08 | 1.51E+08 | ERV_0309149 | MLT1F2,MLT1F2-int | LIL | 1.22 | 5.69 | 4.66 |
| HLA067 | 1 | 1.51E+08 | 1.51E+08 | ERV_0309149 | MLT1F2,MLT1F2-int | LIL | 0.44 | 2.43 | 5.47 |
| HLA078 | 1 | 1.51E+08 | 1.51E+08 | ERV_0309149 | MLT1F2,MLT1F2-int | LIL | 0.89 | 4.08 | 4.58 |
| HLA058 | 1 | 1.52E+08 | 1.52E+08 | ERV_0309763 | HUERS-P1-int,LTR8 | LI | 0.00 | 2.42 | 300.00 |
| HLA058 | 1 | 1.52E+08 | 1.52E+08 | ERV_0309765 | HERV35I-int | I | 0.00 | 2.49 | 300.00 |
| HLA058 | 1 | 1.52E+08 | 1.52E+08 | ERV_0309775 | LTR54,MER57A-int | LI | 0.00 | 2.10 | 300.00 |
| HLA063 | 1 | 1.56E+08 | 1.56E+08 | ERV_0312442 | LTR25-int | I | 1.17 | 2.44 | 2.09 |
| HLA065 | 1 | 1.56E+08 | 1.56E+08 | ERV_0312442 | LTR25-int | I | 0.46 | 1.64 | 3.53 |
| HLA072 | 1 | 1.56E+08 | 1.56E+08 | ERV_0312442 | LTR25-int | I | 0.23 | 3.83 | 16.90 |
| HLA078 | 1 | 1.56E+08 | 1.56E+08 | ERV_0312442 | LTR25-int | I | 0.00 | 1.82 | 300.00 |
| HLA058 | 1 | 1.56E+08 | 1.56E+08 | ERV_0312445 | HERV17-int | I | 1.68 | 9.49 | 5.66 |
| HLA061 | 1 | 1.56E+08 | 1.56E+08 | ERV_0312445 | HERV17-int | I | 1.26 | 4.45 | 3.52 |
| HLA063 | 1 | 1.56E+08 | 1.56E+08 | ERV_0312445 | HERV17-int | I | 1.79 | 6.51 | 3.64 |
| HLA067 | 1 | 1.56E+08 | 1.56E+08 | ERV_0312445 | HERV17-int | I | 1.83 | 12.53 | 6.83 |
| HLA069 | 1 | 1.56E+08 | 1.56E+08 | ERV_0312445 | HERV17-int | I | 1.14 | 3.30 | 2.91 |
| HLA073 | 1 | 1.56E+08 | 1.56E+08 | ERV_0312445 | HERV17-int | I | 0.97 | 8.75 | 9.02 |
| HLA078 | 1 | 1.56E+08 | 1.56E+08 | ERV_0312445 | HERV17-int | I | 1.14 | 48.52 | 42.39 |
| HLA082 | 1 | 1.56E+08 | 1.56E+08 | ERV_0312445 | HERV17-int | I | 0.94 | 4.06 | 4.34 |
| HLA058 | 1 | 1.56E+08 | 1.56E+08 | ERV_0312447 | HERVK-int, LTR5_Hs | LIL | 1.68 | 9.49 | 5.66 |
| HLA061 | 1 | 1.56E+08 | 1.56E+08 | ERV_0312447 | HERVK-int, LTR5_Hs | LIL | 1.26 | 4.45 | 3.52 |
| HLA063 | 1 | 1.56E+08 | 1.56E+08 | ERV_0312447 | HERVK-int, LTR5_Hs | LIL | 1.79 | 6.51 | 3.64 |
| HLA067 | 1 | 1.56E+08 | 1.56E+08 | ERV_0312447 | HERVK-int, LTR5_Hs | LIL | 1.83 | 12.53 | 6.83 |
| HLA069 | 1 | 1.56E+08 | 1.56E+08 | ERV_0312447 | HERVK-int, LTR5_Hs | LIL | 1.14 | 3.30 | 2.91 |
| HLA073 | 1 | 1.56E+08 | 1.56E+08 | ERV_0312447 | HERVK-int, LTR5_Hs | LIL | 0.97 | 8.75 | 9.02 |
| HLA078 | 1 | 1.56E+08 | 1.56E+08 | ERV_0312447 | HERVK-int, LTR5_Hs | LIL | 1.14 | 48.52 | 42.39 |
| HLA082 | 1 | 1.56E+08 | 1.56E+08 | ERV_0312447 | HERVK-int, LTR5_Hs | LIL | 0.94 | 4.06 | 4.34 |
| HLA078 | 1 | 1.56E+08 | 1.56E+08 | ERV_0312452 | LTR25,LTR25-int | LI | 0.04 | 2.10 | 58.00 |
| HLA063 | 1 | 1.56E+08 | 1.56E+08 | ERV_0312458 | HERV4_I-int,MER51A | LIL | 0.77 | 1.70 | 2.22 |
| HLA073 | 1 | 1.56E+08 | 1.56E+08 | ERV_0312458 | HERV4_I-int,MER51A | LIL | 0.72 | 1.71 | 2.38 |
| HLA078 | 1 | 1.56E+08 | 1.56E+08 | ERV_0312458 | HERV4_I-int,MER51A | LIL | 0.68 | 5.22 | 7.68 |
| HLA063 | 1 | 1.56E+08 | 1.56E+08 | ERV_0312459 | HERV4_I-int | I | 0.77 | 1.70 | 2.22 |
| HLA073 | 1 | 1.56E+08 | 1.56E+08 | ERV_0312459 | HERV4_I-int | I | 0.72 | 1.71 | 2.38 |
| HLA078 | 1 | 1.56E+08 | 1.56E+08 | ERV_0312459 | HERV4_I-int | I | 0.68 | 5.22 | 7.68 |
| HLA058 | 1 | 1.56E+08 | 1.56E+08 | ERV_0312929 | HERVK22-int,LTR22E | LIL | 2.85 | 10.82 | 3.79 |
| HLA058 | 1 | 1.56E+08 | 1.56E+08 | ERV_0312931 | HERVK22-int | I | 2.85 | 10.82 | 3.79 |
| HLA063 | 1 | 1.57E+08 | 1.57E+08 | ERV_0313303 | ERVL-E-int | I | 0.00 | 5.19 | 300.00 |
| HLA067 | 1 | 1.57E+08 | 1.57E+08 | ERV_0313303 | ERVL-E-int | I | 0.00 | 2.61 | 300.00 |
| HLA058 | 1 | 1.57E+08 | 1.57E+08 | ERV_0313742 | HERVFH21-int,LTR21A | LIL | 0.00 | 2.54 | 300.00 |
| HLA078 | 1 | 1.57E+08 | 1.57E+08 | ERV_0313742 | HERVFH21-int,LTR21A | LIL | 0.00 | 2.84 | 300.00 |
| HLA058 | 1 | 1.57E+08 | 1.57E+08 | ERV_0313743 | HERVFH21-int | I | 0.00 | 2.54 | 300.00 |
| HLA078 | 1 | 1.57E+08 | 1.57E+08 | ERV_0313743 | HERVFH21-int | I | 0.00 | 2.84 | 300.00 |
| HLA071 | 1 | 1.57E+08 | 1.57E+08 | ERV_0313831 | THE1C,THE1C-int | LIL | 0.59 | 2.03 | 3.45 |
| HLA053 | 1 | 1.58E+08 | 1.58E+08 | ERV_0314035 | ERVL-B4-int,MLT2B1 | LI | 1.06 | 3.79 | 3.57 |
| HLA069 | 1 | 1.58E+08 | 1.58E+08 | ERV_0314035 | ERVL-B4-int,MLT2B1 | LI | 1.10 | 2.96 | 2.70 |
| HLA073 | 1 | 1.58E+08 | 1.58E+08 | ERV_0314035 | ERVL-B4-int,MLT2B1 | LI | 0.82 | 2.06 | 2.50 |
| HLA069 | 1 | 1.58E+08 | 1.58E+08 | ERV_0314274 | LTR23-int | I | 0.28 | 1.99 | 7.05 |
| HLA063 | 1 | 1.6E+08 | 1.6E+08 | ERV_0315340 | ERVL-E-int | I | 2.97 | 7.04 | 2.37 |
| HLA067 | 1 | 1.6E+08 | 1.6E+08 | ERV_0315340 | ERVL-E-int | I | 2.91 | 6.72 | 2.31 |
| HLA069 | 1 | 1.6E+08 | 1.6E+08 | ERV_0315340 | ERVL-E-int | I | 1.99 | 5.71 | 2.87 |
| HLA076 | 1 | 1.6E+08 | 1.6E+08 | ERV_0315340 | ERVL-E-int | I | 1.90 | 4.95 | 2.61 |
| HLA078 | 1 | 1.6E+08 | 1.6E+08 | ERV_0315340 | ERVL-E-int | I | 1.36 | 3.71 | 2.73 |
| HLA067 | 1 | 1.61E+08 | 1.61E+08 | ERV_0316155 | HERVL-int,MLT2Al | LIL | 0.40 | 1.76 | 4.37 |
| HLA058 | 1 | 1.68E+08 | 1.68E+08 | ERV_0320772 | MLT1J1,MLT1J1-int | LI | 3.83 | 14.74 | 3.85 |
| HLA072 | 1 | 1.68E+08 | 1.68E+08 | ERV_0320772 | MLT1J1,MLT1J1-int | LI | 1.59 | 16.75 | 10.51 |
| HLA078 | 1 | 1.68E+08 | 1.68E+08 | ERV_0320772 | MLT1J1,MLT1J1-int | LI | 0.00 | 4.84 | 300.00 |
| HLA058 | 1 | 1.69E+08 | 1.69E+08 | ERV_0321295 | PABL_A,PABL_A-int | LI | 1.04 | 2.71 | 2.61 |
| HLA065 | 1 | 1.69E+08 | 1.69E+08 | ERV_0321295 | PABL_A,PABL_A-int | LI | 0.23 | 4.65 | 19.99 |
| HLA067 | 1 | 1.69E+08 | 1.69E+08 | ERV_0321295 | PABL_A,PABL_A-int | LI | 2.39 | 5.14 | 2.15 |
| HLA073 | 1 | 1.69E+08 | 1.69E+08 | ERV_0321295 | PABL_A,PABL_A-int | LI | 0.81 | 3.95 | 4.85 |
| HLA061 | 1 | 1.71E+08 | 1.71E+08 | ERV_0322179 | MLTIJ-int | I | 0.00 | 2.55 | 300.00 |
| HLA078 | 1 | 1.73E+08 | 1.73E+08 | ERV_0323303 | MER101-int | I | 0.01 | 1.75 | 158.70 |
| HLA078 | 1 | 1.73E+08 | 1.73E+08 | ERV_0323304 | MER101-int | I | 0.01 | 1.75 | 158.70 |
| HLA066 | 1 | 1.73E+08 | 1.73E+08 | ERV_0323669 | ERVL-E-int | I | 0.30 | 1.98 | 6.48 |
| HLA058 | 1 | 1.74E+08 | 1.74E+08 | ERV_0323754 | MLT1G1,MLT1G1-int | LI | 0.00 | 3.92 | 300.00 |
| HLA058 | 1 | 1.74E+08 | 1.74E+08 | ERV_0323793 | MER4-int,MER4A1 | LIL | 0.01 | 2.68 | 279.20 |
| HLA058 | 1 | 1.74E+08 | 1.74E+08 | ERV_0323794 | MER4-int | I | 0.01 | 2.68 | 279.20 |
| HLA058 | 1 | 1.74E+08 | 1.74E+08 | ERV_0323795 | HERVIP10FH-int,LTR10F | LIL | 0.01 | 2.68 | 279.20 |
| HLA058 | 1 | 1.74E+08 | 1.74E+08 | ERV_0323796 | HERVIP10F-int | I | 0.01 | 2.68 | 279.20 |
| HLA053 | 1 | 1.74E+08 | 1.74E+08 | ERV_0324280 | MER61-int | I | 1.53 | 4.95 | 3.23 |
| HLA067 | 1 | 1.74E+08 | 1.74E+08 | ERV_0324280 | MER61-int | I | 0.00 | 4.94 | 300.00 |
| HLA072 | 1 | 1.74E+08 | 1.74E+08 | ERV_0324280 | MER61-int | I | 0.00 | 3.30 | 300.00 |
| HLA058 | 1 | 1.75E+08 | 1.75E+08 | ERV_0324822 | HERV9NC-int,LTR12C | LIL | 0.05 | 1.71 | 32.29 |
| HLA069 | 1 | 1.75E+08 | 1.75E+08 | ERV_0324822 | HERV9NC-int,LTR12C | LIL | 0.00 | 2.79 | 300.00 |
| HLA076 | 1 | 1.75E+08 | 1.75E+08 | ERV_0324822 | HERV9NC-int,LTR12C | LIL | 0.04 | 19.36 | 512.72 |
| HLA078 | 1 | 1.75E+08 | 1.75E+08 | ERV_0324822 | HERV9NC-int,LTR12C | LIL | 0.10 | 2.42 | 23.11 |
| HLA082 | 1 | 1.75E+08 | 1.75E+08 | ERV_0324822 | HERV9NC-int,LTR12C | LIL | 0.00 | 1.74 | 300.00 |
| HLA078 | 1 | 1.76E+08 | 1.76E+08 | ERV_0325479 | MLT1L,MLT1L-int | LI | 0.00 | 1.70 | 300.00 |
| HLA061 | 1 | 1.79E+08 | 1.79E+08 | ERV_0326846 | HERVK9-int,MER9a2 | LIL | 0.67 | 5.09 | 7.64 |
| HLA061 | 1 | 1.82E+08 | 1.82E+08 | ERV_0329344 | MER61-int,MER61B | LIL | 1.42 | 4.65 | 3.26 |
| HLA066 | 1 | 1.82E+08 | 1.82E+08 | ERV_0329344 | MER61-int,MER61B | LIL | 0.09 | 1.79 | 19.78 |
| HLA069 | 1 | 1.82E+08 | 1.82E+08 | ERV_0329344 | MER61-int,MER61B | LIL | 0.26 | 6.78 | 26.57 |
| HLA071 | 1 | 1.82E+08 | 1.82E+08 | ERV_0329344 | MER61-int,MER61B | LIL | 0.09 | 2.12 | 22.86 |
| HLA073 | 1 | 1.82E+08 | 1.82E+08 | ERV_0329344 | MER61-int,MER61B | LIL | 0.13 | 13.08 | 101.36 |
| HLA076 | 1 | 1.82E+08 | 1.82E+08 | ERV_0329344 | MER61-int,MER61B | LIL | 0.63 | 10.76 | 17.01 |
| HLA078 | 1 | 1.82E+08 | 1.82E+08 | ERV_0329344 | MER61-int,MER61B | LIL | 0.05 | 2.69 | 56.38 |
| HLA067 | 1 | 1.82E+08 | 1.82E+08 | ERV_0329491 | ERV3-16A3_I-int | I | 0.00 | 1.98 | 300.00 |
| HLA058 | 1 | 1.91E+08 | 1.91E+08 | ERV_0334734 | MLT1J2-int | I | 0.00 | 2.39 | 300.00 |
| HLA058 | 1 | 1.91E+08 | 1.91E+08 | ERV_0334837 | MLT1J1,MLT1J1-int | LI | 0.50 | 2.06 | 4.13 |
| HLA063 | 1 | 1.93E+08 | 1.93E+08 | ERV_0335925 | ERV3-16A3_I-int | I | 0.00 | 2.00 | 300.00 |
| HLA065 | 1 | 1.94E+08 | 1.94E+08 | ERV_0336177 | MLT1J1,MLT1J1-int | LI | 0.00 | 1.80 | 300.00 |
| HLA065 | 1 | 1.97E+08 | 1.97E+08 | ERV_0337453 | THE1C,THE1C-int | LIL | 1.21 | 4.72 | 3.91 |
| HLA067 | 1 | 1.97E+08 | 1.97E+08 | ERV_0337453 | THE1C,THE1C-int | LIL | 4.37 | 26.56 | 6.08 |
| HLA069 | 1 | 1.97E+08 | 1.97E+08 | ERV_0337453 | THE1C,THE1C-int | LIL | 1.71 | 8.65 | 5.06 |
| HLA082 | 1 | 1.97E+08 | 1.97E+08 | ERV_0337453 | THE1C,THE1C-int | LIL | 2.23 | 13.83 | 6.21 |
| HLA067 | 1 | 1.97E+08 | 1.97E+08 | ERV_0337544 | THE1D,THE1D-int | LI | 1.50 | 32.87 | 21.89 |
| HLA069 | 1 | 1.97E+08 | 1.97E+08 | ERV_0337544 | THE1D,THE1D-int | LI | 0.29 | 2.74 | 9.53 |
| HLA058 | 1 | 1.98E+08 | 1.98E+08 | ERV_0338100 | LTR37-int | I | 0.00 | 4.48 | 300.00 |
| HLA072 | 1 | 2.02E+08 | 2.02E+08 | ERV_0340485 | MER21-int,MER21B,MER21C | LIL | 0.26 | 1.96 | 7.60 |
| HLA067 | 1 | 2.04E+08 | 2.04E+08 | ERV_0342071 | MLT1C,MLT1C-int | LI | 0.43 | 1.85 | 4.30 |
| HLA063 | 1 | 2.04E+08 | 2.04E+08 | ERV_0342381 | MLTIAO,MLTIAO-int | LI | 2.86 | 6.08 | 2.12 |
| HLA065 | 1 | 2.04E+08 | 2.04E+08 | ERV_0342381 | MLTIAO,MLTIAO-int | LI | 0.22 | 1.96 | 8.83 |
| HLA067 | 1 | 2.04E+08 | 2.04E+08 | ERV_0342381 | MLTIAO,MLTIAO-int | LI | 1.60 | 5.45 | 3.41 |
| HLA069 | 1 | 2.04E+08 | 2.04E+08 | ERV_0342381 | MLTIAO,MLTIAO-int | LI | 0.29 | 5.68 | 19.33 |
| HLA078 | 1 | 2.04E+08 | 2.04E+08 | ERV_0342381 | MLTIAO,MLTIAO-int | LI | 0.73 | 2.39 | 3.29 |
| HLA053 | 1 | 2.06E+08 | 2.06E+08 | ERV_0343733 | HERV17-int,LTR17 | LI | 0.01 | 2.28 | 240, 50 |
| HLA053 | 1 | 2.06E+08 | 2.06E+08 | ERV_0343741 | HERVH-int,LTR7B | LIL | 0.00 | 5.85 | 300.00 |
| HLA065 | 1 | 2.06E+08 | 2.06E+08 | ERV_0343741 | HERVH-int,LTR7B | LIL | 0.19 | 2.20 | 11.86 |
| HLA067 | 1 | 2.06E+08 | 2.06E+08 | ERV_0343741 | HERVH-int,LTR7B | LIL | 0.85 | 6.07 | 7.11 |
| HLA072 | 1 | 2.06E+08 | 2.06E+08 | ERV_0343741 | HERVH-int,LTR7B | LIL | 0.75 | 2.31 | 3.08 |
| HLA078 | 1 | 2.06E+08 | 2.06E+08 | ERV_0343741 | HERVH-int,LTR7B | LIL | 0.26 | 1.83 | 7.11 |
| HLA063 | 1 | 2.07E+08 | 2.07E+08 | ERV_0344612 | ERVL-E-int,MLT2F | LI | 5.07 | 11.12 | 2.19 |
| HLA065 | 1 | 2.07E+08 | 2.07E+08 | ERV_0344612 | ERVL-E-int,MLT2F | LI | 0.14 | 2.36 | 16.50 |
| HLA067 | 1 | 2.07E+08 | 2.07E+08 | ERV_0344612 | ERVL-E-int,MLT2F | LI | 1.00 | 7.02 | 7.00 |
| HLA082 | 1 | 2.07E+08 | 2.07E+08 | ERV_0344612 | ERVL-E-int,MLT2F | LI | 2.35 | 6.06 | 2.58 |
| HLA065 | 1 | 2.07E+08 | 2.07E+08 | ERV_0344622 | ERV3-16A3_I-int | I | 0.99 | 6.12 | 6.18 |
| HLA067 | 1 | 2.07E+08 | 2.07E+08 | ERV_0344622 | ERV3-16A3_I-int | I | 11.19 | 67.16 | 6.00 |
| HLA069 | 1 | 2.07E+08 | 2.07E+08 | ERV_0344622 | ERV3-16A3_I-int | I | 4.07 | 39.39 | 9.68 |
| HLA078 | 1 | 2.1E+08 | 2.1E+08 | ERV_0346194 | THE1A,THE1A-int | LIL | 0.00 | 3.32 | 300.00 |
| HLA078 | 1 | 2.11E+08 | 2.11E+08 | ERV_0347136 | MSTA,MSTA-int | LIL | 0.00 | 9.56 | 300.00 |
| HLA067 | 1 | 2.14E+08 | 2.14E+08 | ERV_0349196 | MLT1J2,MLT1J2-int | LIL | 1.40 | 6.16 | 4.39 |
| HLA078 | 1 | 2.14E+08 | 2.14E+08 | ERV_0349196 | MLT1J2,MLT1J2-int | LIL | 0.46 | 2.35 | 5.11 |
| HLA053 | 1 | 2.16E+08 | 2.16E+08 | ERV_0350182 | MER41-int,MER41B | LIL | 1.41 | 5.03 | 3.57 |
| HLA063 | 1 | 2.16E+08 | 2.16E+08 | ERV_0350182 | MER41-int,MER41B | LIL | 1.50 | 3.23 | 2.15 |
| HLA067 | 1 | 2.16E+08 | 2.16E+08 | ERV_0350182 | MER41-int,MER41B | LIL | 0.57 | 4.90 | 8.64 |
| HLA078 | 1 | 2.19E+08 | 2.19E+08 | ERV_0351862 | HERVE_a-int | I | 0.00 | 2.34 | 300.00 |
| HLA078 | 1 | 2.2E+08 | 2.2E+08 | ERV_0352559 | LTR49-int | I | 0.00 | 1.50 | 300.00 |
| HLA078 | 1 | 2.22E+08 | 2.22E+08 | ERV_0353869 | THE1B,THE1B-int | LIL | 0.00 | 9.01 | 300.00 |
| HLA078 | 1 | 2.25E+08 | 2.25E+08 | ERV_0355579 | ERVL-E-int,LTR33 | LI | 0.00 | 1.55 | 300.00 |
| HLA061 | 1 | 2.26E+08 | 2.26E+08 | ERV_0356351 | HERVK14-int,LTR14B | LIL | 0.00 | 2.10 | 300.00 |
| HLA061 | 1 | 2.26E+08 | 2.26E+08 | ERV_0356599 | ERVL-E-int | I | 0.44 | 3.12 | 7.15 |
| HLA069 | 1 | 2.26E+08 | 2.26E+08 | ERV_0356599 | ERVL-E-int | I | 0.38 | 2.12 | 5.56 |
| HLA078 | 1 | 2.26E+08 | 2.26E+08 | ERV_0356599 | ERVL-E-int | I | 0.64 | 7.24 | 11.39 |
| HLA053 | 1 | 2.26E+08 | 2.26E+08 | ERV_0356859 | ERVL-E-int | I | 0.33 | 2.34 | 7.16 |
| HLA078 | 1 | 2.26E+08 | 2.26E+08 | ERV_0356859 | ERVL-E-int | I | 0.00 | 1.93 | 300.00 |
| HLA058 | 1 | 2.27E+08 | 2.27E+08 | ERV_0356997 | HERVL66-int,LTR66 | LIL | 0.78 | 13.69 | 17.66 |
| HLA066 | 1 | 2.27E+08 | 2.27E+08 | ERV_0356997 | HERVL66-int,LTR66 | LIL | 0.70 | 3.15 | 4.53 |
| HLA076 | 1 | 2.27E+08 | 2.27E+08 | ERV_0356997 | HERVL66-int,LTR66 | LIL | 0.68 | 2.32 | 3.43 |
| HLA078 | 1 | 2.27E+08 | 2.27E+08 | ERV_0356997 | HERVL66-int,LTR66 | LIL | 0.09 | 165.43 | 1833.43 |
| HLA065 | 1 | 2.28E+08 | 2.28E+08 | ERV_0357676 | ERVL-B4-int | I | 0.00 | 1.61 | 300.00 |
| HLA061 | 1 | 2.28E+08 | 2.28E+08 | ERV_0357899 | M ER57-int,MER57 A1 | LIL | 0.00 | 2.12 | 300.00 |
| HLA078 | 1 | 2.28E+08 | 2.28E+08 | ERV_0357899 | M ER57-int,MER57 A1 | LIL | 0.05 | 3.21 | 58.34 |
| HLA082 | 1 | 2.28E+08 | 2.28E+08 | ERV_0357899 | M ER57-int,MER57 A1 | LIL | 0.24 | 2.18 | 9.26 |
| HLA061 | 1 | 2.28E+08 | 2.28E+08 | ERV_0357902 | MER57-int | I | 0.00 | 2.12 | 300.00 |
| HLA078 | 1 | 2.28E+08 | 2.28E+08 | ERV_0357902 | MER57-int | I | 0.05 | 3.21 | 58.34 |
| HLA082 | 1 | 2.28E+08 | 2.28E+08 | ERV_0357902 | MER57-int | I | 0.24 | 2.18 | 9.26 |
| HLA061 | 1 | 2.28E+08 | 2.28E+08 | ERV_0357903 | MER57-int | I | 0.00 | 2.12 | 300.00 |
| HLA078 | 1 | 2.28E+08 | 2.28E+08 | ERV_0357903 | MER57-int | I | 0.05 | 3.21 | 58.34 |
| HLA082 | 1 | 2.28E+08 | 2.28E+08 | ERV_0357903 | MER57-int | I | 0.24 | 2.18 | 9.26 |
| HLA078 | 1 | 2.28E+08 | 2.28E+08 | ERV_0358009 | MLT1B,MLT1B-int | LIL | 0.62 | 2.19 | 3.55 |
| HLA078 | 1 | 2.28E+08 | 2.28E+08 | ERV_0358012 | MLT1B-int | I | 0.62 | 2.19 | 3.55 |
| HLA078 | 1 | 2.32E+08 | 2.32E+08 | ERV_0360392 | HERVH-int,LTR7 | LIL | 0.05 | 1.53 | 32.91 |
| HLA066 | 1 | 2.35E+08 | 2.35E+08 | ERV_0362511 | ERVL-E-int | I | 0.00 | 1.80 | 300.00 |
| HLA053 | 1 | 2.38E+08 | 2.38E+08 | ERV_0364789 | MER61-int,MER61A | LIL | 0.28 | 2.56 | 9.25 |
| HLA058 | 1 | 2.38E+08 | 2.38E+08 | ERV_0364789 | MER61-int,MER61A | LIL | 0.00 | 4.36 | 300.00 |
| HLA069 | 1 | 2.38E+08 | 2.38E+08 | ERV_0364789 | MER61-int,MER61A | LIL | 0.70 | 1.80 | 2.55 |
| HLA071 | 1 | 2.38E+08 | 2.38E+08 | ERV_0364789 | MER61-int,MER61A | LIL | 0.63 | 6.93 | 11.08 |
| HLA072 | 1 | 2.38E+08 | 2.38E+08 | ERV_0364789 | MER61-int,MER61A | LIL | 0.06 | 2.83 | 47.22 |
| HLA078 | 1 | 2.38E+08 | 2.38E+08 | ERV_0364789 | MER61-int,MER61A | LIL | 0.05 | 13.05 | 253.17 |
| HLA072 | 1 | 2.44E+08 | 2.44E+08 | ERV_0368467 | HERV16-int | I | 0.00 | 2.37 | 300.00 |
| HLA082 | 1 | 2.44E+08 | 2.44E+08 | ERV_0368467 | HERV16-int | I | 0.00 | 1.70 | 300.00 |
| HLA053 | 1 | 2.46E+08 | 2.46E+08 | ERV_0369651 | THE1D-int | I | 0.00 | 8.24 | 300.00 |
| HLA071 | 1 | 2.46E+08 | 2.46E+08 | ERV_0369651 | THE1D-int | I | 0.64 | 5.82 | 9.08 |
| HLA073 | 1 | 2.46E+08 | 2.46E+08 | ERV_0369651 | THE1D-int | I | 0.00 | 4.84 | 300.00 |
| HLA076 | 1 | 2.46E+08 | 2.46E+08 | ERV_0369651 | THE1D-int | I | 0.00 | 2.23 | 300.00 |
| HLA078 | 1 | 2.46E+08 | 2.46E+08 | ERV_0369651 | THE1D-int | I | 0.00 | 8.80 | 300.00 |
| HLA082 | 1 | 2.46E+08 | 2.46E+08 | ERV_0369651 | THE1D-int | I | 0.00 | 2.06 | 300.00 |
| HLA053 | 1 | 2.46E+08 | 2.46E+08 | ERV_0369652 | THE1-int | I | 0.00 | 8.24 | 300.00 |
| HLA071 | 1 | 2.46E+08 | 2.46E+08 | ERV_0369652 | THE1-int | I | 0.64 | 5.82 | 9.08 |
| HLA073 | 1 | 2.46E+08 | 2.46E+08 | ERV_0369652 | THE1-int | I | 0.00 | 4.84 | 300.00 |
| HLA076 | 1 | 2.46E+08 | 2.46E+08 | ERV_0369652 | THE1-int | I | 0.00 | 2.23 | 300.00 |
| HLA078 | 1 | 2.46E+08 | 2.46E+08 | ERV_0369652 | THE1-int | I | 0.00 | 8.80 | 300.00 |
| HLA082 | 1 | 2.46E+08 | 2.46E+08 | ERV_0369652 | THE1-int | I | 0.00 | 2.06 | 300.00 |
| HLA063 | 1 | 2.47E+08 | 2.47E+08 | ERV_0370122 | HERVH48-int,MER48 | LIL | 0.57 | 1.67 | 2.91 |
| HLA067 | 1 | 2.47E+08 | 2.47E+08 | ERV_0370122 | HERVH48-int,MER48 | LIL | 0.26 | 1.89 | 7.36 |
| HLA071 | 1 | 2.47E+08 | 2.47E+08 | ERV_0370122 | HERVH48-int,MER48 | LIL | 1.47 | 8.92 | 6.07 |
| HLA073 | 1 | 2.47E+08 | 2.47E+08 | ERV_0370122 | HERVH48-int,MER48 | LIL | 1.01 | 2.42 | 2.40 |
| HLA078 | 1 | 2.47E+08 | 2.47E+08 | ERV_0370434 | HERVL74-int | I | 0.28 | 2.38 | 8.62 |
| HLA072 | 1 | 2.47E+08 | 2.47E+08 | ERV_0370442 | THE1-int | I | 0.00 | 2.96 | 300.00 |
| HLA078 | 1 | 2.47E+08 | 2.47E+08 | ERV_0370442 | THE1-int | I | 0.00 | 2.85 | 300.00 |
| HLA058 | 1 | 2.47E+08 | 2.47E+08 | ERV_0370443 | THE1D,THE1D-int | LI | 0.37 | 2.14 | 5.76 |
| HLA072 | 1 | 2.47E+08 | 2.47E+08 | ERV_0370443 | THE1D,THE1D-int | LI | 1.08 | 2.47 | 2.28 |
| HLA061 | 1 | 2.47E+08 | 2.47E+08 | ERV_0370478 | MER4-int,MER4A1 | LI | 0.00 | 9.24 | 300.00 |
| HLA065 | 1 | 2.47E+08 | 2.47E+08 | ERV_0370478 | MER4-int,MER4A1 | LI | 0.11 | 2.20 | 19.39 |
| HLA071 | 1 | 2.47E+08 | 2.47E+08 | ERV_0370478 | MER4-int,MER4A1 | LI | 0.00 | 3.13 | 300.00 |
| HLA078 | 1 | 2.47E+08 | 2.47E+08 | ERV_0370478 | MER4-int,MER4A1 | LI | 0.00 | 3.31 | 300.00 |
| HLA058 | 1 | 2.47E+08 | 2.47E+08 | ERV_0370499 | HERVL40-int | I | 1.11 | 2.72 | 2.45 |
| HLA061 | 1 | 2.47E+08 | 2.47E+08 | ERV_0370499 | HERVL40-int | I | 0.58 | 1.78 | 3.08 |
| HLA065 | 1 | 2.47E+08 | 2.47E+08 | ERV_0370499 | HERVL40-int | I | 0.72 | 1.94 | 2.70 |
| HLA072 | 1 | 2.47E+08 | 2.47E+08 | ERV_0370499 | HERVL40-int | I | 0.50 | 5.82 | 11.68 |
| HLA078 | 1 | 2.47E+08 | 2.47E+08 | ERV_0370499 | HERVL40-int | I | 0.28 | 1.64 | 5.85 |
| HLA069 | 1 | 2.47E+08 | 2.47E+08 | ERV_0370587 | MLT1A0,MLT1A0-int | LIL | 1.16 | 3.36 | 2.90 |
| HLA072 | 1 | 2.47E+08 | 2.47E+08 | ERV_0370587 | MLT1A0,MLT1A0-int | LIL | 1.16 | 2.42 | 2.08 |
| HLA058 | 1 | 2.48E+08 | 2.48E+08 | ERV_0371226 | MLT1-int | I | 0.00 | 2.08 | 300.00 |
| HLA053 | 1 | 2.49E+08 | 2.49E+08 | ERV_0371328 | MER51-int,MER51B | LI | 0.07 | 4.87 | 71.23 |
| HLA069 | 1 | 2.49E+08 | 2.49E+08 | ERV_0371328 | MER51-int,MER51B | LI | 0.00 | 6.89 | 300.00 |
| HLA071 | 1 | 2.49E+08 | 2.49E+08 | ERV_0371328 | MER51-int,MER51B | LI | 0.33 | 7.88 | 23.65 |
| HLA073 | 1 | 2.49E+08 | 2.49E+08 | ERV_0371328 | MER51-int,MER51B | LI | 0.00 | 2.28 | 300.00 |
| HLA078 | 1 | 2.49E+08 | 2.49E+08 | ERV_0371328 | MER51-int,MER51B | LI | 0.00 | 2.68 | 300.00 |
| HLA063 | 10 | 918054 | 918164 | ERV_0372088 | LTR23-int | I | 0.50 | 2.00 | 4.00 |
| HLA072 | 10 | 17216275 | 17216354 | ERV_0387018 | HERVIP10FH-int | I | 1.84 | 4.00 | 2.18 |
| HLA067 | 10 | 17438986 | 17439047 | ERV_0387265 | LTR57-int | I | 0.00 | 3.00 | 300.00 |
| HLA073 | 10 | 17439777 | 17439884 | ERV_0387266 | LTR52-int | I | 0.00 | 2.00 | 300.00 |
| HLA067 | 10 | 1.3E+08 | 1.3E+08 | ERV_0487502 | MLT1L,MLT1L-int | LI | 1.20 | 3.25 | 2.70 |
| HLA078 | 10 | 13650718 | 13650760 | ERV_0497851 | HERVL-int | I | 1.00 | 2.00 | 2.00 |
| HLA067 | 10 | 45460134 | 45460194 | ERV_0516568 | MamGyp-int | I | 0.00 | 2.67 | 300.00 |
| HLA082 | 10 | 45460134 | 45460194 | ERV_0516568 | MamGyp-int | I | 0.00 | 2.00 | 300.00 |
| HLA078 | 10 | 48416034 | 48416181 | ERV_0518349 | ERVL-E-int | I | 0.00 | 2.00 | 300.00 |
| HLA067 | 11 | 30558118 | 30558164 | ERV_0592264 | MER4-int | I | 1.00 | 3.00 | 3.00 |
| HLA058 | 11 | 57714124 | 57714219 | ERV_0607141 | MER34-int | I | 0.00 | 2.00 | 300.00 |
| HLA053 | 11 | 68887856 | 68887871 | ERV_0615645 | MER101-int | I | 0.00 | 2.00 | 300.00 |
| HLA061 | 11 | 68887856 | 68887871 | ERV_0615645 | MER101-int | I | 0.00 | 1.93 | 300.00 |
| HLA072 | 11 | 68887856 | 68887871 | ERV_0615645 | MER101-int | I | 0.00 | 8.00 | 300.00 |
| HLA067 | 11 | 1.14E+08 | 1.14E+08 | ERV_0645559 | MSTA,MSTA-int | LIL | 0.00 | 1.86 | 300.00 |
| HLA072 | 11 | 1.21E+08 | 1.21E+08 | ERV_0650459 | ERVL-E-int | I | 0.00 | 2.00 | 300.00 |
| HLA065 | 11 | 45872343 | 45872457 | ERV_0700157 | MER57-int | I | 0.00 | 1.56 | 300.00 |
| HLA073 | 11 | 1.2E+08 | 1.2E+08 | ERV_0766283 | PRIMA4-int | I | 0.00 | 2.00 | 300.00 |
| HLA069 | 12 | 1.02E+08 | 1.02E+08 | ERV_0867199 | MER65-int | I | 0.00 | 2.80 | 300.00 |
| HLA082 | 13 | 23524412 | 23532857 | ERV_0996915 | HERV9NC-int,LTR12C | LIL | 0.03 | 1.89 | 71.49 |
| HLA053 | 13 | 94251480 | 94251714 | ERV_1056462 | MLT1H-int | I | 0.00 | 2.50 | 300.00 |
| HLA065 | 13 | 91562053 | 91562121 | ERV_1114439 | ERVL-E-int | I | 0.00 | 1.71 | 300.00 |
| HLA053 | 13 | 1E+08 | 1E+08 | ERV_1120019 | MER34-int | I | 0.00 | 2.00 | 300.00 |
| HLA061 | 14 | 50228354 | 50228682 | ERV_1153268 | LTR37-int,LTR37B | LI | 0.00 | 1.51 | 300.00 |
| HLA063 | 14 | 68480483 | 68481099 | ERV_1169312 | MER34B-int | I | 0.00 | 2.25 | 300.00 |
| HLA061 | 14 | 71039247 | 71039880 | ERV_1237824 | MLT1F,MLT1F-int | LI | 0.80 | 2.72 | 3.40 |
| HLA066 | 15 | 70688710 | 70689218 | ERV_1309221 | MLT1J,MLT1J-int | LI | 0.00 | 4.00 | 300.00 |
| HLA058 | 15 | 64435855 | 64435891 | ERV_1367176 | ERVL-int | I | 0.00 | 1.89 | 300.00 |
| HLA065 | 16 | 71708060 | 71708575 | ERV_1461030 | HERVH-int,LTR7 | LI | 0.00 | 1.70 | 300.00 |
| HLA071 | 16 | 2915823 | 2916846 | ERV_1481474 | LTR37-int,LTR37A | LI | 0.90 | 2.18 | 2.44 |
| HLA073 | 17 | 36181655 | 36181703 | ERV_1576684 | MLT-int | I | 0.00 | 1.66 | 300.00 |
| HLA069 | 17 | 76664462 | 76664567 | ERV_1620562 | HERV16-int | I | 0.00 | 1.57 | 300.00 |
| HLA078 | 17 | 14025678 | 14026413 | ERV_1637737 | MER89-int | I | 0.26 | 2.07 | 7.90 |
| HLA072 | 17 | 31087849 | 31087921 | ERV_1647849 | ERVL-int | I | 0.00 | 2.00 | 300.00 |
| HLA065 | 17 | 32342830 | 32342978 | ERV_1649005 | MSTD,MSTD-int | LI | 0.00 | 2.83 | 300.00 |
| HLA072 | 17 | 43249643 | 43249997 | ERV_1657934 | ERV3-16A3_I-int | I | 0.32 | 2.18 | 6.76 |
| HLA069 | 18 | 15199228 | 15199434 | ERV_1760683 | ERVL-E-int | I | 0.00 | 1.96 | 300.00 |
| HLA065 | 19 | 12418791 | 12421276 | ERV_1810824 | MER34-int | I | 0.27 | 1.57 | 5.83 |
| HLA053 | 19 | 15992309 | 15992400 | ERV_1815774 | THE1D-int | I | 0.00 | 2.00 | 300.00 |
| HLA053 | 19 | 23790297 | 23790393 | ERV_1823978 | MER57A-int | I | 0.00 | 2.38 | 300.00 |
| HLA076 | 19 | 43979355 | 43980979 | ERV_1841932 | ERVL-B4-int,MLT2C1 | LI | 1.78 | 3.86 | 2.16 |
| HLA072 | 19 | 51828006 | 51828656 | ERV_1852225 | MLT1J-int | I | 0.81 | 1.70 | 2.09 |
| HLA061 | 19 | 56380585 | 56381210 | ERV_1857590 | ERV3-16A3_I-int | I | 0.00 | 1.72 | 300.00 |
| HLA067 | 19 | 2826502 | 2826655 | ERV_1862034 | MER68-int | I | 0.36 | 1.92 | 5.35 |
| HLA071 | 19 | 9825001 | 9825090 | ERV_1869157 | PABL_B-int | I | 0.46 | 3.00 | 6.51 |
| HLA065 | 19 | 12573722 | 12575780 | ERV_1872113 | LTR52,LTR52-int | LI | 0.82 | 5.33 | 6.54 |
| HLA067 | 19 | 52649340 | 52649401 | ERV_1903573 | HERVIP10B3-int | I | 1.00 | 2.30 | 2.30 |
| HLA066 | 2 | 8437515 | 8440768 | ERV_1915295 | HERV16-int | I | 0.17 | 1.98 | 11.48 |
| HLA067 | 2 | 8437515 | 8440768 | ERV_1915295 | HERV16-int | I | 0.42 | 1.95 | 4.68 |
| HLA072 | 2 | 8437515 | 8440768 | ERV_1915295 | HERV16-int | I | 0.73 | 2.85 | 3.89 |
| HLA067 | 2 | 8441109 | 8441583 | ERV_1915298 | HERV16-int | I | 0.63 | 1.89 | 3.00 |
| HLA069 | 2 | 8441109 | 8441583 | ERV_1915298 | HERV16-int | I | 0.92 | 2.04 | 2.21 |
| HLA072 | 2 | 8441109 | 8441583 | ERV_1915298 | HERV16-int | I | 0.59 | 6.31 | 10.71 |
| HLA063 | 2 | 8828744 | 8829072 | ERV_1915636 | MLT1-int | I | 0.00 | 3.17 | 300.00 |
| HLA067 | 2 | 8828744 | 8829072 | ERV_1915636 | MLT1-int | I | 0.00 | 1.63 | 300.00 |
| HLA078 | 2 | 8828744 | 8829072 | ERV_1915636 | MLT1-int | I | 0.00 | 1.50 | 300.00 |
| HLA061 | 2 | 10003790 | 10004798 | ERV_1916838 | ERVL-E-int,LTR33 | LI | 0.00 | 1.93 | 300.00 |
| HLA066 | 2 | 11425312 | 11426640 | ERV_1918345 | HERVH48-int,MER48 | LI | 0.00 | 1.55 | 300.00 |
| HLA078 | 2 | 11797488 | 11798261 | ERV_1918683 | THE1D,THE1D-int | LI | 1.12 | 7.97 | 7.13 |
| HLA078 | 2 | 11864859 | 11866327 | ERV_1918742 | ERV3-16A3_I-int,LTR16A1 | LI | 0.00 | 14.86 | 300.00 |
| HLA078 | 2 | 12569680 | 12575439 | ERV_1919398 | HERVH-int,LTR7Y | LIL | 0.00 | 1.58 | 300.00 |
| HLA058 | 2 | 12858627 | 12862383 | ERV_1919673 | MLT1F2,MLT1F2-int | LIL | 0.00 | 2.96 | 300.00 |
| HLA058 | 2 | 15304068 | 15305786 | ERV_1921825 | THE1D,THE1D-int | LIL | 1.50 | 3.38 | 2.25 |
| HLA065 | 2 | 15304068 | 15305786 | ERV_1921825 | THE1D,THE1D-int | LIL | 0.18 | 1.90 | 10.75 |
| HLA053 | 2 | 15370591 | 15371353 | ERV_1921898 | THE1D,THE1D-int | LI | 0.33 | 2.31 | 6.91 |
| HLA058 | 2 | 15865782 | 15867868 | ERV_1922377 | MLT1G1,MLT1G1-int | LIL | 0.00 | 1.66 | 300.00 |
| HLA061 | 2 | 15865782 | 15867868 | ERV_1922377 | MLT1G1,MLT1G1-int | LIL | 1.49 | 3.10 | 2.08 |
| HLA063 | 2 | 15865782 | 15867868 | ERV_1922377 | MLT1G1,MLT1G1-int | LIL | 0.63 | 2.74 | 4.37 |
| HLA065 | 2 | 15865782 | 15867868 | ERV_1922377 | MLT1G1,MLT1G1-int | LIL | 0.12 | 2.09 | 17.06 |
| HLA067 | 2 | 15865782 | 15867868 | ERV_1922377 | MLT1G1,MLT1G1-int | LIL | 0.36 | 5.17 | 14.42 |
| HLA053 | 2 | 20268197 | 20268237 | ERV_1926286 | HERVL66-int | I | 0.78 | 2.75 | 3.55 |
| HLA066 | 2 | 20268197 | 20268237 | ERV_1926286 | HERVL66-int | I | 0.00 | 1.85 | 300.00 |
| HLA066 | 2 | 21089108 | 21089599 | ERV_1927021 | MLT1J1,MLT1J1-int | LI | 0.30 | 2.34 | 7.86 |
| HLA067 | 2 | 21089108 | 21089599 | ERV_1927021 | MLT1J1,MLT1J1-int | LI | 1.76 | 5.72 | 3.25 |
| HLA082 | 2 | 21396567 | 21398547 | ERV_1927226 | ERV3-16A3_I-int,LTR16,LTR16A | LIL | 0.00 | 1.89 | 300.00 |
| HLA067 | 2 | 24992340 | 24992445 | ERV_1930418 | MER61-int | I | 0.00 | 1.86 | 300.00 |
| HLA058 | 2 | 25325538 | 25327302 | ERV_1930816 | ERVL-E-int,LTR41 | LI | 0.00 | 1.55 | 300.00 |
| HLA071 | 2 | 25410436 | 25410584 | ERV_1930909 | LTR23-int | I | 0.00 | 1.88 | 300.00 |
| HLA078 | 2 | 27162225 | 27167226 | ERV_1932897 | ERV24_Prim-int,LTR23 | LI | 0.01 | 2.77 | 541.13 |
| HLA072 | 2 | 32353519 | 32355800 | ERV_1938025 | MER31-int,MER31A | LI | 0.19 | 2.49 | 13.10 |
| HLA058 | 2 | 32532875 | 32533724 | ERV_1938188 | MLT1D-int | I | 0.66 | 3.01 | 4.56 |
| HLA061 | 2 | 32532875 | 32533724 | ERV_1938188 | MLT1D-int | I | 0.92 | 1.87 | 2.02 |
| HLA069 | 2 | 32532875 | 32533724 | ERV_1938188 | MLT1D-int | I | 0.28 | 2.15 | 7.64 |
| HLA078 | 2 | 34677193 | 34678131 | ERV_1940030 | Harlequin-int,LTR2B | LI | 1.88 | 16.49 | 8.78 |
| HLA053 | 2 | 36564682 | 36564979 | ERV_1941472 | MLT1J-int | I | 0.00 | 2.73 | 300.00 |
| HLA078 | 2 | 36564682 | 36564979 | ERV_1941472 | MLT1J-int | I | 0.52 | 4.09 | 7.90 |
| HLA065 | 2 | 37160112 | 37160407 | ERV_1942111 | ERVL-B4-int | I | 0.00 | 3.19 | 300.00 |
| HLA053 | 2 | 39219723 | 39220586 | ERV_1944191 | ERVL-E-int | I | 0.26 | 1.78 | 6.75 |
| HLA058 | 2 | 39219723 | 39220586 | ERV_1944191 | ERVL-E-int | I | 0.43 | 4.49 | 10.54 |
| HLA058 | 2 | 42773497 | 42783034 | ERV_1947414 | MER4-int,MER4A1 | LIL | 3.06 | 7.44 | 2.43 |
| HLA065 | 2 | 42773497 | 42783034 | ERV_1947414 | MER4-int,MER4A1 | LIL | 0.55 | 2.63 | 4.77 |
| HLA058 | 2 | 42775270 | 42782558 | ERV_1947416 | MER4-int | I | 3.06 | 7.44 | 2.43 |
| HLA065 | 2 | 42775270 | 42782558 | ERV_1947416 | MER4-int | I | 0.55 | 2.63 | 4.77 |
| HLA073 | 2 | 42835193 | 42835480 | ERV_1947484 | MLT1H-int | I | 0.00 | 2.50 | 300.00 |
| HLA058 | 2 | 43010014 | 43014936 | ERV_1947634 | MLT1J,MLT1J-int | LIL | 0.26 | 2.00 | 7.64 |
| HLA058 | 2 | 46903003 | 46905220 | ERV_1951274 | THE1A,THE1A-int | LIL | 56.2 2 | 475.52 | 8.46 |
| HLA067 | 2 | 46903003 | 46905220 | ERV_1951274 | THE1A,THE1A-int | LIL | 284, 58 | 577.86 | 2.03 |
| HLA069 | 2 | 46903003 | 46905220 | ERV_1951274 | THE1A,THE1A-int | LIL | 150, 81 | 364.24 | 2.42 |
| HLA071 | 2 | 46903003 | 46905220 | ERV_1951274 | THE1A,THE1A-int | LIL | 232, 68 | 601.35 | 2.58 |
| HLA076 | 2 | 46903003 | 46905220 | ERV_1951274 | THE1A,THE1A-int | LIL | 136, 03 | 635.46 | 4.67 |
| HLA053 | 2 | 46918461 | 46919734 | ERV_1951292 | MLT1L,MLT1L-int | LI | 0.87 | 1.77 | 2.04 |
| HLA058 | 2 | 46918461 | 46919734 | ERV_1951292 | MLT1L,MLT1L-int | LI | 0.71 | 1.94 | 2.71 |
| HLA066 | 2 | 46918461 | 46919734 | ERV_1951292 | MLT1L,MLT1L-int | LI | 0.21 | 3.12 | 14.82 |
| HLA078 | 2 | 46918461 | 46919734 | ERV_1951292 | MLT1L,MLT1L-int | LI | 0.93 | 2.80 | 3.01 |
| HLA053 | 2 | 47868611 | 47870150 | ERV_1952334 | ERVL-B4-int,MLT2D | LI | 0.00 | 2.29 | 300.00 |
| HLA053 | 2 | 53704770 | 53705082 | ERV_1957247 | MER101-int | I | 0.05 | 3.57 | 69.63 |
| HLA069 | 2 | 53704770 | 53705082 | ERV_1957247 | MER101-int | I | 0.00 | 2.53 | 300.00 |
| HLA071 | 2 | 53704770 | 53705082 | ERV_1957247 | MER101-int | I | 1.42 | 3.57 | 2.52 |
| HLA053 | 2 | 53756643 | 53761425 | ERV_1957309 | HERV17-int,LTR17 | LIL | 0.81 | 3.53 | 4.37 |
| HLA065 | 2 | 53756643 | 53761425 | ERV_1957309 | HERV17-int,LTR17 | LIL | 0.60 | 2.47 | 4.14 |
| HLA067 | 2 | 53756643 | 53761425 | ERV_1957309 | HERV17-int,LTR17 | LIL | 2.41 | 10.22 | 4.24 |
| HLA065 | 2 | 53846882 | 53847622 | ERV_1957413 | ERVL-E-int | I | 0.00 | 2.44 | 300.00 |
| HLA073 | 2 | 53846882 | 53847622 | ERV_1957413 | ERVL-E-int | I | 0.22 | 1.86 | 8.41 |
| HLA053 | 2 | 54092460 | 54093159 | ERV_1957703 | ERV3-16A3_I-int,LTR16 | LI | 0.00 | 2.14 | 300.00 |
| HLA061 | 2 | 55625410 | 55626717 | ERV_1959131 | ERV3-16A3_I-int,LTR16B1 | LI | 0.49 | 2.91 | 6.01 |
| HLA063 | 2 | 55625410 | 55626717 | ERV_1959131 | ERV3-16A3_I-int,LTR16B1 | LI | 2.03 | 4.33 | 2.13 |
| HLA065 | 2 | 55625410 | 55626717 | ERV_1959131 | ERV3-16A3_I-int,LTR16B1 | LI | 0.11 | 2.88 | 25.23 |
| HLA067 | 2 | 55625410 | 55626717 | ERV_1959131 | ERV3-16A3_I-int,LTR16B1 | LI | 0.52 | 6.38 | 12.29 |
| HLA078 | 2 | 55625410 | 55626717 | ERV_1959131 | ERV3-16A3_I-int,LTR16B1 | LI | 0.04 | 2.66 | 60.02 |
| HLA082 | 2 | 55625410 | 55626717 | ERV_1959131 | ERV3-16A3_I-int,LTR16B1 | LI | 0.76 | 1.60 | 2.11 |
| HLA072 | 2 | 60957288 | 60958158 | ERV_1963425 | HERVL18-int,LTR18A | LI | 0.31 | 2.02 | 6.60 |
| HLA066 | 2 | 61721346 | 61721655 | ERV_1964444 | ERV3-16A3_I-int | I | 0.00 | 2.03 | 300.00 |
| HLA078 | 2 | 61875631 | 61876008 | ERV_1964662 | MER41-int | I | 0.31 | 3.50 | 11.49 |
| HLA053 | 2 | 62730625 | 62730785 | ERV_1965455 | HERVIP10FH-int | I | 2.94 | 13.78 | 4.69 |
| HLA065 | 2 | 62730625 | 62730785 | ERV_1965455 | HERVIP10FH-int | I | 0.00 | 2.81 | 300.00 |
| HLA072 | 2 | 62730625 | 62730785 | ERV_1965455 | HERVIP10FH-int | I | 1.08 | 3.48 | 3.24 |
| HLA058 | 2 | 63988445 | 63997140 | ERV_1966469 | HERVK11-int,MER11B,MER11C | LIL | 0.21 | 5.47 | 26.49 |
| HLA058 | 2 | 64252414 | 64257646 | ERV_1966699 | HERVH-int,LTR7 | LIL | 0.97 | 6.17 | 6.39 |
| HLA078 | 2 | 64252414 | 64257646 | ERV_1966699 | HERVH-int,LTR7 | LIL | 0.74 | 1.73 | 2.33 |
| HLA058 | 2 | 65053217 | 65053895 | ERV_1967539 | ERVL-E-int | I | 0.44 | 1.73 | 3.89 |
| HLA072 | 2 | 65053217 | 65053895 | ERV_1967539 | ERVL-E-int | I | 0.44 | 2.97 | 6.76 |
| HLA067 | 2 | 69318884 | 69318963 | ERV_1971341 | ERVL-B4-int | I | 0.44 | 1.68 | 3.80 |
| HLA076 | 2 | 69318884 | 69318963 | ERV_1971341 | ERVL-B4-int | I | 0.00 | 1.81 | 300.00 |
| HLA065 | 2 | 69450459 | 69450725 | ERV_1971502 | ERV24B_Prim-int | I | 0.10 | 1.96 | 19.33 |
| HLA066 | 2 | 69453399 | 69453528 | ERV_1971511 | MER57-int | I | 0.00 | 2.85 | 300.00 |
| HLA078 | 2 | 69453399 | 69453528 | ERV_1971511 | MER57-int | I | 0.00 | 2.00 | 300.00 |
| HLA078 | 2 | 71388099 | 71395817 | ERV_1973505 | HERVK22-int,LTR22E | LIL | 1.03 | 4.59 | 4.44 |
| HLA072 | 2 | 71444089 | 71446102 | ERV_1973538 | ERVL-E-int | I | 1.35 | 3.91 | 2.89 |
| HLA078 | 2 | 71444089 | 71446102 | ERV_1973538 | ERVL-E-int | I | 0.82 | 1.77 | 2.17 |
| HLA061 | 2 | 72994297 | 72994549 | ERV_1974905 | ERVL-E-int | I | 0.08 | 5.48 | 72.68 |
| HLA072 | 2 | 72994297 | 72994549 | ERV_1974905 | ERVL-E-int | I | 1.12 | 2.50 | 2.23 |
| HLA078 | 2 | 73271213 | 73273836 | ERV_1975221 | MLT1K,MLT1K-int | LIL | 0.07 | 3.16 | 45.26 |
| HLA058 | 2 | 73738001 | 73738129 | ERV_1975566 | MST-int | I | 0.00 | 1.62 | 300.00 |
| HLA061 | 2 | 73738001 | 73738129 | ERV_1975566 | MST-int | I | 0.00 | 1.59 | 300.00 |
| HLA063 | 2 | 73738001 | 73738129 | ERV_1975566 | MST-int | I | 0.00 | 2.81 | 300.00 |
| HLA066 | 2 | 73738001 | 73738129 | ERV_1975566 | MST-int | I | 0.00 | 2.70 | 300.00 |
| HLA069 | 2 | 85817949 | 85818174 | ERV_1985980 | ERVL-E-int | I | 0.00 | 2.27 | 300.00 |
| HLA067 | 2 | 87039336 | 87043487 | ERV_1987156 | LTR28C,MER52-int | LI | 0.40 | 1.61 | 4.01 |
| HLA071 | 2 | 87039336 | 87043487 | ERV_1987156 | LTR28C,MER52-int | LI | 0.37 | 1.85 | 4.99 |
| HLA066 | 2 | 87590144 | 87592567 | ERV_1987621 | MSTA,MSTA-int | LIL | 0.09 | 1.50 | 17.34 |
| HLA078 | 2 | 87590144 | 87592567 | ERV_1987621 | MSTA,MSTA-int | LIL | 0.16 | 2.45 | 15.43 |
| HLA053 | 2 | 88581793 | 88582614 | ERV_1988703 | HERVL-int | I | 0.25 | 5.03 | 20.34 |
| HLA069 | 2 | 88581793 | 88582614 | ERV_1988703 | HERVL-int | I | 1.46 | 3.28 | 2.24 |
| HLA058 | 2 | 88679963 | 88686725 | ERV_1988796 | MER50,MER50-int | LIL | 0.75 | 3.71 | 4.95 |
| HLA061 | 2 | 88679963 | 88686725 | ERV_1988796 | MER50,MER50-int | LIL | 0.68 | 1.65 | 2.42 |
| HLA078 | 2 | 88679963 | 88686725 | ERV_1988796 | MER50,MER50-int | LIL | 0.29 | 3.34 | 11.57 |
| HLA061 | 2 | 90224760 | 90225079 | ERV_1989710 | ERVL-B4-int | I | 0.00 | 5.54 | 300.00 |
| HLA061 | 2 | 90227941 | 90228534 | ERV_1989713 | MER70-int,MER70A | LI | 0.00 | 2.48 | 300.00 |
| HLA061 | 2 | 90228581 | 90231442 | ERV_1989714 | MER66-int,MER66A | LI | 0.00 | 1.57 | 300.00 |
| HLA063 | 2 | 94229830 | 94230277 | ERV_1990538 | MER34B-int,MER34C_ | LI | 0.00 | 1.91 | 300.00 |
| HLA067 | 2 | 94229830 | 94230277 | ERV_1990538 | MER34B-int,MER34C_ | LI | 0.00 | 2.61 | 300.00 |
| HLA065 | 2 | 95154290 | 95156042 | ERV_1991061 | MLT1I,MLT1I-int | LIL | 0.15 | 1.90 | 12.78 |
| HLA078 | 2 | 95154290 | 95156042 | ERV_1991061 | MLT1I,MLT1I-int | LIL | 0.95 | 3.45 | 3.62 |
| HLA058 | 2 | 96233989 | 96239799 | ERV_1991992 | ERV3-16A3_I-int,LTR16A,LTR16B2 | LIL | 0.29 | 2.14 | 7.36 |
| HLA069 | 2 | 96233989 | 96239799 | ERV_1991992 | ERV3-16A3_I-int,LTR16A,LTR16B2 | LIL | 0.76 | 1.73 | 2.27 |
| HLA078 | 2 | 96233989 | 96239799 | ERV_1991992 | ERV3-16A3_I-int,LTR16A,LTR16B2 | LIL | 0.58 | 2.24 | 3.85 |
| HLA058 | 2 | 96235418 | 96238331 | ERV_1991997 | ERVL-E-int | I | 0.29 | 2.14 | 7.36 |
| HLA069 | 2 | 96235418 | 96238331 | ERV_1991997 | ERVL-E-int | I | 0.76 | 1.73 | 2.27 |
| HLA078 | 2 | 96235418 | 96238331 | ERV_1991997 | ERVL-E-int | I | 0.58 | 2.24 | 3.85 |
| HLA072 | 2 | 96315157 | 96315867 | ERV_1992080 | HERVL40-int,LTR40c | LI | 0.95 | 2.36 | 2.49 |
| HLA065 | 2 | 96565704 | 96572402 | ERV_1992320 | MER92-int,MER92D | LI | 0.00 | 1.71 | 300.00 |
| HLA053 | 2 | 99200956 | 99201109 | ERV_1994513 | MER110-int | I | 2.63 | 6.44 | 2.45 |
| HLA058 | 2 | 99200956 | 99201109 | ERV_1994513 | MER110-int | I | 0.00 | 7.93 | 300.00 |
| HLA067 | 2 | 99200956 | 99201109 | ERV_1994513 | MER110-int | I | 0.00 | 11.39 | 300.00 |
| HLA071 | 2 | 99200956 | 99201109 | ERV_1994513 | MER110-int | I | 1.00 | 4.90 | 4.90 |
| HLA072 | 2 | 99200956 | 99201109 | ERV_1994513 | MER110-int | I | 0.45 | 9.18 | 20.35 |
| HLA076 | 2 | 99200956 | 99201109 | ERV_1994513 | MER110-int | I | 2.54 | 10.27 | 4.04 |
| HLA082 | 2 | 99200956 | 99201109 | ERV_1994513 | MER110-int | I | 2.78 | 13.31 | 4.78 |
| HLA053 | 2 | 99201154 | 99201453 | ERV_1994514 | MER89-int | I | 2.62 | 7.65 | 2.92 |
| HLA058 | 2 | 99201154 | 99201453 | ERV_1994514 | MER89-int | I | 0.00 | 7.97 | 300.00 |
| HLA067 | 2 | 99201154 | 99201453 | ERV_1994514 | MER89-int | I | 0.56 | 5.86 | 10.55 |
| HLA071 | 2 | 99201154 | 99201453 | ERV_1994514 | MER89-int | I | 0.52 | 3.63 | 7.05 |
| HLA076 | 2 | 99201154 | 99201453 | ERV_1994514 | MER89-int | I | 2.92 | 7.17 | 2.45 |
| HLA082 | 2 | 99201154 | 99201453 | ERV_1994514 | MER89-int | I | 2.47 | 5.33 | 2.16 |
| HLA063 | 2 | 99236693 | 99238143 | ERV_1994543 | MER21-int | I | 0.88 | 3.54 | 4.02 |
| HLA065 | 2 | 99236693 | 99238143 | ERV_1994543 | MER21-int | I | 2.60 | 6.39 | 2.46 |
| HLA072 | 2 | 99236693 | 99238143 | ERV_1994543 | MER21-int | I | 0.71 | 5.54 | 7.80 |
| HLA058 | 2 | 99238180 | 99238330 | ERV_1994545 | MER21-int | I | 0.23 | 2.20 | 9.71 |
| HLA061 | 2 | 99238180 | 99238330 | ERV_1994545 | MER21-int | I | 0.00 | 4.29 | 300.00 |
| HLA063 | 2 | 99238180 | 99238330 | ERV_1994545 | MER21-int | I | 0.13 | 2.04 | 16.11 |
| HLA065 | 2 | 99238180 | 99238330 | ERV_1994545 | MER21-int | I | 0.00 | 2.03 | 300.00 |
| HLA072 | 2 | 99238180 | 99238330 | ERV_1994545 | MER21-int | I | 0.00 | 6.24 | 300.00 |
| HLA053 | 2 | 99303711 | 99304487 | ERV_1994616 | THE1B,THE1B-int | LIL | 0.75 | 2.78 | 3.70 |
| HLA058 | 2 | 99303711 | 99304487 | ERV_1994616 | THE1B,THE1B-int | LIL | 0.00 | 1.53 | 300.00 |
| HLA058 | 2 | 1E+08 | 1E+08 | ERV_1995473 | HERVL74-int,MER74B | LIL | 0.78 | 8.66 | 11.12 |
| HLA058 | 2 | 1.01E+08 | 1.01E+08 | ERV_1996284 | ERVL-E-int,LTR33 | LI | 0.38 | 4.98 | 13.11 |
| HLA069 | 2 | 1.01E+08 | 1.01E+08 | ERV_1996284 | ERVL-E-int,LTR33 | LI | 0.64 | 1.93 | 3.01 |
| HLA053 | 2 | 1.02E+08 | 1.02E+08 | ERV_1997060 | HERVIP10FH-int | I | 8.68 | 30.40 | 3.50 |
| HLA061 | 2 | 1.09E+08 | 1.09E+08 | ERV_2002448 | Harlequin-int,LTR2 | LIL | 0.46 | 1.54 | 3.34 |
| HLA063 | 2 | 1.09E+08 | 1.09E+08 | ERV_2002448 | Harlequin-int,LTR2 | LIL | 0.16 | 1.67 | 10.72 |
| HLA071 | 2 | 1.09E+08 | 1.09E+08 | ERV_2002448 | Harlequin-int,LTR2 | LIL | 0.42 | 2.06 | 4.96 |
| HLA058 | 2 | 1.1E+08 | 1.1E+08 | ERV_2003903 | ERVL-B4-int,MLT2C1 | LI | 2.66 | 13.03 | 4.91 |
| HLA065 | 2 | 1.1E+08 | 1.1E+08 | ERV_2003903 | ERVL-B4-int,MLT2C1 | LI | 2.60 | 7.31 | 2.81 |
| HLA067 | 2 | 1.1E+08 | 1.1E+08 | ERV_2003903 | ERVL-B4-int,MLT2C1 | LI | 1.05 | 2.91 | 2.78 |
| HLA058 | 2 | 1.13E+08 | 1.13E+08 | ERV_2005804 | HERVL40-int | I | 0.95 | 3.65 | 3.84 |
| HLA065 | 2 | 1.13E+08 | 1.13E+08 | ERV_2005804 | HERVL40-int | I | 1.13 | 3.39 | 2.99 |
| HLA071 | 2 | 1.13E+08 | 1.13E+08 | ERV_2005804 | HERVL40-int | I | 2.40 | 5.19 | 2.16 |
| HLA072 | 2 | 1.13E+08 | 1.13E+08 | ERV_2005804 | HERVL40-int | I | 2.13 | 6.68 | 3.14 |
| HLA073 | 2 | 1.13E+08 | 1.13E+08 | ERV_2005804 | HERVL40-int | I | 1.74 | 4.19 | 2.41 |
| HLA082 | 2 | 1.13E+08 | 1.13E+08 | ERV_2005804 | HERVL40-int | I | 1.01 | 4.71 | 4.69 |
| HLA053 | 2 | 1.13E+08 | 1.13E+08 | ERV_2005885 | ERVL-E-int | I | 0.59 | 2.05 | 3.46 |
| HLA063 | 2 | 1.13E+08 | 1.13E+08 | ERV_2006565 | THE1B,THE1B-int | LIL | 2.40 | 6.42 | 2.67 |
| HLA071 | 2 | 1.13E+08 | 1.13E+08 | ERV_2006565 | THE1B,THE1B-int | LIL | 2.88 | 5.95 | 2.06 |
| HLA073 | 2 | 1.13E+08 | 1.13E+08 | ERV_2006565 | THE1B,THE1B-int | LIL | 0.57 | 2.33 | 4.05 |
| HLA076 | 2 | 1.13E+08 | 1.13E+08 | ERV_2006565 | THE1B,THE1B-int | LIL | 1.48 | 12.31 | 8.32 |
| HLA066 | 2 | 1.14E+08 | 1.14E+08 | ERV_2006715 | MER31-int | I | 1.78 | 5.35 | 3.00 |
| HLA058 | 2 | 1.14E+08 | 1.14E+08 | ERV_2006716 | MER92-int | I | 0.19 | 1.55 | 8.12 |
| HLA073 | 2 | 1.14E+08 | 1.14E+08 | ERV_2006718 | MER92-int | I | 0.45 | 1.54 | 3.45 |
| HLA072 | 2 | 1.17E+08 | 1.17E+08 | ERV_2009540 | MSTA,MSTA-int | LIL | 0.00 | 1.65 | 300.00 |
| HLA066 | 2 | 1.18E+08 | 1.18E+08 | ERV_2010325 | THE1B,THE1B-int | LIL | 0.75 | 1.71 | 2.28 |
| HLA071 | 2 | 1.18E+08 | 1.18E+08 | ERV_2010325 | THE1B,THE1B-int | LIL | 0.31 | 1.75 | 5.59 |
| HLA063 | 2 | 1.21E+08 | 1.21E+08 | ERV_2013434 | MSTA,MSTA-int | LIL | 0.61 | 4.91 | 8.08 |
| HLA065 | 2 | 1.21E+08 | 1.21E+08 | ERV_2013434 | MSTA,MSTA-int | LIL | 1.08 | 5.78 | 5.37 |
| HLA072 | 2 | 1.21E+08 | 1.21E+08 | ERV_2013434 | MSTA,MSTA-int | LIL | 1.35 | 3.34 | 2.48 |
| HLA065 | 2 | 1.28E+08 | 1.28E+08 | ERV_2019273 | ERVL-E-int | I | 0.00 | 3.18 | 300.00 |
| HLA067 | 2 | 1.28E+08 | 1.28E+08 | ERV_2019273 | ERVL-E-int | I | 0.00 | 2.81 | 300.00 |
| HLA058 | 2 | 1.3E+08 | 1.3E+08 | ERV_2021040 | MER101-int,MER65C | LI | 0.00 | 3.30 | 300.00 |
| HLA069 | 2 | 1.3E+08 | 1.3E+08 | ERV_2021067 | MST-int | I | 0.00 | 2.02 | 300.00 |
| HLA069 | 2 | 1.3E+08 | 1.3E+08 | ERV_2021069 | THE1D-int | I | 0.00 | 2.02 | 300.00 |
| HLA058 | 2 | 1.31E+08 | 1.31E+08 | ERV_2022115 | MER4-int | I | 0.00 | 2.12 | 300.00 |
| HLA058 | 2 | 1.31E+08 | 1.31E+08 | ERV_2022123 | MSTA,MSTA-int | LI | 0.00 | 2.83 | 300.00 |
| HLA058 | 2 | 1.31E+08 | 1.31E+08 | ERV_2022124 | THE1D-int | I | 0.00 | 2.83 | 300.00 |
| HLA069 | 2 | 1.32E+08 | 1.32E+08 | ERV_2022303 | ERVL-B4-int,MLT2B1 | LI | 0.00 | 1.90 | 300.00 |
| HLA058 | 2 | 1.32E+08 | 1.32E+08 | ERV_2022331 | MER4-int,MER4A1 | LI | 0.00 | 2.01 | 300.00 |
| HLA058 | 2 | 1.32E+08 | 1.32E+08 | ERV_2022376 | MER4-int | I | 0.66 | 32.96 | 49.76 |
| HLA058 | 2 | 1.36E+08 | 1.36E+08 | ERV_2025737 | MER4B-int,MER4E | LI | 0.29 | 1.54 | 5.32 |
| HLA065 | 2 | 1.36E+08 | 1.36E+08 | ERV_2025737 | MER4B-int,MER4E | LI | 0.43 | 1.79 | 4.12 |
| HLA067 | 2 | 1.43E+08 | 1.43E+08 | ERV_2032133 | THE1B,THE1B-int | LIL | 1.24 | 3.13 | 2.52 |
| HLA058 | 2 | 1.45E+08 | 1.45E+08 | ERV_2033576 | MLT1J1,MLT1J1-int | LI | 0.00 | 4.29 | 300.00 |
| HLA058 | 2 | 1.45E+08 | 1.45E+08 | ERV_2033871 | MLT1F2,MLT1F2-int | LI | 0.00 | 9.49 | 300.00 |
| HLA065 | 2 | 1.6E+08 | 1.6E+08 | ERV_2045461 | LTR37-int | I | 0.31 | 2.46 | 7.98 |
| HLA058 | 2 | 1.7E+08 | 1.7E+08 | ERV_2053727 | MSTB1,MSTB1-int | LIL | 0.30 | 4.20 | 13.87 |
| HLA065 | 2 | 1.7E+08 | 1.7E+08 | ERV_2053727 | MSTB1,MSTB1-int | LIL | 0.33 | 2.40 | 7.39 |
| HLA067 | 2 | 1.7E+08 | 1.7E+08 | ERV_2053727 | MSTB1,MSTB1-int | LIL | 0.48 | 2.49 | 5.16 |
| HLA072 | 2 | 1.7E+08 | 1.7E+08 | ERV_2053727 | MSTB1,MSTB1-int | LIL | 0.17 | 2.93 | 16.81 |
| HLA066 | 2 | 1.7E+08 | 1.7E+08 | ERV_2054017 | MLT1I,MLT1I-int | LI | 0.10 | 1.97 | 20.46 |
| HLA058 | 2 | 1.7E+08 | 1.7E+08 | ERV_2054018 | MLT1I-int | I | 0.00 | 1.97 | 300.00 |
| HLA067 | 2 | 1.71E+08 | 1.71E+08 | ERV_2054890 | MLT1D,MLT1D-int | LI | 0.00 | 1.70 | 300.00 |
| HLA058 | 2 | 1.71E+08 | 1.71E+08 | ERV_2055246 | LTR79,THE1B,THE1B-int | LIL | 0.81 | 2.49 | 3.08 |
| HLA069 | 2 | 1.71E+08 | 1.71E+08 | ERV_2055246 | LTR79,THE1B,THE1B-int | LIL | 0.48 | 1.51 | 3.14 |
| HLA065 | 2 | 1.72E+08 | 1.72E+08 | ERV_2055877 | MLT1D,MLT1D-int | LI | 0.67 | 3.09 | 4.64 |
| HLA072 | 2 | 1.78E+08 | 1.78E+08 | ERV_2060929 | HERV35I-int | I | 0.00 | 1.89 | 300.00 |
| HLA065 | 2 | 1.78E+08 | 1.78E+08 | ERV_2061533 | HERV17-int,LTR17 | LIL | 0.25 | 1.52 | 5.97 |
| HLA071 | 2 | 1.79E+08 | 1.79E+08 | ERV_2062230 | MER41-int,MER41B | LI | 0.61 | 2.81 | 4.60 |
| HLA065 | 2 | 1.86E+08 | 1.86E+08 | ERV_2066852 | THE1A,THE1A-int | LIL | 0.00 | 2.80 | 300.00 |
| HLA066 | 2 | 1.87E+08 | 1.87E+08 | ERV_2067643 | HERVK-int,LTR5B,LTR5_Hs | LIL | 0.95 | 1.97 | 2.07 |
| HLA082 | 2 | 1.87E+08 | 1.87E+08 | ERV_2068265 | LTR37-int,LTR37A | LIL | 0.72 | 1.60 | 2.22 |
| HLA082 | 2 | 1.87E+08 | 1.87E+08 | ERV_2068268 | LTR37-int | I | 0.72 | 1.60 | 2.22 |
| HLA058 | 2 | 1.87E+08 | 1.87E+08 | ERV_2068404 | THE1-int | I | 0.00 | 1.55 | 300.00 |
| HLA065 | 2 | 1.87E+08 | 1.87E+08 | ERV_2068404 | THE1-int | I | 0.00 | 2.08 | 300.00 |
| HLA067 | 2 | 1.87E+08 | 1.87E+08 | ERV_2068404 | THE1-int | I | 0.00 | 1.74 | 300.00 |
| HLA067 | 2 | 1.88E+08 | 1.88E+08 | ERV_2068412 | Harlequin-int,LTR2 | LIL | 2.59 | 5.95 | 2.30 |
| HLA069 | 2 | 1.88E+08 | 1.88E+08 | ERV_2068412 | Harlequin-int,LTR2 | LIL | 1.39 | 3.02 | 2.18 |
| HLA067 | 2 | 1.88E+08 | 1.88E+08 | ERV_2068413 | HERVE_a-int | I | 2.59 | 5.95 | 2.30 |
| HLA069 | 2 | 1.88E+08 | 1.88E+08 | ERV_2068413 | HERVE_a-int | I | 1.39 | 3.02 | 2.18 |
| HLA066 | 2 | 1.91E+08 | 1.91E+08 | ERV_2071264 | MSTB1,MSTB1-int | LIL | 0.17 | 1.62 | 9.26 |
| HLA071 | 2 | 1.91E+08 | 1.91E+08 | ERV_2071264 | MSTB1,MSTB1-int | LIL | 1.31 | 7.04 | 5.36 |
| HLA072 | 2 | 1.91E+08 | 1.91E+08 | ERV_2071264 | MSTB1,MSTB1-int | LIL | 0.76 | 2.29 | 3.02 |
| HLA073 | 2 | 1.91E+08 | 1.91E+08 | ERV_2071264 | MSTB1,MSTB1-int | LIL | 1.13 | 3.78 | 3.33 |
| HLA067 | 2 | 2.01E+08 | 2.01E+08 | ERV_2079453 | ERV3-16A3_I-int | I | 1.61 | 17.15 | 10.64 |
| HLA069 | 2 | 2.01E+08 | 2.01E+08 | ERV_2079453 | ERV3-16A3_I-int | I | 0.09 | 5.18 | 59.88 |
| HLA071 | 2 | 2.01E+08 | 2.01E+08 | ERV_2079453 | ERV3-16A3_I-int | I | 3.53 | 11.49 | 3.26 |
| HLA082 | 2 | 2.01E+08 | 2.01E+08 | ERV_2079453 | ERV3-16A3_I-int | I | 0.98 | 6.85 | 6.97 |
| HLA073 | 2 | 2.01E+08 | 2.01E+08 | ERV_2079783 | MER4-int | I | 0.00 | 1.61 | 300.00 |
| HLA072 | 2 | 2.01E+08 | 2.01E+08 | ERV_2079826 | MER21-int | I | 0.80 | 1.65 | 2.07 |
| HLA065 | 2 | 2.03E+08 | 2.03E+08 | ERV_2082200 | MER92-int | I | 1.31 | 11.42 | 8.69 |
| HLA067 | 2 | 2.06E+08 | 2.06E+08 | ERV_2084847 | MSTB,MSTB-int | LI | 18.3 7 | 51.21 | 2.79 |
| HLA069 | 2 | 2.06E+08 | 2.06E+08 | ERV_2084847 | MSTB,MSTB-int | LI | 13.0 9 | 30.95 | 2.37 |
| HLA076 | 2 | 2.06E+08 | 2.06E+08 | ERV_2084847 | MSTB,MSTB-int | LI | 23.1 6 | 54.92 | 2.37 |
| HLA069 | 2 | 2.09E+08 | 2.09E+08 | ERV_2087240 | ERVL-E-int | I | 0.00 | 2.34 | 300.00 |
| HLA071 | 2 | 2.09E+08 | 2.09E+08 | ERV_2087240 | ERVL-E-int | I | 1.26 | 3.77 | 2.99 |
| HLA065 | 2 | 2.1E+08 | 2.1E+08 | ERV_2088191 | THE1C,THE1C-int | LI | 0.00 | 2.21 | 923.49 |
| HLA072 | 2 | 2.1E+08 | 2.1E+08 | ERV_2088191 | THE1C,THE1C-int | LI | 1.08 | 4.17 | 3.86 |
| HLA065 | 2 | 2.1E+08 | 2.1E+08 | ERV_2088204 | MLT1H2,MLT1H2-int | LI | 0.06 | 2.24 | 35.80 |
| HLA072 | 2 | 2.1E+08 | 2.1E+08 | ERV_2088204 | MLT1H2,MLT1H2-int | LI | 0.14 | 1.81 | 13.03 |
| HLA065 | 2 | 2.1E+08 | 2.1E+08 | ERV_2088206 | MLT1H2-int | I | 0.06 | 2.24 | 35.80 |
| HLA072 | 2 | 2.1E+08 | 2.1E+08 | ERV_2088206 | MLT1H2-int | I | 0.14 | 1.81 | 13.03 |
| HLA063 | 2 | 2.16E+08 | 2.16E+08 | ERV_2093181 | ERV3-16A3_I-int,LTR16B | LIL | 3.70 | 14.64 | 3.96 |
| HLA065 | 2 | 2.16E+08 | 2.16E+08 | ERV_2093181 | ERV3-16A3_I-int,LTR16B | LIL | 1.57 | 7.96 | 5.07 |
| HLA066 | 2 | 2.16E+08 | 2.16E+08 | ERV_2093181 | ERV3-16A3_I-int,LTR16B | LIL | 2.83 | 10.66 | 3.77 |
| HLA067 | 2 | 2.16E+08 | 2.16E+08 | ERV_2093181 | ERV3-16A3_I-int,LTR16B | LIL | 8.14 | 44.25 | 5.44 |
| HLA069 | 2 | 2.16E+08 | 2.16E+08 | ERV_2093181 | ERV3-16A3_I-int,LTR16B | LIL | 1.38 | 4.58 | 3.31 |
| HLA072 | 2 | 2.16E+08 | 2.16E+08 | ERV_2093181 | ERV3-16A3_I-int,LTR16B | LIL | 1.50 | 5.23 | 3.48 |
| HLA073 | 2 | 2.16E+08 | 2.16E+08 | ERV_2093181 | ERV3-16A3_I-int,LTR16B | LIL | 2.18 | 6.71 | 3.08 |
| HLA082 | 2 | 2.16E+08 | 2.16E+08 | ERV_2093181 | ERV3-16A3_I-int,LTR16B | LIL | 3.59 | 7.44 | 2.07 |
| HLA061 | 2 | 2.16E+08 | 2.16E+08 | ERV_2093269 | MLT1F,MLT1F-int | LI | 0.00 | 2.44 | 300.00 |
| HLA063 | 2 | 2.16E+08 | 2.16E+08 | ERV_2093269 | MLT1F,MLT1F-int | LI | 0.00 | 2.10 | 300.00 |
| HLA067 | 2 | 2.16E+08 | 2.16E+08 | ERV_2093269 | MLT1F,MLT1F-int | LI | 0.29 | 1.65 | 5.65 |
| HLA067 | 2 | 2.23E+08 | 2.23E+08 | ERV_2099640 | MER57A-int | I | 0.06 | 1.96 | 30.79 |
| HLA067 | 2 | 2.23E+08 | 2.23E+08 | ERV_2099641 | MER57A-int | I | 0.00 | 2.33 | 300.00 |
| HLA073 | 2 | 2.23E+08 | 2.23E+08 | ERV_2099643 | MER101-int | I | 0.44 | 1.57 | 3.61 |
| HLA065 | 2 | 2.24E+08 | 2.24E+08 | ERV_2100712 | THE1B,THE1B-int | LIL | 0.13 | 2.21 | 17.57 |
| HLA067 | 2 | 2.24E+08 | 2.24E+08 | ERV_2100712 | THE1B,THE1B-int | LIL | 0.84 | 4.21 | 5.03 |
| HLA082 | 2 | 2.24E+08 | 2.24E+08 | ERV_2100712 | THE1B,THE1B-int | LIL | 0.65 | 2.81 | 4.30 |
| HLA067 | 2 | 2.3E+08 | 2.3E+08 | ERV_2106597 | HERVL-int,MLT2A1 | LIL | 0.16 | 4.13 | 26.41 |
| HLA067 | 2 | 2.3E+08 | 2.3E+08 | ERV_2106642 | LTR53-int | I | 2.17 | 5.04 | 2.33 |
| HLA067 | 2 | 2.3E+08 | 2.3E+08 | ERV_2106779 | ERV3-16A3_I-int,LTR16A1 | LI | 2.40 | 7.09 | 2.95 |
| HLA072 | 2 | 2.31E+08 | 2.31E+08 | ERV_2106847 | LOR1-int,LOR1a | LIL | 1.64 | 3.54 | 2.16 |
| HLA072 | 2 | 2.31E+08 | 2.31E+08 | ERV_2106856 | LOR1-int | I | 1.64 | 3.54 | 2.16 |
| HLA065 | 2 | 2.31E+08 | 2.31E+08 | ERV_2106916 | MLT1G,MLT1G-int | LI | 0.00 | 2.20 | 300.00 |
| HLA072 | 2 | 2.32E+08 | 2.32E+08 | ERV_2108130 | THE1A,THE1A-int | LIL | 0.62 | 1.75 | 2.81 |
| HLA072 | 2 | 2.33E+08 | 2.33E+08 | ERV_2109103 | ERVL-E-int | I | 0.00 | 2.12 | 300.00 |
| HLA065 | 2 | 2.33E+08 | 2.33E+08 | ERV_2109456 | MLT1-int | I | 1.18 | 2.67 | 2.27 |
| HLA072 | 2 | 2.33E+08 | 2.33E+08 | ERV_2109456 | MLT1-int | I | 0.34 | 2.17 | 6.34 |
| HLA065 | 2 | 2.33E+08 | 2.33E+08 | ERV_2109457 | MLT1-int | I | 1.18 | 2.67 | 2.27 |
| HLA072 | 2 | 2.33E+08 | 2.33E+08 | ERV_2109457 | MLT1-int | I | 0.34 | 2.17 | 6.34 |
| HLA069 | 2 | 2.34E+08 | 2.34E+08 | ERV_2110157 | THE1C,THE1C-int | LI | 0.16 | 1.61 | 10.35 |
| HLA076 | 2 | 2.34E+08 | 2.34E+08 | ERV_2110157 | THE1C,THE1C-int | LI | 0.00 | 1.73 | 300.00 |
| HLA063 | 2 | 2.34E+08 | 2.34E+08 | ERV_2110158 | THE1C,THE1C-int | LI | 0.47 | 1.52 | 3.27 |
| HLA065 | 2 | 2.34E+08 | 2.34E+08 | ERV_2110158 | THE1C,THE1C-int | LI | 0.00 | 2.19 | 300.00 |
| HLA076 | 2 | 2.34E+08 | 2.34E+08 | ERV_2110158 | THE1C,THE1C-int | LI | 0.95 | 2.55 | 2.68 |
| HLA063 | 2 | 2.34E+08 | 2.34E+08 | ERV_2110163 | THE1C,THE1C-int | LI | 0.65 | 2.13 | 3.29 |
| HLA066 | 2 | 2.34E+08 | 2.34E+08 | ERV_2110163 | THE1C,THE1C-int | LI | 0.58 | 3.19 | 5.54 |
| HLA071 | 2 | 2.34E+08 | 2.34E+08 | ERV_2110163 | THE1C,THE1C-int | LI | 2.59 | 16.89 | 6.51 |
| HLA073 | 2 | 2.34E+08 | 2.34E+08 | ERV_2110163 | THE1C,THE1C-int | LI | 0.96 | 3.48 | 3.63 |
| HLA076 | 2 | 2.34E+08 | 2.34E+08 | ERV_2110163 | THE1C,THE1C-int | LI | 1.76 | 8.21 | 4.67 |
| HLA071 | 2 | 2.34E+08 | 2.34E+08 | ERV_2110164 | THE1C,THE1C-int | LI | 4.79 | 20.25 | 4.23 |
| HLA076 | 2 | 2.34E+08 | 2.34E+08 | ERV_2110164 | THE1C,THE1C-int | LI | 2.69 | 15.98 | 5.95 |
| HLA061 | 2 | 2.35E+08 | 2.35E+08 | ERV_2110936 | MamGyp-int,MamGypLTR1d | LI | 0.41 | 2.46 | 5.96 |
| HLA069 | 2 | 2.35E+08 | 2.35E+08 | ERV_2110936 | MamGyp-int,MamGypLTR1d | LI | 0.00 | 2.18 | 300.00 |
| HLA071 | 2 | 2.35E+08 | 2.35E+08 | ERV_2110936 | MamGyp-int,MamGypLTR1d | LI | 0.14 | 3.33 | 23.93 |
| HLA069 | 2 | 2.35E+08 | 2.35E+08 | ERV_2110985 | MamRep605,THE1B,THE 1B-int | LIL | 0.03 | 2.20 | 73.94 |
| HLA071 | 2 | 2.38E+08 | 2.38E+08 | ERV_2113834 | MLTIJ-int | I | 1.78 | 4.05 | 2.27 |
| HLA082 | 2 | 2.38E+08 | 2.38E+08 | ERV_2113834 | MLTIJ-int | I | 0.00 | 2.61 | 300.00 |
| HLA065 | 2 | 2.38E+08 | 2.38E+08 | ERV_2114006 | ERV3-16A3_I-int | I | 0.00 | 1.98 | 300.00 |
| HLA067 | 2 | 2.38E+08 | 2.38E+08 | ERV_2114006 | ERV3-16A3_I-int | I | 0.00 | 2.57 | 300.00 |
| HLA076 | 2 | 2.38E+08 | 2.38E+08 | ERV_2114006 | ERV3-16A3_I-int | I | 0.00 | 2.23 | 300.00 |
| HLA082 | 2 | 2.38E+08 | 2.38E+08 | ERV_2114006 | ERV3-16A3_I-int | I | 0.32 | 3.40 | 10.48 |
| HLA065 | 2 | 2.41E+08 | 2.41E+08 | ERV_2115527 | LOR1-int,LOR1b | LI | 0.74 | 1.52 | 2.04 |
| HLA067 | 2 | 2.41E+08 | 2.41E+08 | ERV_2115527 | LOR1-int,LOR1b | LI | 0.00 | 1.66 | 300.00 |
| HLA066 | 2 | 2.41E+08 | 2.41E+08 | ERV_2115570 | THE1D,THE1D-int | LIL | 0.44 | 3.95 | 8.97 |
| HLA065 | 2 | 2.41E+08 | 2.41E+08 | ERV_2115723 | MER84,MER84-int | LIL | 0.30 | 1.85 | 6.20 |
| HLA065 | 2 | 2.41E+08 | 2.41E+08 | ERV_2115727 | MER84-int | I | 0.30 | 1.85 | 6.20 |
| HLA065 | 2 | 2.42E+08 | 2.42E+08 | ERV_2116259 | ERVL-B4-int | I | 1.11 | 6.45 | 5.79 |
| HLA066 | 2 | 2.42E+08 | 2.42E+08 | ERV_2116259 | ERVL-B4-int | I | 4.63 | 19.13 | 4.13 |
| HLA067 | 2 | 2.42E+08 | 2.42E+08 | ERV_2116259 | ERVL-B4-int | I | 10.26 | 23.32 | 2.27 |
| HLA069 | 2 | 2.42E+08 | 2.42E+08 | ERV_2116259 | ERVL-B4-int | I | 2.66 | 31.86 | 12.00 |
| HLA071 | 2 | 2.42E+08 | 2.42E+08 | ERV_2116259 | ERVL-B4-int | I | 17.51 | 43.16 | 2.47 |
| HLA073 | 2 | 2.42E+08 | 2.42E+08 | ERV_2116259 | ERVL-B4-int | I | 4.68 | 12.23 | 2.61 |
| HLA076 | 2 | 2.42E+08 | 2.42E+08 | ERV_2116259 | ERVL-B4-int | I | 10.37 | 26.78 | 2.58 |
| HLA058 | 2 | 2.42E+08 | 2.42E+08 | ERV_2116428 | ERVL-B4-int | I | 0.73 | 3.94 | 5.38 |
| HLA061 | 2 | 2.42E+08 | 2.42E+08 | ERV_2116428 | ERVL-B4-int | I | 0.87 | 2.23 | 2.58 |
| HLA067 | 2 | 2.42E+08 | 2.42E+08 | ERV_2116428 | ERVL-B4-int | I | 0.17 | 10.14 | 60.87 |
| HLA073 | 2 | 2.42E+08 | 2.42E+08 | ERV_2116428 | ERVL-B4-int | I | 1.00 | 3.79 | 3.79 |
| HLA072 | 2 | 8297197 | 8302648 | ERV_2120801 | ERV3-16A3_I-int,LTR16A | LI | 0.86 | 5.58 | 6.50 |
| HLA061 | 2 | 10199041 | 10199842 | ERV_2122088 | PABL_A-int | I | 0.97 | 14.15 | 14.57 |
| HLA063 | 2 | 10199041 | 10199842 | ERV_2122088 | PABL_A-int | I | 0.89 | 2.14 | 2.40 |
| HLA067 | 2 | 10199041 | 10199842 | ERV_2122088 | PABL_A-int | I | 0.17 | 1.80 | 10.30 |
| HLA072 | 2 | 10199041 | 10199842 | ERV_2122088 | PABL_A-int | I | 0.85 | 4.73 | 5.59 |
| HLA078 | 2 | 10199041 | 10199842 | ERV_2122088 | PABL_A-int | I | 0.43 | 4.00 | 9.32 |
| HLA061 | 2 | 10200132 | 10202321 | ERV_2122090 | PABL_A-int | I | 0.39 | 4.55 | 11.69 |
| HLA072 | 2 | 10200132 | 10202321 | ERV_2122090 | PABL_A-int | I | 0.35 | 2.02 | 5.78 |
| HLA078 | 2 | 10200132 | 10202321 | ERV_2122090 | PABL_A-int | I | 0.69 | 1.74 | 2.51 |
| HLA063 | 2 | 11436974 | 11437120 | ERV_2123067 | ERVL-E-int | I | 0.00 | 2.41 | 300.00 |
| HLA078 | 2 | 13325730 | 13330417 | ERV_2124376 | ERV3-16A3_I-int,LTR16A | LI | 0.00 | 1.59 | 300.00 |
| HLA078 | 2 | 13431355 | 13434329 | ERV_2124414 | THE1B,THE1B-int | LIL | 0.00 | 18.43 | 300.00 |
| HLA078 | 2 | 13436368 | 13443553 | ERV_2124417 | ERV3-16A3_I-int,LTR16A | LIL | 0.00 | 4.37 | 300.00 |
| HLA078 | 2 | 13437433 | 13438610 | ERV_2124418 | THE1D,THE1D-int | LIL | 0.00 | 4.37 | 300.00 |
| HLA078 | 2 | 13438615 | 13443239 | ERV_2124419 | HERV16-int | I | 0.00 | 4.37 | 300.00 |
| HLA078 | 2 | 13519979 | 13520093 | ERV_2124475 | LTR23-int | I | 0.00 | 273.36 | 300.00 |
| HLA078 | 2 | 13520104 | 13523277 | ERV_2124476 | LTR49-int | I | 0.00 | 30.62 | 300.00 |
| HLA058 | 2 | 13525956 | 13533609 | ERV_2124481 | Harlequin-int,LTR2 | LIL | 0.00 | 2.75 | 700.37 |
| HLA078 | 2 | 13525956 | 13533609 | ERV_2124481 | Harlequin-int,LTR2 | LIL | 0.00 | 81.75 | 300.00 |
| HLA058 | 2 | 13532712 | 13533096 | ERV_2124482 | HERVE_a-int | I | 0.00 | 2.75 | 700.37 |
| HLA078 | 2 | 13532712 | 13533096 | ERV_2124482 | HERVE_a-int | I | 0.00 | 81.75 | 300.00 |
| HLA073 | 2 | 20404559 | 20408382 | ERV_2128633 | MER57A-int | I | 0.00 | 34.84 | 300.00 |
| HLA063 | 2 | 20418623 | 20418884 | ERV_2128644 | MER57A-int | I | 0.00 | 1.70 | 300.00 |
| HLA078 | 2 | 20927545 | 20929807 | ERV_2128971 | THE1D,THE1D-int | LIL | 0.08 | 2.03 | 24.98 |
| HLA065 | 2 | 21102082 | 21102299 | ERV_2129068 | HERV9N-int | I | 0.00 | 5.49 | 300.00 |
| HLA069 | 2 | 21102082 | 21102299 | ERV_2129068 | HERV9N-int | I | 0.00 | 1.60 | 300.00 |
| HLA065 | 2 | 21180498 | 21184746 | ERV_2129138 | ERVL-E-int | I | 0.00 | 1.75 | 300.00 |
| HLA065 | 2 | 24554384 | 24555134 | ERV_2131158 | HUERS-P3-int,LTR9 | LIL | 0.00 | 2.13 | 300.00 |
| HLA053 | 2 | 26256938 | 26259651 | ERV_2132444 | HERVK22-int,LTR22A | LI | 4.64 | 15.94 | 3.44 |
| HLA067 | 2 | 26256938 | 26259651 | ERV_2132444 | HERVK22-int,LTR22A | LI | 3.36 | 12.80 | 3.81 |
| HLA058 | 2 | 26402437 | 26405320 | ERV_2132556 | ERV3-16A3_I-int | I | 0.55 | 2.19 | 3.96 |
| HLA072 | 2 | 28424530 | 28424992 | ERV_2134227 | MLT1J,MLT1J-int | LI | 0.38 | 1.93 | 5.10 |
| HLA076 | 2 | 28424530 | 28424992 | ERV_2134227 | MLT1J,MLT1J-int | LI | 0.29 | 1.78 | 6.23 |
| HLA078 | 2 | 28424530 | 28424992 | ERV_2134227 | MLT1J,MLT1J-int | LI | 0.00 | 1.87 | 300.00 |
| HLA053 | 2 | 31687476 | 31695509 | ERV_2136406 | HUERS-P3-int,LTR9A1 | LIL | 2.38 | 5.44 | 2.28 |
| HLA078 | 2 | 31687476 | 31695509 | ERV_2136406 | HUERS-P3-int,LTR9A1 | LIL | 0.25 | 3.65 | 14.83 |
| HLA053 | 2 | 34737167 | 34738420 | ERV_2138690 | ERV3-16A3_I-int | I | 2.88 | 5.85 | 2.03 |
| HLA078 | 2 | 34737167 | 34738420 | ERV_2138690 | ERV3-16A3_I-int | I | 0.88 | 8.01 | 9.14 |
| HLA078 | 2 | 35632310 | 35633124 | ERV_2139104 | THE1D,THE1D-int | LI | 0.07 | 1.66 | 23.24 |
| HLA065 | 2 | 43089001 | 43092977 | ERV_2143938 | MLT1G1,MLT1G1-int | LIL | 1.93 | 4.65 | 2.40 |
| HLA061 | 2 | 44058911 | 44061035 | ERV_2144621 | THE1C,THE1C-int | LI | 26.54 | 71.10 | 2.68 |
| HLA065 | 2 | 44058911 | 44061035 | ERV_2144621 | THE1C,THE1C-int | LI | 1.29 | 2.76 | 2.13 |
| HLA066 | 2 | 44058911 | 44061035 | ERV_2144621 | THE1C,THE1C-int | LI | 3.74 | 15.94 | 4.27 |
| HLA067 | 2 | 44058911 | 44061035 | ERV_2144621 | THE1C,THE1C-int | LI | 22.08 | 165.80 | 7.51 |
| HLA071 | 2 | 44058911 | 44061035 | ERV_2144621 | THE1C,THE1C-int | LI | 20.98 | 237.43 | 11.32 |
| HLA073 | 2 | 44058911 | 44061035 | ERV_2144621 | THE1C,THE1C-int | LI | 1.06 | 15.25 | 14.40 |
| HLA076 | 2 | 44058911 | 44061035 | ERV_2144621 | THE1C,THE1C-int | LI | 4.06 | 65.78 | 16.22 |
| HLA071 | 2 | 46962700 | 46964031 | ERV_2146615 | MLT1K,MLT1K-int | LI | 0.00 | 1.64 | 300.00 |
| HLA067 | 2 | 46973328 | 46974023 | ERV_2146632 | LTR38-int | I | 0.00 | 1.93 | 300.00 |
| HLA058 | 2 | 47689825 | 47699818 | ERV_2147254 | MER34B-int,MER34C_ | LI | 1.51 | 4.15 | 2.75 |
| HLA058 | 2 | 47691660 | 47699393 | ERV_2147256 | LTR49,LTR49-int | LI | 1.51 | 4.15 | 2.75 |
| HLA058 | 2 | 47694449 | 47696082 | ERV_2147262 | LTR49-int | I | 1.51 | 4.15 | 2.75 |
| HLA071 | 2 | 54938754 | 54938933 | ERV_2151506 | MLTIJ-int | I | 0.00 | 1.85 | 300.00 |
| HLA065 | 2 | 58210161 | 58212447 | ERV_2153309 | THE1B,THE1B-int | LIL | 0.12 | 2.34 | 19.13 |
| HLA072 | 2 | 61063247 | 61063691 | ERV_2154855 | MLT1J2,MLT1J2-int | LI | 0.00 | 2.34 | 300.00 |
| HLA066 | 2 | 63678018 | 63679739 | ERV_2156783 | MLT1J,MLT1J-int | LI | 0.15 | 8.78 | 57.21 |
| HLA069 | 2 | 63678018 | 63679739 | ERV_2156783 | MLT1J,MLT1J-int | LI | 0.00 | 1.82 | 300.00 |
| HLA073 | 2 | 63678018 | 63679739 | ERV_2156783 | MLT1J,MLT1J-int | LI | 0.47 | 7.24 | 15.48 |
| HLA076 | 2 | 63678018 | 63679739 | ERV_2156783 | MLT1J,MLT1J-int | LI | 0.56 | 5.57 | 9.91 |
| HLA058 | 2 | 65044777 | 65046931 | ERV_2157742 | HERVIP10FH-int | I | 0.48 | 2.35 | 4.84 |
| HLA072 | 2 | 65044777 | 65046931 | ERV_2157742 | HERVIP10FH-int | I | 0.29 | 3.33 | 11.38 |
| HLA078 | 2 | 65044777 | 65046931 | ERV_2157742 | HERVIP10FH-int | I | 0.13 | 1.55 | 11.68 |
| HLA072 | 2 | 68493613 | 68497825 | ERV_2159806 | MSTA,MSTA-int | LIL | 0.09 | 1.88 | 20.00 |
| HLA072 | 2 | 69773589 | 69777599 | ERV_2160690 | MER21-int,MER21C | LI | 0.34 | 1.60 | 4.67 |
| HLA058 | 2 | 69789458 | 69799610 | ERV_2160711 | LTR19C,LTR25-int | LIL | 0.29 | 2.26 | 7.79 |
| HLA072 | 2 | 69789458 | 69799610 | ERV_2160711 | LTR19C,LTR25-int | LIL | 0.45 | 1.59 | 3.53 |
| HLA058 | 2 | 69794782 | 69798802 | ERV_2160713 | HUERS-P3-int | I | 0.29 | 2.26 | 7.79 |
| HLA072 | 2 | 69794782 | 69798802 | ERV_2160713 | HUERS-P3-int | I | 0.45 | 1.59 | 3.53 |
| HLA053 | 2 | 72616497 | 72617510 | ERV_2162606 | THE1B,THE1B-int | LI | 0.68 | 1.70 | 2.51 |
| HLA065 | 2 | 72616497 | 72617510 | ERV_2162606 | THE1B,THE1B-int | LI | 0.40 | 2.58 | 6.40 |
| HLA072 | 2 | 72616497 | 72617510 | ERV_2162606 | THE1B,THE1B-int | LI | 0.00 | 3.39 | 300.00 |
| HLA072 | 2 | 73738942 | 73742448 | ERV_2163374 | MER70-int,MER70A | LIL | 0.57 | 1.78 | 3.11 |
| HLA058 | 2 | 75385796 | 75386233 | ERV_2164528 | PABL_A-int | I | 0.00 | 2.23 | 300.00 |
| HLA076 | 2 | 79163171 | 79170353 | ERV_2166416 | HERV17-int,LTR17 | LIL | 0.02 | 4.11 | 221.89 |
| HLA078 | 2 | 79163171 | 79170353 | ERV_2166416 | HERV17-int,LTR17 | LIL | 0.00 | 9.80 | 300.00 |
| HLA065 | 2 | 81753539 | 81756070 | ERV_2167912 | THE1A,THE1A-int | LIL | 0.01 | 2.81 | 296.60 |
| HLA058 | 2 | 84470760 | 84477426 | ERV_2169282 | HERVK14-int,LTR14B | LIL | 0.13 | 1.77 | 13.44 |
| HLA058 | 2 | 85290829 | 85290944 | ERV_2169950 | MLT-int | I | 0.50 | 3.74 | 7.41 |
| HLA066 | 2 | 85290829 | 85290944 | ERV_2169950 | MLT-int | I | 0.00 | 1.97 | 300.00 |
| HLA066 | 2 | 85290954 | 85291520 | ERV_2169951 | MLT1-int | I | 0.00 | 2.04 | 300.00 |
| HLA058 | 2 | 85378291 | 85384270 | ERV_2170026 | MER4-int,MER4A1 | LIL | 0.78 | 1.58 | 2.03 |
| HLA061 | 2 | 85378291 | 85384270 | ERV_2170026 | MER4-int,MER4A1 | LIL | 0.56 | 1.58 | 2.83 |
| HLA065 | 2 | 85378291 | 85384270 | ERV_2170026 | MER4-int,MER4A1 | LIL | 0.71 | 2.35 | 3.32 |
| HLA067 | 2 | 85378291 | 85384270 | ERV_2170026 | MER4-int,MER4A1 | LIL | 0.49 | 1.50 | 3.06 |
| HLA072 | 2 | 85378291 | 85384270 | ERV_2170026 | MER4-int,MER4A1 | LIL | 0.90 | 2.21 | 2.45 |
| HLA078 | 2 | 85378291 | 85384270 | ERV_2170026 | MER4-int,MER4A1 | LIL | 0.53 | 1.72 | 3.23 |
| HLA065 | 2 | 85452806 | 85452853 | ERV_2170087 | HERV9N-int | I | 0.00 | 3.00 | 300.00 |
| HLA078 | 2 | 86499539 | 86501852 | ERV_2170923 | MSTA,MSTA-int | LIL | 16.76 | 44.89 | 2.68 |
| HLA078 | 2 | 86500001 | 86501489 | ERV_2170924 | MSTA-int | I | 16.76 | 44.89 | 2.68 |
| HLA078 | 2 | 87725211 | 87730585 | ERV_2171742 | LTR27D,MER52-int | LI | 0.77 | 2.22 | 2.87 |
| HLA069 | 2 | 95838110 | 95838757 | ERV_2174448 | MSTB,MSTB-int | LI | 0.00 | 2.28 | 300.00 |
| HLA069 | 2 | 96409606 | 96409797 | ERV_2174746 | HERVIP10FH-int | I | 0.00 | 1.87 | 300.00 |
| HLA053 | 2 | 97751434 | 97752090 | ERV_2175600 | MER110-int | I | 0.00 | 2.46 | 300.00 |
| HLA058 | 2 | 1.01E+08 | 1.01E+08 | ERV_2177691 | MLT1B,MLT1B-int | LI | 0.28 | 2.49 | 8.99 |
| HLA058 | 2 | 1.02E+08 | 1.02E+08 | ERV_2178182 | THE1C,THE1C-int | LIL | 0.00 | 2.47 | 300.00 |
| HLA065 | 2 | 1.08E+08 | 1.08E+08 | ERV_2181534 | ERV3-16A3_I-int | I | 0.00 | 3.51 | 300.00 |
| HLA072 | 2 | 1.08E+08 | 1.08E+08 | ERV_2181543 | ERVL-E-int,LTR33 | LI | 0.00 | 1.90 | 300.00 |
| HLA058 | 2 | 1.08E+08 | 1.08E+08 | ERV_2181629 | ERV3-16A3_I-int | I | 0.00 | 2.01 | 300.00 |
| HLA072 | 2 | 1.09E+08 | 1.09E+08 | ERV_2182084 | MLT-int | I | 0.99 | 2.98 | 3.01 |
| HLA058 | 2 | 1.1E+08 | 1.1E+08 | ERV_2182844 | ERVL-B4-int,MLT2C1 | LI | 0.99 | 10.93 | 11.08 |
| HLA065 | 2 | 1.1E+08 | 1.1E+08 | ERV_2182844 | ERVL-B4-int,MLT2C1 | LI | 2.07 | 5.08 | 2.45 |
| HLA067 | 2 | 1.1E+08 | 1.1E+08 | ERV_2182844 | ERVL-B4-int,MLT2C1 | LI | 0.95 | 2.74 | 2.88 |
| HLA069 | 2 | 1.12E+08 | 1.12E+08 | ERV_2184003 | HERV17-int,LTR17 | LIL | 8.86 | 20.02 | 2.26 |
| HLA076 | 2 | 1.12E+08 | 1.12E+08 | ERV_2184003 | HERV17-int,LTR17 | LIL | 5.91 | 14.56 | 2.47 |
| HLA071 | 2 | 1.13E+08 | 1.13E+O8 | ERV_2184406 | ERV3-16A3_I-int | I | 0.52 | 6.01 | 11.52 |
| HLA076 | 2 | 1.13E+08 | 1.13E+O8 | ERV_2185042 | MER57-int | I | 0.00 | 3.74 | 300.00 |
| HLA065 | 2 | 1.14E+08 | 1.14E+08 | ERV_2185119 | ERVL-B4-int | I | 0.00 | 1.63 | 300.00 |
| HLA073 | 2 | 1.17E+08 | 1.17E+08 | ERV_2187017 | THE1D-int | I | 0.00 | 2.00 | 300.00 |
| HLA066 | 2 | 1.22E+08 | 1.22E+08 | ERV_2190813 | THE1-int | I | 0.00 | 2.41 | 300.00 |
| HLA082 | 2 | 1.26E+08 | 1.26E+08 | ERV_2192784 | THE1-int | I | 0.00 | 1.79 | 300.00 |
| HLA069 | 2 | 1.3E+08 | 1.3E+08 | ERV_2195123 | MSTA,MSTA-int | LIL | 0.00 | 2.02 | 300.00 |
| HLA069 | 2 | 1.3E+08 | 1.3E+08 | ERV_2195124 | THE1D-int | I | 0.00 | 2.02 | 300.00 |
| HLA069 | 2 | 1.3E+08 | 1.3E+08 | ERV_2195125 | MSTA,MSTA-int | LIL | 0.00 | 2.02 | 300.00 |
| HLA069 | 2 | 1.3E+08 | 1.3E+08 | ERV_2195127 | MSTA,MSTA-int | LI | 0.00 | 1.58 | 300.00 |
| HLA072 | 2 | 1.3E+08 | 1.3E+08 | ERV_2195127 | MSTA,MSTA-int | LI | 0.00 | 1.91 | 300.00 |
| HLA082 | 2 | 1.3E+08 | 1.3E+08 | ERV_2195127 | MSTA,MSTA-int | LI | 0.00 | 1.65 | 300.00 |
| HLA058 | 2 | 1.3E+08 | 1.3E+08 | ERV_2195130 | MER4-int | I | 0.00 | 4.24 | 300.00 |
| HLA058 | 2 | 1.3E+08 | 1.3E+08 | ERV_2195346 | MER4-int | I | 0.00 | 1.73 | 300.00 |
| HLA058 | 2 | 1.31E+08 | 1.31E+08 | ERV_2195901 | MST-int | I | 0.00 | 2.83 | 300.00 |
| HLA058 | 2 | 1.32E+08 | 1.32E+08 | ERV_2196151 | ERV3-16A3_I-int | I | 0.83 | 9.17 | 11.09 |
| HLA069 | 2 | 1.35E+08 | 1.35E+08 | ERV_2197824 | HERV9-int | I | 0.00 | 1.54 | 300.00 |
| HLA053 | 2 | 1.35E+08 | 1.35E+08 | ERV_2197879 | MLT1F,MLT1F-int | LIL | 0.66 | 8.10 | 12.21 |
| HLA067 | 2 | 1.35E+08 | 1.35E+08 | ERV_2197879 | MLT1F,MLT1F-int | LIL | 1.63 | 12.71 | 7.81 |
| HLA071 | 2 | 1.35E+08 | 1.35E+08 | ERV_2197879 | MLT1F,MLT1F-int | LIL | 5.50 | 11.00 | 2.00 |
| HLA076 | 2 | 1.35E+08 | 1.35E+08 | ERV_2197879 | MLT1F,MLT1F-int | LIL | 4.44 | 10.72 | 2.41 |
| HLA063 | 2 | 1.36E+08 | 1.36E+08 | ERV_2198524 | MER57A-int | I | 0.00 | 1.97 | 300.00 |
| HLA061 | 2 | 1.37E+08 | 1.37E+08 | ERV_2199370 | MLT1J2,MLT1J2-int | LIL | 0.00 | 1.62 | 300.00 |
| HLA065 | 2 | 1.38E+08 | 1.38E+08 | ERV_2200163 | THE1D,THE1D-int | LI | 0.56 | 2.81 | 5.00 |
| HLA072 | 2 | 1.38E+08 | 1.38E+08 | ERV_2200163 | THE1D,THE1D-int | LI | 0.72 | 5.99 | 8.26 |
| HLA058 | 2 | 1.4E+08 | 1.4E+08 | ERV_2200826 | LOR1-int | I | 0.00 | 1.76 | 384.70 |
| HLA058 | 2 | 1.4E+08 | 1.4E+08 | ERV_2200827 | LTR57-int | I | 0.00 | 1.76 | 384.70 |
| HLA053 | 2 | 1.43E+08 | 1.43E+08 | ERV_2202308 | HERVH48-int,MER48 | LIL | 1.52 | 5.00 | 3.30 |
| HLA067 | 2 | 1.43E+08 | 1.43E+08 | ERV_2202308 | HERVH48-int,MER48 | LIL | 0.61 | 4.94 | 8.08 |
| HLA065 | 2 | 1.43E+08 | 1.43E+08 | ERV_2202309 | MER57-int,MER57B1 | LIL | 0.68 | 4.30 | 6.35 |
| HLA067 | 2 | 1.43E+08 | 1.43E+08 | ERV_2202309 | MER57-int,MER57B1 | LIL | 1.65 | 4.36 | 2.63 |
| HLA067 | 2 | 1.5E+08 | 1.5E+08 | ERV_2206205 | ERV3-16A3_I-int,LTR16 | LI | 0.48 | 1.57 | 3.27 |
| HLA073 | 2 | 1.51E+08 | 1.51E+08 | ERV_2206706 | LTR57-int | I | 1.98 | 9.92 | 5.00 |
| HLA073 | 2 | 1.51E+08 | 1.51E+08 | ERV_2206707 | MER65-int | I | 1.98 | 9.92 | 5.00 |
| HLA053 | 2 | 1.61E+08 | 1.61E+08 | ERV_2212182 | MLT1A1,MLT1A1-int | LI | 0.58 | 2.09 | 3.58 |
| HLA066 | 2 | 1.62E+08 | 1.62E+08 | ERV_2212769 | MLT1B,MLT1B-int | LI | 3.00 | 7.51 | 2.50 |
| HLA072 | 2 | 1.62E+08 | 1.62E+08 | ERV_2212769 | MLT1B,MLT1B-int | LI | 3.59 | 8.34 | 2.32 |
| HLA073 | 2 | 1.62E+08 | 1.62E+08 | ERV_2212769 | MLT1B,MLT1B-int | LI | 2.24 | 5.70 | 2.54 |
| HLA066 | 2 | 1.62E+08 | 1.62E+08 | ERV_2212771 | MLT1-int | I | 3.00 | 7.51 | 2.50 |
| HLA072 | 2 | 1.62E+08 | 1.62E+08 | ERV_2212771 | MLT1-int | I | 3.59 | 8.34 | 2.32 |
| HLA073 | 2 | 1.62E+08 | 1.62E+08 | ERV_2212771 | MLT1-int | I | 2.24 | 5.70 | 2.54 |
| HLA067 | 2 | 1.65E+08 | 1.65E+08 | ERV_2213915 | HERV17-int,LTR17 | LI | 0.34 | 1.77 | 5.27 |
| HLA076 | 2 | 1.65E+08 | 1.65E+08 | ERV_2213915 | HERV17-int,LTR17 | LI | 0.07 | 1.89 | 26.94 |
| HLA058 | 2 | 1.66E+08 | 1.66E+08 | ERV_2214571 | THE1B,THE1B-int | LIL | 0.00 | 1.53 | 300.00 |
| HLA058 | 2 | 1.67E+08 | 1.67E+08 | ERV_2215318 | MamGyp-int,MamGypLTR1c | LI | 0.07 | 1.83 | 26.52 |
| HLA058 | 2 | 1.68E+08 | 1.68E+08 | ERV_2215422 | MLTIJ-int | I | 0.00 | 2.13 | 300.00 |
| HLA067 | 2 | 1.69E+08 | 1.69E+08 | ERV_2216221 | THE1D,THE1D-int | LIL | 8.15 | 22.45 | 2.75 |
| HLA069 | 2 | 1.69E+08 | 1.69E+08 | ERV_2216221 | THE1D,THE1D-int | LIL | 0.27 | 2.22 | 8.23 |
| HLA058 | 2 | 1.7E+08 | 1.7E+08 | ERV_2216651 | THE1A,THE1A-int | LIL | 14.3 7 | 31.54 | 2.19 |
| HLA082 | 2 | 1.7E+08 | 1.7E+08 | ERV_2216651 | THE1A,THE1A-int | LIL | 9.65 | 24.87 | 2.58 |
| HLA058 | 2 | 1.7E+08 | 1.7E+08 | ERV_2216668 | HERVL18-int,LTR18B | LIL | 0.00 | 16.17 | 300.00 |
| HLA072 | 2 | 1.7E+08 | 1.7E+08 | ERV_2216668 | HERVL18-int,LTR18B | LIL | 0.05 | 1.61 | 32.10 |
| HLA061 | 2 | 1.71E+08 | 1.71E+08 | ERV_2217362 | HERVK11D-int,MER11D | LIL | 0.09 | 2.62 | 27.63 |
| HLA063 | 2 | 1.71E+08 | 1.71E+08 | ERV_2217362 | HERVK11D-int,MER11D | LIL | 0.07 | 4.03 | 56.71 |
| HLA071 | 2 | 1.71E+08 | 1.71E+08 | ERV_2217362 | HERVK11D-int,MER11D | LIL | 0.19 | 2.96 | 15.71 |
| HLA082 | 2 | 1.74E+08 | 1.74E+08 | ERV_2220021 | THE1B,THE1B-int | LIL | 0.17 | 2.23 | 13.13 |
| HLA069 | 2 | 1.75E+08 | 1.75E+08 | ERV_2220286 | LTR49-int | I | 0.00 | 4.39 | 300.00 |
| HLA067 | 2 | 1.83E+08 | 1.83E+08 | ERV_2224792 | MLT1E2,MLT1E2-int | LI | 0.01 | 1.52 | 143.63 |
| HLA072 | 2 | 1.83E+08 | 1.83E+08 | ERV_2224792 | MLT1E2,MLT1E2-int | LI | 0.00 | 1.56 | 300.00 |
| HLA067 | 2 | 1.87E+08 | 1.87E+08 | ERV_2226945 | THE1B,THE1B-int | LI | 0.00 | 2.70 | 300.00 |
| HLA067 | 2 | 1.88E+08 | 1.88E+08 | ERV_2226967 | HERVH-int,LTR7 | LIL | 0.68 | 3.31 | 4.85 |
| HLA072 | 2 | 1.91E+08 | 1.91E+08 | ERV_2228579 | PABL_A,PABL_A-int | LI | 0.07 | 1.54 | 23.40 |
| HLA069 | 2 | 1.91E+08 | 1.91E+08 | ERV_2229108 | HERVH-int,LTR7B | LI | 0.01 | 2.18 | 190.28 |
| HLA082 | 2 | 1.91E+08 | 1.91E+08 | ERV_2229108 | HERVH-int,LTR7B | LI | 0.88 | 2.68 | 3.04 |
| HLA071 | 2 | 1.94E+08 | 1.94E+08 | ERV_2230157 | MER61-int,MER61A | LI | 0.01 | 1.65 | 206.31 |
| HLA076 | 2 | 2E+08 | 2E+08 | ERV_2233678 | LTR37-int | I | 0.09 | 2.18 | 24.33 |
| HLA067 | 2 | 2.01E+08 | 2.01E+08 | ERV_2234623 | MER76,MER76-int | LI | 0.91 | 2.57 | 2.83 |
| HLA071 | 2 | 2.01E+08 | 2.01E+08 | ERV_2234623 | MER76,MER76-int | LI | 0.61 | 4.40 | 7.18 |
| HLA061 | 2 | 2.1E+08 | 2.1E+08 | ERV_2240288 | MER4B-int | I | 0.00 | 2.18 | 300.00 |
| HLA066 | 2 | 2.1E+08 | 2.1E+08 | ERV_2240288 | MER4B-int | I | 0.00 | 2.34 | 300.00 |
| HLA063 | 2 | 2.1E+08 | 2.1E+08 | ERV_2240582 | MLT1C,MLT1C-int | LI | 0.62 | 2.95 | 4.73 |
| HLA065 | 2 | 2.1E+08 | 2.1E+08 | ERV_2240582 | MLT1C,MLT1C-int | LI | 2.06 | 7.14 | 3.46 |
| HLA072 | 2 | 2.1E+08 | 2.1E+08 | ERV_2240582 | MLT1C,MLT1C-int | LI | 1.07 | 2.82 | 2.62 |
| HLA073 | 2 | 2.1E+08 | 2.1E+08 | ERV_2240582 | MLT1C,MLT1C-int | LI | 0.94 | 2.27 | 2.41 |
| HLA078 | 2 | 2.1E+08 | 2.1E+08 | ERV_2240582 | MLT1C,MLT1C-int | LI | 0.23 | 2.00 | 8.71 |
| HLA071 | 2 | 2.11E+08 | 2.11E+08 | ERV_2240827 | THE1C,THE1C-int | LIL | 0.40 | 4.54 | 11.36 |
| HLA073 | 2 | 2.16E+08 | 2.16E+08 | ERV_2243717 | ERV3-16A3_I-int,LTR16A | LI | 0.38 | 1.82 | 4.77 |
| HLA076 | 2 | 2.16E+08 | 2.16E+08 | ERV_2243717 | ERV3-16A3_I-int,LTR16A | LI | 0.62 | 1.53 | 2.48 |
| HLA067 | 2 | 2.16E+08 | 2.16E+08 | ERV_2243795 | HERVK14-int | I | 0.00 | 1.66 | 300.00 |
| HLA071 | 2 | 2.16E+08 | 2.16E+08 | ERV_2243795 | HERVK14-int | I | 1.25 | 2.85 | 2.27 |
| HLA076 | 2 | 2.16E+08 | 2.16E+08 | ERV_2243795 | HERVK14-int | I | 0.00 | 1.94 | 300.00 |
| HLA063 | 2 | 2.16E+08 | 2.16E+08 | ERV_2243896 | LOR1-int | I | 3.44 | 7.59 | 2.21 |
| HLA065 | 2 | 2.16E+08 | 2.16E+08 | ERV_2243896 | LOR1-int | I | 1.74 | 8.10 | 4.66 |
| HLA072 | 2 | 2.16E+08 | 2.16E+08 | ERV_2243896 | LOR1-int | I | 3.88 | 11.89 | 3.07 |
| HLA076 | 2 | 2.16E+08 | 2.16E+08 | ERV_2243896 | LOR1-int | I | 3.88 | 7.93 | 2.04 |
| HLA082 | 2 | 2.16E+08 | 2.16E+08 | ERV_2243896 | LOR1-int | I | 4.09 | 12.95 | 3.16 |
| HLA072 | 2 | 2.16E+08 | 2.16E+08 | ERV_2243897 | MER101-int | I | 1.62 | 3.62 | 2.23 |
| HLA082 | 2 | 2.16E+08 | 2.16E+08 | ERV_2243897 | MER101-int | I | 0.78 | 4.64 | 5.97 |
| HLA072 | 2 | 2.16E+08 | 2.16E+08 | ERV_2243898 | LOR1-int | I | 1.62 | 3.62 | 2.23 |
| HLA082 | 2 | 2.16E+08 | 2.16E+08 | ERV_2243898 | LOR1-int | I | 0.78 | 4.64 | 5.97 |
| HLA072 | 2 | 2.16E+08 | 2.16E+08 | ERV_2243899 | LTR57-int | I | 1.62 | 3.62 | 2.23 |
| HLA082 | 2 | 2.16E+08 | 2.16E+08 | ERV_2243899 | LTR57-int | I | 0.78 | 4.64 | 5.97 |
| HLA072 | 2 | 2.19E+08 | 2.19E+08 | ERV_2245866 | MER41E,MER66-int | LI | 0.82 | 1.97 | 2.40 |
| HLA067 | 2 | 2.19E+08 | 2.19E+08 | ERV_2245878 | MER57-int,MER57B1 | LI | 0.42 | 1.72 | 4.07 |
| HLA067 | 2 | 2.19E+08 | 2.19E+08 | ERV_2245883 | MER61-int,MER61B | LI | 0.43 | 1.66 | 3.90 |
| HLA071 | 2 | 2.19E+08 | 2.19E+08 | ERV_2245883 | MER61-int,MER61B | LI | 0.83 | 2.61 | 3.15 |
| HLA065 | 2 | 2.19E+08 | 2.19E+08 | ERV_2246273 | ERV3-16A3_I-int | I | 0.00 | 2.00 | 300.00 |
| HLA066 | 2 | 2.19E+08 | 2.19E+08 | ERV_2246273 | ERV3-16A3_I-int | I | 1.99 | 8.03 | 4.03 |
| HLA076 | 2 | 2.19E+08 | 2.19E+08 | ERV_2246273 | ERV3-16A3_I-int | I | 3.31 | 10.53 | 3.18 |
| HLA082 | 2 | 2.19E+08 | 2.19E+08 | ERV_2246273 | ERV3-16A3_I-int | I | 0.97 | 3.13 | 3.23 |
| HLA065 | 2 | 2.3E+08 | 2.3E+08 | ERV_2253045 | MER101-int | I | 0.02 | 1.84 | 75.64 |
| HLA065 | 2 | 2.3E+08 | 2.3E+08 | ERV_2253176 | ERV3-16A3_I-int | I | 0.00 | 1.80 | 300.00 |
| HLA063 | 2 | 2.31E+08 | 2.31E+08 | ERV_2253347 | LTR49-int | I | 0.63 | 2.20 | 3.51 |
| HLA076 | 2 | 2.31E+08 | 2.31E+08 | ERV_2253370 | MSTA,MSTA-int | LIL | 0.87 | 2.88 | 3.33 |
| HLA063 | 2 | 2.33E+08 | 2.33E+08 | ERV_2254973 | ERVL-B4-int | I | 6.69 | 13.82 | 2.07 |
| HLA066 | 2 | 2.33E+08 | 2.33E+08 | ERV_2254973 | ERVL-B4-int | I | 3.21 | 13.14 | 4.09 |
| HLA073 | 2 | 2.33E+08 | 2.33E+08 | ERV_2254973 | ERVL-B4-int | I | 1.40 | 6.51 | 4.67 |
| HLA076 | 2 | 2.33E+08 | 2.33E+08 | ERV_2254973 | ERVL-B4-int | I | 6.55 | 15.35 | 2.34 |
| HLA065 | 2 | 2.33E+08 | 2.33E+08 | ERV_2255245 | ERVL-E-int | I | 0.00 | 6.88 | 300.00 |
| HLA067 | 2 | 2.33E+08 | 2.33E+08 | ERV_2255245 | ERVL-E-int | I | 0.32 | 2.74 | 8.61 |
| HLA072 | 2 | 2.33E+08 | 2.33E+08 | ERV_2255245 | ERVL-E-int | I | 1.11 | 2.69 | 2.42 |
| HLA082 | 2 | 2.33E+08 | 2.33E+08 | ERV_2255245 | ERVL-E-int | I | 0.00 | 1.89 | 300.00 |
| HLA065 | 2 | 2.33E+08 | 2.33E+08 | ERV_2255251 | ERVL-B4-int | I | 1.40 | 3.56 | 2.54 |
| HLA065 | 2 | 2.33E+08 | 2.33E+08 | ERV_2255279 | MLT1A0,MLT1A0-int | LIL | 1.18 | 2.67 | 2.27 |
| HLA072 | 2 | 2.33E+08 | 2.33E+08 | ERV_2255279 | MLT1A0,MLT1A0-int | LIL | 0.34 | 2.17 | 6.34 |
| HLA061 | 2 | 2.41E+08 | 2.41E+08 | ERV_2259829 | MLT1J-int | I | 0.00 | 2.76 | 300.00 |
| HLA065 | 2 | 2.41E+08 | 2.41E+08 | ERV_2259955 | ERVL-E-int | I | 0.00 | 1.96 | 300.00 |
| HLA067 | 2 | 2.41E+08 | 2.41E+08 | ERV_2259955 | ERVL-E-int | I | 0.63 | 7.67 | 12.22 |
| HLA071 | 2 | 2.41E+08 | 2.41E+08 | ERV_2259955 | ERVL-E-int | I | 0.49 | 1.82 | 3.72 |
| HLA072 | 2 | 2.41E+08 | 2.41E+08 | ERV_2259955 | ERVL-E-int | I | 0.00 | 2.22 | 300.00 |
| HLA063 | 2 | 2.41E+08 | 2.41E+08 | ERV_2259957 | ERVL-E-int | I | 2.79 | 6.63 | 2.38 |
| HLA066 | 2 | 2.41E+08 | 2.41E+08 | ERV_2259957 | ERVL-E-int | I | 0.66 | 3.51 | 5.32 |
| HLA067 | 2 | 2.41E+08 | 2.41E+08 | ERV_2259957 | ERVL-E-int | I | 11.5 6 | 25.67 | 2.22 |
| HLA069 | 2 | 2.41E+08 | 2.41E+08 | ERV_2259957 | ERVL-E-int | I | 0.73 | 5.29 | 7.26 |
| HLA072 | 2 | 2.41E+08 | 2.41E+08 | ERV_2259957 | ERVL-E-int | I | 1.51 | 9.18 | 6.07 |
| HLA067 | 2 | 2.42E+08 | 2.42E+08 | ERV_2260518 | PRIMA4-int,PRIMA4_LTR | LI | 0.12 | 2.62 | 22.29 |
| HLA069 | 2 | 2.42E+08 | 2.42E+08 | ERV_2260691 | MER31-int | I | 0.61 | 1.58 | 2.58 |
| HLA082 | 2 | 2.42E+08 | 2.42E+08 | ERV_2260692 | MER34B-int,MER34C_ | LI | 0.55 | 3.20 | 5.76 |
| HLA065 | 20 | 44037742 | 44038081 | ERV_2300711 | THE1D,THE1D-int | LI | 0.00 | 2.00 | 300.00 |
| HLA067 | 20 | 18313019 | 18313139 | ERV_2335472 | LTR37-int | I | 0.00 | 4.13 | 300.00 |
| HLA063 | 20 | 30412172 | 30412388 | ERV_2340811 | MER57-int | I | 0.77 | 2.50 | 3.25 |
| HLA067 | 22 | 11923323 | 11923379 | ERV_2423890 | THE1D-int | I | 0.00 | 1.66 | 300.00 |
| HLA063 | 22 | 36215797 | 36215947 | ERV_2445987 | PRIMA4-int | I | 0.29 | 2.00 | 6.98 |
| HLA058 | 22 | 47787334 | 47787754 | ERV_2459028 | THE1D-int | I | 0.00 | 2.80 | 300.00 |
| HLA065 | 3 | 861491 | 861718 | ERV_2490988 | HERVE_a-int | I | 1.97 | 4.27 | 2.17 |
| HLA082 | 3 | 8647481 | 8647600 | ERV_2498025 | ERVL-B4-int | I | 0.00 | 1.75 | 300.00 |
| HLA063 | 3 | 11812212 | 11812318 | ERV_2501713 | HERV16-int | I | 1.49 | 6.88 | 4.61 |
| HLA069 | 3 | 11812212 | 11812318 | ERV_2501713 | HERV16-int | I | 0.00 | 1.59 | 300.00 |
| HLA072 | 3 | 11812212 | 11812318 | ERV_2501713 | HERV16-int | I | 1.39 | 3.05 | 2.20 |
| HLA082 | 3 | 11812212 | 11812318 | ERV_2501713 | HERV16-int | I | 1.44 | 3.50 | 2.42 |
| HLA069 | 3 | 11812373 | 11813017 | ERV_2501714 | ERV3-16A3_I-int | I | 0.00 | 1.72 | 300.00 |
| HLA072 | 3 | 11812373 | 11813017 | ERV_2501714 | ERV3-16A3_I-int | I | 0.65 | 3.49 | 5.38 |
| HLA082 | 3 | 11812373 | 11813017 | ERV_2501714 | ERV3-16A3_I-int | I | 0.58 | 1.69 | 2.94 |
| HLA082 | 3 | 32469211 | 32469367 | ERV_2520070 | ERV3-16A3_I-int | I | 0.62 | 1.71 | 2.74 |
| HLA069 | 3 | 36985489 | 36986389 | ERV_2524084 | MLT1I,MLT1I-int | LI | 0.32 | 2.22 | 6.85 |
| HLA072 | 3 | 36985489 | 36986389 | ERV_2524084 | MLT1I,MLT1I-int | LI | 1.03 | 3.01 | 2.91 |
| HLA082 | 3 | 36985489 | 36986389 | ERV_2524084 | MLT1I,MLT1I-int | LI | 0.69 | 1.68 | 2.45 |
| HLA065 | 3 | 37380152 | 37380435 | ERV_2524480 | MLT1H-int | I | 0.07 | 4.99 | 70.55 |
| HLA065 | 3 | 38417327 | 38417585 | ERV_2525275 | MLT1H-int | I | 33.64 | 69.38 | 2.06 |
| HLA067 | 3 | 38420760 | 38427256 | ERV_2525282 | HERVK9-int,MER9B | LIL | 8.60 | 29.33 | 3.41 |
| HLA065 | 3 | 38451965 | 38452175 | ERV_2525297 | ERVL-E-int | I | 0.58 | 5.57 | 9.66 |
| HLA071 | 3 | 38451965 | 38452175 | ERV_2525297 | ERVL-E-int | I | 0.63 | 8.50 | 13.43 |
| HLA072 | 3 | 39421775 | 39422013 | ERV_2526146 | MLT1J-int | I | 0.00 | 4.30 | 300.00 |
| HLA061 | 3 | 39788550 | 39789234 | ERV_2526446 | MLT1H,MLT1H-int | LI | 0.00 | 3.20 | 300.00 |
| HLA071 | 3 | 39788550 | 39789234 | ERV_2526446 | MLT1H,MLT1H-int | LI | 1.00 | 6.70 | 6.72 |
| HLA073 | 3 | 39788550 | 39789234 | ERV_2526446 | MLT1H,MLT1H-int | LI | 0.00 | 2.29 | 300.00 |
| HLA076 | 3 | 39788550 | 39789234 | ERV_2526446 | MLT1H,MLT1H-int | LI | 0.00 | 9.04 | 300.00 |
| HLA071 | 3 | 39794777 | 39794897 | ERV_2526450 | MER89-int | I | 0.14 | 3.10 | 21.60 |
| HLA076 | 3 | 39794777 | 39794897 | ERV_2526450 | MER89-int | I | 0.30 | 2.58 | 8.75 |
| HLA071 | 3 | 40262923 | 40264376 | ERV_2526760 | MER31-int,MER31B | LI | 1.32 | 3.25 | 2.47 |
| HLA071 | 3 | 40263670 | 40263767 | ERV_2526763 | MER34B-int | I | 1.32 | 3.25 | 2.47 |
| HLA063 | 3 | 40264428 | 40265037 | ERV_2526765 | MER101-int | I | 1.30 | 2.99 | 2.30 |
| HLA071 | 3 | 40264428 | 40265037 | ERV_2526765 | MER101-int | I | 0.40 | 2.21 | 5.58 |
| HLA065 | 3 | 40489918 | 40490509 | ERV_2527012 | MSTD,MSTD-int | LI | 5.41 | 14.54 | 2.69 |
| HLA066 | 3 | 40489918 | 40490509 | ERV_2527012 | MSTD,MSTD-int | LI | 6.41 | 15.49 | 2.42 |
| HLA071 | 3 | 40489918 | 40490509 | ERV_2527012 | MSTD,MSTD-int | LI | 6.37 | 22.94 | 3.60 |
| HLA065 | 3 | 41683025 | 41685909 | ERV_2527968 | HERV351-int,LTR35,LTR35A | LIL | 0.30 | 2.37 | 8.03 |
| HLA069 | 3 | 42896033 | 42900433 | ERV_2529208 | MER50-int,MER50C | LI | 0.99 | 8.00 | 8.10 |
| HLA073 | 3 | 42896033 | 42900433 | ERV_2529208 | MER50-int,MER50C | LI | 1.36 | 3.77 | 2.78 |
| HLA073 | 3 | 44425387 | 44425473 | ERV_2530470 | MER89-int | I | 0.92 | 2.06 | 2.24 |
| HLA065 | 3 | 44493044 | 44494098 | ERV_2530539 | MLT-int | I | 3.25 | 7.58 | 2.33 |
| HLA069 | 3 | 44750216 | 44751991 | ERV_2530766 | LOR1-int | I | 15.55 | 34.07 | 2.19 |
| HLA071 | 3 | 44750216 | 44751991 | ERV_2530766 | LOR1-int | I | 26.74 | 93.89 | 3.51 |
| HLA061 | 3 | 47175379 | 47175910 | ERV_2533028 | MSTA,MSTA-int | LI | 0.44 | 2.41 | 5.53 |
| HLA063 | 3 | 47175379 | 47175910 | ERV_2533028 | MSTA,MSTA-int | LI | 0.21 | 1.54 | 7.32 |
| HLA066 | 3 | 47175379 | 47175910 | ERV_2533028 | MSTA,MSTA-int | LI | 0.00 | 4.26 | 300.00 |
| HLA072 | 3 | 47175379 | 47175910 | ERV_2533028 | MSTA,MSTA-int | LI | 0.00 | 6.52 | 300.00 |
| HLA072 | 3 | 47176108 | 47178714 | ERV_2533029 | MER57A-int | I | 0.56 | 3.53 | 6.36 |
| HLA072 | 3 | 48282075 | 48283201 | ERV_2534510 | ERVL-E-int,LTR16E2 | LI | 0.00 | 1.53 | 300.00 |
| HLA063 | 3 | 50584334 | 50584921 | ERV_2536895 | MLT1D,MLT1D-int | LI | 9.19 | 28.40 | 3.09 |
| HLA072 | 3 | 60051427 | 60052420 | ERV_2545650 | MER66-int,MER66B | LI | 0.00 | 2.83 | 300.00 |
| HLA069 | 3 | 63851897 | 63852287 | ERV_2549412 | MST-int | I | 0.00 | 1.69 | 300.00 |
| HLA061 | 3 | 75624158 | 75624604 | ERV_2560894 | MER31-int | I | 0.00 | 3.43 | 300.00 |
| HLA061 | 3 | 75625169 | 75625901 | ERV_2560895 | MER92-int | I | 0.52 | 1.89 | 3.62 |
| HLA065 | 3 | 93938055 | 93944071 | ERV_2572887 | HERVH-int,LTR7 | LIL | 0.43 | 1.78 | 4.12 |
| HLA065 | 3 | 93961783 | 93969033 | ERV_2572903 | ERVL-E-int | I | 0.68 | 2.32 | 3.42 |
| HLA066 | 3 | 1E+08 | 1E+08 | ERV_2577538 | MLT1J-int | I | 0.46 | 1.98 | 4.27 |
| HLA071 | 3 | 1E+08 | 1E+08 | ERV_2577538 | MLT1J-int | I | 0.72 | 1.93 | 2.67 |
| HLA071 | 3 | 1E+08 | 1E+08 | ERV_2577667 | MLT1-int | I | 0.84 | 3.89 | 4.62 |
| HLA071 | 3 | 1E+08 | 1E+08 | ERV_2577668 | MLT-int | I | 0.84 | 3.89 | 4.62 |
| HLA072 | 3 | 1.02E+08 | 1.02E+08 | ERV_2578677 | HERVK-int,LTR5_Hs | LIL | 4.89 | 10.78 | 2.20 |
| HLA073 | 3 | 1.02E+08 | 1.02E+08 | ERV_2578677 | HERVK-int,LTR5_Hs | LIL | 4.98 | 11.32 | 2.27 |
| HLA065 | 3 | 1.05E+08 | 1.05E+08 | ERV_2581505 | ERVL-E-int | I | 0.00 | 2.59 | 300.00 |
| HLA072 | 3 | 1.05E+08 | 1.05E+08 | ERV_2581505 | ERVL-E-int | I | 0.51 | 2.13 | 4.20 |
| HLA061 | 3 | 1.13E+08 | 1.13E+08 | ERV_2587430 | HERVL-int,MLT2A1 | LI | 0.78 | 3.39 | 4.36 |
| HLA065 | 3 | 1.13E+08 | 1.13E+08 | ERV_2587918 | THE1A,THE1A-int | LIL | 0.00 | 2.37 | 300.00 |
| HLA072 | 3 | 1.13E+08 | 1.13E+08 | ERV_2587918 | THE1A,THE1A-int | LIL | 0.07 | 2.93 | 44.96 |
| HLA063 | 3 | 1.21E+08 | 1.21E+08 | ERV_2594376 | MER4B-int,MER4D1 | LIL | 13.57 | 28.83 | 2.12 |
| HLA065 | 3 | 1.21E+08 | 1.21E+08 | ERV_2594376 | MER4B-int,MER4D1 | LIL | 1.72 | 11.62 | 6.75 |
| HLA066 | 3 | 1.22E+08 | 1.22E+08 | ERV_2595570 | THE1C,THE1C-int | LI | 0.00 | 3.75 | 300.00 |
| HLA061 | 3 | 1.23E+08 | 1.23E+08 | ERV_2595957 | MLT1J-int | I | 3.88 | 7.86 | 2.03 |
| HLA065 | 3 | 1.23E+08 | 1.23E+08 | ERV_2595957 | MLT1J-int | I | 2.71 | 8.49 | 3.13 |
| HLA066 | 3 | 1.23E+08 | 1.23E+08 | ERV_2595957 | MLT1J-int | I | 6.58 | 17.96 | 2.73 |
| HLA065 | 3 | 1.26E+08 | 1.26E+08 | ERV_2599024 | HERVL-int,MLT2A2 | LI | 0.56 | 1.92 | 3.42 |
| HLA073 | 3 | 1.26E+08 | 1.26E+08 | ERV_2599429 | MER4B-int,MER4E | LI | 4.73 | 13.73 | 2.90 |
| HLA076 | 3 | 1.26E+08 | 1.26E+08 | ERV_2599499 | LTR25-int | I | 0.34 | 2.46 | 7.14 |
| HLA076 | 3 | 1.26E+08 | 1.26E+08 | ERV_2599500 | HERVFH19-int,LTR19B | LI | 0.34 | 2.46 | 7.14 |
| HLA061 | 3 | 1.26E+08 | 1.26E+08 | ERV_2599552 | HERVH-int,LTR7C | LI | 0.20 | 2.30 | 11.35 |
| HLA076 | 3 | 1.26E+08 | 1.26E+08 | ERV_2599552 | HERVH-int,LTR7C | LI | 0.25 | 6.61 | 26.70 |
| HLA066 | 3 | 1.28E+08 | 1.28E+08 | ERV_2601368 | MLT1C,MLT1C-int | LI | 0.00 | 1.57 | 300.00 |
| HLA065 | 3 | 1.29E+08 | 1.29E+08 | ERV_2601873 | THE1B,THE1B-int | LIL | 3.60 | 7.29 | 2.03 |
| HLA065 | 3 | 1.31E+08 | 1.31E+08 | ERV_2604096 | HERV4_I-int,MER51E | LI | 0.48 | 1.85 | 3.87 |
| HLA065 | 3 | 1.36E+08 | 1.36E+08 | ERV_2608801 | HERV16-int | I | 0.00 | 6.90 | 300.00 |
| HLA066 | 3 | 1.41E+08 | 1.41E+08 | ERV_2612781 | ERVL-B4-int | I | 0.00 | 2.93 | 300.00 |
| HLA066 | 3 | 1.43E+08 | 1.43E+08 | ERV_2614808 | MLT1O,MLT1O-int | LI | 0.06 | 6.56 | 105.79 |
| HLA076 | 3 | 1.43E+08 | 1.43E+08 | ERV_2614808 | MLT1O,MLT1O-int | LI | 0.00 | 6.22 | 300.00 |
| HLA065 | 3 | 1.47E+08 | 1.47E+08 | ERV_2617661 | MLT1A,MLT1A-int | LI | 4.58 | 9.61 | 2.10 |
| HLA065 | 3 | 1.47E+08 | 1.47E+08 | ERV_2617662 | MLT1A-int | I | 0.96 | 7.89 | 8.19 |
| HLA065 | 3 | 1.57E+08 | 1.57E+08 | ERV_2626701 | MLT1I,MLT1I-int | LI | 0.00 | 2.45 | 300.00 |
| HLA065 | 3 | 1.79E+08 | 1.79E+08 | ERV_2644692 | LTR49,LTR49-int | LIL | 0.32 | 1.69 | 5.32 |
| HLA065 | 3 | 1.79E+08 | 1.79E+08 | ERV_2644693 | MER34-int | I | 0.32 | 1.69 | 5.32 |
| HLA065 | 3 | 1.79E+08 | 1.79E+08 | ERV_2644697 | MER4B-int | I | 0.32 | 1.69 | 5.32 |
| HLA065 | 3 | 1.89E+08 | 1.89E+08 | ERV_2654773 | MLT1K-int | I | 1.16 | 4.88 | 4.21 |
| HLA076 | 3 | 1.89E+08 | 1.89E+08 | ERV_2655007 | ERVL-E-int | I | 0.42 | 2.05 | 4.85 |
| HLA065 | 3 | 1.95E+08 | 1.95E+08 | ERV_2660642 | THE1D,THE1D-int | LIL | 0.73 | 2.27 | 3.11 |
| HLA065 | 3 | 1.96E+08 | 1.96E+08 | ERV_2661061 | MER4-int,MER4A1 | LI | 2.16 | 8.31 | 3.84 |
| HLA065 | 3 | 1.96E+08 | 1.96E+08 | ERV_2661062 | MER4-int | I | 3.62 | 15.79 | 4.36 |
| HLA072 | 3 | 8951757 | 8952322 | ERV_2669662 | MLT1A,MLT1A-int | LI | 0.00 | 1.67 | 300.00 |
| HLA066 | 3 | 24355876 | 24356722 | ERV_2680511 | MLT-int | I | 0.35 | 1.92 | 5.47 |
| HLA066 | 3 | 24356459 | 24356557 | ERV_2680512 | MLT1F-int | I | 0.35 | 1.92 | 5.47 |
| HLA072 | 3 | 32460939 | 32466915 | ERV_2685671 | HERVH-int,LTR7 | LIL | 0.52 | 1.54 | 2.95 |
| HLA071 | 3 | 39792563 | 39795951 | ERV_2690420 | MER89,MER89-int | LIL | 0.14 | 3.10 | 21.60 |
| HLA076 | 3 | 39792563 | 39795951 | ERV_2690420 | MER89,MER89-int | LIL | 0.30 | 2.58 | 8.75 |
| HLA065 | 3 | 39843529 | 39845079 | ERV_2690449 | ERVL-E-int | I | 0.00 | 2.80 | 300.00 |
| HLA069 | 3 | 42918197 | 42924975 | ERV_2692477 | LTR25-int,MER61D | LI | 0.65 | 1.60 | 2.47 |
| HLA069 | 3 | 42924026 | 42924859 | ERV_2692480 | MER61-int | I | 0.65 | 1.60 | 2.47 |
| HLA065 | 3 | 44344658 | 44351310 | ERV_2693392 | HERVH-int,LTR7 | LI | 0.67 | 1.57 | 2.34 |
| HLA065 | 3 | 44432310 | 44439444 | ERV_2693437 | ERVL-B4-int,MLT2B4 | LI | 1.87 | 5.29 | 2.82 |
| HLA072 | 3 | 44432310 | 44439444 | ERV_2693437 | ERVL-B4-int,MLT2B4 | LI | 3.71 | 10.50 | 2.83 |
| HLA065 | 3 | 44437398 | 44438010 | ERV_2693443 | MER41C,PRIMA41-int | LIL | 1.87 | 5.29 | 2.82 |
| HLA072 | 3 | 44437398 | 44438010 | ERV_2693443 | MER41C,PRIMA41-int | LIL | 3.71 | 10.50 | 2.83 |
| HLA061 | 3 | 44482590 | 44492743 | ERV_2693473 | MER70-int,MER70C | LI | 1.81 | 4.42 | 2.44 |
| HLA072 | 3 | 44482590 | 44492743 | ERV_2693473 | MER70-int,MER70C | LI | 1.68 | 3.83 | 2.27 |
| HLA072 | 3 | 46417276 | 46417895 | ERV_2694938 | HERVL18-int,LTR18A | LI | 0.79 | 5.02 | 6.39 |
| HLA061 | 3 | 46828945 | 46829022 | ERV_2695182 | MLT1J-int | I | 0.58 | 3.92 | 6.71 |
| HLA063 | 3 | 46828945 | 46829022 | ERV_2695182 | MLT1J-int | I | 0.00 | 1.88 | 300.00 |
| HLA072 | 3 | 47185793 | 47187326 | ERV_2695395 | HERV16-int | I | 0.30 | 1.57 | 5.26 |
| HLA071 | 3 | 52363617 | 52364198 | ERV_2699363 | MLT1K,MLT1K-int | LI | 0.58 | 1.71 | 2.95 |
| HLA063 | 3 | 57549534 | 57550026 | ERV_2702721 | MER57A-int | I | 0.49 | 1.79 | 3.65 |
| HLA072 | 3 | 57549534 | 57550026 | ERV_2702721 | MER57A-int | I | 0.28 | 1.76 | 6.28 |
| HLA061 | 3 | 57550341 | 57552448 | ERV_2702723 | MER57A-int | I | 0.55 | 1.81 | 3.27 |
| HLA061 | 3 | 57552438 | 57553637 | ERV_2702728 | MER57A-int | I | 0.55 | 1.81 | 3.27 |
| HLA065 | 3 | 68991745 | 68994028 | ERV_2710761 | THE1A,THE1A-int | LIL | 0.23 | 2.29 | 10.09 |
| HLA063 | 3 | 1E+08 | 1E+08 | ERV_2725792 | MSTA,MSTA-int | LIL | 0.30 | 2.04 | 6.79 |
| HLA072 | 3 | 1.14E+08 | 1.14E+08 | ERV_2733312 | THE1C,THE1C-int | LIL | 0.47 | 2.87 | 6.13 |
| HLA065 | 3 | 1.21E+08 | 1.21E+08 | ERV_2737080 | HERVIP10FH-int,LTR10F | LIL | 0.37 | 1.78 | 4.83 |
| HLA071 | 3 | 1.21E+08 | 1.21E+08 | ERV_2737106 | MSTA,MSTA-int | LIL | 3.81 | 25.23 | 6.63 |
| HLA076 | 3 | 1.21E+08 | 1.21E+08 | ERV_2737106 | MSTA,MSTA-int | LIL | 6.62 | 17.57 | 2.65 |
| HLA065 | 3 | 1.23E+08 | 1.23E+08 | ERV_2738763 | ERVL-E-int | I | 0.00 | 2.27 | 300.00 |
| HLA065 | 3 | 1.24E+08 | 1.24E+08 | ERV_2738987 | THE1D,THE1D-int | LI | 1.11 | 2.36 | 2.13 |
| HLA071 | 3 | 1.24E+08 | 1.24E+08 | ERV_2738987 | THE1D,THE1D-int | LI | 4.17 | 16.16 | 3.88 |
| HLA072 | 3 | 1.24E+08 | 1.24E+08 | ERV_2738987 | THE1D,THE1D-int | LI | 0.65 | 1.67 | 2.59 |
| HLA073 | 3 | 1.24E+08 | 1.24E+08 | ERV_2738987 | THE1D,THE1D-int | LI | 0.56 | 4.40 | 7.86 |
| HLA065 | 3 | 1.26E+08 | 1.26E+08 | ERV_2740512 | HERVK-int,LTRSA | LI | 0.32 | 2.01 | 6.21 |
| HLA072 | 3 | 1.26E+08 | 1.26E+08 | ERV_2740512 | HERVK-int,LTRSA | LI | 0.18 | 3.67 | 20.43 |
| HLA066 | 3 | 1.26E+08 | 1.26E+08 | ERV_2740711 | MER57A-int,MER57C2 | LI | 0.22 | 1.92 | 8.65 |
| HLA076 | 3 | 1.26E+08 | 1.26E+08 | ERV_2740741 | HUERS-P2-int | I | 0.03 | 3.33 | 122.79 |
| HLA076 | 3 | 1.27E+08 | 1.27E+08 | ERV_2740977 | LTR52,LTR52-int | LI | 4.37 | 12.82 | 2.93 |
| HLA076 | 3 | 1.27E+08 | 1.27E+08 | ERV_2740978 | LTR52-int | I | 4.37 | 12.82 | 2.93 |
| HLA066 | 3 | 1.29E+08 | 1.29E+08 | ERV_2743068 | THE1A,THE1A-int | LIL | 2.75 | 6.06 | 2.20 |
| HLA067 | 3 | 1.39E+08 | 1.39E+08 | ERV_2749370 | HERVIP10FH-int | I | 0.00 | 2.00 | 300.00 |
| HLA063 | 3 | 1.46E+08 | 1.46E+08 | ERV_2753989 | MLT1D-int | I | 0.32 | 1.84 | 5.75 |
| HLA065 | 3 | 1.46E+08 | 1.46E+08 | ERV_2753989 | MLT1D-int | I | 0.14 | 4.03 | 28.07 |
| HLA076 | 3 | 1.56E+08 | 1.56E+08 | ERV_2759388 | MER41E,MER66-int | LI | 0.81 | 2.26 | 2.80 |
| HLA076 | 3 | 1.89E+08 | 1.89E+08 | ERV_2780386 | ERVL-E-int | I | 0.42 | 2.05 | 4.85 |
| HLA065 | 3 | 1.91E+08 | 1.91E+08 | ERV_2781142 | MER61-int,MER61A,MER61B | LIL | 0.68 | 4.28 | 6.27 |
| HLA065 | 3 | 1.98E+08 | 1.98E+08 | ERV_2785913 | MER4-int,MER4A1 | LI | 2.64 | 7.73 | 2.92 |
| HLA065 | 3 | 1.98E+08 | 1.98E+08 | ERV_2786124 | ERVL-E-int | I | 4.90 | 11.10 | 2.27 |
| HLA065 | 4 | 174515 | 175475 | ERV_2786516 | HERV4_I-int | I | 0.00 | 2.02 | 300.00 |
| HLA065 | 4 | 476656 | 477099 | ERV_2786754 | MER50-int | I | 0.00 | 1.67 | 300.00 |
| HLA065 | 4 | 980908 | 982755 | ERV_2787126 | HERVL74-int | I | 0.63 | 2.22 | 3.53 |
| HLA065 | 4 | 2477152 | 2479737 | ERV_2788260 | THE1B,THE1B-int | LIL | 0.61 | 1.65 | 2.72 |
| HLA065 | 4 | 2728490 | 2730727 | ERV_2788485 | LTR39-int | I | 0.92 | 2.30 | 2.51 |
| HLA065 | 4 | 8358459 | 8364122 | ERV_2793809 | MER41-int,MER41A | LIL | 0.87 | 2.49 | 2.86 |
| HLA076 | 4 | 8358459 | 8364122 | ERV_2793809 | MER41-int,MER41A | LIL | 2.70 | 9.82 | 3.64 |
| HLA065 | 4 | 8361456 | 8363579 | ERV_2793811 | MER41-int | I | 0.87 | 2.49 | 2.86 |
| HLA076 | 4 | 8361456 | 8363579 | ERV_2793811 | MER41-int | I | 2.70 | 9.82 | 3.64 |
| HLA065 | 4 | 9791305 | 9791937 | ERV_2795012 | MLT1F2,MLT1F2-int | LI | 1.25 | 5.23 | 4.20 |
| HLA065 | 4 | 17914855 | 17917663 | ERV_2802968 | MLT1F1,MLT1F1-int | LI | 1.37 | 7.85 | 5.72 |
| HLA065 | 4 | 23764968 | 23766385 | ERV_2808448 | MLT1L,MLT1L-int | LI | 1.50 | 11.96 | 7.98 |
| HLA058 | 4 | 1.23E+08 | 1.23E+08 | ERV_2890471 | ERVL-E-int | I | 0.00 | 1.66 | 300.00 |
| HLA061 | 4 | 1.23E+08 | 1.23E+08 | ERV_2890471 | ERVL-E-int | I | 0.00 | 1.93 | 300.00 |
| HLA065 | 4 | 491737 | 498501 | ERV_2945944 | MER4-int,MER4A1 | LI | 1.05 | 2.89 | 2.75 |
| HLA065 | 4 | 496166 | 497422 | ERV_2945948 | PRIMA4-int | I | 1.05 | 2.89 | 2.75 |
| HLA065 | 4 | 1690895 | 1691651 | ERV_2946462 | MLT1F-int | I | 0.00 | 2.27 | 300.00 |
| HLA065 | 4 | 6679767 | 6680232 | ERV_2950166 | MamGyp-int | I | 0.69 | 1.78 | 2.59 |
| HLA065 | 4 | 23759869 | 23760755 | ERV_2961152 | THE1B,THE1B-int | LI | 0.19 | 2.30 | 12.21 |
| HLA071 | 4 | 34178068 | 34180422 | ERV_2967337 | MamGyp-int | I | 0.30 | 2.09 | 6.95 |
| HLA071 | 5 | 1824816 | 1825059 | ERV_3053759 | ERVL-E-int | I | 0.00 | 2.11 | 300.00 |
| HLA058 | 5 | 18731989 | 18732283 | ERV_3066933 | ERV3-16A3_I-int | I | 0.49 | 2.69 | 5.46 |
| HLA058 | 5 | 28911600 | 28913194 | ERV_3075518 | MSTB,MSTB-int | LIL | 0.00 | 1.50 | 300.00 |
| HLA063 | 5 | 1.19E+08 | 1.19E+08 | ERV_3146773 | MLT1-int | I | 0.00 | 2.00 | 300.00 |
| HLA053 | 5 | 1.6E+08 | 1.6E+08 | ERV_3184264 | ERVL-E-int | I | 0.00 | 3.00 | 300.00 |
| HLA061 | 5 | 1.6E+08 | 1.6E+08 | ERV_3184264 | ERVL-E-int | I | 0.00 | 2.00 | 300.00 |
| HLA072 | 5 | 80135391 | 80135557 | ERV_3248382 | MLT1H-int | I | 0.00 | 2.50 | 300.00 |
| HLA072 | 5 | 1.29E+08 | 1.29E+08 | ERV_3274286 | PRIMA4-int | I | 0.00 | 2.00 | 300.00 |
| HLA071 | 5 | 1.46E+08 | 1.46E+08 | ERV_3285587 | PRIMA4-int | I | 0.47 | 2.58 | 5.47 |
| HLA072 | 6 | 29736067 | 29736195 | ERV_3331421 | ERVL-E-int | I | 1.49 | 4.00 | 2.68 |
| HLA072 | 6 | 1.09E+08 | 1.09E+08 | ERV_3376476 | LTR37-int | I | 0.20 | 1.51 | 7.40 |
| HLA067 | 6 | 35700080 | 35700151 | ERV_3444673 | MLT1H-int | I | 0.00 | 2.00 | 300.00 |
| HLA067 | 7 | 67292254 | 67292308 | ERV_3611239 | MER70-int | I | 1.00 | 3.15 | 3.15 |
| HLA065 | 7 | 75320503 | 75320629 | ERV_3621168 | MER84-int | I | 0.00 | 2.17 | 300.00 |
| HLA082 | 7 | 92486794 | 92486920 | ERV_3635952 | MLTIJ-int | I | 0.00 | 1.67 | 300.00 |
| HLA078 | 7 | 1.46E+08 | 1.46E+08 | ERV_3683626 | MLT1J2,MLT1J2-int | LI | 0.00 | 2.20 | 300.00 |
| HLA069 | 7 | 29410698 | 29410844 | ERV_3712658 | MER4-int | I | 0.00 | 1.60 | 300.00 |
| HLA058 | 7 | 39660894 | 39663183 | ERV_3718999 | THE1B,THE1B-int | LIL | 0.01 | 1.71 | 186.86 |
| HLA067 | 7 | 73156078 | 73156138 | ERV_3738996 | PRIMA4-int | I | 0.00 | 2.00 | 300.00 |
| HLA065 | 7 | 1.21E+08 | 1.21E+08 | ERV_3769128 | MER57A-int | I | 0.00 | 4.71 | 300.00 |
| HLA063 | 7 | 1.57E+08 | 1.57E+08 | ERV_3791405 | ERVL-B4-int | I | 0.00 | 1.52 | 300.00 |
| HLA061 | 8 | 1.26E+08 | 1.26E+08 | ERV_3900344 | ERVL-E-int | I | 0.43 | 1.58 | 3.67 |
| HLA066 | 8 | 1.41E+08 | 1.41E+08 | ERV_3915441 | ERVL-E-int | I | 0.25 | 2.21 | 9.01 |
| HLA065 | 8 | 12732389 | 12732421 | ERV_3926223 | HUERS-P3-int | I | 0.00 | 3.00 | 300.00 |
| HLA063 | 8 | 60397280 | 60397864 | ERV_3954233 | ERV3-16A3_I-int | I | 0.00 | 1.53 | 300.00 |
| HLA053 | 8 | 1.3E+08 | 1.3E+08 | ERV_3997466 | MER21-int | I | 0.00 | 2.00 | 300.00 |
| HLA061 | 9 | 1.12E+08 | 1.12E+08 | ERV_4089269 | MER4-int | I | 0.77 | 2.00 | 2.59 |
| HLA073 | 9 | 1.37E+08 | 1.37E+08 | ERV_4116210 | MER101-int | I | 0.00 | 2.00 | 300.00 |
| HLA072 | 9 | 35831502 | 35831706 | ERV_4137812 | ERVL-B4-int | I | 0.00 | 1.96 | 300.00 |
| HLA053 | X | 27677766 | 27677862 | ERV_4223114 | ERVL-E-int | I | 0.00 | 3.00 | 300.00 |
| HLA073 | X | 1.54E+08 | 1.54E+08 | ERV_4320593 | MLT1-int | I | 0.00 | 2.00 | 300.00 |
| HLA082 | X | 1.54E+08 | 1.54E+08 | ERV_4320593 | MLT1-int | I | 0.00 | 1.58 | 300.00 |
| HLA082 | X | 68836462 | 68836528 | ERV_4364403 | MamGyp-int | I | 0.00 | 1.67 | 300.00 |
| HLA065 | X | 71784021 | 71784077 | ERV_4366533 | HERVK11-int | I | 0.00 | 2.00 | 300.00 |

SAMPLE = sample code; CHROM = chromosome; START = start nucleotide position on chromosome; STOP = stop nucleotide position on chromosome; ID = ERV identification number; NAME = ERV type name; Subtype = region in the ERV sequence; NT = expression level in non-tumor tissues; T = expression level in tumor tissues; ratio T vs. NT = ratio between expression level in tumor vs. non-tumor tissues.

The over-expressed sequences in more than 50% of the analyzed samples are shown in Table 2 and were selected to identify specific internal gene sequences of HERVs.

TABLE 2

Expression of individual ERVs in the liver cancer samples.

| ID | HLA053 | HLA 058 | HLA 061 | HLA 063 | HLA 065 | HLA 066 | HLA 067 | HLA 069 | HLA071 | HLA 072 | HLA 073 | HLA076 | HLA078 | HLA0 82 |
|---|---|---|---|---|---|---|---|---|---|---|---|---|---|---|
| ERV_0312447 | | X | X | X | | | X | X | | | X | | X | X |
| ERV_0312445 | | X | X | X | | | X | X | | | X | | X | X |
| ERV_0127104 | | X | X | X | | X | X | X | | | X | | X | |
| ERV_0168088 | X | | | | X | | X | X | | | | X | X | X |
| ERV_0168090 | X | | | | X | | X | X | | | | X | X | X |
| ERV_0227869 | | X | | | X | X | X | X | | | X | X | X | |
| ERV_0300843 | | X | | | | X | X | X | X | | | X | X | X |
| ERV_0308616 | | X | X | X | | X | X | X | | | X | | X | |
| ERV_2093181 | | | | X | X | X | X | X | | X | X | | | X |

### Protein Sequences of HERVs Genes

The nucleotide sequences of the HERVd database in Table 2 were translated and aligned with Blast to identify specific retroviral open-reading frames (ORF), that is, to verify the specific identity of the HERV proteins. In the vast majority of cases such ORFs have not been identified. Only for three sequences (ID: ERV_0312447, ERV_0312445, having SEQ ID NO: 1 and ERV_0308616, having SEQ ID NO: 2) ORFs corresponding to retroviral proteins were identified, in particular these sequences were found to be corresponding to genes of the HERV-K. In particular, the sequences ERV 0312447, ERV_0312445 were found to be identical to each other and identical (100% homology) with the Pol sequence of member 7 of the HERV-K (aa 85-1459) and have sequence:

MLTDLRAVNAVIQPMGPLQPGLPSPAMIPKDWPLIIIDLKDCFFTIPLAE
QDCEKFAFTIPAINNKEPATRFQWKVLPQGMLNSPTICQTFVGRALQPVR
EKFSDCYIIHYIDDILCAAETRDKLIDCYTFLQAEVANAGLAIASDKIQT
STPFHYLGMQIENRKIKQQKIEIRKDTLKTLNDFQKLLGDINWIRPTLGI
PTYAMSNLFSILRGDSDLNSKRILTPEATKEIKLVEEKIQSAQINRIDPL
APLQLLIFATAHSPTGIIIQNTDLVEWSFLPHSTVKTFTLYLDQIATLIG
QTRLRIIKLCGNDPDKIVVPLTKEQVRQAFINSGAWQIGLANFVGIIDNH
YPKTKIFQFLKLTTWILPKITRREPLENALTVFTDGSSNGKAAYTGPKER
VIKTPYQSAQRAELVAVITVLQDFDQPINIISDSAYVVQATRDVETALIK
YSMDDQLNQLFNLLQQTVRKRNFPFYITHIRAHTNLPGPLTKANEQADLL
VSSALIKAQELHALTHVNAAGLKNKFDVTWKQAKDIVQHCTQCQILHLPT
QEAGVNPRGLCPNALWQMDVTHVPSFGRLSYVHVTVDTYSHFIWATCQTG
ESTSHVKKHLLSCFAVMGVPEKIKTDNGPGYCSKAFQKFLSQWKISHTTG
IPYNSQGQAIVERTNRTLKTQLVKQKEGGDSKECTTPQMQLNLALYTLNF
LNIYRNQTTTSAEQHLTGKKNSPHEGKLIWWKDNKNKTWEIGKVITWGRG
FACVSPGENQLPVWIPTRHLKFYNEPIGDAKKRASTEMVTPVTWMDNPIE
IYVNDSVWVPGPIDDRCPAKPEEEGMMINISIGYRYPPICLGRAPGCLMP
AVQNWLVEVPTVSPISRFTYHMVSGMSLRPRVNYLQDFSYQRSLKFRPKG
KPCPKEIPKESKNTEVLVWEECVANSAVILQNNEFGTIIDWAPRGQFYHN
CSGQTQSCPSAQVSPAVDSDLTESLDKHKHKKLQSFYPWEWGEKGISTPR
PKIVSPVSGPEHPELWRLTVASHHIRIWSGNQTLETRDCKPFYTIDLNSS
LTVPLQSCVKPPYMLVVGNIVIKPDSQTITCENCRLLTCIDSTFNWQHRI
LLVRAREGVWIPVSMDRPWEASPSVHILTEVLKGVLNRSKRFIFTLIAVI
MGLIAVTATAAVAGVALHSSVQSVNFVNDWQKNSTRLWNSQSSIDQKLAN
QINDLRQTVIWMGDRLMSLEHRFQLQCDWNTSDFCITPQIYNESEHHWDM
VRRHLQGREDNLTLDISKLKEQIFEASKAHLNLVPGTEAIAGVADGLANL
NPVTWVKTIGSTTIINLILILVCLFCLLLVCRCTQQLRRDSDHRERAMMT
MAVLSKRKGGNVGKSKRDQIVTVSV(SEQ ID NO:1).

### The Sequence ERV_0308616, With Sequence

MDDQLNQLFNLLQQTVRKRNFPFYITHIRAHTNLPGPLTKANEPADLLVS
SAFIKAQKFHALTHVNAAGLKNKFDVTWKQAKDIVRHCTQCQVLHLPTQE
AGVNPRGLCPNALRQMDVTHSKAFQKFLSQWKISHTTGIPYNSQGQAIVE
RTNRTLKTQLVKVPSFGRLLYVHVTVDTYSHFIWATCQTGESTSHVKKHL
LPCFAVMRVPEKIKTDNGAGYC(SEQ ID NO:2),

shows 96% homology with the Pol sequence of the Member 6 of HERV-K (aa 537-716; https://www.uniprot.org/uniprot/Q9BXR3) with sequence

MDDQLNQLFNLLQQTVRKRNFPFYITHIRAHTNLPGPLTKANEQADLLVS
SALIKAQELHALTHVNAAGLKNKFDVTWKQAKDIVQHCTQCQVLHLPTQE
AGVNPRGLCPNALWQMDVTHSKAFQKFLSQWKISHTTGIPYNSQGQAIVE
RTNRTLKTQLVKVPSFGRLSYVHVTVDTYSHFIWATCQTGESTSHVKKHL
LSCFAVMGVPEKIKTDNGPGYC(SEQ ID NO:3).

### Prediction of HERVs Epitopes

The Pol sequences identified in the HCC samples (SEQ ID NO: 1 and SEQ ID NO: 2) were used for the prediction of 9 amino acid epitopes by NetMHCpan version 4.0. Antigens were selected for each class I HLA with predicted affinity <100 nM, corresponding to strong binders (SB). Below, in Table 3, the sequences of the predicted HERV epitopes or peptides are reported, together with the references SEQ ID NO.

Peptides common to all three analyzed sequences are highlighted in bold type.

TABLE 3

| SEQ ID NO: | ID | HLA | PEPTIDE | SCORE | AFF(nM) | %RANK | BINDLEVEL |
|---|---|---|---|---|---|---|---|
| 4 | ERV_312447 | HLA-B*40:01 | AELVAVITV | 0.65 | 43.40 | 0.12 | SB |
| 4 | ERV_312445 | HLA-B*40:01 | AELVAVITV | 0.65 | 43.40 | 0.12 | SB |
| 4 | ERV_0308616 | HLA-B*40:01 | AELVAVITV | 0.65 | 43.40 | 0.12 | SB |
| 5 | ERV_312447 | HLA-A*02:01 | AMIPKDWPL | 0.85 | 4.80 | 0.03 | SB |
| 5 | ERV_312445 | HLA-A*02:01 | AMIPKDWPL | 0.85 | 4.80 | 0.03 | SB |
| 6 | ERV_312447 | HLA-B*15:01 | AMSNLFSIL | 0.61 | 64.60 | 0.35 | SB |
| 6 | ERV_312445 | HLA-B*15:01 | AMSNLFSIL | 0.61 | 64.60 | 0.35 | SB |
| 7 | ERV_312447 | HLA-B*15:01 | AQAITGVVL | 0.59 | 81.40 | 0.44 | SB |
| 7 | ERV_312445 | HLA-B*15:01 | AQAITGVVL | 0.59 | 81.40 | 0.44 | SB |
| 8 | ERV_312447 | HLA-B*15:01 | AQINRIDPL | 0.59 | 88.50 | 0.47 | SB |
| 8 | ERV_312445 | HLA-B*15:01 | AQINRIDPL | 0.59 | 88.50 | 0.47 | SB |
| 9 | ERV_312447 | HLA-B*27:05 | ARYKSFSIK | 0.58 | 90.20 | 0.30 | SB |
| 9 | ERV_312445 | HLA-B*27:05 | ARYKSFSIK | 0.58 | 90.20 | 0.30 | SB |
| 10 | ERV_0308616 | HLA-B*07:02 | ATRDVKTAL | 0.60 | 79.70 | 0.25 | SB |
| 11 | ERV_312447 | HLA-A*02:01 | AVIMGLIAV | 0.66 | 37.60 | 0.49 | SB |
| 11 | ERV_312445 | HLA-A*02:01 | AVIMGLIAV | 0.66 | 37.60 | 0.49 | SB |
| 12 | ERV_312447 | HLA-B*15:01 | AVNAVIQPM | 0.58 | 89.60 | 0.48 | SB |
| 12 | ERV_312445 | HLA-B*15:01 | AVNAVIQPM | 0.58 | 89.60 | 0.48 | SB |
| 13 | ERV_312447 | HLA-A*02:01 | AVQNWLVEV | 0.71 | 22.20 | 0.30 | SB |
| 13 | ERV_312445 | HLA-A*02:01 | AVQNWLVEV | 0.71 | 22.20 | 0.30 | SB |
| 14 | ERV_312447 | HLA-B*58:01 | CLMPAVQNW | 0.71 | 23.80 | 0.16 | SB |
| 14 | ERV_312445 | HLA-B*58:01 | CLMPAVQNW | 0.71 | 23.80 | 0.16 | SB |
| 15 | ERV_312447 | HLA-B*08:01 | EARYKSFSI | 0.69 | 29.40 | 0.05 | SB |
| 15 | ERV_312445 | HLA-B*08:01 | EARYKSFSI | 0.69 | 29.40 | 0.05 | SB |
| 16 | ERV_312447 | HLA-B*39:01 | ERAMMTMAV | 0.62 | 58.40 | 0.06 | SB |
| 16 | ERV_312445 | HLA-B*39:01 | ERAMMTMAV | 0.62 | 58.40 | 0.06 | SB |
| 17 | ERV_312447 | HLA-A*02:01 | FINSGAWQI | 0.75 | 14.50 | 0.18 | SB |
| 17 | ERV_312445 | HLA-A*02:01 | FINSGAWQI | 0.75 | 14.50 | 0.18 | SB |
| 17 | ERV_0308616 | HLA-A*02:01 | FINSGAWQI | 0.75 | 14.50 | 0.18 | SB |
| 18 | ERV_312447 | HLA-B*08:01 | FLKLTTWIL | 0.65 | 45.80 | 0.08 | SB |
| 18 | ERV_312445 | HLA-B*08:01 | FLKLTTWIL | 0.65 | 45.80 | 0.08 | SB |
| 18 | ERV_0308616 | HLA-B*08:01 | FLKLTTWIL | 0.65 | 45.80 | 0.08 | SB |
| 19 | ERV_312447 | HLA-A*02:01 | FLQAEVANA | 0.75 | 14.80 | 0.18 | SB |
| 19 | ERV_312445 | HLA-A*02:01 | FLQAEVANA | 0.75 | 14.80 | 0.18 | SB |
| 20 | ERV_312447 | HLA-A*02:01 | FLQFKTWWI | 0.83 | 6.30 | 0.05 | SB |
| 20 | ERV_312445 | HLA-A*02:01 | FLQFKTWWI | 0.83 | 6.30 | 0.05 | SB |
| 21 | ERV_312447 | HLA-A*01:01 | FSDCYIIHY | 0.82 | 7.00 | 0.01 | SB |
| 21 | ERV_312445 | HLA-A*01:01 | FSDCYIIHY | 0.82 | 7.00 | 0.01 | SB |
| 22 | ERV_312447 | HLA-B*58:01 | FSYQRSLKF | 0.62 | 64.10 | 0.35 | SB |
| 22 | ERV_312447 | HLA-B*15:01 | FSYQRSLKF | 0.67 | 35.40 | 0.17 | SB |
| 22 | ERV_312445 | HLA-B*58:01 | FSYQRSLKF | 0.62 | 64.10 | 0.35 | SB |
| 22 | ERV_312445 | HLA-B*15:01 | FSYQRSLKF | 0.67 | 35.40 | 0.17 | SB |
| 23 | ERV_312447 | HLA-A*26:01 | FTYHMVSGM | 0.66 | 39.10 | 0.02 | SB |
| 23 | ERV_312445 | HLA-A*26:01 | FTYHMVSGM | 0.66 | 39.10 | 0.02 | SB |
| 24 | ERV_312447 | HLA-A*02:01 | FVGRALQPV | 0.74 | 16.90 | 0.22 | SB |
| 24 | ERV_312445 | HLA-A*02:01 | FVGRALQPV | 0.74 | 16.90 | 0.22 | SB |
| 25 | ERV_312447 | HLA-B*40:01 | GENQLPVWI | 0.60 | 73.00 | 0.20 | SB |
| 25 | ERV_312445 | HLA-B*40:01 | GENQLPVWI | 0.60 | 73.00 | 0.20 | SB |
| 26 | ERV_312447 | HLA-B*08:01 | HLKKNCPVL | 0.59 | 85.30 | 0.15 | SB |
| 26 | ERV_312445 | HLA-B*08:01 | HLKKNCPVL | 0.59 | 85.30 | 0.15 | SB |
| 27 | ERV_0308616 | HLA-B*15:01 | HLLPCFAVM | 0.60 | 72.60 | 0.39 | SB |
| 27 | ERV_312447 | HLA-B*15:01 | HLLSCFAVM | 0.62 | 59.30 | 0.32 | SB |
| 27 | ERV_312445 | HLA-B*15:01 | HLLSCFAVM | 0.62 | 59.30 | 0.32 | SB |
| 28 | ERV_312447 | HLA-B*07:02 | HPELWRLTV | 0.66 | 41.30 | 0.15 | SB |
| 28 | ERV_312445 | HLA-B*07:02 | HPELWRLTV | 0.66 | 41.30 | 0.15 | SB |
| 29 | ERV_312447 | HLA-B*39:01 | HRERAMMTM | 0.62 | 62.40 | 0.07 | SB |
| 29 | ERV_312445 | HLA-B*39:01 | HRERAMMTM | 0.62 | 62.40 | 0.07 | SB |
| 30 | ERV_312447 | HLA-B*27:05 | HRILLVRAR | 0.67 | 36.80 | 0.10 | SB |
| 30 | ERV_312445 | HLA-B*27:05 | HRILLVRAR | 0.67 | 36.80 | 0.10 | SB |
| 31 | ERV_312447 | HLA-B*40:01 | IEIYVNDSV | 0.61 | 67.90 | 0.19 | SB |
| 31 | ERV_312445 | HLA-B*40:01 | IEIYVNDSV | 0.61 | 67.90 | 0.19 | SB |
| 32 | ERV_0308616 | HLA-B*08:01 | IKAQKFHAL | 0.58 | 97.50 | 0.17 | SB |
| 33 | ERV_312447 | HLA-A*02:01 | ILTEVLKGV | 0.76 | 14.10 | 0.17 | SB |
| 33 | ERV_312445 | HLA-A*02:01 | ILTEVLKGV | 0.76 | 14.10 | 0.17 | SB |
| 34 | ERV_312447 | HLA-B*07:02 | IPTYAMSNL | 0.62 | 64.10 | 0.21 | SB |
| 34 | ERV_312445 | HLA-B*07:02 | IPTYAMSNL | 0.62 | 64.10 | 0.21 | SB |
| 35 | ERV_312447 | HLA-B*15:01 | IQTSTPFHY | 0.61 | 71.70 | 0.39 | SB |
| 35 | ERV_312445 | HLA-B*15:01 | IQTSTPFHY | 0.61 | 71.70 | 0.39 | SB |
| 36 | ERV_312447 | HLA-B*15:01 | ISHTTGIPY | 0.66 | 40.30 | 0.20 | SB |
| 36 | ERV_312445 | HLA-B*15:01 | ISHTTGIPY | 0.66 | 40.30 | 0.20 | SB |
| 36 | ERV_0308616 | HLA-B*15:01 | ISHTTGIPY | 0.66 | 40.30 | 0.20 | SB |
| 37 | ERV_312447 | HLA-B*58:01 | KAMLMAQAI | 0.62 | 59.70 | 0.33 | SB |
| 37 | ERV_312445 | HLA-B*58:01 | KAMLMAQAI | 0.62 | 59.70 | 0.33 | SB |
| 38 | ERV_312447 | HLA-B*40:01 | KESKNTEVL | 0.69 | 27.80 | 0.07 | SB |
| 38 | ERV_312445 | HLA-B*40:01 | KESKNTEVL | 0.69 | 27.80 | 0.07 | SB |
| 39 | ERV_312447 | HLA-A*02:01 | KLIDCYTFL | 0.84 | 5.60 | 0.04 | SB |
| 39 | ERV_312445 | HLA-A*02:01 | KLIDCYTFL | 0.84 | 5.60 | 0.04 | SB |
| 40 | ERV_312447 | HLA-A*03:01 | KLIWWKDNK | 0.63 | 53.10 | 0.20 | SB |
| 40 | ERV_312445 | HLA-A*03:01 | KLIWWKDNK | 0.63 | 53.10 | 0.20 | SB |
| 41 | ERV_312447 | HLA-A*02:01 | KLKEQIFEA | 0.72 | 19.80 | 0.27 | SB |
| 41 | ERV_312445 | HLA-A*02:01 | KLKEQIFEA | 0.72 | 19.80 | 0.27 | SB |
| 42 | ERV_312447 | HLA-A*02:01 | KLLGDINWI | 0.81 | 8.00 | 0.08 | SB |
| 42 | ERV_312445 | HLA-A*02:01 | KLLGDINWI | 0.81 | 8.00 | 0.08 | SB |
| 43 | ERV_312447 | HLA-A*03:01 | KLTTWILPK | 0.78 | 10.90 | 0.02 | SB |
| 43 | ERV_312445 | HLA-A*03:01 | KLTTWILPK | 0.78 | 10.90 | 0.02 | SB |
| 43 | ERV_0308616 | HLA-A*03:01 | KLTTWILPK | 0.78 | 10.90 | 0.02 | SB |
| 44 | ERV_312447 | HLA-B*07:02 | KPLKGKVPA | 0.63 | 53.20 | 0.18 | SB |
| 44 | ERV_312445 | HLA-B*07:02 | KPLKGKVPA | 0.63 | 53.20 | 0.18 | SB |
| 45 | ERV_312447 | HLA-B*15:01 | KQYGPNSPY | 0.79 | 9.30 | 0.02 | SB |
| 45 | ERV_312445 | HLA-B*15:01 | KQYGPNSPY | 0.79 | 9.30 | 0.02 | SB |
| 46 | ERV_312447 | HLA-B*27:05 | KRFIFTLIA | 0.58 | 93.20 | 0.31 | SB |
| 46 | ERV_312445 | HLA-B*27:05 | KRFIFTLIA | 0.58 | 93.20 | 0.31 | SB |
| 47 | ERV_312447 | HLA-A*03:01 | KSFSIKMLK | 0.78 | 10.50 | 0.02 | SB |
| 47 | ERV_312445 | HLA-A*03:01 | KSFSIKMLK | 0.78 | 10.50 | 0.02 | SB |
| 48 | ERV_312447 | HLA-B*58:01 | KSSLSPSQF | 0.67 | 34.80 | 0.22 | SB |
| 48 | ERV_312445 | HLA-B*58:01 | KSSLSPSQF | 0.67 | 34.80 | 0.22 | SB |
| 49 | ERV_0308616 | HLA-B*58:01 | KTALIKYSM | 0.62 | 62.50 | 0.34 | SB |
| 50 | ERV_312447 | HLA-A*03:01 | KTKIFQFLK | 0.71 | 22.40 | 0.06 | SB |
| 50 | ERV_312445 | HLA-A*03:01 | KTKIFQFLK | 0.71 | 22.40 | 0.06 | SB |
| 50 | ERV_0308616 | HLA-A*03:01 | KTKIFQFLK | 0.71 | 22.40 | 0.06 | SB |
| 51 | ERV_312447 | HLA-B*15:01 | KVIVELMAY | 0.62 | 63.70 | 0.34 | SB |
| 51 | ERV_312445 | HLA-B*15:01 | KVIVELMAY | 0.62 | 63.70 | 0.34 | SB |
| 52 | ERV_312447 | HLA-B*58:01 | LANLNPVTW | 0.82 | 7.00 | 0.03 | SB |
| 53 | ERV_312447 | HLA-B*15:01 | LLTCIDSTF | 0.66 | 40.00 | 0.20 | SB |
| 53 | ERV_312445 | HLA-B*15:01 | LLTCIDSTF | 0.66 | 40.00 | 0.20 | SB |
| 54 | ERV_0308616 | HLA-A*03:01 | LLVSSAFIK | 0.61 | 66.60 | 0.25 | SB |
| 54 | ERV_312447 | HLA-A*03:01 | LLVSSALIK | 0.62 | 63.50 | 0.24 | SB |
| 54 | ERV_312445 | HLA-A*03:01 | LLVSSALIK | 0.62 | 63.50 | 0.24 | SB |
| 55 | ERV_312447 | HLA-A*02:01 | LMAQAITGV | 0.81 | 7.50 | 0.08 | SB |
| 55 | ERV_312445 | HLA-A*02:01 | LMAQAITGV | 0.81 | 7.50 | 0.08 | SB |
| 56 | ERV_312447 | HLA-B*07:02 | LPHSTVKTF | 0.60 | 79.00 | 0.25 | SB |
| 56 | ERV_312445 | HLA-B*07:02 | LPHSTVKTF | 0.60 | 79.00 | 0.25 | SB |
| 57 | ERV_312447 | HLA-B*07:02 | LPKQKLEAL | 0.74 | 17.30 | 0.06 | SB |
| 57 | ERV_312447 | HLA-B*08:01 | LPKQKLEAL | 0.72 | 19.90 | 0.03 | SB |
| 57 | ERV_312445 | HLA-B*07:02 | LPKQKLEAL | 0.74 | 17.30 | 0.06 | SB |
| 57 | ERV_312445 | HLA-B*08:01 | LPKQKLEAL | 0.72 | 19.90 | 0.03 | SB |
| 58 | ERV_312447 | HLA-B*15:01 | LQSCVKPPY | 0.70 | 25.70 | 0.12 | SB |
| 58 | ERV_312445 | HLA-B*15:01 | LQSCVKPPY | 0.70 | 25.70 | 0.12 | SB |
| 59 | ERV_312447 | HLA-B*58:01 | LQSFYPWEW | 0.68 | 30.40 | 0.20 | SB |
| 59 | ERV_312445 | HLA-B*58:01 | LQSFYPWEW | 0.68 | 30.40 | 0.20 | SB |
| 60 | ERV_312447 | HLA-A*24:02 | LYLDQIATL | 0.58 | 98.90 | 0.21 | SB |
| 60 | ERV_312445 | HLA-A*24:02 | LYLDQIATL | 0.58 | 98.90 | 0.21 | SB |
| 61 | ERV_312447 | HLA-A*24:02 | LYTLNFLNI | 0.64 | 50.80 | 0.12 | SB |
| 61 | ERV_312445 | HLA-A*24:02 | LYTLNFLNI | 0.64 | 50.80 | 0.12 | SB |
| 62 | ERV_312447 | HLA-A*03:01 | MMINISIGY | 0.62 | 60.40 | 0.23 | SB |
| 62 | ERV_312447 | HLA-B*15:01 | MMINISIGY | 0.81 | 8.20 | 0.01 | SB |
| 62 | ERV_312445 | HLA-A*03:01 | MMINISIGY | 0.62 | 60.40 | 0.23 | SB |
| 62 | ERV_312445 | HLA-B*15:01 | MMINISIGY | 0.81 | 8.20 | 0.01 | SB |
| 63 | ERV_312447 | HLA-A*03:01 | MMTMAVLSK | 0.72 | 19.90 | 0.05 | SB |
| 63 | ERV_312445 | HLA-A*03:01 | MMTMAVLSK | 0.72 | 19.90 | 0.05 | SB |
| 64 | ERV_312447 | HLA-B*39:01 | NRIDPLAPL | 0.76 | 13.50 | 0.01 | SB |
| 64 | ERV_312445 | HLA-B*39:01 | NRIDPLAPL | 0.76 | 13.50 | 0.01 | SB |
| 65 | ERV_312447 | H LA-A*24:02 | PYMLVVGNI | 0.61 | 66.00 | 0.15 | SB |
| 65 | ERV_312445 | HLA-A*24:02 | PYMLVVGNI | 0.61 | 66.00 | 0.15 | SB |
| 66 | ERV_312447 | HLA-B*58:01 | QAFINSGAW | 0.70 | 26.40 | 0.18 | SB |
| 66 | ERV_312445 | HLA-B*58:01 | QAFINSGAW | 0.70 | 26.40 | 0.18 | SB |
| 66 | ERV_0308616 | HLA-B*58:01 | QAFINSGAW | 0.70 | 26.40 | 0.18 | SB |
| 67 | ERV_312447 | HLA-B*07:02 | QPGLPSPAM | 0.64 | 46.80 | 0.16 | SB |
| 67 | ERV_312445 | HLA-B*07:02 | QPGLPSPAM | 0.64 | 46.80 | 0.16 | SB |
| 68 | ERV_312447 | HLA-B*58:01 | QSVNFVNDW | 0.75 | 15.00 | 0.10 | SB |
| 68 | ERV_312445 | HLA-B*58:01 | QSVNFVNDW | 0.75 | 15.00 | 0.10 | SB |
| 69 | ERV_312447 | HLA-B*07:02 | RAMMTMAVL | 0.67 | 33.90 | 0.12 | SB |
| 69 | ERV_312447 | HLA-B*58:01 | RAMMTMAVL | 0.63 | 53.70 | 0.30 | SB |
| 69 | ERV_312445 | HLA-B*07:02 | RAMMTMAVL | 0.67 | 33.90 | 0.12 | SB |
| 69 | ERV_312445 | HLA-B*58:01 | RAMMTMAVL | 0.63 | 53.70 | 0.30 | SB |
| 70 | ERV_312447 | HLA-B*40:01 | REDNLTLDI | 0.61 | 64.50 | 0.18 | SB |
| 70 | ERV_312445 | HLA-B*40:01 | REDNLTLDI | 0.61 | 64.50 | 0.18 | SB |
| 71 | ERV_312447 | HLA-B*58:01 | RGLCPNALW | 0.76 | 13.90 | 0.09 | SB |
| 71 | ERV_312445 | HLA-B*58:01 | RGLCPNALW | 0.76 | 13.90 | 0.09 | SB |
| 72 | ERV_312447 | HLA-A*03:01 | RILTPEATK | 0.64 | 49.80 | 0.19 | SB |
| 72 | ERV_312445 | HLA-A*03:01 | RILTPEATK | 0.64 | 49.80 | 0.19 | SB |
| 73 | ERV_312447 | HLA-A*02:01 | RLIPYDWEI | 0.88 | 3.70 | 0.02 | SB |
| 73 | ERV_312445 | HLA-A*02:01 | RLIPYDWEI | 0.88 | 3.70 | 0.02 | SB |
| 74 | ERV_0308616 | HLA-A*02:01 | RLLYVHVTV | 0.70 | 24.70 | 0.34 | SB |
| 75 | ERV_312447 | HLA-B*15:01 | RLMSLEHRF | 0.61 | 67.00 | 0.36 | SB |
| 75 | ERV_312445 | HLA-B*15:01 | RLMSLEHRF | 0.61 | 67.00 | 0.36 | SB |
| 76 | ERV_312447 | HLA-A*02:01 | RLSYVHVTV | 0.73 | 18.10 | 0.24 | SB |
| 76 | ERV_312445 | HLA-A*02:01 | RLSYVHVTV | 0.73 | 18.10 | 0.24 | SB |
| 77 | ERV_312447 | HLA-A*02:01 | RMLTDLRAV | 0.72 | 20.40 | 0.28 | SB |
| 77 | ERV_312445 | HLA-A*02:01 | RMLTDLRAV | 0.72 | 20.40 | 0.28 | SB |
| 78 | ERV_312447 | HLA-B*07:02 | RPWEASPSV | 0.70 | 26.60 | 0.10 | SB |
| 78 | ERV_312445 | HLA-B*07:02 | RPWEASPSV | 0.70 | 26.60 | 0.10 | SB |
| 79 | ERV_0308616 | HLA-B*27:05 | RRAELVAVI | 0.68 | 32.60 | 0.07 | SB |
| 80 | ERV_312447 | HLA-B*27:05 | RRNRLSFLG | 0.61 | 65.30 | 0.22 | SB |
| 80 | ERV_312445 | HLA-B*27:05 | RRNRLSFLG | 0.61 | 65.30 | 0.22 | SB |
| 81 | ERV_312447 | HLA-A*03:01 | RTLKTQLVK | 0.65 | 45.50 | 0.17 | SB |
| 81 | ERV_312445 | HLA-A*03:01 | RTLKTQLVK | 0.65 | 45.50 | 0.17 | SB |
| 81 | ERV_0308616 | HLA-A*03:01 | RTLKTQLVK | 0.65 | 45.50 | 0.17 | SB |
| 82 | ERV_312447 | HLA-B*08:01 | SLRPRVNYL | 0.75 | 14.30 | 0.01 | SB |
| 82 | ERV_312445 | HLA-B*08:01 | SLRPRVNYL | 0.75 | 14.30 | 0.01 | SB |
| 83 | ERV_312447 | HLA-A*02:01 | SMDDQLNQL | 0.72 | 21.60 | 0.30 | SB |
| 83 | ERV_312445 | HLA-A*02:01 | SMDDQLNQL | 0.72 | 21.60 | 0.30 | SB |
| 83 | ERV_0308616 | HLA-A*02:01 | SMDDQLNQL | 0.72 | 21.60 | 0.30 | SB |
| 84 | ERV_312447 | HLA-A*26:01 | STVKTFTLY | 0.68 | 33.30 | 0.02 | SB |
| 84 | ERV_312447 | HLA-B*15:01 | STVKTFTLY | 0.62 | 60.40 | 0.32 | SB |
| 84 | ERV_312445 | HLA-A*26:01 | STVKTFTLY | 0.68 | 33.30 | 0.02 | SB |
| 84 | ERV_312445 | HLA-B*15:01 | STVKTFTLY | 0.62 | 60.40 | 0.32 | SB |
| 85 | ERV_312447 | HLA-B*58:01 | TCIDSTFNW | 0.67 | 36.10 | 0.23 | SB |
| 85 | ERV_312445 | HLA-B*58:01 | TCIDSTFNW | 0.67 | 36.10 | 0.23 | SB |
| 86 | ERV_312447 | HLA-B*39:01 | THIRAHTNL | 0.62 | 60.90 | 0.07 | SB |
| 86 | ERV_312445 | HLA-B*39:01 | THIRAHTNL | 0.62 | 60.90 | 0.07 | SB |
| 86 | ERV_0308616 | HLA-B*39:01 | THIRAHTNL | 0.62 | 60.90 | 0.07 | SB |
| 87 | ERV_312447 | HLA-A*02:01 | TLIAVIMGL | 0.77 | 12.40 | 0.15 | SB |
| 87 | ERV_312445 | HLA-A*02:01 | TLIAVIMGL | 0.77 | 12.40 | 0.15 | SB |
| 88 | ERV_312447 | HLA-B*08:01 | TVRKRNFPF | 0.68 | 31.30 | 0.05 | SB |
| 88 | ERV_312445 | HLA-B*08:01 | TVRKRNFPF | 0.68 | 31.30 | 0.05 | SB |
| 88 | ERV_0308616 | HLA-B*08:01 | TVRKRNFPF | 0.68 | 31.30 | 0.05 | SB |
| 89 | ERV_312447 | HLA-B*58:01 | VASHHIRIW | 0.70 | 26.90 | 0.18 | SB |
| 89 | ERV_312445 | HLA-B*58:01 | VASHHIRIW | 0.70 | 26.90 | 0.18 | SB |
| 90 | ERV_312447 | HLA-B*40:01 | VEVPTVSPI | 0.62 | 60.20 | 0.17 | SB |
| 90 | ERV_312445 | HLA-B*40:01 | VEVPTVSPI | 0.62 | 60.20 | 0.17 | SB |
| 91 | ERV_312447 | HLA-B*15:01 | VTVDTYSHF | 0.64 | 50.20 | 0.25 | SB |
| 91 | ERV_312445 | HLA-B*15:01 | VTVDTYSHF | 0.64 | 50.20 | 0.25 | SB |
| 91 | ERV_0308616 | HLA-B*15:01 | VTVDTYSHF | 0.64 | 50.20 | 0.25 | SB |
| 92 | ERV_312447 | HLA-B*40:01 | WEASPSVHI | 0.66 | 41.70 | 0.12 | SB |
| 92 | ERV_312445 | HLA-B*40:01 | WEASPSVHI | 0.66 | 41.70 | 0.12 | SB |
| 93 | ERV_312447 | HLA-B*40:01 | WEILAKSSL | 0.78 | 10.30 | 0.01 | SB |
| 93 | ERV_312445 | HLA-B*40:01 | WEILAKSSL | 0.78 | 10.30 | 0.01 | SB |
| 94 | ERV_312447 | HLA-A*01:01 | WMDNPIEIY | 0.63 | 55.90 | 0.05 | SB |
| 94 | ERV_312445 | HLA-A*01:01 | WMDNPIEIY | 0.63 | 55.90 | 0.05 | SB |
| 95 | ERV_312447 | HLA-A*02:01 | WMGDRLMSL | 0.77 | 12.30 | 0.15 | SB |
| 95 | ERV_312447 | HLA-B*08:01 | WMGDRLMSL | 0.76 | 13.90 | 0.01 | SB |
| 95 | ERV_312445 | HLA-A*02:01 | WMGDRLMSL | 0.77 | 12.30 | 0.15 | SB |
| 95 | ERV_312445 | HLA-B*08:01 | WMGDRLMSL | 0.76 | 13.90 | 0.01 | SB |
| 96 | ERV_312447 | HLA-A*02:01 | WQIGLANFV | 0.69 | 28.50 | 0.39 | SB |
| 96 | ERV_312445 | HLA-A*02:01 | WQIGLANFV | 0.69 | 28.50 | 0.39 | SB |
| 96 | ERV_0308616 | HLA-A*02:01 | WQIGLANFV | 0.69 | 28.50 | 0.39 | SB |
| 97 | ERV_312447 | HLA-A*03:01 | WVNQWPLPK | 0.60 | 73.70 | 0.28 | SB |
| 97 | ERV_312445 | HLA-A*03:01 | WVNQWPLPK | 0.60 | 73.70 | 0.28 | SB |
| 98 | ERV_312447 | HLA-A*02:01 | YAMSNLFSI | 0.84 | 5.50 | 0.04 | SB |
| 98 | ERV_312447 | HLA-B*39:01 | YAMSNLFSI | 0.65 | 43.30 | 0.04 | SB |
| 98 | ERV_312447 | HLA-B*58:01 | YAMSNLFSI | 0.64 | 47.10 | 0.27 | SB |
| 98 | ERV_312445 | HLA-A*02:01 | YAMSNLFSI | 0.84 | 5.50 | 0.04 | SB |
| 98 | ERV_312445 | HLA-B*39:01 | YAMSNLFSI | 0.65 | 43.30 | 0.04 | SB |
| 98 | ERV_312445 | HLA-B*58:01 | YAMSNLFSI | 0.64 | 47.10 | 0.27 | SB |
| 99 | ERV_312447 | HLA-B*39:01 | YHMVSGMSL | 0.88 | 3.60 | 0.00 | SB |
| 99 | ERV_312445 | HLA-B*39:01 | YHMVSGMSL | 0.88 | 3.60 | 0.00 | SB |
| 100 | ERV_312447 | HLA-A*02:01 | YIDDILCAA | 0.70 | 25.80 | 0.35 | SB |
| 100 | ERV_312445 | HLA-A*02:01 | YIDDILCAA | 0.70 | 25.80 | 0.35 | SB |
| 101 | ERV_312447 | HLA-A*02:01 | YLDQIATLI | 0.79 | 10.20 | 0.12 | SB |
| 101 | ERV_312445 | HLA-A*02:01 | YLDQIATLI | 0.79 | 10.20 | 0.12 | SB |
| 102 | ERV_312447 | HLA-B*08:01 | YPKTKIFQF | 0.65 | 42.70 | 0.07 | SB |
| 102 | ERV_312445 | HLA-B*08:01 | YPKTKIFQF | 0.65 | 42.70 | 0.07 | SB |
| 102 | ERV_0308616 | HLA-B*08:01 | YPKTKIFQF | 0.65 | 42.70 | 0.07 | SB |
| 103 | ERV_312447 | HLA-B*07:02 | YPQPPTRRL | 0.73 | 19.20 | 0.07 | SB |
| 103 | ERV_312445 | HLA-B*07:02 | YPQPPTRRL | 0.73 | 19.20 | 0.07 | SB |
| 104 | ERV_0308616 | HLA-B*08:01 | YQLARRAEL | 0.66 | 41.50 | 0.07 | SB |
| 104 | ERV_0308616 | HLA-B*39:01 | YQLARRAEL | 0.62 | 60.60 | 0.07 | SB |
| 105 | ERV_312447 | HLA-B*39:01 | YQSAQRAEL | 0.67 | 37.30 | 0.03 | SB |
| 105 | ERV_312445 | HLA-B*39:01 | YQSAQRAEL | 0.67 | 37.30 | 0.03 | SB |
| 106 | ERV_312447 | HLA-B*39:01 | YQYWPPAEL | 0.78 | 11.20 | 0.01 | SB |
| 106 | ERV_312445 | HLA-B*39:01 | YQYWPPAEL | 0.77 | 11.20 | 0.01 | SB |
| 107 | ERV_312447 | HLA-B*15:01 | YVHVTVDTY | 0.62 | 60.50 | 0.32 | SB |
| 107 | ERV_312445 | HLA-B*15:01 | YVHVTVDTY | 0.62 | 60.50 | 0.32 | SB |
| 107 | ERV_0308616 | HLA-B*15:01 | YVHVTVDTY | 0.62 | 60.50 | 0.32 | SB |

SEQ ID NO = peptide sequence progressive number; ID = ERV identification number; HLA = HLA used for peptide prediction; PEPTIDE = peptide sequence; SCORE = The raw prediction score; AFF(nM) = affinity of the peptide to the HLA, expressed in nM; %RANK = Rank of the predicted binding score compared to a set of random natural peptides; BINDLEVEL = The peptide is identified as a strong binder if the %Rank is below 0.5%

### Affinity and Stability Assays of Peptides to HLA * 02: 01

It was possible to experimentally verify the binding and stability of the predicted epitopes for the HLA-A * 0201 allele. The cell line T2 positive for HLA A * 02 was used which, having a deficit in the intracellular processing of peptides, does not have epitopes on the cell surface associated with HLA. Consequently, this cell line can be used to confirm binding and binding stability of specific epitopes for such HLA. The experiments have shown that all the peptides predicted to be specific for HLA * 02: 01 bind the allele and are presented on the surface of T2 cells (FIG. 1). Furthermore, the dissociation kinetics of the peptide from HLA showed that all peptide-HLA complexes have a dissociation value of 50% between 6 and 8 hours (T6 and T8), which indicates a strong bond stability. This was observed for the predicted peptides specific for HLA-A * 02: 01 and not for those specific for other HLA-A (FIG. 2).

### IFN_{γ} ELiSpot Assays with PBMCs from HCC Patients Positive for HLA-A*02:01 and For HLA-A *24:02

PBMCs from HCC patients positive for HLA-A*02:01 and for HLA-A*24:02 were purified and reacted with peptides predicted to be specific for the two alleles. The result of the IFN_{γ} ELiSpot assay demonstrates that patients with HCC have a proportion of circulating T lymphocytes capable of recognizing only the HERVs peptides predicted for HLA*02:01 and HLA-A*24:02 (FIG. 3).

### Example 2. Bioinformatic Analysis to Identify HCC-Specific Overexpressed Proteins Identification of HCC-Related Proteins for TAA Identification

In order to identify proteins specifically overexpressed in HCC and not detected in normal liver cells, the Normal Tissue dataset available at the Human Protein Atlas (https://www.proteinatlas.org/about/download) was interrogated. More than 15.000 proteins were screened and 5446 of them were found not detected into hepatocytes and bile duct cells of normal liver. In order to evaluate if those proteins were expressed in liver tumor, the Pathology dataset was analyzed and a total of 3283 proteins were found to be expressed in the liver tumor samples, at different levels. In particular, only a discrete number of such proteins shows high expression in more than one sample of the Protein Atlas (310, 9.45%) while the vast majority of them shows high expression in one or no sample at all (2973, 90.55%) (FIG. 4). To verify the tumor-specificity of such proteins, Normal Tissue dataset was re-examined to exclude the expression of these proteins in all other normal cells. The analysis identified 40 proteins with different expression levels in HCC that were absent in all normal tissues (Table 4).

TABLE 4

Expression levels of each protein in liver cancer as defined in the Human Protein Atlas (https://www.proteinatlas.org/humanproteome/pathology)

| Protein name | UNIPROT ID | LIVER CANCER EXPRESSION LEVELS | | | | Normal Tissues expression levels* |
|---|---|---|---|---|---|---|
| | | High | Medium | Low | Not detected | |
| ISG15 | P05161 | 9 | 2 | 0 | 0 | NOT DET |
| KLC1 | Q07866 | 8 | 3 | 0 | 0 | NOT DET |
| CAPN7 | Q9Y6W3 | 7 | 3 | 0 | 2 | NOT DET |
| SERPINF2 | P08697 | 6 | 4 | 1 | 1 | NOT DET |
| ORM1 | P02763 | 5 | 5 | 1 | 1 | NOT DET |
| ORM2 | P19652 | 5 | 5 | 1 | 1 | NOT DET |
| SEMA3A | Q14563 | 5 | 7 | 0 | 0 | NOT DET |
| FGF17 | O60258 | 5 | 4 | 0 | 3 | NOT DET |
| IL1R2 | P27930 | 5 | 7 | 0 | 0 | NOT DET |
| MASP2 | 000187 | 5 | 4 | 3 | 0 | NOT DET |
| ANGPT2 | 015123 | 4 | 2 | 2 | 3 | NOT DET |
| CLEC3B | P05452 | 4 | 1 | 2 | 2 | NOT DET |
| ADAMTSL2 | Q86TH1 | 3 | 3 | 2 | 4 | NOT DET |
| AFP | P02771 | 3 | 5 | 3 | 0 | NOT DET |
| APOC2 | P02655 | 3 | 5 | 0 | 4 | NOT DET |
| C4BPA | P04003 | 3 | 3 | 0 | 5 | NOT DET |
| CFB | P00751 | 3 | 7 | 1 | 0 | NOT DET |
| SERPINA7 | P05543 | 3 | 7 | 1 | 0 | NOT DET |
| FAM186B | Q8IYM0 | 3 | 3 | 2 | 4 | NOT DET |
| BMP6 | P22004 | 2 | 8 | 0 | 0 | NOT DET |
| C7 | P10643 | 2 | 9 | 1 | 0 | NOT DET |
| CRLF1 | O75462 | 2 | 2 | 6 | 2 | NOT DET |
| F2 | P00734 | 2 | 6 | 2 | 1 | NOT DET |
| LRG1 | P02750 | 2 | 6 | 1 | 1 | NOT DET |
| C1QTNF12 | Q5T7M4 | 2 | 2 | 2 | 6 | NOT DET |
| MDK | P21741 | 2 | 1 | 3 | 5 | NOT DET |
| APOC1 | P02654 | 1 | 4 | 2 | 5 | NOT DET |
| CHRD | Q9H2X0 | 1 | 9 | 1 | 1 | NOT DET |
| CP | P00450 | 1 | 5 | 6 | 0 | NOT DET |
| DYRK4 | Q9NR20 | 1 | 11 | 0 | 0 | NOT DET |
| ITIH4 | Q14624 | 1 | 4 | 6 | 1 | NOT DET |
| PLTP | P55058 | 1 | 10 | 1 | 0 | NOT DET |
| PON1 | P27169 | 1 | 5 | 3 | 0 | NOT DET |
| TNFRSF1B | P20333 | 1 | 8 | 2 | 0 | NOT DET |
| ADAMTS15 | Q8TE58 | 1 | 7 | 2 | 2 | NOT DET |
| KLK14 | Q9P0G3 | 1 | 9 | 2 | 0 | NOT DET |
| PEBP4 | Q96S96 | 1 | 4 | 4 | 3 | NOT DET |
| PI15 | O43692 | 1 | 2 | 3 | 6 | NOT DET |
| RGPD8 | 014715 | 1 | 4 | 2 | 4 | NOT DET |
| RSPO2 | Q6UXX9 | 1 | 5 | 4 | 2 | NOT DET |

*Normal tissue data from The Human Protein Atlas https://www.proteinatlas.org/about/download

### Survival Analysis

A survival analysis was performed for each of the selected 40 proteins in order to identify those which are associated with a poor prognosis in HCC patients at a statistical significance. A total of 9 proteins were identified with such a characteristic (Table 5). The average 5-year survival in HCC patients with high expression of all 9 proteins was 34.33% compared to 53.88% in HCC patients with low expression (FIG. 5A). In particular, considering the high expression levels, the protein associated with the worst 5-year survival was DYRK4 (21%) and the one associated with the best survival was CAPN7 (41%) (FIGS. 5B and C).

TABLE 5

| PROTEIN | Best expression cut off-p score | PROGNOSIS |
|---|---|---|
| ISG15 | 0.041 | unfavorable |
| KLC1 | 0.0012 | unfavorable |
| CAPN7 | 0.0022 | unfavorable |
| SEMA3A | 0.0029 | unfavorable |
| BMP6 | 0.0016 | unfavorable |
| C1QTNF12 (FAM132A) | 0.0027 | unfavorable |
| MDK | 0.0057 | unfavorable |
| DYRK4 | 0.020 | unfavorable |
| PLTP | 0.017 | unfavorable |

https://www.proteinatlas.org/

### Gene Expression Analysis

The gene expression of the selected 9 proteins was assessed by RNASeq analysis performed on 24 paired HCC samples and matched non tumoral liver tissues. The results showed a trend of a higher expression level in cancer tissues for ISG15, CAPN7, SEMA3A and DYRK4 genes. The MDK gene showed the most relevant increased expression in cancer tissues with a p value < 0.0001. On the contrary, KLC1, BMP6, C1QTNF12 and PLTP genes showed higher expression levels in the normal tissues. In order to confirm such results on a much larger dataset, we took advantage of publicly available data (http://gent2.appex.kr/gent2/). Gene expression levels from 691 liver cancer tissues were compare to 297 normal tissues. The analysis confirmed the data generated on the 24 HCC samples of our cohort and were supported by high statistical p value, except for SEMA3A and C1QTNF12 genes (FIGS. 6A and B). The gene expression levels did not show a significant correlation with the protein expression reported at the Protein Atlas, although the ISG15 gene was the one to show the highest expression at both gene and protein.

### Peptide Prediction

In order to identify potential tumor associated antigens (TAAs) derived from the 9 overexpressed proteins associated with poor prognosis in HCC, the NetMHCpan software was used to predict 9 aa-long epitopes (nonamers) associated with the HLA-A*02:01 and 24:02 alleles. The number of nonamers varied for each protein, according to the length of sequence, deriving from overlapping peptides along the protein with a shift of one amino acid. Only a discrete number of strong (SB) and weak (WB) binders were predicted for each protein associated with the two alleles (as determined with the algorithm NetMHCPan http://www.cbs.dtu.dk/services/NetMHCpan/). Overall, considering both alleles, the MDK protein showed the lowest (3 SB and 2 WB) and the CAPN7 protein showed the highest (25 SB and 47 WB) cumulative number of binders (Table 6). Among the SB, only those with a predicted affinity lower than 100 nM were selected and further subdivided in three groups characterized by affinity <10 nM, 10< affinity <50 nM, affinity >50 nM (FIG. 7). Indeed, only those with a predicted affinity <50 nM have been previously shown to have a 100% concordance with ex vivo binding assay (Petrizzo et al., JTM 2018, Unique true predicted neoantigens (TPNAs) correlates with anti-tumor immune control in HCC patients. Petrizzo A, Tagliamonte M, Mauriello A, Costa V, Aprile M, Esposito R, Caporale A, Luciano A, Arra C, Tornesello ML, Buonaguro FM, Buonaguro L. J Transl Med. 2018 Oct 19;16(1):286. doi: 10.1186/s12967-018-1662-9. PMID: 30340600). According to such a classification, 12 peptides with affinity <10 nM and 24 peptides with 10< affinity <50 nM were identified in both HLA-A alleles (FIG. 7). The NetMHCstabpan software was used to implement the prediction analysis with information on the binding stability of the peptides to the HLA molecule, expressed as half life time in hours (Thalf) (Table 7). According to this combined analysis, a handful number of predicted epitopes were classified as SB, with a Thalf ranging from 1.96 hrs (the HLA-A*24:02 linked peptide VHMKDFFYF from the DYRK4 protein) to 61.9 hrs (the HLA-A*24:02 linked peptide VYSACSFTF from the CAPN7 protein) (Table 7).

TABLE 6

For each protein are indicated the number of strong binders (SB) and weak binders (WB) predicted to bind each of the two HLA.

| PROTEIN | HLA-A | SB | WB |
|---|---|---|---|
| ISG15 | HLA-A*02:01 | 5 | 8 |
| | HLA-A*24:02 | 0 | 3 |
| MDK | HLA-A*02:01 | 3 | 1 |
| | HLA-A*24:02 | 0 | 1 |
| KLC1 | HLA-A*02:01 | 9 | 9 |
| | HLA-A*24:02 | 4 | 6 |
| CAPN7 | HLA-A*02:01 | 10 | 20 |
| | HLA-A*24:02 | 15 | 27 |
| SEMA3A | HLA-A*02:01 | 5 | 7 |
| | HLA-A*24:02 | 8 | 33 |
| BMP6 | HLA-A*02:01 | 5 | 13 |
| | HLA-A*24:02 | 3 | 11 |
| C1QTNF12 | HLA-A*02:01 | 5 | 10 |
| | HLA-A*24:02 | 2 | 5 |
| DYRK4 | HLA-A*02:01 | 2 | 11 |
| | HLA-A*24:02 | 7 | 18 |
| PLTP | HLA-A*02:01 | 7 | 22 |
| | HLA-A*24:02 | 9 | 16 |

TABLE 7

For each protein are indicated the aminoacide sequence of peptide predicted to bind each of the two HLA. AFF = affinity of binding to HLA expressed in nM; Thalf = stability of binding expressed in hours; Binder = strong binder (SB); weak binder (WB) according to the NetMHCstabpan predictive tool.

| HLA-A*02:01 | | | | | HLA-A*24:02 | | | | |
|---|---|---|---|---|---|---|---|---|---|
| PROTEIN | SEQ | AFF | Thalf | Binder | PROTEIN | SEQ | AFF | Thalf | Binder |
| ISG15 | MLAGNEFQV | 4.35 | 5.22 | SB | | | | | |
| | GVQDDLFWL | 69.76 | 0.7 | WB | | | | | |
| | ALQDRVPLA | 70.13 | 3.09 | WB | | | | | |
| | RLAVHPSGV | 82.29 | 4.73 | WB | | | | | |
| | RLTQTVAHL | 90.38 | 6.75 | WB | | | | | |
| MDK | ALLALTSAV | 10.06 | 9.85 | SB | | | | | |
| | LLLTLLALL | 11.34 | 4.69 | WB | | | | | |
| | FLLLTLLAL | 17.89 | 5.1 | WB | | | | | |
| KLC1 | TMLNILALV | 5.14 | 17.22 | SB | KLC1 | YYYQRALEI | 12.69 | 4.06 | SB |
| | ALSNHLNAV | 10.73 | 18.84 | SB | | | | | |
| | VMMALSNHL | 11.55 | 3.64 | WB | | | | | |
| | ILQSLLETL | 31.65 | 4.15 | WB | | | | | |
| | KLGPDDPNV | 46.49 | 1.97 | WB | | | | | |
| | SLLETLKCL | 53.81 | 3.24 | WB | | | | | |
| | KQLNNLALL | 62.31 | 1.95 | WB | | | | | |
| | GLSEAQVMM | 93.94 | 1.08 | WB | | | | | |
| CAPN7 | RQYSVGFEV | 18.12 | 3.19 | SB | CAPN7 | VYSACSFTF | 3.83 | 61.9 | SB |
| | VIIDDQLPV | 20.16 | 4.74 | WB | | IYTVSSFSI | 14.92 | 5.44 | SB |
| | KITEYLERV | 26.12 | 15.36 | SB | | IFNIIPSTF | 32.18 | 3.11 | SB |
| | KIDNGIFWI | 28.17 | 1.75 | WB | | YYDVIYLSW | 53.61 | 5.88 | SB |
| | KQNTIHYTV | 84.06 | 3.03 | WB | | | | | |
| SEMA3A | AMYNPVFPM | 8.47 | 2.41 | WB | SEMA3A | VWYRDFMQL | 63.51 | 0.6 | WB |
| | TLLKVTLEV | 10.07 | 5.76 | SB | | LYSGTAADF | 84.34 | 2.2 | SB |
| | KLLTASLLI | 13.26 | 3 | WB | | LYACGTGAF | 91.7 | 2.72 | SB |
| | TMDEFCEQV | 23.43 | 6.96 | WB | | | | | |
| | RIVCLFWGV | 15.92 | 4.94 | SB | | | | | |
| BMP6 | FMLDLYNAL | 3.7 | 3.17 | SB | BMP6 | KQPFMVAFF | 43.81 | 0.57 | WB |
| | MVMSFVNLV | 4.72 | 11.07 | SB | | | | | |
| | FLNDADMVM | 10.28 | 2.04 | WB | | | | | |
| | FLISIYQVL | 11.1 | 4.82 | SB | | | | | |
| C1QTNF12 | FQFSASLHV | 7.76 | 5.7 | SB | | | | | |
| | LLHEFQELL | 16.18 | 4.18 | WB | | | | | |
| | LLGPQLVLL | 37.45 | 2.61 | WB | | | | | |
| | TLLHEFQEL | 54.09 | 1.44 | n/a | | | | | |
| DYRK4 | VILGHPYDV | 62.7 | 1.88 | WB | DYRK4 | FYFRNHFCI | 12.53 | 22.36 | SB |
| | SLGCITAEL | 31.08 | 1.72 | WB | | VYTYIQSRF | 22.25 | 13.91 | SB |
| | | | | | | VHMKDFFYF | 34.95 | 1.96 | SB |
| PLTP | ALFGALFLA | 4.79 | 7.31 | SB | PLTP | VYVAFSEFF | 7.21 | 14.55 | SB |
| | LLNSLLDTV | 9.24 | 13.38 | SB | | TYFGSIVLL | 60.74 | 2.09 | SB |
| | ALIPLQAPL | 21.34 | 4.21 | WB | | LYHAGTVLL | 68.06 | 2.92 | SB |
| | FLEQELETI | 99.88 | 3.04 | n/a | | TFITSGMRF | 99.93 | 1.31 | WB |
| | LLNQQICPV | 10.28 | 34.15 | SB | | | | | |
| | YINASAEGV | 92.99 | 2.15 | WB | | | | | |

### Peptide Similarity to Pathogen Epitopes

Predicted strong binder epitopes were screened for homology to human viral sequences in BLAST. Several peptides from a broad range of human viruses were found to share high homology with those derived from the HCC proteins. However, only four of them were predicted to be strong binders to HLA-A*0201 with a Thalf >3 hrs (Table 8). A predicted epitope derived from the Human Calicivirus showed 6/9 identical residues with an epitope from the ISG15. A predicted epitope derived from the HCV and one derived from the Human Adenovirus 4 showed 7/9 and 8/9 identical residues with two epitopes from the MDK. A predicted epitope derived from the HBV showed 8/9 identical residues with an epitope from the C1QTNF12. Strikingly, the epitope derived from the HCV shows affinity and stability to the HLA-A*02:01 significantly higher than the corresponding TAA (3.11 vs. 10.06 nM; 45.58 vs. 9.85 hours) (Table 8). Likewise, only three viral peptides were predicted to bind the HLA-A*2402 molecule but with low affinity (>70 nM) and/or low stability (<1.5 hrs) (Table 8).

TABLE 8

| HLA | PROTEIN | SEQUENCE | VIRAL PROTEIN | AFF | Thalf | BINDING |
|---|---|---|---|---|---|---|
| A*02:01 | ISG15 | MLAGNEFQV | | 4.35 | 5.22 | SB |
| | | MLAGNAFTA | capsid protein Human calicivirus | 13.86 | 3.53 | WB |
| | MDK | ALLALTSAV | | 10.06 | 9.85 | SB |
| | | ALMAFTSAV | polyprotein HCV seq ID: AID60264.1 | 3.11 | 45.58 | SB |
| | MDK | LLLTLLALL | | 11.34 | 4.69 | WB |
| | | LLLTLLLLL | E3 14.5 kDa protein [Human adenovirus E4] Seq ID: AGT51280.1 | 17.75 | 4.41 | WB |
| | C1QTNF12 | LLGPQLVLL | | 37.45 | 2.61 | WB |
| | | LLGPLLVLL | S protein HBV Seq ID: AUF41974.1 | 20.91 | 3.38 | WB |
| A*24:02 | CAPN7 | VYSACSFTF | | 3.83 | 61.9 | SB |
| | | GSPACTFTF | protein UL29 CMV Seq ID: | 658.27 | 0.65 | WB |
| | CAPN7 | IYTVSSFSI | | 14.92 | 5.44 | SB |
| | | SYTVSSFQV | nonstructural protein 1 [Influenza A virus] Seq ID: QEM33605.1 | 352.63 | 1.06 | WB |
| | DYRK4 | FYFRNHFCI | | 12.53 | 22.36 | SB |
| | | DYFRNQFKI | polymerase basic protein 2 Influenza A virus Seq ID: AAV33795.1 | 77.75 | 1.39 | WB |

### Epitope Modeling and Molecular Docking

In order to verify that predicted paired TAA and viral epitopes share similar contact residues with both the HLA molecule and the TCR, epitope modeling and molecular docking were performed for each paired peptides. This was possible only for HLA-A*0201 restricted epitopes, due to the lack of crystallized structures including both HLA and TCR for other alleles deposited in the PDB. The 1AO7 crystallized complex including the HTLV-I TAX epitope was used as general template to conduct the analyses. As example, the ISG15 MLAGNEFQV and the human calicivirus MLAGNAFTA peptides show a similar interacting pattern between the residues of both peptides and the HLA as well as the β chain of the TCR. Differences are represented by the substitution of non-polar A with a negative-charged E in position 6 which substantially changes the contact pattern with the α chain of the TCR. Furthermore, the two additional conservative polar (T8Q) and non polar (A9V) substitutions do not change the contact pattern with the TCR (FIG. 8). This would suggest that the TCR clones targeting the two peptides would share only the same β chain.

The present invention relates to a mix comprising at least one peptide from the groups of peptides identified above, namely the HERV-TSAs and the HCC-TAAs and peptides from human viruses with high homology with those derived from the HCC proteins.

In particular, preferred peptides from the HERV-TSAs (Table 3) include:

1) FINSGAWQI (SEQ ID. NO 17)

2) KLTTWILPK (SEQ ID NO 43)

3) KTKIFQFLK (SEQ ID. NO 50)

4) PYMLVVGNI (SEQ ID. NO 65)

5) SMDDQLNQL (SEQ ID. NO 83)

6) WQIGLANFV (SEQ ID. NO 96).

Preferred peptides from the HCC-TAAs (Table 7) include:

1) Seq ISG15 MLAGNEFQV (SEQ ID. NO 108)

2) Seq KLC1 TMLNILALV (SEQ ID. NO 109)

3) Seq KLC1 YYYQRALEI (SEQ ID. NO 110)

4) Seq CAPN7 KITEYLERV (SEQ ID. NO 111)

5) Seq CAPN7 VYSACSFTF (SEQ ID. NO 112)

6) Seq SEMA3A TLLKVTLEV (SEQ ID. NO 113)

7) Seq SEMA3A LYSGTAADF (SEQ ID. NO 114)

8) Seq BMP6 MVMSFVNLV (SEQ ID. NO 115)

9) Seq DYRK4 FYFRNHFCI (SEQ ID. NO 116)

10) Seq PLTP LLNQQICPV (SEQ ID. NO 117)

11) Seq PLTP VYVAFSEFF (SEQ ID. NO 118)

Preferred peptides from the human viruses with high homology with those derived from the HCC proteins (Table 8) include:

12) Seq ISG15 MLAGNAFTA (SEQ ID. NO 119)

13) Seq MDK ALMAFTSAV (SEQ ID. NO 120)

14) Seq DYRK4 DYFRNQFKI (SEQ ID. NO 121)

Further preferred peptides from the HCC-TAAs (Table 7) include:

15) Seq MDK ALLALTSAV (SEQ ID. NO 122)

16) Seq KLC1 ALSNHLNAV (SEQ ID. NO 123)

17) Seq CAPN7 RQYSVGFEV (SEQ ID. NO 124)

18) Seq CAPN7 IYTVSSFSI (SEQ ID. NO 125)

19) Seq CAPN7 IFNIIPSTF (SEQ ID. NO 126)

20) Seq CAPN7 YYDVIYLSW (SEQ ID. NO 127)

21) Seq SEMA3A RIVCLFWGV (SEQ ID. NO 128)

22) Seq SEMA3A LYACGTGAF (SEQ ID. NO 129)

23) Seq BMP6 FMLDLYNAL (SEQ ID. NO 130)

24) Seq BMP6 FLISIYQVL (SEQ ID. NO 131)

25) Seq C1QTNF12 FQFSASLHV (SEQ ID. NO 132)

26) Seq DYRK4 VYTYIQSRF (SEQ ID. NO 133)

27) Seq DYRK4 VHMKDFFYF (SEQ ID. NO 134)

28) Seq PLTP ALFGALFLA (SEQ ID. NO 135)

29) Seq PLTP LLNSLLDTV (SEQ ID. NO 136)

30) Seq PLTP TYFGSIVLL (SEQ ID. NO 137)

31) Seq PLTP LYHAGTVLL (SEQ ID. NO 138)

Further preferred peptides from the human viruses with high homology with those derived from the HCC proteins (Table 8) include:

32) Seq MDK LLLTLLLLL (SEQ ID. NO 139)

33) Seq MDK LLGPLLVLL (SEQ ID. NO 140)

### REFERENCES

1. Hollingsworth RE, Jansen K: Turning the corner on therapeutic cancer vaccines. NPJ Vaccines 2019, 4: 7.

2. Chianese-Bullock KA, Woodson EM, Tao H, Boerner SA, Smolkin M, Grosh WW et al.: Autoimmune toxicities associated with the administration of antitumor vaccines and low-dose interleukin-2. J Immunother 2005, 28: 412-419.

3. Sanderson K, Scotland R, Lee P, Liu D, Groshen S, Snively J et al.: Autoimmunity in a phase I trial of a fully human anti-cytotoxic T-lymphocyte antigen-4 monoclonal antibody with multiple melanoma peptides and Montanide ISA 51 for patients with resected stages III and IV melanoma. J Clin Oncol 2005, 23: 741-750.

4. Coulie PG, Van den Eynde BJ, van der BP, Boon T: Tumour antigens recognized by T lymphocytes: at the core of cancer immunotherapy. Nat Rev Cancer 2014, 14: 135-146.

5. Fikes JD, Sette A: Design of multi-epitope, analogue-based cancer vaccines. Expert Opin Biol Ther 2003, 3: 985-993.

6. Schumacher TN, Scheper W, Kvistborg P: Cancer Neoantigens. Annu Rev Immunol 2019, 37: 173-200.

7. Stronen E, Toebes M, Kelderman S, van Buuren MM, Yang W, van RN et al.: Targeting of cancer neoantigens with donor-derived T cell receptor repertoires. Science 2016, 352: 1337-1341.

8. Ott PA, Hu Z, Keskin DB, Shukla SA, Sun J, Bozym DJ et al.: An immunogenic personal neoantigen vaccine for patients with melanoma. Nature 2017, 547: 217-221.

9. Sahin U, Derhovanessian E, Miller M, Kloke BP, Simon P, Lower M et al.: Personalized RNA mutanome vaccines mobilize poly-specific therapeutic immunity against cancer. Nature 2017, 547: 222-226.

10. Jones PA, Ohtani H, Chakravarthy A, De Carvalho DD: Epigenetic therapy in immune-oncology. NatRev Cancer 2019, 19: 151-161.

11. Attermann AS, Bjerregaard AM, Saini SK, Gronbaek K, Hadrup SR: Human endogenous retroviruses and their implication for immunotherapeutics of cancer. Ann Oncol 2018, 29: 2183-2191.

12. Johnson WE: Origins and evolutionary consequences of ancient endogenous retroviruses. Nat Rev Microbiol 2019, 17: 355-370.

13. Mayer J, Blomberg J, Seal RL: A revised nomenclature for transcribed human endogenous retroviral loci. Mob DNA 2011, 2: 7.

14. Kassiotis G, Stoye JP: Making a virtue of necessity: the pleiotropic role of human endogenous retroviruses in cancer. Philos Trans R Soc Lond B Biol Sci 2017, 372.

15. Kassiotis G, Stoye JP: Making a virtue of necessity: the pleiotropic role of human endogenous retroviruses in cancer. Philos Trans R Soc Lond B Biol Sci 2017, 372.

16. Rebollo R, Romanish MT, Mager DL: Transposable elements: an abundant and natural source of regulatory sequences for host genes. Annu Rev Genet 2012, 46: 21-42.

17. Boese A, Sauter M, Galli U, Best B, Herbst H, Mayer J et al.: Human endogenous retrovirus protein cORF supports cell transformation and associates with the promyelocytic leukemia zinc finger protein. Oncogene 2000, 19: 4328-4336.

18. Kudo-Saito C, Yura M, Yamamoto R, Kawakami Y: Induction of immunoregulatory CD271+ cells by metastatic tumor cells that express human endogenous retrovirus H. Cancer Res 2014, 74: 1361-1370.

19. Grabski DF, Hu Y, Sharma M, Rasmussen SK: Close to the Bedside: A Systematic Review of Endogenous Retroviruses and Their Impact in Oncology. J Surg Res 2019, 240: 145-155.

20. Ma W, Hong Z, Liu H, Chen X, Ding L, Liu Z et al.: Human Endogenous Retroviruses-K (HML-2) Expression Is Correlated with Prognosis and Progress of Hepatocellular Carcinoma. Biomed Res Int 2016, 2016: 8201642.

21. Balogh J, Victor D, III, Asham EH, Burroughs SG, Boktour M, Saharia A et al.: Hepatocellular carcinoma: a review. J Hepatocell Carcinoma 2016, 3: 41-53.

22. Fong ZV, Tanabe KK: The clinical management of hepatocellular carcinoma in the United States, Europe, and Asia: a comprehensive and evidence-based comparison and review. Cancer 2014, 120: 2824-2838.

23. European Association for the Study of the Liver: EASL Clinical Practice Guidelines: Management of hepatocellular carcinoma. J Hepatol 2018, 69: 182-236.

24. Fong ZV, Tanabe KK: The clinical management of hepatocellular carcinoma in the United States, Europe, and Asia: a comprehensive and evidence-based comparison and review. Cancer 2014, 120: 2824-2838.

25. Lencioni R, Crocetti L: Local-regional treatment of hepatocellular carcinoma. Radiology 2012, 262: 43-58.

26. Villanueva A, Hernandez-Gea V, Llovet JM: Medical therapies for hepatocellular carcinoma: a critical view of the evidence. Nat Rev Gastroenterol Hepatol 2013, 10: 34-42.

27. Chan SL, Mok T, Ma BB: Management of hepatocellular carcinoma: beyond sorafenib. Curr Oncol Rep 2012, 14: 257-266. Kudo Met al., Lancet. 2018 Mar 24;391(10126):1163-1173. doi: 10.1016/S0140-6736(18)30207-1: Lenvatinib versus sorafenib in first-line treatment of patients with unresectable hepatocellular carcinoma: a randomised phase 3 non-inferiority trial.

28. Bruix J, Qin S, Merle P, Granito A, Huang YH, Bodoky G et al.: Regorafenib for patients with hepatocellular carcinoma who progressed on sorafenib treatment (RESORCE): a randomised, double-blind, placebo-controlled, phase 3 trial. Lancet 2017, 389: 56-66.

29. Buonaguro L, Mauriello A, Cavalluzzo B, Petrizzo A, Tagliamonte M: Immunotherapy in hepatocellular carcinoma. Ann Hepatol 2019, 18: 291-297.

30. Kim D, Pertea G, Trapnell C, Pimentel H, Kelley R, Salzberg SL: TopHat2: accurate alignment of transcriptomes in the presence of insertions, deletions and gene fusions. Genome Biol 2013, 14: R36.

31. Russo F, Angelini C: RNASeqGUI: a GUI for analysing RNA-Seq data. Bioinformatics 2014, 30: 2514-2516.

1. A composition comprising at least a peptide or an expression vector that induces the expression of said at least one peptide, said peptide consisting of an amino acid (aa) sequence from 9 to 30 aa residues and comprising an epitope sequence selected from the group consisting of: MLAGNAFTA (SEQ ID NO. 119), ALMAFTSAV (SEQ ID NO. 120), DYFRNQFKI (SEQ ID NO. 121), MLAGNEFQV (SEQ ID NO. 108), TMLNILALV (SEQ ID NO. 109), YYYQRALEI (SEQ ID NO. 110), KITEYLERV (SEQ ID NO. 111), VYSACSFTF (SEQ ID NO. 112), TLLKVTLEV (SEQ ID NO. 113), LYSGTAADF (SEQ ID NO. 114), MVMSFVNLV (SEQ ID NO. 115), FYFRNHFCI (SEQ ID NO. 116), LLNQQICPV (SEQ ID NO. 117), and VYVAFSEFF (SEQ ID NO. 118), and a pharmaceutically acceptable vehicle or excipient.

2. The composition according to claim 1 wherein said epitope sequence is selected from the group consisting of: MLAGNAFTA (SEQ ID NO. 119), ALMAFTSAV (SEQ ID NO. 120), and DYFRNQFKI (SEQ ID NO. 121).

3. The composition according to claim 1 further comprising or expressing a peptide consisting of an aa sequence of 9 to 30 aa and comprising an epitope sequence selected from the group consisting of: ALLALTSAV (SEQ ID NO. 122), ALSNHLNAV (SEQ ID NO. 123), RQYSVGFEV (SEQ ID NO. 124), IYTVSSFSI (SEQ ID NO. 125), IFNIIPSTF (SEQ ID NO. 126), YYDVIYLSW (SEQ ID NO. 127), RIVCLFWGV (SEQ ID NO. 128), LYACGTGAF (SEQ ID NO. 129), FMLDLYNAL (SEQ ID NO. 130), FLISIYQVL (SEQ ID NO. 131), FQFSASLHV (SEQ ID NO. 132), VYTYIQSRF (SEQ ID NO. 133), VHMKDFFYF (SEQ ID NO. 134), ALFGALFLA (SEQ ID NO. 135), LLNSLLDTV (SEQ ID NO. 136), TYFGSIVLL (SEQ ID NO. 137), LYHAGTVLL (SEQ ID NO. 138), LLLTLLLLL (SEQ ID NO. 139), and LLGPLLVLL(SEQ ID NO. 140).

4. The composition according to claim 1 further comprising or expressing a peptide consisting of an aa sequence of 9 to 30 aa and comprising an epitope sequence selected from the group consisting of: FINSGAWQI (SEQ ID NO. 17), KLTTWILPK (SEQ ID NO. 43), KTKIFQFLK (SEQ ID NO. 50), PYMLVVGNI (SEQ ID NO. 65), SMDDQLNQL (SEQ ID NO. 83), WQIGLANFV (SEQ ID NO. 96).

5. The composition according to claim 1 further comprising or expressing a peptide of 9 to 30 amino acids comprising an epitope sequence selected from the group consisting of SEQ ID NO. 4-16, SEQ ID NO. 18-42, SEQ ID NO. 44-49, SEQ ID NO. 51-64, SEQ ID NO. 66-82, SEQ ID NO. 84-95 and SEQ ID NO. 97-107.

6. The composition according to claim 4 comprising:
a. an epitope sequence selected from the group consisting of: MLAGNAFTA (SEQ ID NO. 119), ALMAFTSAV (SEQ ID NO. 120), DYFRNQFKI (SEQ ID NO. 121); and
b. an epitope sequence selected from the group consisting of: MLAGNEFQV (SEQ ID NO. 108), TMLNILALV (SEQ ID NO. 109), YYYQRALEI (SEQ ID NO. 110), KITEYLERV (SEQ ID NO. 111), VYSACSFTF (SEQ ID NO. 112), TLLKVTLEV (SEQ ID NO. 113), LYSGTAADF (SEQ ID NO. 114), MVMSFVNLV (SEQ ID NO. 115), FYFRNHFCI (SEQ ID NO. 116), LLNQQICPV (SEQ ID NO. 117), VYVAFSEFF (SEQ ID NO. 118); and
c. an epitope sequence selected from the group consisting of: FINSGAWQI (SEQ ID NO. 17), KLTTWILPK (SEQ ID N0.43), KTKIFQFLK (SEQ ID NO. 50), PYMLVVGNI (SEQ ID NO. 65), SMDDQLNQL (SEQ ID NO. 83), WQIGLANFV (SEQ ID NO. 96).

7. The composition according to claim 1 wherein thepeptide consists of an amino acid (aa) sequence from 9 to 20 aa residues.

8. The composition according to claim 1 wherein said peptide is bound or inserted into an antibody which is specific for dendritic cells.

9. An engineered cell comprising a recombinant protein, or a polynucleotide encoding a recombinant protein, a receptor, wherein the recombinant protein specifically binds to a least peptide of the composition.

10. A method for the treatment and/or prevention of cancer, comprising administering a composition of claim 1 to a patient in need of such treatment and/or prevention.

11. The method according to claim 10, further comprising administering in combination a further anticancer therapeutic agent.

12. A vaccine or immunogenic composition comprising the composition of claim 1 and a pharmaceutically acceptable vehicle or excipient.

13. A method for the treatment and/or prevention of cancer, the vaccine or immunogenic composition of claim 12 to a patient in need of such treatment and/or prevention.

14. An isolated peptide selected from the group consisting of the peptides having aa sequences selected from SEQ ID NO. 4-140 or of the peptides having an aa sequence of 9 to 30 aa and comprising at least one of SEQ ID NO 108-140, wherein said isolated peptide is able to bind MHC class I and/or MHC class II.

15. The peptide according to claim 14 wherein said peptide is bound or inserted into an antibody which is specific for dendritic cells.

16. The method of claim 10, wherein the cancer is liver cancer.

17. The method of claim 16, wherein the liver cancer is hepatocellular carcinoma.

18. A method for the treatment and/or prevention of cancer comprising administering the engineered cell of claim 9 to a patient in need of such treatment and/or prevention.

19. The method of claim 18, wherein the cancer is liver cancer.

20. The method of claim 19, wherein the liver cancer is hepatocellular carcinoma.


SEQUENCE LISTING (141 sequences)

SEQ ID NO: 1 - protein, 1375 aa, Homo sapiens
MLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHYIDDILCAAETRDKLIDCYTFLQAEVANAGLAIASDKIQTSTPFHYLGMQIENRKIKQQKIEIRKDTLKTLNDFQKLLGDINWIRPTLGIPTYAMSNLFSILRGDSDLNSKRILTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVPLTKEQVRQAFINSGAWQIGLANFVGIIDNHYPKTKIFQFLKLTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSALIKAQELHALTHVNAAGLKNKFDVTWKQAKDIVQHCTQCQILHLPTQEAGVNPRGLCPNALWQMDVTHVPSFGRLSYVHVTVDTYSHFIWATCQTGESTSHVKKHLLSCFAVMGVPEKIKTDNGPGYCSKAFQKFLSQWKISHTTGIPYNSQGQAIVERTNRTLKTQLVKQKEGGDSKECTTPQMQLNLALYTLNFLNIYRNQTTTSAEQHLTGKKNSPHEGKLIWWKDNKNKTWEIGKVITWGRGFACVSPGENQLPVWIPTRHLKFYNEPIGDAKKRASTEMVTPVTWMDNPIEIYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDCKPFYTIDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 2 - protein, 222 aa, Homo Sapiens
MDDQLNQLFNLLQQTVRKRNFPFYITHIRAHTNLPGPLTKANEPADLLVSSAFIKAQKFHALTHVNAAGLKNKFDVTWKQAKDIVRHCTQCQVLHLPTQEAGVNPRGLCPNALRQMDVTHSKAFQKFLSQWKISHTTGIPYNSQGQAIVERTNRTLKTQLVKVPSFGRLLYVHVTVDTYSHFIWATCQTGESTSHVKKHLLPCFAVMRVPEKIKTDNGAGYC

SEQ ID NO: 3 - protein, 222 aa, Homo Sapiens
MDDQLNQLFNLLQQTVRKRNFPFYITHIRAHTNLPGPLTKANEQADLLVSSALIKAQELHALTHVNAAGLKNKFDVTWKQAKDIVQHCTQCQVLHLPTQEAGVNPRGLCPNALWQMDVTHSKAFQKFLSQWKISHTTGIPYNSQGQAIVERTNRTLKTQLVKVPSFGRLSYVHVTVDTYSHFIWATCQTGESTSHVKKHLLSCFAVMGVPEKIKTDNGPGYC

SEQ ID NO: 4 - protein, 9 aa, Homo Sapiens
AELVAVITV

SEQ ID NO: 5 - protein, 9 aa, Homo Sapiens
AMIPKDWPL

SEQ ID NO: 6 - protein, 9 aa, Homo Sapiens
AMSNLFSIL

SEQ ID NO: 7 - protein, 9 aa, Homo Sapiens
AQAITGVVL

SEQ ID NO: 8 - protein, 9 aa, Homo Sapiens
AQINRIDPL

SEQ ID NO: 9 - protein, 9 aa, Homo Sapiens
ARYKSFSIK

SEQ ID NO: 10 - protein, 9 aa, Homo Sapiens
ATRDVKTAL

SEQ ID NO: 11 - protein, 9 aa, Homo Sapiens
AVIMGLIAV

SEQ ID NO: 12 - protein, 9 aa, Homo Sapiens
AVNAVIQPM

SEQ ID NO: 13 - protein, 9 aa, Homo Sapiens
AVQNWLVEV

SEQ ID NO: 14 - protein, 9 aa, Homo Sapiens
CLMPAVQNW

SEQ ID NO: 15 - protein, 9 aa, Homo Sapiens
EARYKSFSI

SEQ ID NO: 16 - protein, 9 aa, Homo Sapiens
ERAMMTMAV

SEQ ID NO: 17 - protein, 9 aa, Homo Sapiens
FINSGAWQI

SEQ ID NO: 18 - protein, 9 aa, Homo Sapiens
FLKLTTWIL

SEQ ID NO: 19 - protein, 9 aa, Homo Sapiens
FLQAEVANA

SEQ ID NO: 20 - protein, 9 aa, Homo Sapiens
FLQFKTWWI

SEQ ID NO: 21 - protein, 9 aa, Homo Sapiens
FSDCYIIHY

SEQ ID NO: 22 - protein, 9 aa, Homo Sapiens
FSYQRSLKF

SEQ ID NO: 23 - protein, 9 aa, Homo Sapiens
FTYHMVSGM

SEQ ID NO: 24 - protein, 9 aa, Homo Sapiens
GENQLPVWI

SEQ ID NO: 25 - protein, 9 aa, Homo Sapiens
GENQLPVWI

SEQ ID NO: 26 - protein, 9 aa, Homo Sapiens
HLKKNCPVL

SEQ ID NO: 27 - protein, 9 aa, Homo Sapiens
HPELWRLTV

SEQ ID NO: 28 - protein, 9 aa, Homo Sapiens
HPELWRLTV

SEQ ID NO: 29 - protein, 9 aa, Homo Sapiens
HRERAMMTM

SEQ ID NO: 30 - protein, 9 aa, Homo Sapiens
HRILLVRAR

SEQ ID NO: 31 - protein, 9 aa, Homo Sapiens
IEIYVNDSV

SEQ ID NO: 32 - protein, 9 aa, Homo Sapiens
IKAQKFHAL

SEQ ID NO: 33 - protein, 9 aa, Homo Sapiens
ILTEVLKGV

SEQ ID NO: 34 - protein, 9 aa, Homo Sapiens
IQTSTPFHY

SEQ ID NO: 35 - protein, 9 aa, Homo Sapiens
IQTSTPFHY

SEQ ID NO: 36 - protein, 9 aa, Homo Sapiens
ISHTTGIPY

SEQ ID NO: 37 - protein, 9 aa, Homo Sapiens
KAMLMAQAI

SEQ ID NO: 38 - protein, 9 aa, Homo Sapiens
KESKNTEVL

SEQ ID NO: 39 - protein, 9 aa, Homo Sapiens
KLIDCYTFL

SEQ ID NO: 40 - protein, 9 aa, Homo Sapiens
KLIWWKDNK

SEQ ID NO: 41 - protein, 9 aa, Homo Sapiens
KLKEQIFEA

SEQ ID NO: 42 - protein, 9 aa, Homo Sapiens
KLLGDINWI

SEQ ID NO: 43 - protein, 9 aa, Homo Sapiens
KLTTWILPK

SEQ ID NO: 44 - protein, 9 aa, Homo Sapiens
KPLKGKVPA

SEQ ID NO: 45 - protein, 9 aa, Homo Sapiens
KQYGPNSPY

SEQ ID NO: 46 - protein, 9 aa, Homo Sapiens
KRFIFTLIA

SEQ ID NO: 47 - protein, 9 aa, Homo Sapiens
KSFSIKMLK

SEQ ID NO: 48 - protein, 9 aa, Homo Sapiens
KSSLSPSQF

SEQ ID NO: 49 - protein, 9 aa, Homo Sapiens
KTALIKYSM

SEQ ID NO: 50 - protein, 9 aa, Homo Sapiens
KTKIFQFLK

SEQ ID NO: 51 - protein, 9 aa, Homo Sapiens
KVIVELMAY

SEQ ID NO: 52 - protein, 9 aa, Homo Sapiens
LANLNPVTW

SEQ ID NO: 53 - protein, 9 aa, Homo Sapiens
LLTCIDSTF

SEQ ID NO: 54 - protein, 9 aa, Homo Sapiens
LLVSSAFIK

SEQ ID NO: 55 - protein, 9 aa, Homo Sapiens
LMAQAITGV

SEQ ID NO: 56 - protein, 9 aa, Homo Sapiens
LPHSTVKTF

SEQ ID NO: 57 - protein, 9 aa, Homo Sapiens
LPKQKLEAL

SEQ ID NO: 58 - protein, 9 aa, Homo Sapiens
LQSCVKPPY

SEQ ID NO: 59 - protein, 9 aa, Homo Sapiens
LQSFYPWEW

SEQ ID NO: 60 - protein, 9 aa, Homo Sapiens
LYLDQIATL

SEQ ID NO: 61 - protein, 9 aa, Homo Sapiens
LYTLNFLNI

SEQ ID NO: 62 - protein, 9 aa, Homo Sapiens
MMINISIGY

SEQ ID NO: 63 - protein, 9 aa, Homo Sapiens
MMTMAVLSK

SEQ ID NO: 64 - protein, 9 aa, Homo Sapiens
NRIDPLAPL

SEQ ID NO: 65 - protein, 9 aa, Homo Sapiens
PYMLVVGNI

SEQ ID NO: 66 - protein, 9 aa, Homo Sapiens
QAFINSGAW

SEQ ID NO: 67 - protein, 9 aa, Homo Sapiens
QPGLPSPAM

SEQ ID NO: 68 - protein, 9 aa, Homo Sapiens
QSVNFVNDW

SEQ ID NO: 69 - protein, 9 aa, Homo Sapiens
RAMMTMAVL

SEQ ID NO: 70 - protein, 9 aa, Homo Sapiens
REDNLTLDI

SEQ ID NO: 71 - protein, 9 aa, Homo Sapiens
RGLCPNALW

SEQ ID NO: 72 - protein, 9 aa, Homo Sapiens
RILTPEATK

SEQ ID NO: 73 - protein, 9 aa, Homo Sapiens
RLIPYDWEI

SEQ ID NO: 74 - protein, 9 aa, Homo Sapiens
RLLYVHVTV

SEQ ID NO: 75 - protein, 9 aa, Homo Sapiens
RLMSLEHRF

SEQ ID NO: 76 - protein, 9 aa, Homo Sapiens
RLSYVHVTV

SEQ ID NO: 77 - protein, 9 aa, Homo Sapiens
RMLTDLRAV

SEQ ID NO: 78 - protein, 9 aa, Homo Sapiens
RPWEASPSV

SEQ ID NO: 79 - protein, 9 aa, Homo Sapiens
RRAELVAVI

SEQ ID NO: 80 - protein, 9 aa, Homo Sapiens
RRNRLSFLG

SEQ ID NO: 81 - protein, 9 aa, Homo Sapiens
RTLKTQLVK

SEQ ID NO: 82 - protein, 9 aa, Homo Sapiens
SLRPRVNYL

SEQ ID NO: 83 - protein, 9 aa, Homo Sapiens
SMDDQLNQL

SEQ ID NO: 84 - protein, 9 aa, Homo Sapiens
STVKTFTLY

SEQ ID NO: 85 - protein, 9 aa, Homo Sapiens
TCIDSTFNW

SEQ ID NO: 86 - protein, 9 aa, Homo Sapiens
THIRAHTNL

SEQ ID NO: 87 - protein, 9 aa, Homo Sapiens
TLIAVIMGL

SEQ ID NO: 88 - protein, 9 aa, Homo Sapiens
TVRKRNFPF

SEQ ID NO: 89 - protein, 9 aa, Homo Sapiens
VASHHIRIW

SEQ ID NO: 90 - protein, 9 aa, Homo Sapiens
VEVPTVSPI

SEQ ID NO: 91 - protein, 9 aa, Homo Sapiens
VTVDTYSHF

SEQ ID NO: 92 - protein, 9 aa, Homo Sapiens
WEASPSVHI

SEQ ID NO: 93 - protein, 9 aa, Homo Sapiens
WEILAKSSL

SEQ ID NO: 94 - protein, 9 aa, Homo Sapiens
WMDNPIEIY

SEQ ID NO: 95 - protein, 9 aa, Homo Sapiens
WMGDRLMSL

SEQ ID NO: 96 - protein, 9 aa, Homo Sapiens
WQIGLANFV

SEQ ID NO: 97 - protein, 9 aa, Homo Sapiens
WVNQWPLPK

SEQ ID NO: 98 - protein, 9 aa, Homo Sapiens
YAMSNLFSI

SEQ ID NO: 99 - protein, 9 aa, Homo Sapiens
YHMVSGMSL

SEQ ID NO: 100 - protein, 9 aa, Homo Sapiens
YIDDILCAA

SEQ ID NO: 101 - protein, 9 aa, Homo Sapiens
YLDQIATLI

SEQ ID NO: 102 - protein, 9 aa, Homo Sapiens
YPKTKIFQF

SEQ ID NO: 103 - protein, 9 aa, Homo Sapiens
YPQPPTRRL

SEQ ID NO: 104 - protein, 9 aa, Homo Sapiens
YQLARRAEL

SEQ ID NO: 105 - protein, 9 aa, Homo Sapiens
YQSAQRAEL

SEQ ID NO: 106 - protein, 9 aa, Homo Sapiens
YQYWPPAEL

SEQ ID NO: 107 - protein, 9 aa, Homo Sapiens
YVHVTVDTY

SEQ ID NO: 108 - protein, 9 aa, Homo Sapiens
MLAGNEFQV

SEQ ID NO: 109 - protein, 9 aa, Homo Sapiens
TMLNILALV

SEQ ID NO: 110 - protein, 9 aa, Homo Sapiens
YYYQRALEI

SEQ ID NO: 111 - protein, 9 aa, Homo Sapiens
KITEYLERV

SEQ ID NO: 112 - protein, 9 aa, Homo Sapiens
VYSACSFTF

SEQ ID NO: 113 - protein, 9 aa, Homo Sapiens
TLLKVTLEV

SEQ ID NO: 114 - protein, 9 aa, Homo Sapiens
LYSGTAADF

SEQ ID NO: 115 - protein, 9 aa, Homo Sapiens
MVMSFVNLV

SEQ ID NO: 116 - protein, 9 aa, Homo Sapiens
FYFRNHFCI

SEQ ID NO: 117 - protein, 9 aa, Homo Sapiens
LLNQQICPV

SEQ ID NO: 118 - protein, 9 aa, Homo Sapiens
VYVAFSEFF

SEQ ID NO: 119 - protein, 9 aa, human calicivirus
MLAGNAFTA

SEQ ID NO: 120 - protein, 9 aa, Hepatitis C virus
ALMAFTSAV

SEQ ID NO: 121 - protein, 9 aa, Influenza A virus
DYFRNQFKI

SEQ ID NO: 122 - protein, 9 aa, Homo Sapiens
ALLALTSAV

SEQ ID NO: 123 - protein, 9 aa, Homo Sapiens
ALSNHLNAV

SEQ ID NO: 124 - protein, 9 aa, Homo Sapiens
RQYSVGFEV

SEQ ID NO: 125 - protein, 9 aa, Homo Sapiens
IYTVSSFSI

SEQ ID NO: 126 - protein, 9 aa, Homo Sapiens
IFNIIPSTF

SEQ ID NO: 127 - protein, 9 aa, Homo Sapiens
YYDVIYLSW

SEQ ID NO: 128 - protein, 9 aa, Homo Sapiens
RIVCLFWGV

SEQ ID NO: 129 - protein, 9 aa, Homo Sapiens
LYACGTGAF

SEQ ID NO: 130 - protein, 9 aa, Homo Sapiens
FMLDLYNAL

SEQ ID NO: 131 - protein, 9 aa, Homo Sapiens
FLISIYQVL

SEQ ID NO: 132 - protein, 9 aa, Homo Sapiens
FQFSASLHV

SEQ ID NO: 133 - protein, 9 aa, Homo Sapiens
VYTYIQSRF

SEQ ID NO: 134 - protein, 9 aa, Homo Sapiens
VHMKDFFYF

SEQ ID NO: 135 - protein, 9 aa, Homo Sapiens
ALFGALFLA

SEQ ID NO: 136 - protein, 9 aa, Homo Sapiens
LLNSLLDTV

SEQ ID NO: 137 - protein, 9 aa, Homo Sapiens
TYFGSIVLL

SEQ ID NO: 138 - protein, 9 aa, Homo Sapiens
LYHAGTVLL

SEQ ID NO: 139 - protein, 9 aa, Human adenovirus type 4
LLLTLLLLL

SEQ ID NO: 140 - protein, 9 aa, Hepatitis B virus
LLGPLLVLL

SEQ ID NO: 141 - protein, 9 aa, Homo Sapiens
KLQAGTVFV